(19) United States
(12) Patent Application Publication
(10) Pub. No.: US 2020/0123127 A1
(43) Pub. Date: Apr. 23, 2020

(54) REMOVAL OF SENESCENCE-ASSOCIATED MACROPHAGES

(71) Applicant: EVERON BIOSCIENCES, INC., Buffalo, NY (US)

(72) Inventors: Andrei GUDKOV, East Aurora, NY (US); Alexander POLINSKY, Brookline, MA (US); Vitaly BALAN, Buffalo, NY (US); Olga CHERNOVA, Buffalo, NY (US); Brandon M. HALL, Buffalo, NY (US); Pavel G. KOMAROV, Orchard Park, NY (US); Denis N. KAZYULKIN, Nahabino, Moskovskaya Oblast (RU)

(21) Appl. No.: 16/096,601

(22) PCT Filed: Apr. 25, 2017
(86) PCT No.: PCT/US17/29372

§ 371 Date: Oct. 25, 2018

Related U.S. Application Data
(60) Provisional application No. 62/485,253, filed on Apr. 13, 2017.
(60) Provisional application No. 62/358,449, filed on Jul. 5, 2016.
(60) Provisional application No. 62/327,166, filed on Apr. 25, 2016.

(51) Int. Cl. C07D 333/72; C07D 495/10; C07D 495/04; C07D 239/84; C07D 207/06; C07D 215/54; C07D 277/82; C07D 409/12; G01N 33/50; A61P 35/00; A61K 31/4439; A61K 31/7076; A61K 31/52

(52) U.S. Cl. CPC C07D 333/72; C07D 495/10; C07D 495/04; C07D 239/84; C07D 207/06; C07D 215/54; A61K 31/52; C07D 409/12; G01N 33/5008; A61P 35/00; A61K 31/4439; A61K 31/7076; C07D 277/82

(57) ABSTRACT

In various aspects and embodiments provided are compounds, compositions and methods relating to aging, senescent cells (SCs) and/or senescence associate macrophages (SAMs). In certain aspects and embodiments provided are compounds and compositions that selectively kill or reprogram senescent cells (SCs) and or senescence associate macrophages (SAMs) and associated methods. In some embodiments, the compounds compositions and methods treat or reverse aging organism Normal tissue and/or age-related diseases.

### CROSS-REFERENCE TO RELATED APPLICATIONS

This application claims the benefit of priority to U.S. Provisional application No. 62/327,166, filed Apr. 25, 2016, to U.S. Provisional application No. 62/358,449, filed Jul. 5, 2016, and to U.S. Provisional application No. 62/485,253, filed Apr. 13, 2017, the disclosures of each of which are incorporated herein by reference.

### TECHNICAL FIELD

The present disclosure in various aspects and embodiments relates to compositions and methods for selective removal and modulation of the phenotype of cells that accumulate and contribute to age-related diseases.

### BACKGROUND ART

A unifying symptom that develops with age is sterile chronic systemic inflammation, named “inflammaging” [7, 8], which contributes to development of cancer, metabolic diseases, and other age-related pathologies [9-16]. Senescent cell accumulation with age is thought to be a major source of chronic inflammation underlying age-related diseases [10,20,39]. In fact, the amounts of cells expressing SC marker [positive for p16 (Ink4a)] gradually increases during mouse life [40] (FIG. 1A, B). However, the reasons for age-dependent differences in the amounts of SCs in tissues are not well understood. One explanation is that in young organisms, SCs are cleared by the innate immune system [3,41,42]. The scientific concept of aging and age-related diseases being caused by pathological secretion of accumulated senescent cells has been described in detail in the following: (Campisi, 1998; Davalos, Coppe, Campisi, & Desprez, 2010; Freund, Orjalo, Desprez, & Campisi, 2010; Laberge et al., 2012; Laberge, Awad, Campisi, & Desprez, 2011; Weyand, Fulbright, & Goronzy, 2003). This idea was experimentally tested by Baker et al. [21,23] and Demaria et al. [22] who used an artificial mouse-based model of premature aging to assess the possibility of reverting age-associated phenotypes by selective eradication of senescent cells.

The predominant senescent cell-associated antigen appears to be “p16Ink4a” (Campisi, J. Cellular, Trends Cell Biol. 11, S27-31 (2001) includes a link between P16(Ink4a) and cell senescence; and e.g. Liu Y, Sanoff H K, Cho H, Burd C E, Torrice C, Ibrahim J G, Thomas N E, Sharpless N E (August 2009), Aging Cell 8 (4): 439-48 reports an increase in P16(Ink4a) expression with age suggesting a role of this gene in senescence.

### SUMMARY OF THE EMBODIMENTS

In some aspects and embodiments the present disclosure provides compounds and methods of making and using the compounds. For example, the compounds can be used for selective partial or complete eradication of senescent cells, or for removing, killing or reprogramming senescence-associated macrophages (SAMs).

As used herein, unless otherwise indicated, the term “alkyl group,” unless otherwise stated, refers to branched or unbranched hydrocarbons. Examples of such alkyl groups include methyl groups, ethyl groups, propyl groups, butyl groups, iso-propyl groups, sec-butyl, tert-butyl groups, and the like. For example, the alkyl group is a C₁ to C₆ alkyl group, including all integer numbers of carbons and ranges of numbers of carbons there between. Alkyl groups can be substituted with various other functional groups. The alkyl groups may be substituted or unsubstituted.

As used herein, unless otherwise indicated, “alkenyl group” refers to a C₂-C₄ hydrocarbon group, including all integer numbers of carbons and ranges of numbers of carbons therebetween, having at least one carbon-carbon double bond. The alkenyl groups may be substituted or unsubstituted. As used herein, unless otherwise indicated, “alkynyl group” refers to a C₂-C₄ hydrocarbon groups, including all integer numbers of carbons and ranges of numbers of carbons therebetween, having at least one carbon-carbon triple bond The alkynyl groups may be substituted or unsubstituted.

As used herein, unless otherwise indicated, “aryl group” refers to a C₃-C₁₈ aromatic group, including all integer numbers of carbons and ranges of numbers of carbons therebetween. The aryl groups may be substituted or unsubstituted.

As used herein, unless otherwise indicated, “alkoxy group” refers to a

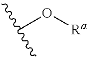

group where R^{a} is a linear, branched or cyclic C₁-C₆ alkyl group, including all integer numbers of carbons and ranges of numbers of carbons therebetween. For example, suitable alkoxy groups include methoxy, ethoxy, propoxy, iso-propoxy, butoxy, sec-butoxy, tert-butoxy, and hexoxy groups. The alkoxy groups may be substituted or unsubstituted.

As used herein, unless otherwise indicated, “amino group” refers to a

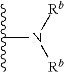

group where each R^{b} is selected independently from the group consisting of hydrogen atom, substituted or unsubstituted C₁-C₆ alkyl, including all integer numbers of carbons and ranges of numbers of carbons therebetween, substituted or unsubstituted phenyl, substituted or unsubstituted heteroaryl, substituted or unsubstituted heterocyclyl, substituted carbonyl, substituted sulfonyl, haloalkyl, and substituted or unsubstituted benzyl groups.

As used herein, unless otherwise indicated, the term “cycloalkyl group” refers to a C₃-C₂₀ cyclic hydrocarbon group, including all integer numbers of carbons and ranges of numbers of carbons therebetween, e.g., cyclopropyl, cyclobutyl, cyclohexyl, and cyclopentyl groups. Cycloalkyl groups can be saturated or partially unsaturated ring systems. The cycloalkyl groups may be substituted or unsubstituted.

As used herein, unless otherwise indicated, the term “halogen” refers to fluorine, chlorine, bromine, and iodine, and the term “halo” means fluoro group (—F), chloro group (—Cl), bromo group (—Br), and iodo group (—I).

As used herein, unless otherwise indicated, the term “heteroaryl group” refers to a C₁-C₂₀ monocyclic, bicyclic or polycyclic ring system, including all integer numbers of carbons and ranges of numbers of carbons therebetween, comprising one, two or more aromatic rings and containing at least one nitrogen, oxygen or sulfur atom in an aromatic ring. The heteroaryl groups may be substituted or unsubstituted. Examples of heteroaryl groups include benzofuranyl, thienyl, furyl, pyridyl, pyrimidyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl groups.

As used herein, unless otherwise indicated, the term “heterocyclic group” refers to C₁-C₂₀ cyclic groups containing one or more heteroatoms (e.g., N, O, S, or the like) as part of the ring structure. The heterocyclic groups may be substituted or unsubstituted.

As used herein, unless otherwise indicated, the term “alkylaryl group” refers to alkyl-substituted aryl groups. The alkylaryl groups may be substituted or unsubstituted.

As used herein, unless otherwise indicated, the term “arylalkyl group” refers to aryl-substituted alkyl groups. The arylalkyl groups may be substituted or unsubstituted.

As used herein, unless otherwise indicated, the term “arylalkenyl group” refers to aryl-substituted alkenyl groups. The arylalkenyl groups may be substituted or unsubstituted.

As used herein, unless otherwise indicated, the term “arylalkynyl group” refers to aryl-substituted alkynyl groups. The arylalkynyl groups may be substituted or unsubstituted.

As used herein, unless otherwise indicated, the term “cycloalkylalkyl group” refers to a cycloalkyl-substituted alkyl groups. The cycloalkylalkyl groups may be substituted or unsubstituted.

As used herein, unless otherwise indicated, the term “hydroxyalkyl group,” refers to one or two hydroxyl-substituted alkyl groups. The hydroxyalkyl groups may be substituted or unsubstituted.

As used herein, unless otherwise indicated, the term “alkoxyalkyl group” refers to an alkoxy-substituted alkyl group. The alkoxyalkyl groups may be substituted or unsubstituted.

As used herein, unless otherwise indicated, the term “heteroarylalkyl” refers to a heteroaryl-substituted alkyl group. The heteroarylalkyl groups may be substituted or unsubstituted.

As used herein, unless otherwise indicated, the term “heterocycloalkyl” refers to a heterocyclo group attached to the parent molecular moiety through an alkyl group.

As used herein, substituted refers to any one or more hydrogens on the designated atom or group is replaced with a selection from the indicated group, provided that the designated atom's normal valence is not exceeded. Suitable substituents include alkyl groups, alkenyl groups, alkynyl groups, alkoxy groups, alkoxyalkyl groups, alkylaryl groups, aryl groups, arylalkyl groups, arylalkenyl groups, arylalkynyl groups, amino groups, azido groups, cycloalkyl groups, cycloalkylalkyl groups, cyano groups, isocyano groups, halide groups, heteroaryl groups, heteroarylalkyl groups, heterocyclyl groups, heterocycloalkyl groups, hydroxyalkyl, nitro groups,

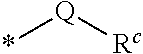

group, where R^{c} is independently selected from prodrug substituents, hydrogen atom, alkyl groups, amino groups, aryl groups, heteroaryl groups, heterocyclyl groups, and hydroxyl groups and Q is selected from O, S, C(═O), S(═O), S(═O)₂, and

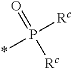

where R^{c} as defined above.

In an aspect, the present disclosure provides heterocyclic compounds. In various examples, the compounds are substituted thiophene compounds. The compounds can be made by methods described herein. The compounds can be used in methods disclosed herein.

In various embodiments, compounds of the present disclosure have the following structure/formula:

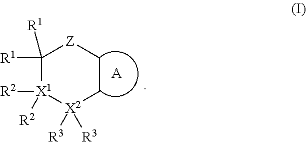

In this structure, A, together with the two carbons from the adjacent ring, is a fused heterocycle. Z is selected from the group consisting of C═N—OR⁵, wherein R⁵ is H or a prodrug substituent. X¹ is C or N. X² is C, O, or a spacer (e.g., —C(═O)—, —C(N═OR⁶)—, —C(═O)CH₂—, —C(N═OR⁶)CH₂—, —CH₂C(═O)—, —CH₂C(N═OR⁶)—, where R⁶ is —H or un(substituted) C₁₋₆ alkyl. Each R¹ is independently selected from the group consisting of H, C₁₋₆ alkyl, and C₃₋₁₈ aryl, or both R¹ groups may, together with the carbon they are attached to, form a C₃-C₁₀ spirocycle. Each R² is independently selected from the group consisting of H, C₁₋₆ alkyl, C₃₋₁₈ aryl, C₁-C₂₀ heteroaryl, and C(O)N(R⁷)₂, or both R¹ groups may, together with the carbon they are attached to, form a C₃-C₁₀ spirocycle, where R⁷ is independently selected from the group consisting-hydrogen, substituted or unsubstituted C₁-C₆ alkyl, substituted or unsubstituted alkynyl, substituted or unsubstituted aryl, substituted or unsubstituted heteroaryl, substituted or unsubstituted saturated heterocyclyl, substituted or unsubstituted partially-saturated heterocyclyl, substituted or unsubstituted saturated C₃₋₂₀ carbocycyl and substituted or unsubstituted partially-saturated C₃-C₂₀ carbocycyl, or both R⁹ substituents, together with the N atom they are attached to, form a heterocycle, which may additionally contains a further heteroatom(s) selected from N, O or S. Each R³ is independently selected from the group consisting of H, C₁-C₆ alkyl, C₃₋₁₈ aryl, C₁-C₂₀ heteroaryl; and any two of R¹, R², R³, and/or substituents of A may be linked together with a linker to form a macrocycle.

The linker connects any two of R¹, R², R³, and/or substituents of A together to form a macrocycle. For example, the linker has the following structure/formula:

—X³—(CR⁹R¹⁰)ₙ—X⁴—(CR⁹R¹⁰)ₘ—X⁵—, where X³, X⁴, X⁵ are independently selected from the group consisting of:

O, NR⁸, C(R⁶)₂, and

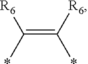

each R⁹ and R¹⁰ are independently selected from the group consisting of: hydrogen, substituted or unsubstituted C₁-C₄ alkyl, NR⁸, and OR⁶, and n, m=0-6.

In various examples, A in formula (I) above is selected from the group consisting of:

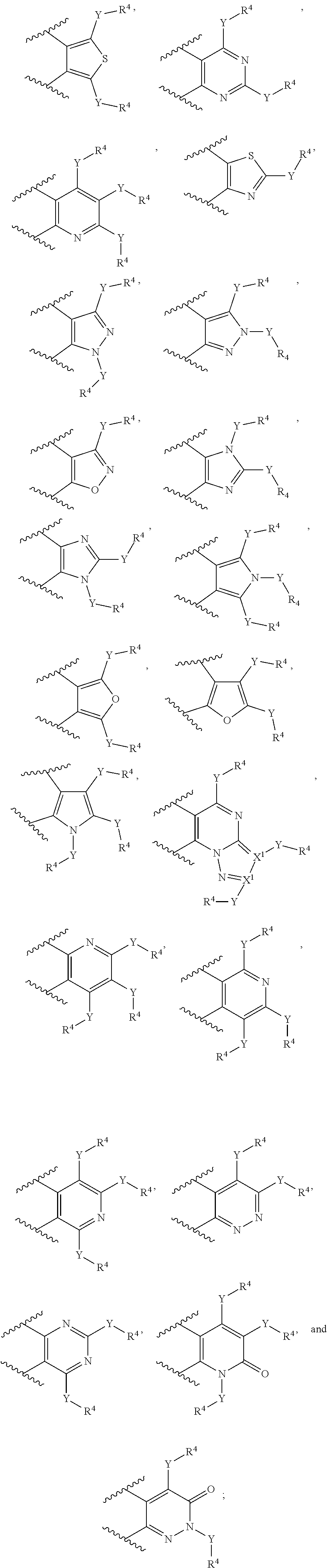

Each Y is independently selected from the group consisting of direct bond, O, S, S(═O), S(═O)₂, S(═O)(═NR⁸), C(═O), C(═O)O,

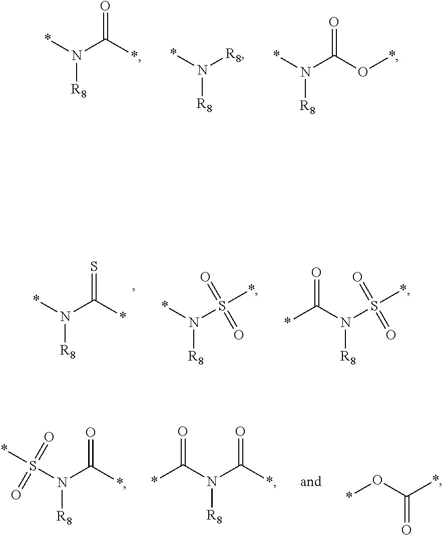

wherein each R⁸ is independently —H or un(substituted) C₁₋₆ alkyl, C₃₋₈ cycloalkyl; and each R⁴ is independently selected from hydrogen, halide, —CN, NO₂, substituted or unsubstituted C₁₋₆ alkyl, substituted or unsubstituted alkenyl, substituted or unsubstituted alkynyl, substituted or unsubstituted alkoxyl, substituted or unsubstituted aralkyl, substituted or unsubstituted hydroxyalkyl, substituted or unsubstituted alkoxyalkyl, substituted or unsubstituted cycloalkyl, substituted or unsubstituted C₃₋₁₈ aryl; substituted or unsubstituted, C₃₋₈ cycloalkyl, substituted or unsubstituted cycloalkylalkyl, substituted or unsubstituted C₁₋₂₀ heteroaryl, substituted or unsubstituted heterocyclyl, substituted or unsubstituted heterocyclylalkyl, substituted or unsubstituted heteroarylalkyl and —N(R⁷)₂. In the Y groups above, each group with an asterisk(s) can be connected with ring A via left-hand position, marked with asterix.

In various examples, the heterocyclic compound has one of the following structures:

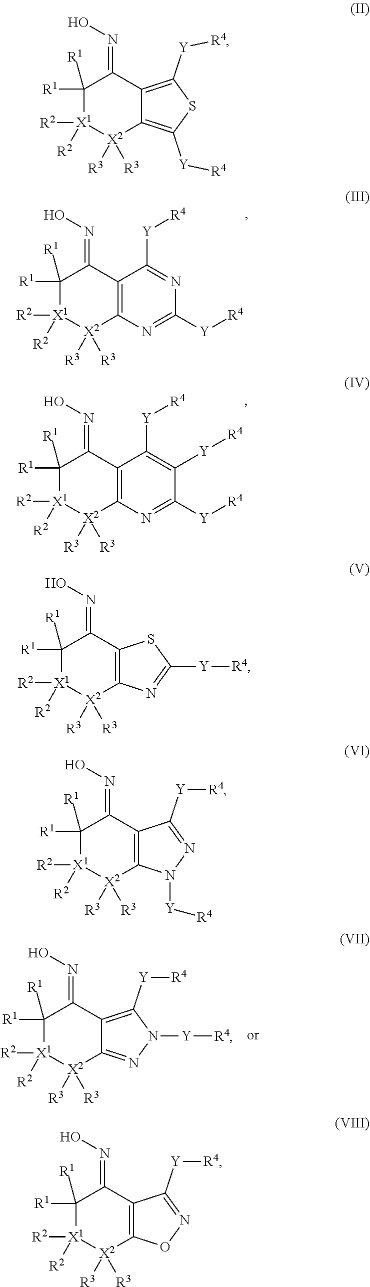

where the various groups are as defined for formula (I).

In some embodiments provided is a heterocyclic compound of Formula (Ha)

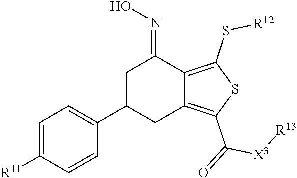

R¹¹ is selected from: halogen, substituted or unsubstituted C₁₋₆ alkyl, substituted or unsubstituted C₁₋₆ alkoxyl;
R¹² is selected from: substituted or unsubstituted phenyl, substituted or unsubstituted benzyl, substituted or unsubstituted phenethyl;
X³ is selected from O or NH;
R¹³ is selected from substituted or unsubstituted C₁₋₆ alkyl, substituted or unsubstituted C₃₋₉ cycloalkyl, substituted or unsubstituted phenyl, substituted or unsubstituted benzyl, substituted or unsubstituted methylheterocyclyl, substituted or unsubstituted methyl heteroaryl;

In some embodiments provided is a heterocyclic compound of Formula (IIIa)

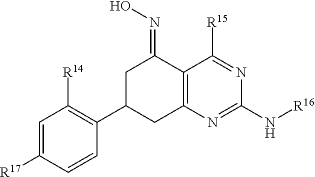

R¹⁴ is selected from: halogen, substituted or unsubstituted C₁₋₆ alkyl, substituted or unsubstituted C₁₋₆ alkoxyl, substituted or unsubstituted C₆ aryl;
R′⁵ is selected from: H, substituted or unsubstituted C₁₋₆ alkyl, substituted or unsubstituted C₁₋₆ alkoxyl;
R¹⁶ is selected from: substituted or unsubstituted phenyl, substituted or unsubstituted benzyl, substituted or unsubstituted 3-phenylpropan-1-yl, substituted or unsubstituted 3-heteroarylpropan-1-yl, substituted or unsubstituted 2-phenoxyethyl, substituted or unsubstituted 2-heteroaryloxyethyl;
R¹⁷ is selected from: halogen, substituted or unsubstituted C₁₋₆ alkyl, substituted or unsubstituted C₃₋₉ cycloalkyl, substituted or unsubstituted C₂₋₄ alkynyl, substituted or unsubstituted C₁₋₆ alkoxyl, substituted or unsubstituted phenyl;

In some embodiments provided is a heterocyclic compound of Formula (IVa)

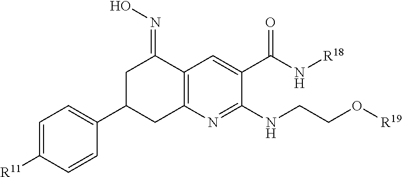

R¹¹ as defined above;
R¹⁸ selected from substituted or unsubstituted phenyl, substituted or unsubstituted heteroaryl, substituted or unsubstituted C₃₋₉ cycloalkyl;
R¹⁸ selected from substituted or unsubstituted phenyl, substituted or unsubstituted heteroaryl;

In certain embodiments, a heterocyclic compound as provided herein has the following structure:

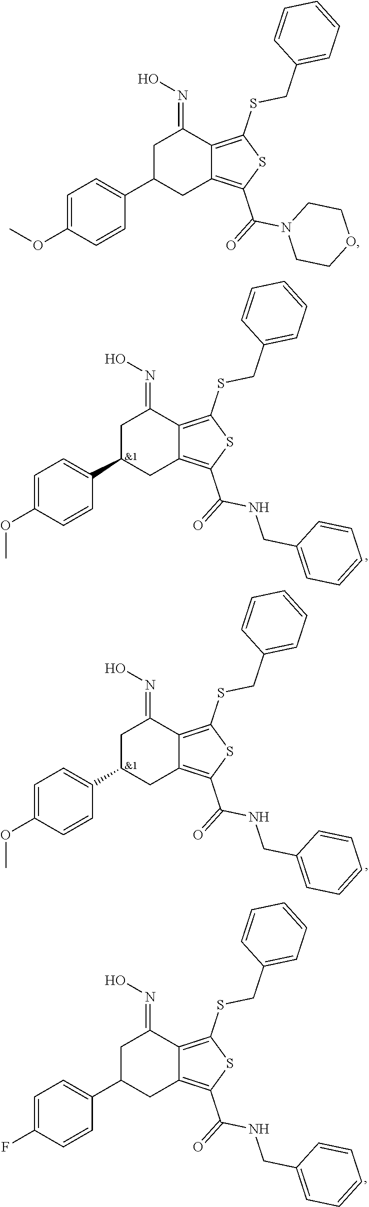

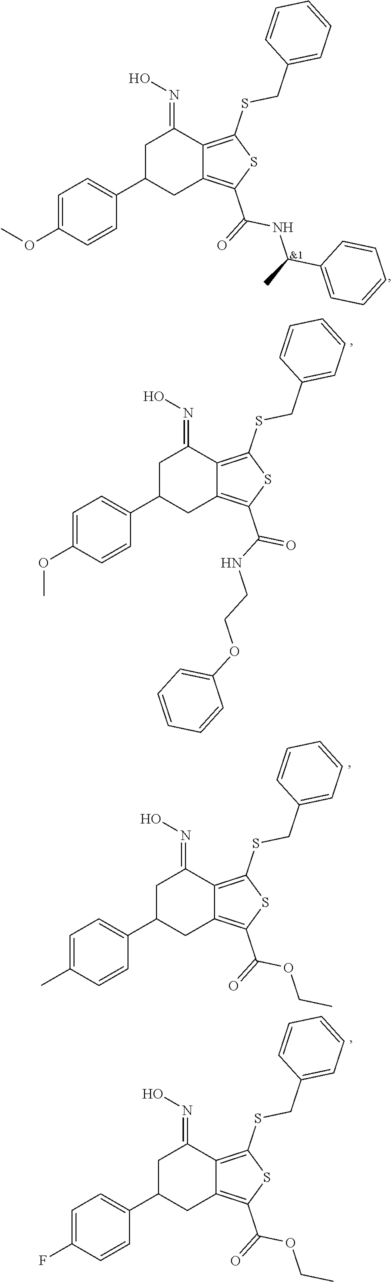

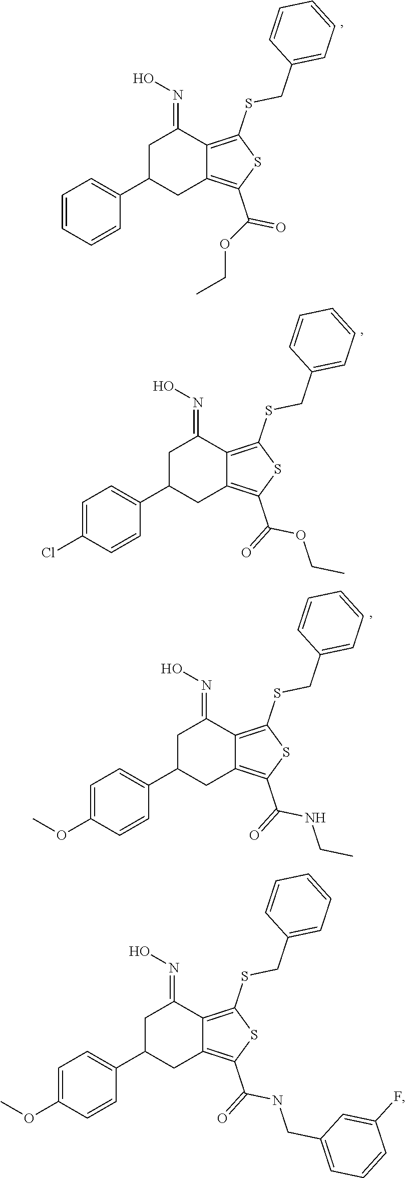

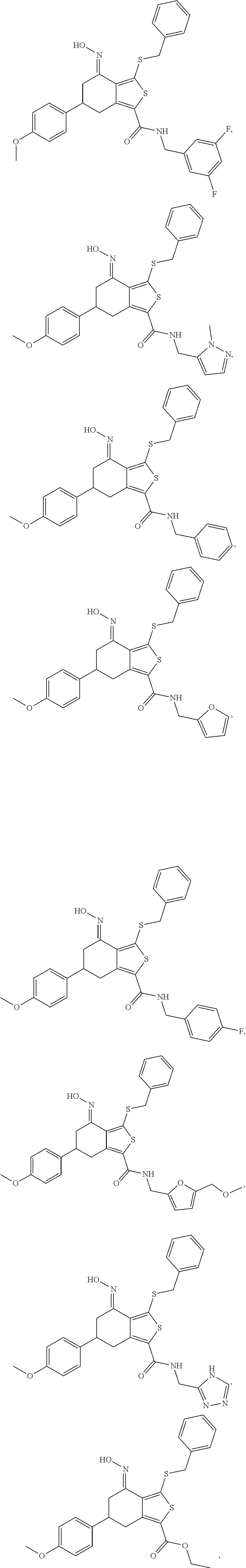

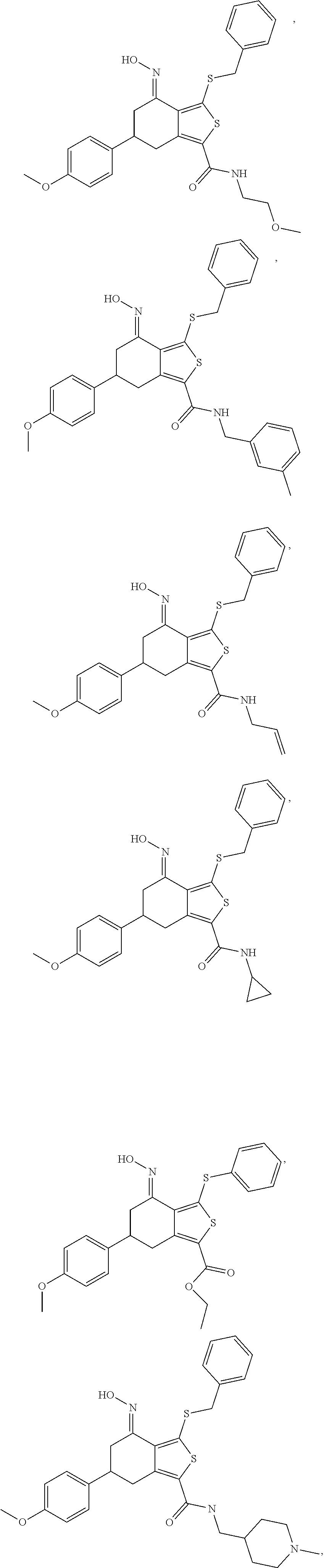

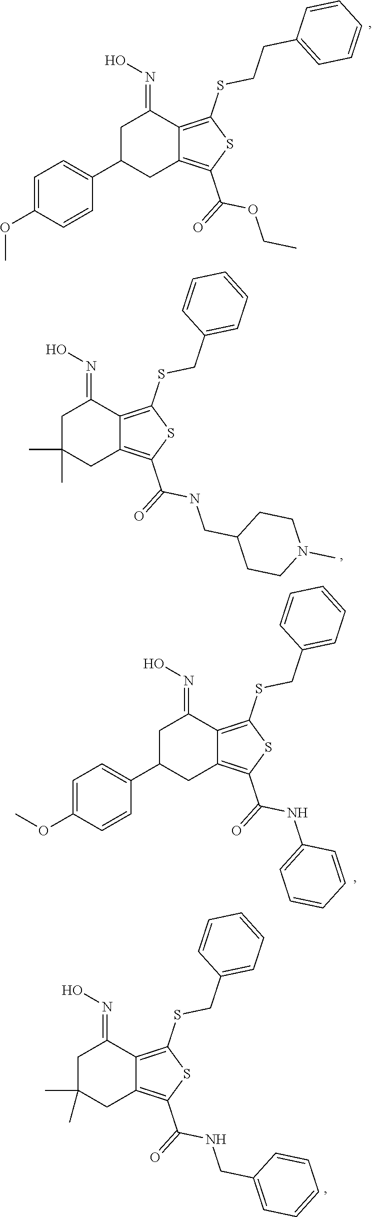

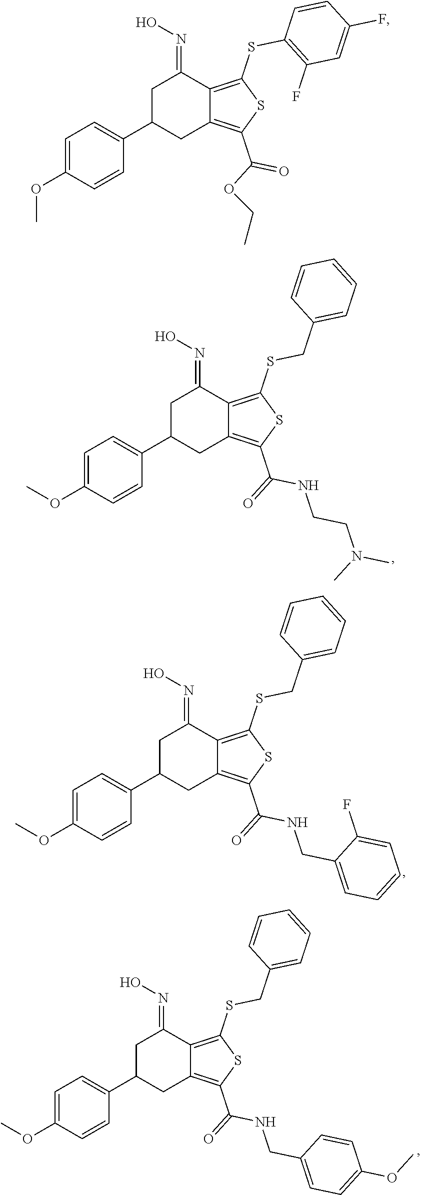

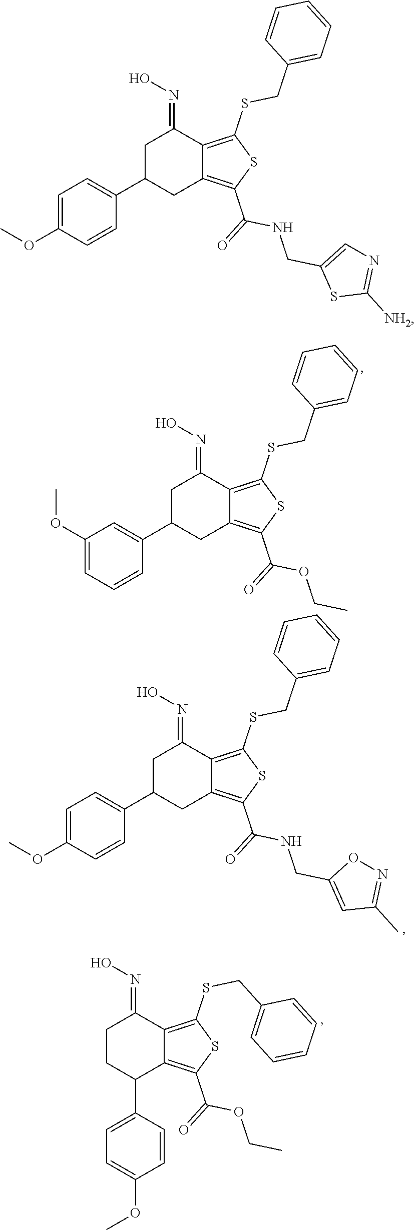

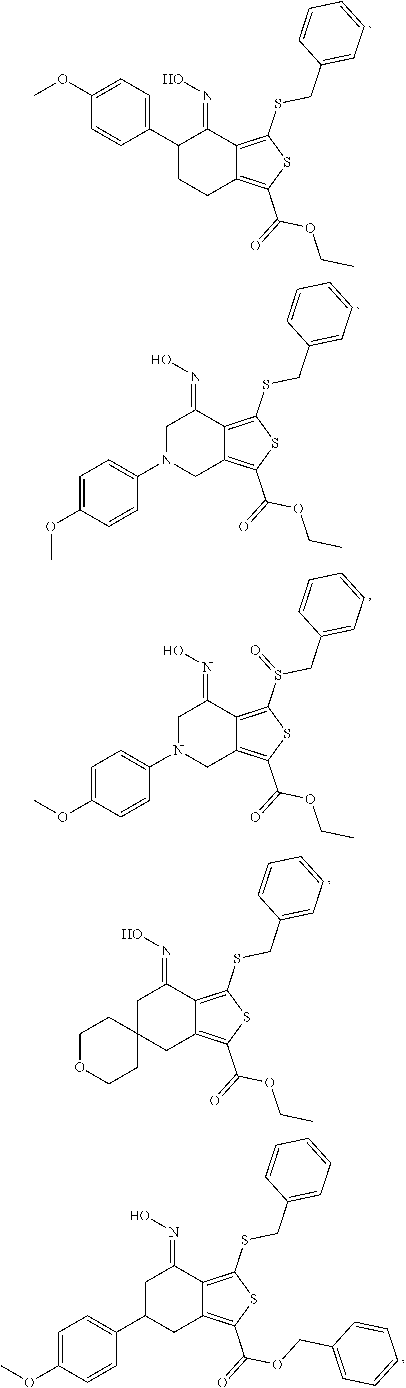

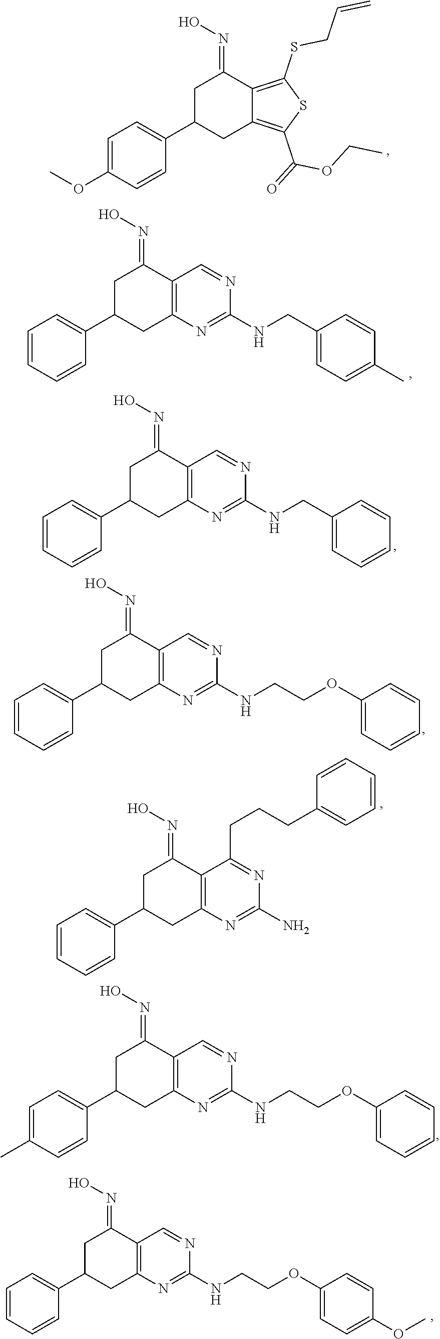

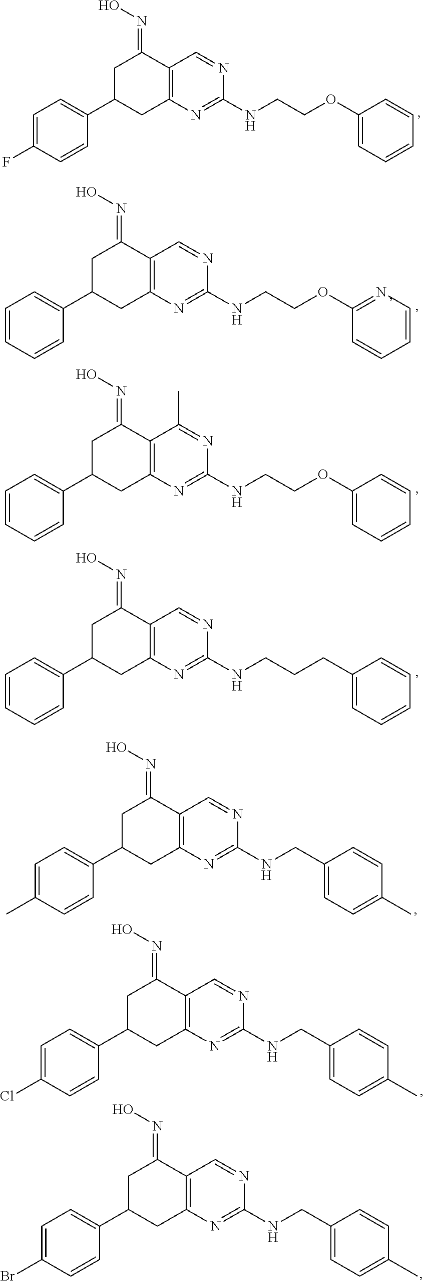

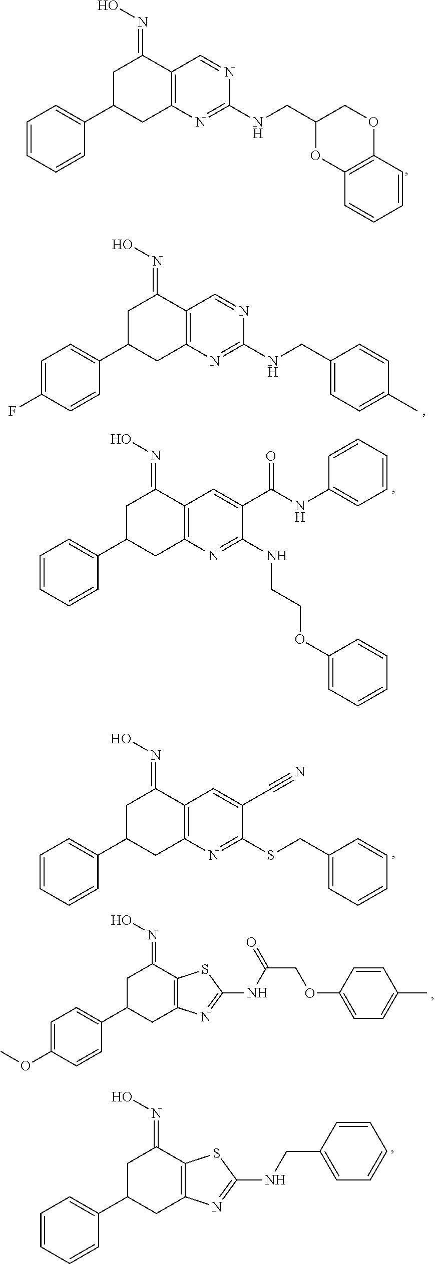

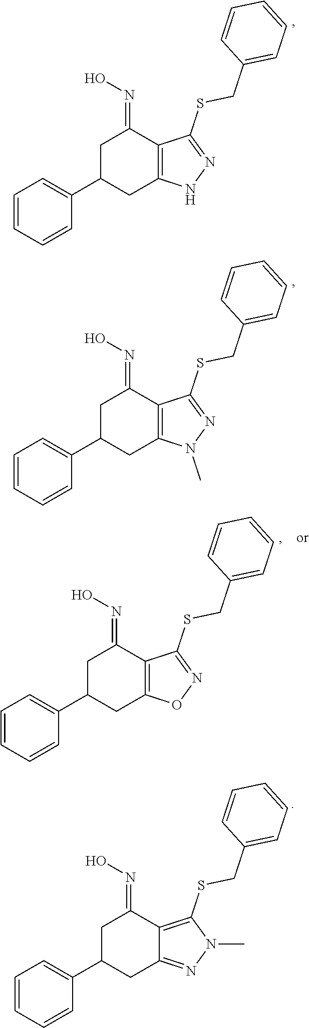 [SMILES: O/N=C1\CC(c2ccccc2)Cc2[nH]nc(SCc3ccccc3)c21 ; Cn1nc(SCc2ccccc2)c2c1CC(c1ccccc1)C/C2=N\O ; O/N=C1\CC(c2ccccc2)Cc2onc(SCc3ccccc3)c21 ; Cn1nc2c(c1SCc1ccccc1)/C(=N/O)CC(c1ccccc1)C2]

In certain embodiments, a heterocyclic compound as provided herein has the following structure:

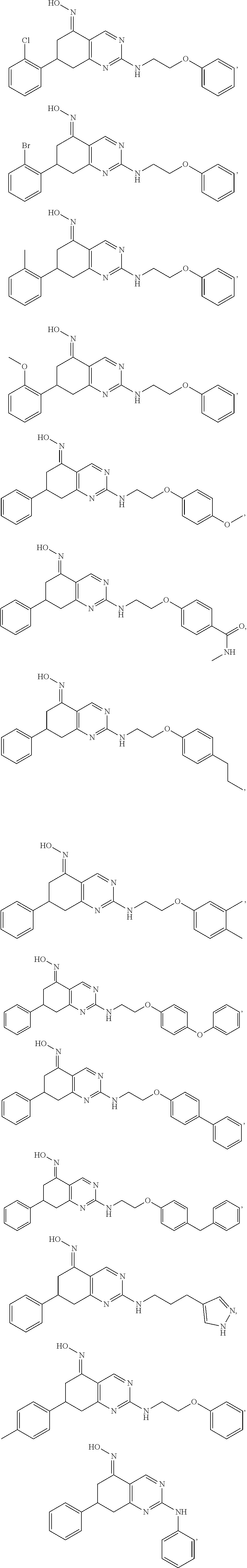

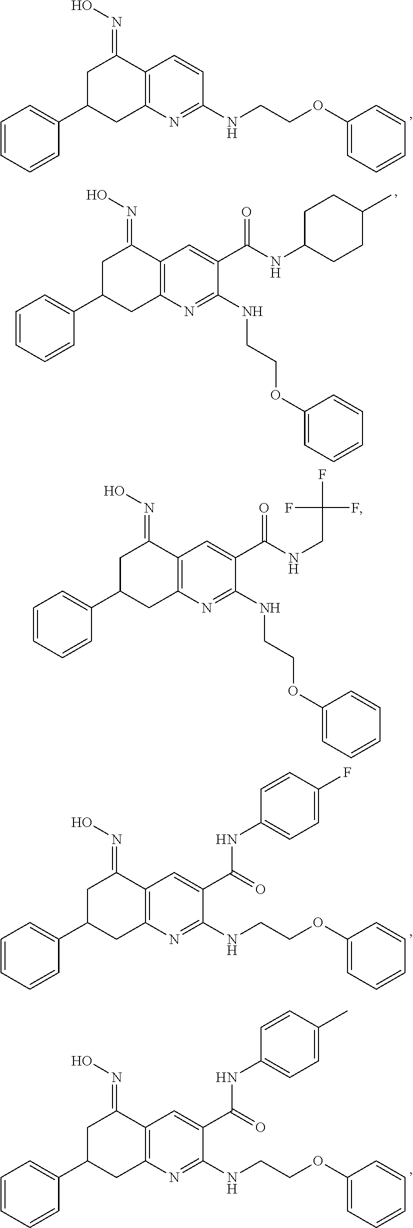

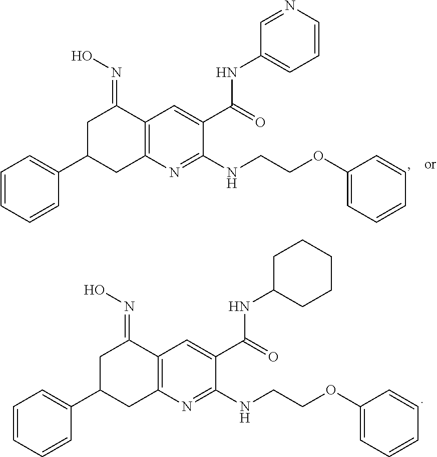 [SMILES: O=C(Nc1cccnc1)c1cc2c(nc1NCCOc1ccccc1)CC(c1ccccc1)C/C2=N\O ; O=C(NC1CCCCC1)c1cc2c(nc1NCCOc1ccccc1)CC(c1ccccc1)C/C2=N\O]

In various examples, one or more of the heteroatoms, such as, for example, nitrogen and sulfur, of a compound of the present disclosure are oxidized to form N-oxides or sulfoxides and sulfones, respectively, and/or one or more of the nitrogen in one or more heterocycle of a compound of the present disclosure are quaternized.

The present disclosure includes all possible stereoisomers and geometric isomers of a compound of the present disclosure. The present disclosure includes both racemic compounds and optically active isomers. When a compound of the present disclosure is desired as a single enantiomer, it can be obtained either by resolution of the final product or by stereospecific synthesis from either isomerically pure starting material or use of a chiral auxiliary reagent, for example, see Z. Ma et al., Tetrahedron: Asymmetry, 8(6), pages 883-888 (1997). Resolution of the final product, an intermediate, or a starting material can be achieved by any suitable method known in the art. Additionally, in situations where tautomers of a compound of the present disclosure are possible, the present disclosure is intended to include all tautomeric forms of the compounds.

Prodrugs of a compound of the present disclosure also can be used as the compound in a method of the present disclosure. It is well established that a prodrug approach, wherein a compound is derivatized (e.g., to include a moiety that renders the compound into a form suitable for formulation and/or administration, then released as a drug in vivo, has been successfully employed to transiently (e.g., bioreversibly) alter the physicochemical properties of the compound (see, H. Bundgaard, Ed., “Design of Prodrugs,” Elsevier, Amsterdam, (1985); R. B. Silverman, “The Organic Chemistry of Drug Design and Drug Action,” Academic Press, San Diego, chapter 8, (1992); K M Hillgren et al., Med. Res. Rev., 15, 83 (1995)).

Compounds of the present disclosure can contain one or more functional groups. The functional groups, if desired or necessary, can be modified to provide a prodrug. Suitable prodrugs include, for example, acid derivatives, such as amides and esters. It also is appreciated by those skilled in the art that N-oxides can be used as a prodrug. A moiety that provides a prodrug is a prodrug substituent.

Compounds of the disclosure can be salts. Accordingly, in various examples a compound is a salt of the compound. It can be desirable that the salt be a pharmaceutically acceptable salt of compounds of the disclosure. As used herein, the term “pharmaceutically acceptable salts” refers to salts or zwitterionic forms of a compound of the present disclosure. Salts of compounds of the present disclosure can be prepared during the final isolation and purification of the compounds or separately by reacting the compound with an acid having a suitable cation. The pharmaceutically acceptable salts of a compound of the present disclosure are acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts of compounds of the disclosure include, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulphonate, and p-toluenesulfonate salts. In addition, available amino groups present in the compounds of the disclosure can be quatemized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference to compounds of the present disclosure appearing herein is intended to include a compound of the present disclosure as well as pharmaceutically acceptable salts, hydrates, or prodrugs thereof.

In an aspect, the present disclosure provides compositions. The compositions comprise one or more heterocyclic compounds of the present disclosure. Compositions comprising at least one compound of the disclosure include, for example, pharmaceutical preparations.

Compositions comprising a compound of the disclosure and a pharmaceutical carrier can be prepared at a patient's bedside, or by a pharmaceutical manufacture. In either case, the compositions or their ingredient can be provided in any suitable container, such as a sealed sterile vial or ampoule, and may be further packaged to include instruction documents for use by a pharmacist, physician or other health care provider. The compositions can be provided as a liquid, or as a lyophilized or powder form that can be reconstituted if necessary when ready for use. In particular, the compositions can be provided in combination with any suitable delivery form or vehicle, examples of which include, for example, liquids, caplets, capsules, tablets, inhalants or aerosol, etc. The delivery devices may comprise components that facilitate release of the pharmaceutical agents over certain time periods and/or intervals, and can include compositions that enhance delivery of the pharmaceuticals, such as nanoparticle, microsphere or liposome formulations, a variety of which are known in the art and are commercially available. Further, each composition described herein can comprise one or more pharmaceutical agents.

The compositions described herein can include one or more standard pharmaceutically acceptable carriers. Pharmaceutically acceptable carriers are determined in part by the particular composition being administered, as well as by the particular method used to administer the composition. Accordingly, there are a wide variety of suitable formulations of pharmaceutical compositions of the present disclosure. Some examples of pharmaceutically acceptable carriers can be found in: Remington: The Science and Practice of Pharmacy (2005) 21st Edition, Philadelphia, Pa. Lippincott Williams & Wilkins. Effective formulations include oral and nasal formulations, formulations for parenteral administration, and compositions formulated for with extended release.

Formulations suitable for oral administration can consist of (a) liquid solutions, such as an effective amount of a compound of the present disclosure suspended in diluents, such as water, saline or PEG 400; (b) capsules, sachets, depots or tablets, each containing a predetermined amount of the active ingredient, as liquids, solids, granules or gelatin; (c) suspensions in an appropriate liquid; (d) suitable emulsions; and (e) patches. The liquid solutions described above can be sterile solutions. The pharmaceutical forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

In some aspects and embodiments, present disclosure pertains at least in part to a method for removing senescence-associated macrophages (SAMs) in a mixed population of cells comprising SAMs, the method comprising selectively inducing SAM cell death in the mixed population of cells.

In some embodiments, SAM cell death is selectively induced by delivering to the mixed population of cells an agent which is selectively ingested by SAMs such that SAMs are removed from the mixed population.

In some embodiments, the agent comprises a delivery vehicle and a drug which is active when ingested by a cell.

In some embodiments, the drug comprises a poison active inside a cell, a prodrug which is activatable inside a cell, an RNA or polypeptide toxic to a cell, an RNA or polypeptide which inhibits NFκB.

In some embodiments, the delivery vehicle comprises a liposome, a nanoparticle, an antibody, an expression vector comprising a gene encoding an RNA or polypeptide toxic to a cell, an expression vector comprising a gene encoding an RNA or polypeptide which inhibits NFκB.

In some embodiments, the mixed population of cells is present in vitro in culture.

In some embodiments, the mixed population of cells is present in a mammal.

The disclosure also pertains in various aspects and embodiments to a method for removing senescence-associated macrophages (SAMs) in a mixed population of senescent cells (SCs) and SAMs, the method comprising selectively inducing SAM cell death in the mixed population of cells.

In some embodiments, SAM cell death is selectively induced by delivering to the mixed population of cells an agent which is selectively ingested by SAMs by not by SCs such that SAMs are removed from the mixed population.

In some embodiments, the agent comprises a delivery vehicle and a drug which is active when ingested by a cell.

In some embodiments, the drug comprises a poison active inside a cell (chlodronate, rapamycin), a prodrug which is activatable inside a cell, an RNA or polypeptide toxic to a cell, an RNA or polypeptide which inhibits NFκB.

In some embodiments, the delivery vehicle comprises a liposome, a nanoparticle, an antibody, an expression vector comprising a gene encoding an RNA or polypeptide toxic to a cell, an expression vector comprising a gene encoding an RNA or polypeptide which inhibits NFκB.

In some embodiments, the mixed population of cells is present in vitro in culture.

In some embodiments, the mixed population of cells is present in a mammal.

The disclosure also relates at least in part to a method of selectively killing SAMs in a mammal comprising administering to the mammal a pharmaceutical composition comprising an agent which is toxic upon ingestion by a mammalian cell and a delivery vehicle which provides said agent to said cell for ingestion.

The disclosure in some aspects and embodiments also pertains to a method of treating or preventing an age-related disease in a mammal, the method comprising administering an agent capable of reducing, eradicating, or reprogramming senescence-associated macrophages (SAMs) in a mammal, thereby treating the disease.

In some embodiments, the mammal is a human.

In some embodiments, the disease is cancer, age-related disease, tobacco-related disease, or skin wrinkles.

In some embodiments, the cancer is prostate cancer, colon cancer, lung cancer, squamous cell cancer of the head and neck, esophageal cancer, hepatocellular carcinoma, gastric cancer, pancreatic cancer, ovarian cancer, or breast cancer.

In some embodiments, the age-related or tobacco-related disease is cardiovascular disease, cerebrovascular disease, peripheral vascular disease, Alzheimer's disease, osteoarthritis, cardiac diastolic dysfunction, benign prostatic hypertrophy, aortic aneurysm, atherosclerosis, emphysema, or diabetes.

In some embodiments, the agent is selected from the group consisting of TA-4812, OT-82, interferon-alpha, interferon-beta, poly(I:C) RNA, activators of toll-like receptor 3 (TLR3), activators of toll-like receptor 4 (TLR4), ruxolitinib, momelotinib, JAK1/JAK2 inhibitors, AS1517499, STATE inhibitors, IL-4 neutralizing antibodies, IL-4 receptor neutralizing antibodies, IL-13 neutralizing antibodies, IL-13 receptor neutralizing antibodies, TA-08209-1, and TA-08210-1.

In some embodiments, the agent comprises a delivery vehicle.

In some embodiments, the delivery vehicle is a liposome or nanoparticle.

The disclosure also pertains at least in part to a method of reducing chronic systemic inflammation in an aging adult, comprising administering an agent capable of reducing or eradicating senescence-associated macrophages (SAMs) in a mammal, thereby reducing the inflammation.

The compositions and methods of the present disclosure may in certain aspects and embodiments be useful to develop treatment of pathologies associated with SAM accumulation by therapeutic engagement of innate immune response capable of recognition and eradication of SAMs using (i) immunostimulators, such as agonists of innate immunity receptors (i.e., Toll-like receptors, NOD receptors), (ii) specific cellular components of immunity (i.e., NK-cells, non-SAM macrophages), (iii) agents capable of activating specific components of immunity directed against SAMs (i.e., nanoparticles or liposomes inducing general phagocytosis in macrophages), (iv) cell therapies that involve systemic delivery into organism components of immunity that directly or indirectly lead to eradication of SAMs (i.e., non-SAM macrophages, NK cells, etc.), (v) substitution of immune system by hematopoietic stem cell (or the whole bone marrow) transplantation (using preserved autologous bone marrow or stem cells or these from a matching donor).

The disclosure in some aspects and embodiments further provides compositions that can destroy and kill senescence associated macrophages (SAM) in a population of mixed cells. The compositions include but are not limited to small molecule compounds, synthetic organic compounds, Antigen-specific binding polypeptides, Inhibitors of CDK8/CDK19, Inhibitors of apoptosis, Pharmacological inhibitors of NF-kB, Macrolide drugs, antibiotics, chemotoxic drugs and prodrug versions of the same.

The present disclosure also pertains in certain aspects and embodiments to a method of treating a disease related to or caused by cellular senescence in a mammal comprising administering to the mammal a pharmaceutical composition comprising: (a) an agent that is toxic to a cell upon ingestion and a delivery vehicle.

The present disclosure also pertains at least in part to a method for identifying an agent that selectively removes SAMs from a mixed population of cells comprising SAMs, the method comprising (a) contacting a plurality of candidate agents with a mixed population of SAMs and senescent cells; and (b) determining selective removal of SAMs from the mixed population, thereby identifying the agent.

In some embodiments, the agent comprises a small molecule, a peptide, a polypeptide, a toxin, an oligonucleotide, an RNA.

The present disclosure in various aspects and embodiments also pertains to a method of assessing chronic systemic inflammation in a mammal, the method comprising detecting the presence of senescence-associated macrophages (SAMs) in a mammal, wherein the presence of SAMs is indicative of chronic systemic inflammation.

The present disclosure in some aspects and embodiments also pertains to a method of reprogramming senescence-associated macrophages (SAMs) in a mixed population of cells, the method comprising selectively reversing or reducing the SAM phenotype in the mixed population of cells.

In some embodiments, the SAM phenotype is selectively reversed or reduced by delivering to the mixed population of cells an agent capable of reducing SAM expression of p16, β-galactosidase, or both.

In some embodiments, the agent is selected from the group consisting of TA-4812, OT-82, interferon-alpha, interferon-beta, poly(I:C) dsRNA mimetic, activators of toll-like receptor 3 (TLR3), activators of toll-like receptor 4 (TLR4), ruxolitinib, momelotinib, JAK1/JAK2 inhibitors, AS1517499, STAT6 inhibitors, IL-4 neutralizing antibodies, IL-4 receptor neutralizing antibodies, IL-13 neutralizing antibodies, and IL-13 receptor neutralizing antibodies.

In some embodiments, the agent further comprises a delivery vehicle.

In some embodiments, the delivery vehicle is a liposome or nanoparticle.

In certain aspects and embodiments, the present disclosure also provides a composition comprising an agent active in macrophages, the composition being configured to be ingested or selectively activated by the macrophages.

In some embodiments, the composition comprises a delivery vehicle and the agent and the delivery vehicle facilitates the ingestion of the agent by the macrophages.

In some embodiments, the delivery vehicle comprises a liposome or a nanoparticle.

In some embodiments, the agent is selected from the group consisting of TA-4812, OT-82, interferon-alpha, interferon-beta, poly(I:C) RNA, activators of toll-like receptor 3 (TLR3), activators of toll-like receptor 4 (TLR4), ruxolitinib, momelotinib, JAK1/JAK2 inhibitors, AS1517499, STAT6 inhibitors, IL-4 neutralizing antibodies, IL-4 receptor neutralizing antibodies, IL-13 neutralizing antibodies, IL-13 receptor neutralizing antibodies, TA-08209-1, and TA-08210-1.

In some embodiments, the present disclosure also provides a composition comprising an agent active in macrophages, the composition being configured to be ingested or selectively activated by the macrophages, and active in macrophages that are β-galactosidase positive and inactive against non-macrophage cell types.

In some embodiments, the composition comprises a delivery vehicle and the agent, and the delivery vehicle facilitates ingestion of the agent by the macrophages.

In some embodiments, the delivery vehicle comprises a liposome or a nanoparticle.

In some embodiments, the agent comprises a prodrug that becomes active in the macrophages following activation within the macrophages that are β-galactosidase positive and is inactive macrophages that are not β-galactosidase positive.

In some embodiments, the prodrug comprises a drug selected from the group consisting of TA-4812, OT-82, interferon-alpha, interferon-beta, poly(I:C) RNA, activators of toll-like receptor 3 (TLR3), activators of toll-like receptor 4 (TLR4), ruxolitinib, momelotinib, JAK1/JAK2 inhibitors, AS1517499, STAT6 inhibitors, IL-4 neutralizing antibodies, IL-4 receptor neutralizing antibodies, IL-13 neutralizing antibodies, IL-13 receptor neutralizing antibodies, TA-08209-1, and TA-08210-1.

In some embodiments, the invention also provides a composition comprising an agent active in macrophages and capable of killing or reprogramming SAMs, the composition being configured to be ingested or selectively activated by the macrophages, and active in macrophages that are β-galactosidase positive and inactive against non-macrophage cell types.

In some embodiments, the composition comprises a delivery vehicle and the agent, and the delivery vehicle facilitates ingestion of the agent by the macrophages.

In some embodiments, the delivery vehicle comprises a liposome or a nanoparticle.

In some embodiments, the agent comprises a prodrug that becomes active in the macrophages that are β-galactosidase positive and is inactive against non-macrophage cell types.

In some embodiments, the prodrug comprises a drug selected from the group consisting of TA-4812, OT-82, interferon-alpha, interferon-beta, poly(I:C) RNA, activators of toll-like receptor 3 (TLR3), activators of toll-like receptor 4 (TLR4), ruxolitinib, momelotinib, JAK1/JAK2 inhibitors, AS1517499, STAT6 inhibitors, IL-4 neutralizing antibodies, IL-4 receptor neutralizing antibodies, IL-13 neutralizing antibodies, IL-13 receptor neutralizing antibodies, TA-08209-1, and TA-08210-1.

The present disclosure also pertains in certain aspects and embodiments to a method of removing SAMs from a mixed population of cells, the method comprising delivering to the mixed population of cells a first agent capable of modulating the polarization status of the SAMs and a second agent that is toxic the SAMs.

In some embodiments, the first agent causes the SAMs to be more susceptible to the toxic effects of the second agent.

In some embodiments, the first agent polarizes SAMs to an M1 phenotype and the second agent is selectively toxic to the M1 phenotype SAMs.

In some embodiments, first agent is selected from the group consisting of interferon-alpha, interferon-beta, interferon-gamma, toll-like receptor 3 (TLR3) agonists, toll-like receptor 4 (TLR4) agonists, and poly(I:C) RNA.

In an aspect, the present disclosure provides methods of using the heterocyclic compounds or compositions of the present disclosure. For example, the compounds or compositions are used to kill senescent cells, delay one or more feature of aging in an individual/subject. Anti-aging effects include but are not limited to prophylaxis and/or therapy of one or more age-related diseases.

In various examples, the disclosure includes killing senescent cells that are irreversibly arrested and are accumulated with age in tissues of an individual. The senescent cells can be cells of connective or epithelial tissue, or irreversibly arrested melanocytes, or irreversibly arrested tumor cells (spontaneously or following radiation or chemotherapy), or other cells. In various embodiments, eradication of senescent cells comprises eradicating the cells systemically in the whole organism, organ-specifically (e.g., in the skin), or a tumor, such as following conventional cancer treatment by radiation or chemotherapy.

For example, the selective eradication can be done to prevent or treat age-related diseases such as Alzheimer's disease, type II diabetes, macular degeneration, chronic inflammation-based pathologies (e.g., arthritis), and/or to prevent development of cancer types known to be associated with aging (e.g., prostate cancer, melanoma, lung cancer, colon cancer, etc.), and/or with the purpose to restore function and morphology of aging tissues (e.g., skin or prostate), and/or with the purpose to improve morphology of tissue impaired by accumulated senescent cells (e.g., cosmetic treatment of pigmented skin lesions), and/or with the purpose to improve the outcome of cancer treatment by radiation or chemotherapy, and/or with the purpose to prevent recurrent and metastatic disease in cancer patients by elimination of dormant cancer cells. The disclosure is suitable for prophylaxis and/or therapy of human and non-human animal diseases and ageing and age-related disorders.

In various examples, the disclosure relates to the selective eradication of senescent cells and/or SAMS is in an individual suspected of having or at risk for developing an age-related disease, including but not necessarily limited to Alzheimer's disease, Type II diabetes, macular degeneration, or a disease comprising chronic inflammation, including but not necessarily limited to arthritis. In various other examples, the subject of the therapy of the present disclosure is in need of or is undergoing treatment for cancer, including but not necessarily limited to prostate cancer, melanoma, lung cancer, sarcoma, breast cancer, and colon cancer. In various other examples, the individual is in need of therapy for tissue impaired by accumulated senescent cells, such as senescent cells that are present in a pigmented skin lesion.

The compounds or compositions can be administered to a mammal, including humans and non-human mammals. Non-human mammals treated using the present methods include domesticated animals (i.e., canine, feline, murine, rodentia, and lagomorpha) and agricultural animals (bovine, equine, ovine, porcine). In various examples, the individual to whom a compound or composition is administered is an individual who is at risk for, is suspected of having or has been diagnosed with cancer, and/or an age-related disease.

In an example, administering a compound or composition as described herein improves the outcome of a cancer treatment (e.g., radiation treatment and/or chemotherapy) of a mammal. In certain examples, the mammal is in need of treatment for a metastatic cancer, and by practicing a method of this disclosure, dormant cancer cells in the individual are killed. In an example, the lifespan of an individual is increased subsequent to administering a compound or composition as disclosed herein.

The compounds or compositions of the present disclosure can be used in methods for partial or complete eradication of senescent cells in an individual (e.g., one or more types of cells in an individual) comprising administering one or more compounds of the present disclosure to an individual with one or more senescent cells for a period of time sufficient to partially or completely eradicate senescent cells in an individual.

For example, a method of selectively killing one or more senescent cells (e.g., one or more types of senescent) in an individual (e.g., all of or part of the senescent cells in an individual) in need thereof comprises administering to the individual a composition comprising a therapeutically effective amount of one or more of the heterocyclic compounds or compositions comprising one or more heterocyclic compounds. In various examples, the senescent cells are senescent due to replicative cellular senescence, premature cellular senescence, or therapy induced senescence. The senescent cells can be from an age-related pathology.

For example, a method for delaying at least one feature of aging in an individual comprises administering a composition comprising a therapeutically effective amount of one or more of the heterocyclic compounds or compositions comprising one or more heterocyclic compounds. For example, the individual has a received DNA-damaging therapy.

For example, a method of treating an age-related disease or condition comprises administering a composition comprising a therapeutically effective amount of one or more of the heterocyclic compounds or compositions comprising one or more heterocyclic compounds. In an example, the age-related disease or condition is not cancer. In various examples, the age-related disease or condition is a degenerative disease or a function-decreasing disorder.

For example, a method of killing therapy-induced senescent cells comprises administering a composition comprising a therapeutically effective amount of one or more of the heterocyclic compounds or compositions comprising one or more heterocyclic compounds to an individual that has received DNA-damaging therapy. The administration results in killing therapy induced-senescent cells in normal and tumor tissues following DNA-damaging therapy.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The file of this patent contains at least one drawing executed in color. Copies of this patent with color drawings will be provided by the Office upon request and payment of the necessary fee. The foregoing features of embodiments will be more readily understood by reference to the following detailed description, taken with reference to the accompanying drawings, in which:

FIG. 1. Schematic of hypothetical model of in vivo accumulation of p16(INK4a)/β-gal^{pH6}-positive cells in naturally aged organisms. In young mammals (top panel), the secretion of SASP by p16(INK4a)/β-gal^{pH6}-positive senescent cells facilitates the attraction of innate immune components necessary for efficient targeting and destruction of senescent cells. Senescent cell secretions activate recruited macrophages, inducing a p16(INK4a)/β-gal^{pH6}-positive phenotype. After the successful eradication of SCs, inflammatory factors subside and tissue homeostasis resumes. This resolution results in the loss of p16(INK4a)/β-gal^{pH6}-positive cells from the tissue, as macrophages migrate from the tissue, are cleared, or discharge their activated state. However, in old animals (bottom panel), impairments in innate immunity result in the inability to efficiently recognize or destroy SCs. This chronic, inflammatory state initiated by SCs results in persistent activation of macrophages. Their accumulation can be a manifestation of unresolved innate immune response leading to chronic sterile systemic inflammation typical for aged organisms. This results in the amplification of senescence-associated phenotypes within the tissue, namely p16(Ink4a) expression, β-gal^{pH6} activity, and persistence of pro-inflammatory cytokines

FIG. 2. Compound A1.1 selectively kills senescent human neonatal dermal fibroblasts (NDF) cells in a dose- and time-dependent manner. (FIG. 2A) Normal proliferating and quiescent NDF cells, and the cells made senescent via exposure to genotoxic agents (doxorubicin, bleomycin and gamma-irradiation) were incubated with increasing concentrations of A1.1. At 72 hours post-treatment cell viability was measured via resazurin viability assay. (FIG. 2B) Compound A1.1 selectively kills senescent NDF cells in time-dependent manner discriminating between acute genotoxic stress and senescence. Acutely damaged NDF cells were irradiated with 4 Gy gamma-irradiation 24 hours before A1.1 treatment. Senescent NDF cells were treated with bleomycin or irradiated with 15 Gy and incubated for 14 days to induce senescence before treatment with A1.1. Proliferating acutely damaged and senescent NDF cell were treated with 1.875-30 μM A1.1 and viable cells were measured at 72 hours after treatment. Average and standard deviation are shown.

FIG. 3. Activity of A1.1 and analog A1.12 in a model of dnPTEN-induced senescence in RWPE-1 normal prostate epithelial cells. A population of transduced RWPE-1 cells containing doxycycline-inducible dnPTEN was senesced via 1 ug/ml doxycycline for 7 days. Cells which bypass senescence continued to proliferate to form a confluent monolayer. The mixed population of senescent and proliferating cells was treated with A1.1 and A1.12 at 10 uM for 72 hours. SA-beta-Gal staining was used to monitor the amount of senescent cells of treatment groups compared to non-treated control. In addition, methylene blue was used to quantitate overall cell mass, which was comprised mostly of the non-senescent, proliferating cells. Treatment with the anti-senescent compounds led to a substantial decrease in SA-beta-Gal staining, while the amount of methylene blue staining was less affected.

FIG. 4. Efficacy of A1.1 analogs in the senescence-enhanced B16 metastasis model. (A) Quantification of metastatic nodules by H&E morphometric analysis, and the resulting reduction in tumor burden (%) compared to vehicle control. (B) Quantitation morphometric analysis of lungs excised from irradiated mice treated with analogs of A1.1 post-inoculation of B16 cell intravenously, along with quantitation of morphometric analysis, are provided. (C) In vitro testing of A1.1 and analogs against the growth and viability of B16 cells, as assessed by the resazurin viability assay after 72 hour exposure to compounds compared to non-treated controls.

FIG. 5. Characterization of macrophages that accumulate in the peritoneal cavity of mice at several days after injection of alginate particles carrying senescent cells (SC). On day 15, macrophages accumulate that acquire the SAM phenotype (p16(Ink4a)/β-gal^{pH6}-positive). (A) Cells isolated from peritoneal cavities of mice at different time points after injection of SC-carrying alginate beads sorted by type (CD11b (monocytes marker)). (B) Evaluation of expression of major cytokines, secreted by classical macrophages M1 and M2.

FIG. 6. Macrophages elicited by alginate-encapsulated SCs possess a modulatable M2-like phenotype. Gene expression analysis of macrophage polarization markers (M1, Nos2 and Il1b; M2, Arg1) of alginate bead model (AB model)-elicited peritoneal macrophages from wild type mice via qRT-PCR. A) mRNA expression of Nos2 and Arg1 in AB-elicited macrophages adherence-selected from peritoneal lavage, as compared to expression in bone marrow-derived macrophages polarized M1 (IFN-γ for 24 hrs; M1 ctrl) or M2 (IL-4 for 24 hours; M2 ctrl). Gapdh expression was used an internal reference gene control. B) Peritoneal macrophages elicited by our alginate bead model were treated ex vivo with immunomodulatory agents. qRT-PCR analysis of mRNA expression of indicated genes was normalized to β2-microglobulin (B2m) expression following 72 hour incubation with M1-inducing stimuli (LPS at 1 ng/mL+IFN-γ at 10 ng/mL) or M2-inducing cytokines (IL-4 at 20 ng/mL+IL-13 at 10 ng/mL). Fold change in gene expression following treatment is depicted relative to non-treated controls.

FIG. 7. Immunomodulatory regulation of p16(Ink4a) and SAβG in macrophages. Peritoneal lavage cells elicited by alginate-encapsulated SCs from p16^{Ink4a/Luc} mice were treated ex vivo with immunomodulatory agents for 72 hours. A) p16 promoter-dependent luciferase activity (black bars) and 3-galactosidase activity (via 4-MUG hydrolysis) (gray bars) were measured following treatment with M1- and M2-polarizing stimuli: LPS at 1 ng/mL, IFN-γ at 10 ng/mL, LPS/IFN-γ co-treatment, Poly(I:C) at 10 μg/mL, IFN-α at 100 U/mL, IL-4 at 20 ng/mL, IL-13 at 10 ng/ml, and IL-4/IL-13 co-treatment. Results are shown as the mean±standard deviation for at least 3 independent experiments, with statistical significance between treated compared to non-treated samples depicted. B) Dose-dependent response of JAK1/2 inhibitor Ruxolitinib on luciferase activity in elicited macrophages in the presence (gray bars) or absence (black bars) of IL-4 (10 ng/mL) stimulation. Enzyme activities are expressed as the percent activity relative to non-treated (NT) controls per cell. Statistical significance between IL-4 stimulated and non-stimulated cells at each concentration of ruxolitinib is shown.

FIG. 8. Immunomodulatory regulation of p16(Ink4a) and SAβG in macrophages. Peritoneal lavage cells elicited by alginate-encapsulated SCs from p16^{Ink4a/Luc} mice were treated ex vivo with immunomodulatory agents for up to 72 hours. (A) mRNA expression of p16(Ink4a) and β-galactosidase (Glb1) in macrophages (relative to B2m expression) with or without LPS stimulation for 72 hours analyzed via qRT-PCR, normalized to non-treated controls. (B) Microphotograph of SAβG-stained adherence-selected macrophages with or without stimulation with LPS (1 ng/mL) for 72 hours (10× objective). (C&D) Kinetics of p16(Ink4a) promoter-dependent luciferase activity per cell with or without LPS stimulation (C) or IL-4 stimulation (D), normalized to activity from non-treated cells at time zero. Results are shown as the mean±standard deviation for at least 3 experiments. Statistical significance with respect to non-treated control at time zero is indicated. (E) Luciferase activity and (3-galactosidase activity (via 4-MUG hydrolysis) from proteose peptone-elicited lavage cells following stimulation with IL-4 (20 ng/mL) for 72 hours, normalized to non-treated controls. (F) Viability following 72 hour dose-response to JAK1/2 inhibitor Ruxolitinib in the presence or absence of IL-4 (10 ng/mL) stimulation, measured by CyQuant Direct DNA-based viability assay. Viability normalized to respective controls lacking Ruxolitinib. (G&H) Dose-response of STAT6 inhibitor AS1517499 against luciferase activity (G) and viability via CyQuant Direct assay (H) following 72 hour treatment in the presence and absence of IL-4 (10 ng/mL). Results normalized to respective controls (with or without IL-4) lacking STAT6 inhibitor. Statistical significance is calculated with respect to the difference between IL-4 stimulated and non-stimulated cells at each concentration of STAT6 inhibitor. (I) Relative luciferase activity per cell following repolarization with M1- and M2-inducing agents. Macrophages were left non-treated (NT) or treated with either LPS (1 ng/ml) or IL-4 (20 ng/ml) for 72 hours (days 1-3), as indicated. For each treatment set, samples were collected at 72 hours (no further treatment; days 4-6=x). Alternatively, cells were washed and placed in fresh medium (−), medium containing LPS (1 ng/mL) and IFN-γ (10 ng/mL), or medium containing IL-4 (20 ng/mL) and IL-13 (10 ng/mL) and incubated for an additional 72 hours prior to sample collection (as indicated for days 4-6). Luciferase activity is expressed as the percent activity per cell relative to non-treated (NT) controls after the first 72 hours.

FIG. 9. Effect of CYT387 on p16 expression. Shown is the effect of CYT387 (momelotinib) on expression of p16(Ink4a) in SC-containing alginate bead-elicited macrophages from p16-Luc mice, in the presence and absence of IL-4 stimulation. Similar to ruxolitinib and STAT6 inhibitor, CYT387 abrogates IL-4-induced p16(Ink4a) expression.

FIG. 10. Elevated p16(Ink4a) and β-galactosidase is regulated by immunomodulatory agents in macrophages, but not in mesenchymal SCs. Primary cultures of adipose-derived mesenchymal stromal cells (AdMSC) isolated from p16^{Ink4a/Luc} mice were irradiated (20 Gy) and cultured for 10 days for senescence induction. Mock irradiated cells were used as a proliferating cell control. Response of senescent and proliferating AdMSCs to immunomodulatory agents were compared to that of peritoneal lavage cells elicited by our alginate bead model. A-D) Dose-response curves of LPS (top panels: A&D) and Poly(I:C) (bottom panels: C&D) on p16(Ink4a) promoter-dependent luciferase activity (left panels: A&C) and beta-galactosidase activity measured via 4-MUG hydrolysis (right panels: B&D) after 72 hr treatment. No effect on viability was observed via CyQuant Direct assay (>80% viability). Results are shown as the mean±standard deviation for at least 3 experiments.

FIG. 11. OT-82 effects on survival and frailty index of old mice. (A) Male and female mice were treated with OT-82 or with vehicle alone as a negative control. Survival was measured over the course of six treatments. These experiments showed that OT-82 has no significant effects on survival under the tested conditions. (B) Frailty index (FI) results for chronically aged mice revealed a positive effect for OT-82 treated male mice over untreated mice (intact) or mice treated with vehicle alone. (C) shows the chemical structure of OT-82 (product of Oncotaris, Buffalo, N.Y.) (disclosed as compound TT-03582 in U.S. Publication 2016/0347748 (published Dec. 1, 2016)).

FIG. 12. Effects of asenins TA-3899 and TA-4812 on survival and frailty index of old mice. Results are provided on a background of FI of mice of the similar age, aged normally without any treatments (black color). Difference between FI of males receiving 3899 and intact mice is statistically significant (p=0.02).

FIG. 13. Effect of TA-4812 on β-gal expression and cell viability. Asenins TA-04812-1, was assayed for its effects on SAM viability, proliferation, and β-gal expression. SAM viability was determined using the resazurin cell viability assay (Res). Cell proliferation was assessed using the CyQuant assay (CyQUANT® Direct Cell proliferation Assay, ThermoFisher Scientific, Waltham, Mass., Catalog # C35011, performed according to manufacturer's instructions) (CyQ). β-gal production was assessed using the MUG assay, a quantitative assay to determine the level of β-gal expression using 4-methylumbelliferylgalactopyranoside (MUG) as substrate. Compared to untreated SAMs, Asenins TA-04812-1 was able to reprogram SAM phenotype by reducing β-gal expression at concentrations as low as 2.5 μM while having little to no effect on cell viability.

FIG. 14. Effects of OT-82 on macrophages. (A) While being almost non-toxic, OT-82 significantly downregulates expression of luciferase marker p16 in SAMs (effect of asenin TA-04907 which has no effect on luciferase marker, but instead inhibits β-gal is provided for comparison). (B) OT-82 is not toxic to SAMs by itself, and this is not affected by M2-phenotype inducer IL-4. However the combination of OT-82 and inducers of M1 phenotype (LPS and gamma-interferon) is toxic to SAMs.

FIG. 15. Effects of TA-08209-1 and TA-08210-1 on SAM viability. Shown is a dose response of the effects of TA-08209-1 and its galactose linked prodrug analogue TA-08210-1 on SAM viability.

### DETAILED DESCRIPTION OF SPECIFIC EMBODIMENTS

A “macrophage” (MΦ) is a type of white blood cell that ingests foreign particles, infectious microorganisms, cellular debris, cancer cells, and damaged or dying cells by a process called phagocytosis. A macrophage is capable of cell division, proliferation and tissue motility. Macrophages are professional phagocytes found in essentially all tissues where they take various forms (with various names, e.g., histiocytes, Kupffer cells, alveolar macrophages, microglia, and others), but all are part of the mononuclear phagocyte system. Besides phagocytosis, they play a critical role in nonspecific defense (innate immunity) and also help initiate specific defense mechanisms (adaptive immunity) by recruiting other immune cells, such as lymphocytes, and through antigen presentation (e.g. to T lymphocytes). Activated macrophages may encourage inflammation (called M1 macrophages), whereas those that decrease inflammation and encourage tissue repair are called M2 macrophages. This difference is reflected in their metabolism; M1 macrophages have the unique ability to metabolize arginine to the “killer” molecule nitric oxide, whereas rodent M2 macrophages have the unique ability to metabolize arginine to the “repair” molecule ornithine. Macrophages can secrete pro-inflammatory cytokines (encouraging inflammation) or anti-inflammatory cytokines (which decrease inflammation and encourage tissue repair). In mice, macrophages include those originating from yolk sac erythromyeloid progenitors, including both cMyb-positive and cMyb-negative stages, and those originating from circulating bone marrow-derived monocytes. Human macrophages are about 21 micrometers in diameter. They can be identified using flow cytometry or immunohistochemical staining by their specific expression of proteins such as CD14, CD40, CD11b, CD64, F4/80 (mice)/EMR1 (human), lysozyme M, MAC-1/MAC-3 and CD68.

SAM (Senescence-associated macrophages) defines a subset of macrophages which are p16(Ink4a)/β-gal^{pH6}-positive, and which accumulate with age. p16(Ink4a)/β-gal^{pH6}-positive macrophages are capable of cell division and phagocytosis. Induction of the SAM phenotype may represent a specific type of macrophage activation or differentiation. Thus, p16(Ink4a)/β-gal^{pH6}-positive macrophages accumulating in tissues of old mice may not necessarily be activated by SCs, but are found within aged (i.e. senescent) organisms. In some embodiments, SAMs express p16(Ink4a) and β-gal^{pH6} independently of p53, and express substantially similar amounts of p16(Ink4a) and β-gal^{pH6} in both p53 knockout mice (p53^{-/-}) and in mice possessing functional copies of wildtype p53 (p53^{+/+}). In some embodiments, SAMs do not exhibit cytoplasmic localization of HMGB1, a marker of mesenchymal SCs. SAMs are removed from a population of mixed cells which includes SAMs and SCs by treatment of the population with a delivery vehicle that comprises a toxin, such as clodronate, because SAMs phagocytose the toxin and SCs do not. A SAM expresses cell surface markers characteristic of a macrophage, including CD11b and F4/80. In some embodiments, SAM expression levels of p16(Ink4a) and/or β-gal^{pH6} is able to be reversibly modulated in response to immunomodulatory agents, e.g., poly(I:C), IFN-α, IFN-β, and various other agents and small molecules described herein. In some embodiments, SAMs express equal or greater amounts of interleukin-12 subunit beta (IL-12p40) than M1 macrophages and express less nitric oxide synthase (iNOS) than M1 macrophages. In some embodiments, SAMs express equal or greater amounts of arginase (Arg1) than M2 macrophages and express less interleukin-10 (IL-10) than M2 macrophages. In some embodiments, SAMs are unable to induce tumorigenesis or stimulate tumor establishment, unlike tumor-associated macrophages (TAMs).

“Mixed Population of Cells” will include SAMs (positive for CD11b and F4/80), and may also include one or more types of cells, some non limiting examples of such cells are p16(Ink4a)-positive and/or β-gal^{pH6}-positive and which cannot divide/proliferate, cells which are of hematopoietic origin, B lymphocytes (CD19⁺), eosinophils (CD19⁻ CD11b⁺ CD170⁺, neutrophils (CD11b⁺ Ly-6G⁺), CD11b⁻ cells (e.g. T lymphocytes) and CD11b⁺ F4/80⁻ cells (e.g. monocytes, NK cells). More generally mixed populations are all other cell types found in tissues, including macrophages which also do not have SAM-specific phenotype.

“Inflammation” is a normal response to a variety of acute stresses on the body, including infection, fever and injury. Other types of inflammation include increased levels of pro-inflammatory cytokines found within tissues and systemically in plasma. Inflammation may be associated with infections, but it occurs in response to virtually any type of injury or threat, including physical trauma, cold, burns from radiation, heat or corrosive materials, chemical irritants, bacterial or viral pathogens, localized oxygen deprivation (ischemia) or reperfusion (sudden reinfusion of oxygen to ischemic tissue), and others. It includes the classic symptoms of redness, heat, swelling, and pain, and may be accompanied by decreased function of the inflamed organ or tissue. It is a generalized reaction involving several effects that may tend to combat an injurious agent that may be present at the site where an injury or threat was detected, or it may tend to contain the injury or threat to its initial location, to keep it from spreading rapidly Inflammation is a self-defensive reaction aimed at eliminating or neutralizing injurious stimuli, and restoring tissue integrity. Like peripheral inflammation, neuroinflammation can become a harmful process, and it is now widely accepted that it may contributes to the pathogenesis of many central nervous system disorders. CNS inflammation is commonly associated with some degree of tissue damage including, loss of myelin sheaths or loss of axons, and is a central theme in human patients with MS. The level of inflammation can be quantified by performing a simple blood test for a particular compound called C-reactive protein, or CRP.

“Sterile chronic systemic inflammation”, named “inflammaging” [7,8] in an adult is a characteristic of aging. Chronic inflammation causes damage over time to organ systems like the heart, brain and kidneys, leading to disability or premature death. Blood vessels that supply these organs are vulnerable to inflammation, leading to vessel wall-thickening and narrowing of the blood passageway. Elevated CRP levels, measured over time, are an indicator of chronic inflammation in humans. Studies have shown that elevated levels of CRP correlate with an increased risk of heart attack and stroke. Aging is an intricate process that results from a combination of environmental, genetic, epigenetic, and stochastic factors. A chronic proinflammatory status is a pervasive feature of aging. This chronic, low-grade, systemic inflammation occurring in the absence of overt infection (sterile inflammation) has been defined as “inflammaging” and represents a significant risk factor for morbidity and mortality in the elderly. Prattichizzo et al in (Inflammaging” as a Druggable Target: A Senescence-Associated Secretory Phenotype-Centered View of Type 2 Diabetes) Oxid Med Cell Longev. 2016 and Nasi et al in (Aging and inflammation in patients with HIV infection), Clin Exp Immunol. 2016 May 20, explore the connection between aging and inflammation.

The term “age-related disease” includes but is not limited to a disease in an adult such as cancer, a metabolic disease, cardiovascular disease, tobacco-related disease, or skin wrinkles. Cancer includes but is not limited to prostate cancer, colon cancer, lung cancer, squamous cell cancer of the head and neck, esophageal cancer, hepatocellular carcinoma, gastric cancer, pancreatic cancer, ovarian cancer, or breast cancer. Age-related or tobacco-related disease includes cardiovascular disease, cerebrovascular disease, peripheral vascular disease, Alzheimer's disease, osteoarthritis, cardiac diastolic dysfunction, benign prostatic hypertrophy, aortic aneurysm, or emphysema.

The term “rejuvenation” (for example, following removal or elimination of p16(Ink4a)/β-gal^{pH6}-positive macrophages) refers to the results of reducing or preventing the progress of aging and/or reducing or preventing the progress of an age-related disease. The term “rejuvenating” refers to a process of improving parameters of frailty index and/or other markers of aging cell phenotypes or markers of age-related disease states, e.g., improved muscle endurance or strength, improved glucose tolerance, decreased presence of systemic or local inflammatory cytokines, improved mitochondrial function, and erasing epigenetic modifications participating in the cellular aging phenotype. A process of rejuvenation is observed when at least one of these parameters or markers of aging cell phenotypes is reduced or suppressed in an organism or in aged or senescent cell types due to the rejuvenating process. In some embodiments, the loss of at least one of the markers identified as having increased expression in adipose tissue macrophages (ATMs) from aged mice (Garg, S. K. et al. Crit Rev Immunol. 2014; 34(1):1-14.): CD11c, CD206, Mgl1, IL-6, TNF-alpha, Nos2, Ccr-7, IL-12, Arg1, Ccl-2, Ccr-1, Ccr-5, Ccr-9, Mcp-1, Cxcr-3, IL-1beta may also be considered a sign of rejuvenation.

With respect to the agents described herein, the terms “modulate” and “modulation” refers to the upregulation (i.e., activation or stimulation) or downregulation (i.e., inhibition or suppression) of a response. A “modulator” is an agent, compound, or molecule that modulates, and may be, for example, an agonist, antagonist, activator, stimulator, suppressor, or inhibitor. The terms “inhibit”, “reduce”, remove as used herein refer to any inhibition, reduction, decrease, suppression, downregulation, or prevention in expression or activity and include partial or complete inhibition of activity. Partial inhibition can imply a level of expression or activity that is, for example, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% of the uninhibited expression or activity. The terms “eliminate” or “eradicate” indicate a complete reduction of activity. The terms “activate” or “induce” are used herein to refer to any activation, induction, increase, stimulation, or upregulation in expression or activity and include partial activation of activity, such as, for example, an increase of at least 5%, at least 10%, at least 20%, at least 40%, at least 60%, at least 80%, at least 100%, at least 150%, at least 200%, of the expression or activity in the absence of the agonist. Under certain circumstances, the practice of the methods described herein may result in at least a 5%, 10%, 15%, 20%, 30%, 40%, 50%, 60%, 70%, 80%, 90%, 95%, 98% or 99% reduction in the SAM population. A reduction in the SAM population, whether in a pure or mixed population of SAMs, means to reduce the number of SAM cells, whether measured directly or via expression of a detectable gene product.

The term “gene product” as used herein means an RNA (for example, a messenger RNA (mRNA)) or protein that is encoded by the gene. The term “expression” is used herein to mean the process by which a polypeptide is produced from DNA. The process involves the transcription of the gene into mRNA and the translation of this mRNA into a polypeptide. Depending on the context in which used, “expression” may refer to the production of mRNA, protein, or both.

The term “senescence” is used herein in reference to cells (“senescent cells”, “SC”) which are incapable of further cell division. Cellular senescence is characterized by growth cycle arrest in the G1 phase, absence of S phase and lifespan control by multiple dominant genes. Commonly used criteria to establish cell senescence include, but are not limited to such factors as cellular enlargement and flattening, cessation of proliferation as measured by the failure to increase the number of cells in culture over a two-week period, failure to subculture or form colonies at clonal density, and lack of significant incorporation of [.sup.3 H] thymidine. Although senescent cells remain viable for long time periods, they cannot be stimulated to enter the S phase of the cell cycle by any combination of growth factors or physiological mitogens. Cells which are normally capable of proliferation in vitro can be epigenetically reprogrammed by exposure to genotoxic (i.e., irradiation, chemotherapeutic drugs, etc.) or oncogenic (activation of dominant oncogenes) stresses [25,26] and such cells are characterized by permanent cell cycle arrest, unresolved constitutive DNA damage response and constitutive activation of NF-κB that drives the expression and production of a series of bioactive, largely proinflammatory factors (SASP). Senescent cells may remain viable for long time periods (many months), during which RNA and protein are synthesized. SCs express the p16(Ink4a) gene and exhibit acidic β-galactosidase activity detectable at a more neutral pH (β-gal^{pH6}).

As used herein, the term “proliferation” and “growth” are used interchangeably to indicate cell division.

As used herein, the term “quiescent” is used in reference to cells which are not proliferating, but which can be stimulated to enter the S stage in the cell cycle when appropriate growth factors are present.

As used herein the term “terminally differentiated” is used in reference to cells and tissues which have reached their final stages of development. For example, terminally differentiated cells are those which have attained the specialized characteristics associated with a particular cell type.

As used herein, the term “immortal phenotype” refers to cells and cell lines which have essentially infinite lifespans. Typically, immortal cell lines (e.g. continuous cell lines) are those which have been transformed and lack such characteristics commonly associated with normal cells as contact inhibition, an increased growth rate, etc.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro.

As used herein the term “S phase,” or “S stage” refer to the phase of the cell growth cycle in which DNA synthesis occurs. The remaining stages of the cell cycle are indicated as “M phase” in reference to mitosis, “G.sub.1,” in reference to the growth phase occurring after mitosis but before the S phase, and “G.sub.2,” used in reference to a second growth phase which occurs after DNA synthesis.

As used herein, the term “stain” and “dye” refer to any color which results is used as an indicator for the presence of a particular compound. These terms also refer to any compound which imparts color to a structure or compound. It is contemplated that this color production be the result of enzymatic or other activity. For example, the reaction of .beta.-galactosidase on such substrates as X-gal results in the staining of cells which express this enzymatic activity.

As used herein, the term “β-galactosidase substrate” refers to any natural or artificial (e.g., synthetic) compound upon which .beta.-galactosidase may act. This term encompasses the various commercially available .beta.-galactosidase substrates, including, but not limited to: 5-bromo-4-chloro-3-indolyl-.beta.-D-galactoside; 5-bromo-4-chloro-3-indolyl-.beta.-D-galactopyranoside (i.e., X-gal, available from numerous chemical suppliers, such as Sigma, Research Organics, and other companies); 5-bromoindolyl-.beta.-D-galactopyranoside; ortho-nitrophenyl-.beta.-D-galactopyranoside (i.e., ONPG); and fluorescein di-.beta.-D-galactopyranosides (FDG), as well as carboxyfluorescein compounds (e.g., C.sub.12 FDG) available from Molecular Probes. It is further not intended that the present invention be limited to the color produced. For example, some compounds such as X-gal produce a blue color upon reaction with .beta.-galactosidase, while others such as ONPG, produce a yellow color. In addition, other stains and dyes are contemplated for use in the present invention as indicators of .beta.-galactosidase activity.

The phrase “escape from senescence” refers to the reversion of immortal cell lines with introduced chromosomes associated with senescence, to their original immortal phenotype. This term also refers to any cells, whether or not they are cell lines, previously associated with the senescent phenotype, which are immortal.

The present methods are also applied therapeutically to deplete SAMs endogenous to a subject in need of treatment. The terms “reduce”, “deplete” or “eradicate” p16(Ink4a)/β-gal^{pH6}-positive macrophages refers to a reduction in the number of viable SAMs. Without wishing to be bound by theory, the result of eliminating or depleting SAMs is to reduce the effect of these cells on their neighboring environment, such as to reduce pro-inflammatory cytokine secretion which generates a toxic local environment that affects the health of neighboring cells and promotes cell aging and disease associated with aging.

The term “selectively toxic to SAMs” means that the binding agent is at least 2 times more toxic to a SAM compared to an another cell, for example an SC and alternatively 3 times greater, 5 times greater or at least one order of magnitude greater (e.g., at least 2, 3, 4 or 5 orders of magnitude greater). For instance, the cell toxicity of an antibody that binds a SAM is preferably at least twice its toxicity for a non-SAM macrophage.

“Reversing the SAM phenotype,” “reducing the SAM phenotype,” “modulating SAM phenotype,” and “reprogramming SAM macrophages/phenotype” defines reducing or decreasing the expression of a gene or marker expressed by SAMs. In some embodiments reversing, reducing, modulating, or reprogramming SAMs may lead to a reduction in the expression of p16(Ink4a) and/or β-gal^{pH6} in SAMs. In addition to p16(Ink4a) and/or β-gal^{pH6}, the following markers have been identified as having increased expression in adipose tissue macrophages (ATMs) from aged mice (Garg, S. K. et al. Crit Rev Immunol. 2014; 34(1):1-14.): CD11c, CD206, Mgl1, IL-6, TNF-alpha, Nos2, Ccr-7, IL-12, Arg1, Ccl-2, Ccr-1, Ccr-5, Ccr-9, Mcp-1, Cxcr-3, IL-1beta. In some embodiments, modulation expression of at least one of these markers may also be considered reversing, reducing, modulating, or reprogramming SAM phenotype. In other embodiments, SAM phenotype may be reversed, reduced, modulated, or reprogrammed by minimizing SAM secretion of bioactive molecules which may, or may not, affect expression of p16(Ink4a) and/or β-gal^{pH6}. SAM phenotype may be reversed, reduced, modulated, or reprogrammed by treating SAMs with an agent capable of reversing or reducing SAM phenotype(s). In some embodiments, SAM phenotype may be reversed, reduced, modulated, or reprogrammed by suppression of other non-SAM cell types, e.g., T cells and senescent cells, and/or their secretion of bioactive molecules in the microenvironment which give rise to the SAM phenotype. In some embodiments, SAM phenotype may be reversed, reduced, or reprogrammed using immunomodulatory agents, e.g., poly(I:C) RNA, IFN-α, IFN-β, neutralizing antibodies against SAM secretions, and various other agents and small molecules described herein. In some embodiments, SAM phenotype is reversed, reduced, or reprogrammed when SAM p16(Ink4a) and/or β-gal^{pH6} expression levels are less than 90% that of untreated SAMs. In some embodiments, SAM p16(Ink4a) and/or β-gal^{pH6} expression levels are less than 80%, less than 70%, less than 60%, less than 50%, less than 40%, less than 30%, less than 20%, or less than 10% that of untreated SAMs.

“Non-macrophage cell types” are cell types that do not phagocytose large foreign particles and are not CD11b⁺ and F4/80⁺. In some embodiments, non-macrophage cell types are not p16(Ink4a)/β-gal^{pH6}-positive. In some embodiments, non-macrophage cell types are non-hematopoietic in origin (CD45-negative cells). In some embodiments, non-macrophage cell types include fibroblasts, mesenchymal cells, lymphocytes, B cells, T cells, stromal cells, and epithelial cells.

As used herein, “modulating the polarization status of SAMs” refers to increasing or decreasing the expression of one or more markers associated with the M1 or M2 macrophage phenotype. Markers associated with the M1 macrophage phenotype include nitric oxide synthase (iNOS) and interleukin-12 subunit beta (IL-12p40). Markers associated with the M1 macrophage phenotype include arginase (Arg1) and IL-10. In some embodiments, polarizing SAMs to an M1 phenotype refers to increasing the expression of one or more markers associated with the M1 macrophage phenotype. In some embodiments, polarizing SAMs to an M1 phenotype refers to decreasing the expression of one or more markers associated with the M2 macrophage phenotype. In some embodiments, polarizing SAMs to an M2 phenotype refers to increasing the expression of one or more markers associated with the M2 macrophage phenotype. In some embodiments, polarizing SAMs to an M2 phenotype refers to decreasing the expression of one or more markers associated with the M1 macrophage phenotype.

As used herein, “TA-3899,” “TA-03899,” “TA-3899-1,” and “TA-03899-1” are used interchangeably to refer to compound A1.11 (Ethyl 1-(benzylthio)-7-(hydroxyimino)-5-(4-methoxyphenyl)-4,5,6,7-tetrahydro-thieno[3,4-c]pyridine-3-carboxylate), the structure of which is provided herein.

As used herein, “TA-4812,” “TA-04812,” “TA-4812-1,” and “TA-04812-1” are used interchangeably to refer to compound A1.6 (3-(Benzylthio)-N-(3-fluorobenzyl)-4-(hydroxyimino)-6-(4-methoxyphenyl)-4,5,6,7-tetrahydrobenzo[c]thiophene-1-carboxamide), the structure of which is provided herein.

“Selective-binding” agents are agents that bind a target epitope, and bind proteins that present the target epitope in a solvent-accessible orientation, with an affinity that is at least 2 times greater than the affinity with which they bind a different, unrelated epitope, such as 3 times greater, 5 times greater or at least one order of magnitude greater (e.g., at least 2, 3, 4 or 5 orders of magnitude greater). For instance, the binding affinity of an antibody that binds a SAM is preferably at least twice its binding affinity for an SC. Relative binding affinities can be determined, and the selective binding agent or antibody so selected, on the basis of assays and techniques that generally are well established in the art for this purpose.

As used herein, the term “a disorder” or “a disease” refers to any derangement or abnormality of function; a morbid physical or mental state. See Dorland's Illustrated Medical Dictionary, (W.B. Saunders Co. 27th ed. 1988).

As used herein, the term “treating” or “treatment” of any disease or disorder refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

As used herein, the term “abnormal” refers to an activity or feature which differs from a normal activity or feature. As used herein, the term “abnormal activity” refers to an activity which differs from the activity of the wild-type or native gene or protein, or which differs from the activity of the gene or protein in a healthy subject. The abnormal activity can be stronger or weaker than the normal activity. In one embodiment, the “abnormal activity” includes the abnormal (either over- or under-) production of mRNA transcribed from a gene. In another embodiment, the “abnormal activity” includes the abnormal (either over- or under-) production of polypeptide from a gene. In another embodiment, the abnormal activity refers to a level of a mRNA or polypeptide that is different from a normal level of the mRNA or polypeptide by about 15%, about 25%, about 35%, about 50%, about 65%, about 85%, about 100% or greater. In some embodiments, the abnormal level of the mRNA or polypeptide can be either higher or lower than the normal level of the mRNA or polypeptide. Yet in another embodiment, the abnormal activity refers to functional activity of a protein that is different from a normal activity of the wild-type protein. In some embodiments, the abnormal activity can be stronger or weaker than the normal activity. In some embodiments, the abnormal activity is due to the mutations in the corresponding gene, and the mutations can be in the coding region of the gene or non-coding regions such as transcriptional promoter regions. The mutations can be substitutions, deletions, insertions.

A “target” protein or gene is one which is present in or on a target cell and required for its function. Therefore, a protein or gene which is present on all cells in a population of mixed cells but which is only required for function of a subpopulation of cells qualifies as a target protein or gene of the subpopulation only.

“Therapeutically effective amount” as used herein means the amount of a compound or composition (such as described herein) that causes at least one desirable change in a cell, population of cells, tissue, individual, patient or the like. In some embodiments a therapeutically effective amount as used herein means the amount of a compound or composition (such as described herein) that prevents or provides a clinically significant change in a disease or condition (e.g., reduce by at least about 30 percent, at least about 50 percent, or at least about 90 percent) or in one or more features of a disease or condition described herein. In some embodiments a therapeutically effective amount of a compound or composition is the amount of the compound or composition comprising one or more of the compounds administered to an individual (e.g., in a single dose, in multiple doses, in all of the doses, in a single administration, in multiple administrations, or in all of the administrations) kills all of or a least a portion of one or more types of SAMs in an individual.

The present disclosure provides for selective removal, reduction, elimination or neutralization of SAMs by relying on their phagocytic ability. That is, a drug or prodrug which is toxic upon ingestion by a cell is introduced to the cell using a delivery vehicle which permits a SAM to phagocytose the drug or prodrug. Therefore, selectivity is based on SAM ability to perform phagocytosis.

In an aspect, the present disclosure provides methods of using the heterocyclic compounds or compositions of the present disclosure. For example, the compounds or compositions are used to kill SAMs, delay one or more feature of aging in an individual/subject. Anti-aging effects include but are not limited to prophylaxis and/or therapy of one or more age-related diseases.

In various examples, the disclosure includes killing SAMs which are defined as senescent-associated macrophages, where senescence is aged tissue, with or without direct contribution of SCs accumulation within tissue. In various embodiments, eradication of SAMs comprises eradicating the cells systemically in the whole organism, organ-specifically (e.g., in the skin), or a tumor, such as following conventional cancer treatment by radiation or chemotherapy.

For example, the selective eradication can be done to prevent or treat age-related diseases such as Alzheimer's disease, type II diabetes, macular degeneration, chronic inflammation-based pathologies (e.g., arthritis), and/or to prevent development of cancer types known to be associated with aging (e.g., prostate cancer, melanoma, lung cancer, colon cancer, etc.), and/or with the purpose to restore function and morphology of aging tissues (e.g., skin or prostate), and/or with the purpose to improve morphology of tissue impaired by accumulated SAMs (e.g., cosmetic treatment of pigmented skin lesions), and/or with the purpose to improve the outcome of cancer treatment by radiation or chemotherapy, and/or with the purpose to reduce the risk of metastatic engraftment in cancer patients by elimination of dormant cancer cells. The disclosure is suitable for prophylaxis and/or therapy of human and non-human animal diseases and ageing and age-related disorders.

In various examples, the disclosure relates to the selective eradication of SAMs is in an individual suspected of having or at risk for developing an age-related disease, including but not necessarily limited to Alzheimer's disease, Type II diabetes, macular degeneration, or a disease comprising chronic inflammation, including but not necessarily limited to arthritis, pulmonary disease, cardiovascular disease. In various other examples, the subject of the therapy of the present disclosure is in need of or is undergoing treatment for cancer, including but not necessarily limited to prostate cancer, melanoma, lung cancer, sarcoma, breast cancer, and colon cancer. In various other examples, the individual is in need of therapy for tissue impaired by accumulated SAMs, such as SAMs that are present in a pigmented skin lesion. In various other examples, the subject of the therapy described in the present disclosure has undergone anti-cancer treatments in childhood and are in need of treatment for long-term consequences of such therapies.

In an example, administering a compound or composition as described herein improves the outcome of a cancer treatment (e.g., radiation treatment and/or chemotherapy) of a mammal. In certain examples, the mammal is in need of treatment for a metastatic cancer, and by practicing a method of this disclosure, dormant cancer cells in the individual are killed. In an example, the lifespan of an individual is increased subsequent to administering a compound or composition as disclosed herein.

The compounds or compositions of the present disclosure can be used in methods for partial or complete eradication of SAMs in an individual (e.g., one or more types of cells in an individual) comprising administering one or more compounds of the present disclosure to an individual with one or more SAMs for a period of time sufficient to partially or completely eradicate SAMs in an individual.

For example, a method of treating an age-related disease or condition may comprise administering a composition comprising a therapeutically effective amount of one or more of the heterocyclic compounds or compositions comprising one or more heterocyclic compounds. In an example, the age-related disease or condition is not cancer. In various examples, the age-related disease or condition is a degenerative disease or a function-decreasing disorder.

For example, a method of killing therapy-induced SAMs comprises administering a composition comprising a therapeutically effective amount of one or more of the heterocyclic compounds or compositions comprising one or more heterocyclic compounds to an individual that has received DNA-damaging therapy. The administration results in killing therapy induced-SAMs in normal and tumor tissues following DNA-damaging therapy.

Delivery Vehicles:

A delivery vehicle useful according to the invention includes but is not limited to liposomes, macrophage delivery vehicles such as apoptotic cells, neutrophils, bacteria, spoiled erythrocytes, apoptotic immunocytes, viruses and synthetic nanoparticles. A delivery vehicle useful according to the invention includes any vehicle capable of encapsulating or being conjugated to a drug and also being recognized by a macrophage and phagocytosed by macrophages resulting in the release of drugs.

The in vivo efficacy of any drug depends upon the efficiency with which it is delivered to the relevant cellular compartment in the target cells. Anionic liposomes, which entrap the (antisense oligonucleotide) ASO inside lipidic membranes, have been demonstrated to be an efficient delivery vehicle for targeting ASOs to Kupffer cells. (Ponnappa, B. C., et al., J. Liposome Res. 8: 521-535, 1998). Ninety minutes post intravenous injection, over 50% of liposome-encapsulated ASO was distributed in macrophage-rich organs, liver (40%) and spleen (10%), while incorporation into other organs such as muscle, heart, brain, lungs, kidneys and testes was minimal (Ponnappa, B. C., et al., J. Liposome Res. 8: 521-535, 1998). In the liver, over 65% of the ASO was found in Kupffer cells, accounting for a 200-fold enrichment of ASO in Kupffer cells versus that in the combined body tissues. (Ponnappa, B. C., et al., J. Liposome Res. 8: 521-535, 1998).

One aspect of the present invention is to provide a liposome composition as a delivery vehicle for a therapeutically active agent is encapsulated into a liposome having lipid components and a cholesterol. The liposome is administered to the mammal, thereby delivering the liposome to a macrophage. The liposome is taken up by the macrophage, and subsequently fuses with a lysosome in the macrophage, thereby destabilizing the liposome fused with the lysosome in the macrophage, and releasing the therapeutic agent into the cytosol of the macrophage.

Therapeutic agents delivered by the liposomes into cells are any compound or composition of matter that can be encapsulated in liposomes and administered to a mammal, preferably humans. Liposomes can be loaded with therapeutic agents by solubilizing the agent in the lipid or aqueous phase used to prepare the vesicles. Alternatively, ionizable therapeutic agents can be loaded into liposomes by first forming the liposomes, establishing an electrochemical potential, e.g., by way of a pH gradient, across the outermost liposomal bilayer, and then adding the ionizable agent to the aqueous medium external to the liposome (see Bally et al. U.S. Pat. No. 5,077,056, the contents of which are incorporated herein by reference).

Therapeutic agents can have therapeutic activity in mammals, and can also be administered for diagnostic purposes. Therapeutic agents which may be associated with this invention's liposome include, but are not limited to: antiviral agents such as acyclovir, zidovudine and the interferons; antibacterial agents such as aminoglycosides, cephalosporins and tetracyclines; antifungal agents such as polyene antibiotics, imidazoles and triazoles; antimetabolic agents such as folic acid, and purine and pyrimidine analogs; antineoplastic agents such as the anthracycline antibiotics and plant alkaloids; carbohydrates, e.g., sugars and starches; amino acids, peptides, proteins such as cell receptor proteins, immunoglobulins, enzymes, hormones, neurotransmitters and glycoproteins; dyes; radiolabels such as radioisotopes and radioisotope-labeled compounds; radiopaque compounds; fluorescent compounds; mydriatic compounds; bronchodilators; nucleic acid sequences such as messenger RNA, cDNA, genomic DNA and plasmids; anti-inflammatory agents, such as curcumin and the like.

The amount of the encapsulated drug in the liposome of the invention that will be effective in the treatment of a particular disorder or condition will depend on the nature of the disorder or condition, and is determined by standard clinical techniques. In addition, in vitro assays may optionally be employed to help identify optimal dosage ranges. The precise dose to be employed in the formulation also will depend on the route of administration, and the seriousness of the disease or disorder, and will be decided according to the judgment of the practitioner and each patient's circumstances. Effective doses may be extrapolated from dose-response curves derived from in vitro or animal model test systems. The method of delivery is by the intravenous route. The liposomes may be administered, for example, by infusion or bolus injection, and may be administered together with other biologically active agents. The route of administration is dependent on the formulation of the liposomes, some of known methods of administration include but not limited to gastrointestinal/enteral, epidural, intracerebral, interacerebroventricular, epicutaneous, sublingual, buccal, extra-amniotic, nasal, intraarterial, intraarticular, intracardiac, intracavernous, intradermal, intralesional, intramuscular, intraocular, intraosseous, intraperitoneal, intrathecal, intrauterine, intravaginal, intravenous, intravesical, intravitreal, subcutaneous, transdermal, transmucosal administration.

Macrophages contain scavenger receptors that recognize an array of negative charges such as in anionic liposomes, thereby allowing for the efficient uptake of the liposomes of the present invention. (Ponnappa, B. C., et al., J. Liposome Res. 8: 521-535, 1998; Bautista, A. P., et al., J. Leukocyte Biol. 55: 321-327, 1994). Sequestration by macrophages, of which the Kupffer cells in the liver are an example, is further facilitated by the large size of the liposomes. (Alino, S. F., et al., Biochem Res. Com. 192: 174-181, 1993). Liposomes having a diameter larger than 200 nm are preferred as the size prevents them from crossing the fenestrations in the endothelial barrier of the liver and spleen sinusoids. This barrier has the highest permeability of tissue capillaries. As macrophages migrate towards areas of infection/inflammation, the larger liposomes allows for efficient delivery of the therapeutic agent to the macrophages. Consequently, fusion of the liposomes with the endosomal membrane will release the contents into the cytosol prior to the lysosomal enzymes becoming optimally active, thereby circumventing any damage to the therapeutic agent within the liposome.

SAMolytic” compounds are small molecules for selective killing of (Senescence associated macrophages) SAMs

The following abbreviations are used in the examples:
nt=not tested
NDF=human neonatal dermal fibroblasts
NDFsen.bleo=senescent human neonatal dermal fibroblasts (senescence bleomycin-induced)
NDFsen.irr=senescent human neonatal dermal fibroblasts (senescence irradiation-induced)
NDFprolif=proliferating human neonatal dermal fibroblasts
RDF=rat dermal fibroblasts
RDFsen.bleo=senescent adult rat dermal fibroblasts (senescence bleomycin-induced)
RDFsen.irr=senescent adult rat dermal fibroblasts (senescence irradiation-induced)
RDFprolif=proliferating adult rat dermal fibroblasts

The following are preferred compounds for selectively killing SAMs:

[1]. A compound having the following formula:

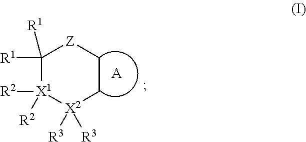

wherein:

A, together with the two carbons from the adjacent ring, is a fused heterocycle;

Z is selected from the group consisting of C═N—OR⁵, wherein R⁵ is H or a prodrug substituent;

X¹ is C or N;

X² is C, O, or a spacer (e.g., —C(═O)—, —C(N═OR⁶)—, —C(═O)CH₂—, —C(N═OR⁶)CH₂—, —CH₂C(═O)—, —CH₂C(N═OR⁶)—), wherein R⁶ is —H or substituted or unsubstituted C₁ to C₆ alkyl group;

each R¹ is independently selected from the group consisting of H, C₁ to C₆ alkyl group, and C₃ to C₁₈ aryl group, or both R¹ groups may, together with the carbon they are attached to, form a C₃ to C₁₀ spirocycle group;

each R² is independently selected from the group consisting of H, C₁ to C₆ alkyl group, C₃ to C₁₈ aryl group, C₁-C₂₀ heteroaryl group, and C(O)N(R⁷)₂, or both R¹ groups may, together with the carbon they are attached to, form a C₃-C₁₀ spirocycle group, wherein R⁷ is independently selected from the group consisting of —H, substituted or unsubstituted C₁-C₆ alkyl group, substituted or unsubstituted alkynyl group, substituted or unsubstituted aryl group, substituted or unsubstituted heteroaryl group, substituted or unsubstituted saturated heterocyclyl group, substituted or unsubstituted partially-saturated heterocyclyl group, substituted or unsubstituted saturated C₃ to C₂₀ carbocyclyl group and substituted or unsubstituted partially-saturated C₃ to C C₂₀ carbocycylyl group, or both R⁹ substituents, together with the N atom they are attached to, form a heterocycle group, which may additionally contains a further heteroatom(s) selected from the group consisting of N, O, and S;

each R³ is independently selected from the group consisting of H, C₁ to C₆ alkyl group, C₃ to C₁₈ aryl group, and C₁ to C₂₀ heteroaryl group; and

any two of R¹, R², R³, and/or substituents of A may be linked together with a linker to form a macrocycle group,

wherein one or more of the heteroatoms, such as, for example, nitrogen and sulfur, may optionally be oxidized to form N-oxides or sulfoxides and sulfones, respectively, and/or one or more nitrogen in one or more heterocycle may be quaternized, or a salt (e.g., a pharmaceutically acceptable salt) of the compound.

[2]. The compound of [1], wherein A is selected from the group consisting of:

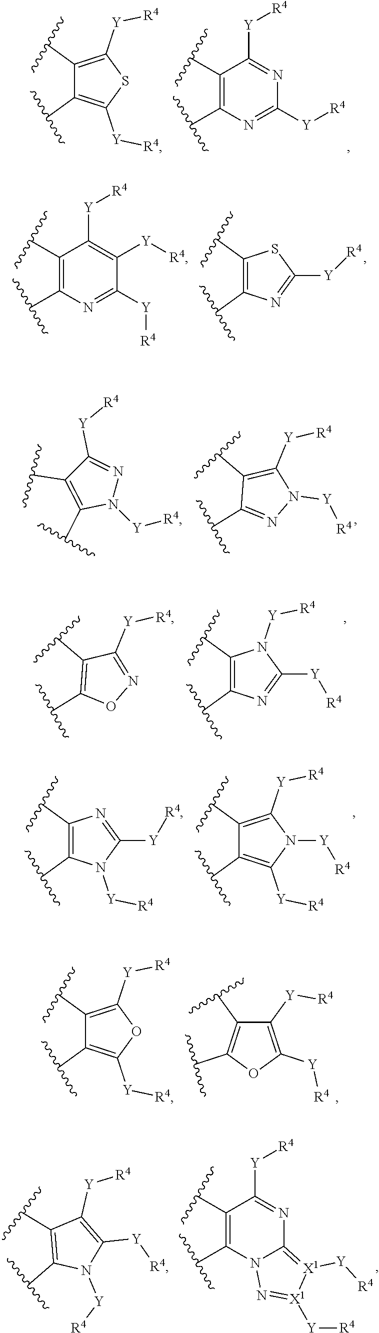

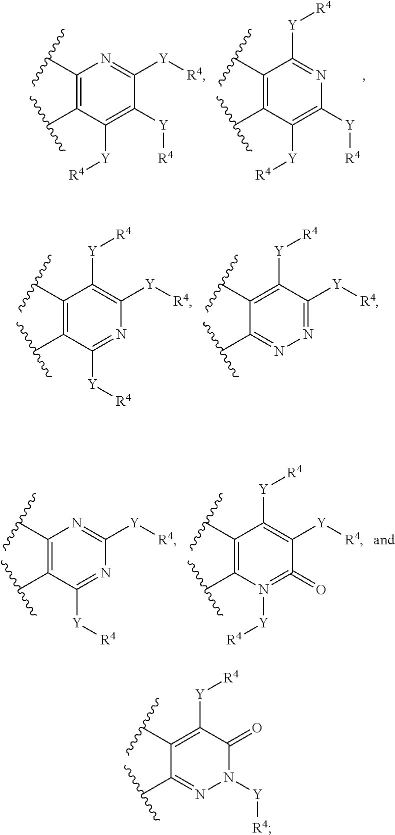

wherein each Y is independently selected from the group consisting of direct bond, O, S, S(═O), S(═O)₂, S(═O)(═NR⁸), C(═O), C(═O)O,

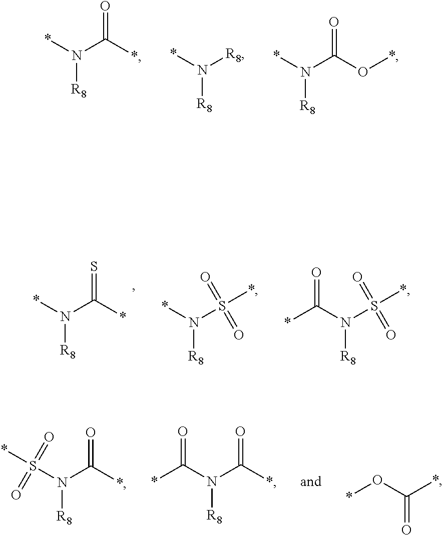

wherein each R⁸ is independently —H or substituted or unsubstituted C₁ to C₆ alkyl group, or C₃ to C₂₀ cycloalkyl group; and each R⁴ is independently selected from the group consisting of H, halide, CN, NO₂, substituted or unsubstituted C₁ to C₆ alkyl group, substituted or unsubstituted C₂ to C₄ alkenyl group, substituted or unsubstituted C₂ to C₄ alkynyl group, substituted or unsubstituted C₁ to C₆ alkoxyl group, substituted or unsubstituted C₁ to C₆ arylalkyl group, substituted or unsubstituted C₁ to C₆ hydroxyalkyl group, substituted or unsubstituted C₁ to C₆ alkoxyalkyl group, substituted or unsubstituted C₃ to C₂₀ cycloalkyl group, substituted or unsubstituted C₃ to C₁₈ aryl group, substituted or unsubstituted C₁ to C₆ cycloalkylalkyl group, substituted or unsubstituted C₁ to C₂₀ heteroaryl group, substituted or unsubstituted C₁ to C₂₀ heterocyclyl group, substituted or unsubstituted C₁ to C₆ heterocyclylalkyl group, substituted or unsubstituted C₁ to C₆ heteroarylalkylgroup, and —N(R⁷)₂.

[3]. The compound of [1] and [2], wherein the compound is selected from the group consisting of:

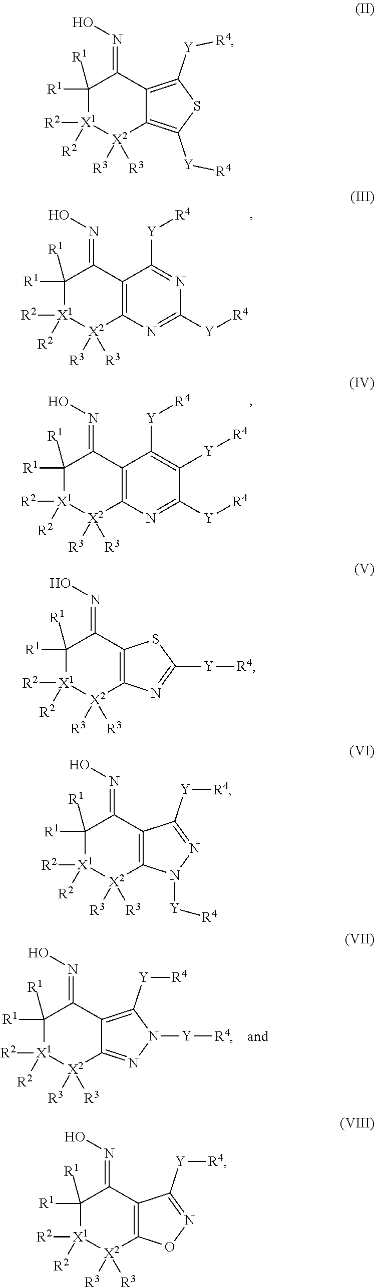

[4]. The compound of [1-3], wherein the compound is selected from the group consisting of:

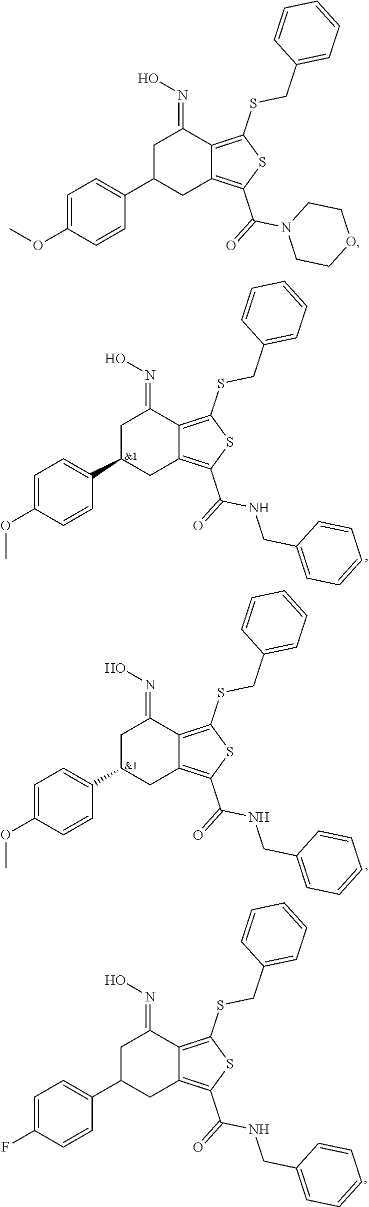

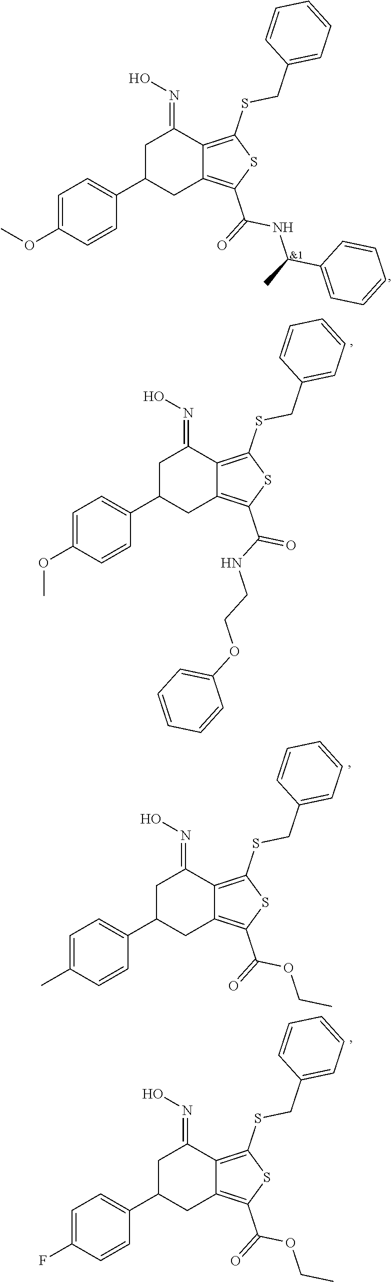

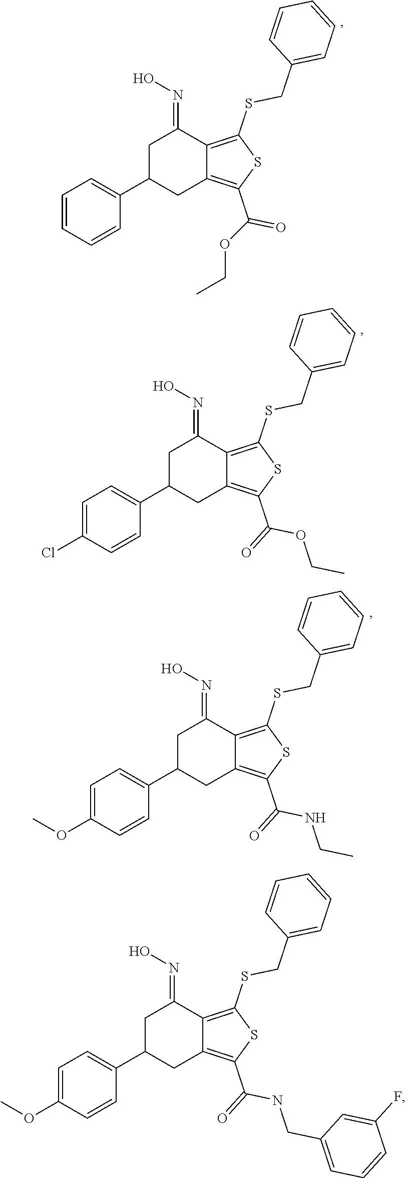

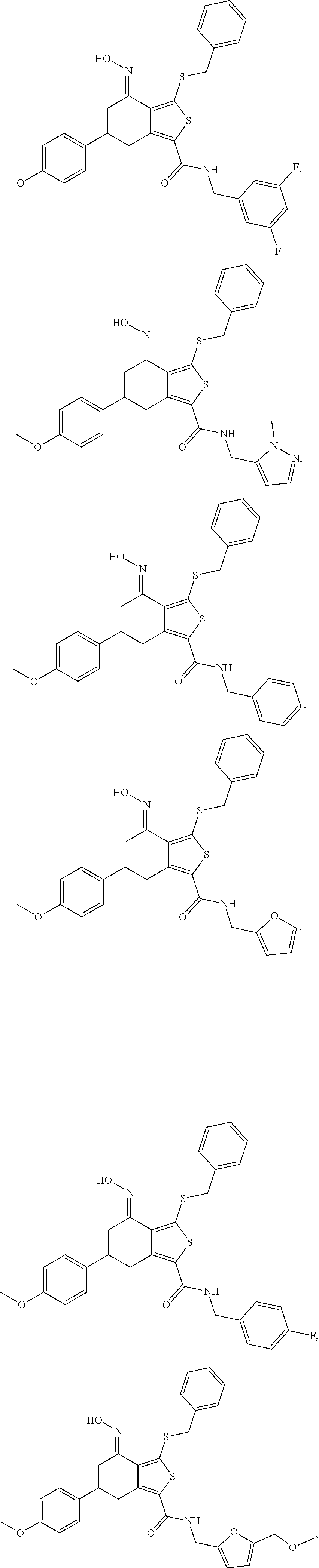

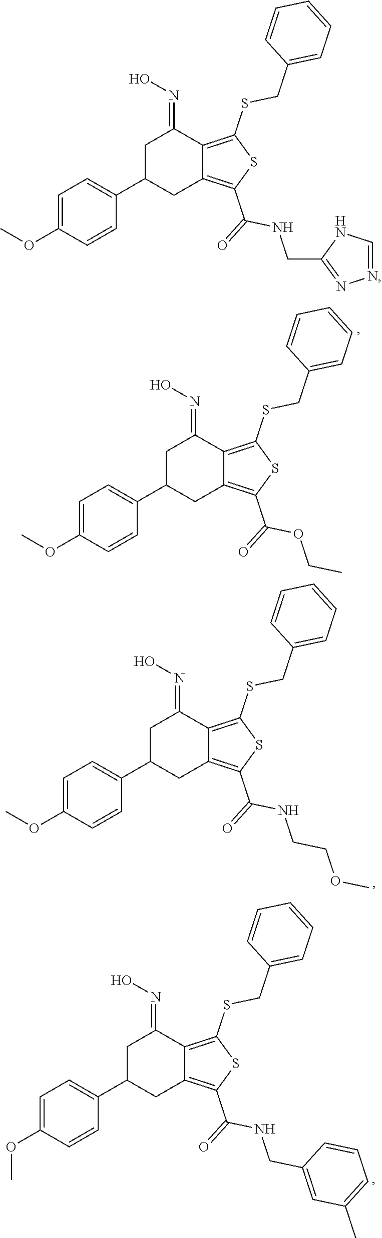

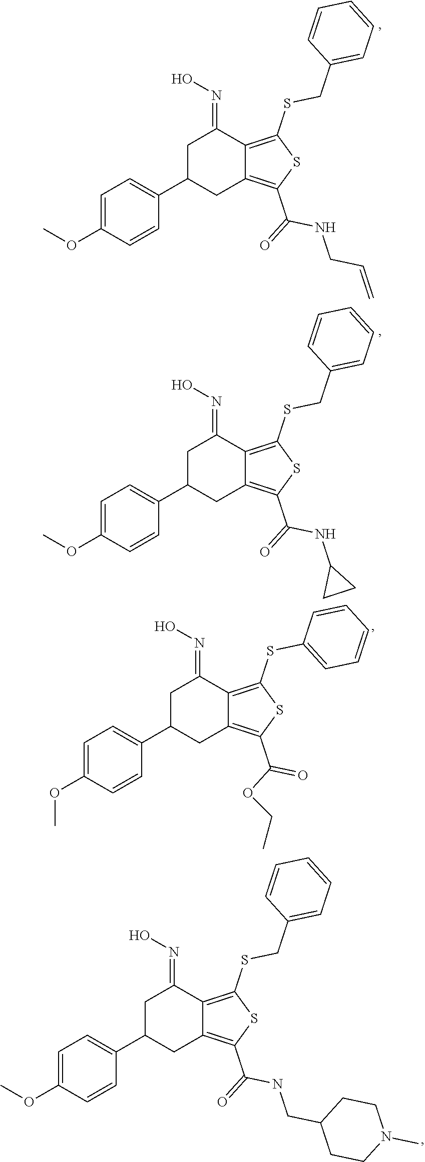

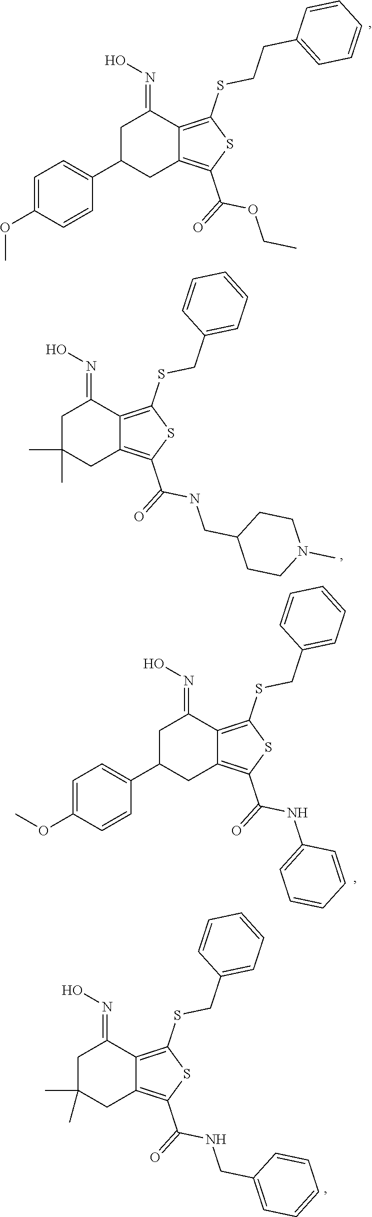

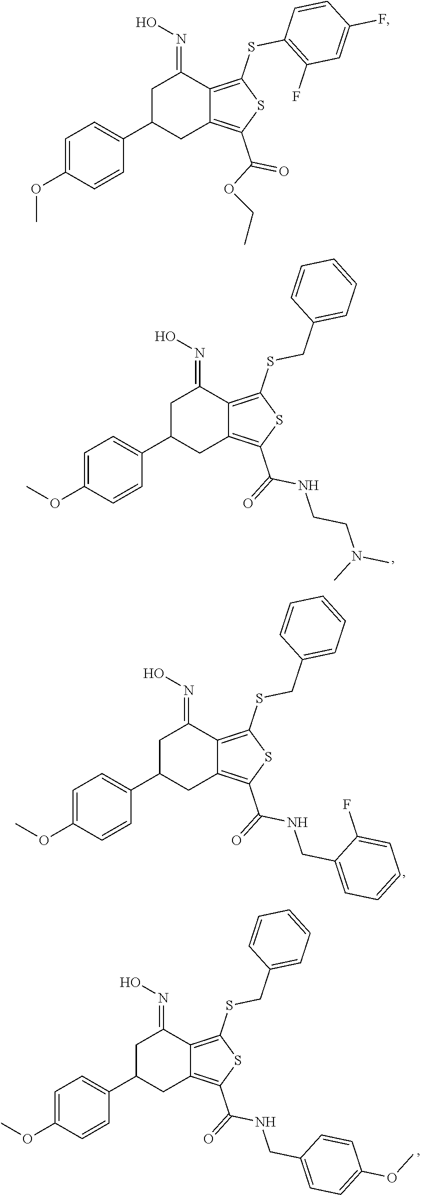

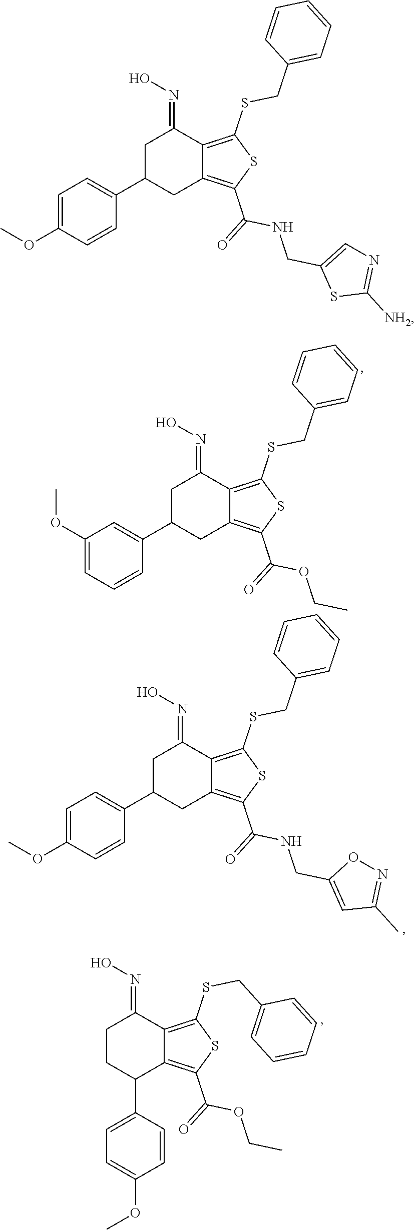

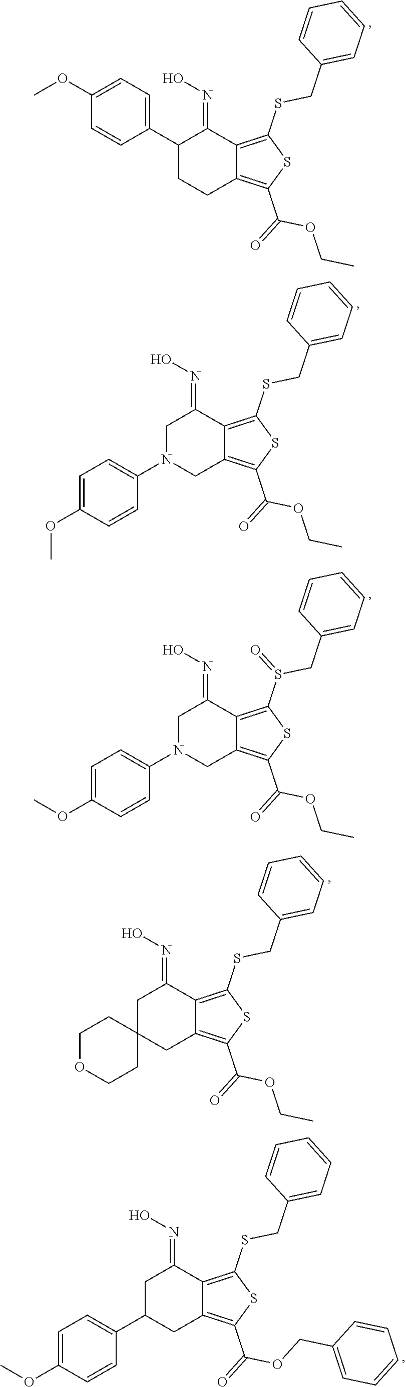

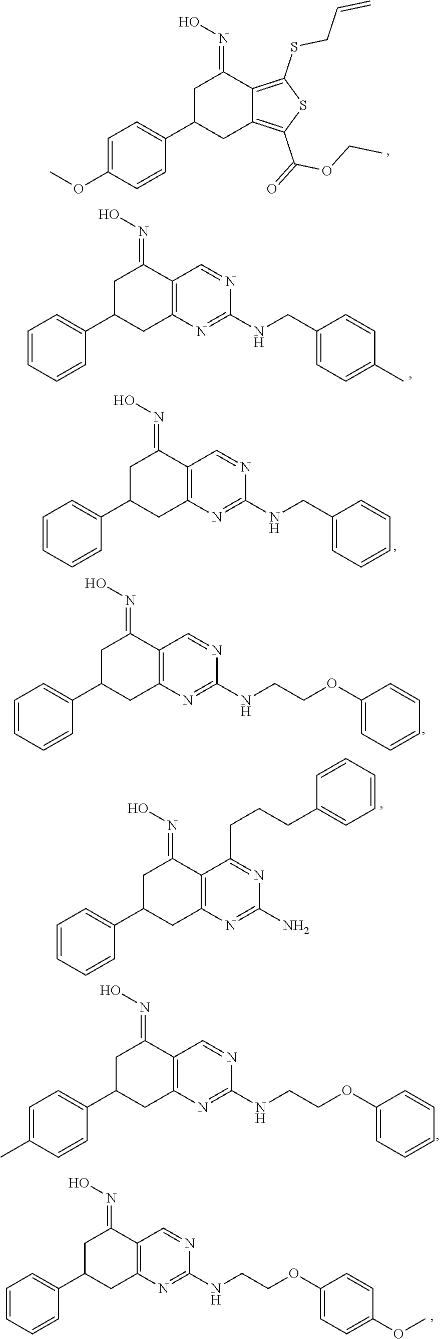

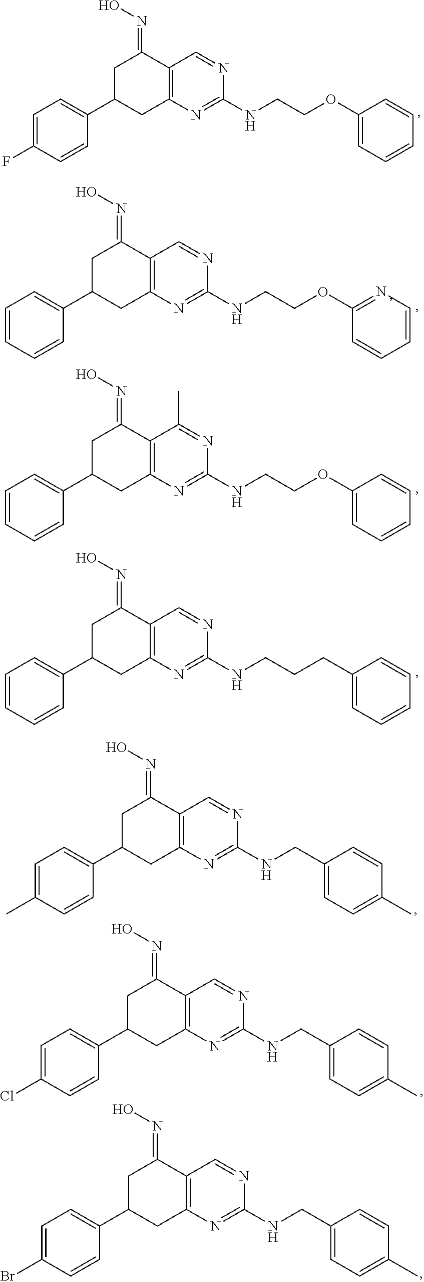

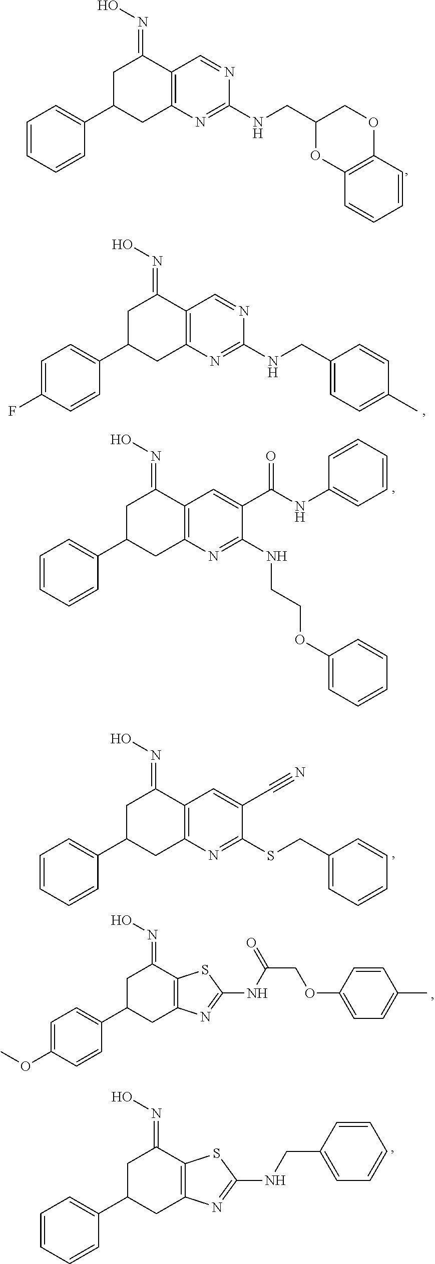

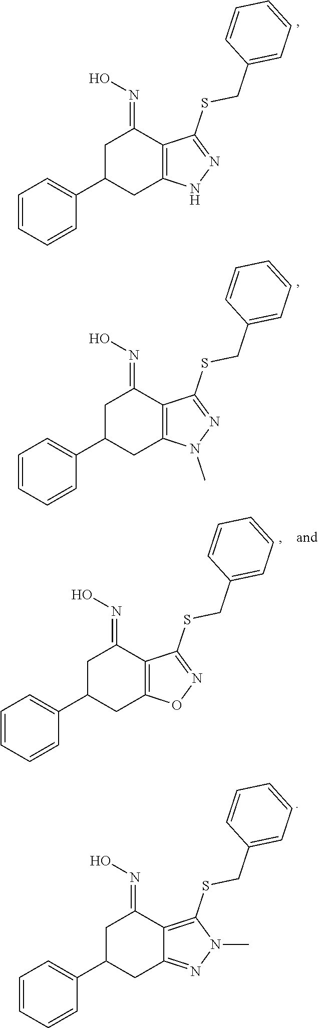

Bisphosphonates:

Non-nitrogen-containing bisphosphonates: Etidronate (Didronel), Clodronate (Bonefos, Loron), Tiludronate (Skelid). The non-nitrogenous bisphosphonates (disphosphonates) are metabolized in the cell to compounds that replace the terminal pyrophosphate moiety of ATP, forming a non-functional molecule that competes with adenosine triphosphate (ATP) in the cellular energy metabolism. The macrophage initiates apoptosis and dies. Nitrogen-containing bisphosphonates: Pamidronate (APD, Aredia), Neridronate (Nerixia), Olpadronate, Alendronate (Fosamax), Ibandronate (Boniva), Risedronate (Actonel), Zoledronate (Zometa, Aclasta). Nitrogenous bisphosphonates block the enzyme farnesyl diphosphate synthase (FPPS) in the HMG-CoA reductase pathway. Bisphosphonates that contain isoprene chains at the R1 or R2 position can impart specificity for inhibition of GGPS1

A Macrolide Drug.

Macrolides are a class of antibiotics found in streptomycetes. They are natural lactones with a large ring, consisting of 14 to 20 atoms. Macrolides bind to the 50S subunit of the bacterial ribosome and inhibit ribosomal translocation, leading to inhibition of bacterial protein synthesis. Their action is primarily bacteriostatic but may be bactericidal at high concentrations, or depending on the type of microorganism. Macrolides mainly affect gram-positive cocci and intracellular pathogens such as mycoplasma, chlamydia, and legionella. A few examples of macrolide drugs include but not limited to Azithromycin, Clarithromycin, Erythromycin, Fidaxomicin, Telithromycin, Carbomycin A, Josamycin, Kitasamycin, Midecamycin/midecamycin acetate, Oleandomycin, Solithromycin, Spiramycin, Troleandomycin, Tylosin/tylocine and Roxithromycin.

Sirolimus (INN/USAN), also known as rapamycin, is a macrolide (one of a group of drugs containing a macrolide ring) produced by the bacterium Streptomyces hygroscopicus. Analogs include everolimus or temsirolimus Sirolimus inhibits IL-2 and other cytokines receptor-dependent signal transduction mechanisms, via action on mTOR. The mode of action of sirolimus is to bind the cytosolic protein FK-binding protein 12 (FKBP12) in a manner similar to tacrolimus. Unlike the tacrolimus-FKBP12 complex, which inhibits calcineurin (PP2B), the sirolimus-FKBP12 complex inhibits the mTOR (mechanistic (formerly mammalian) Target Of Rapamycin, rapamycin being another name for sirolimus) pathway by directly binding to mTOR Complex 1 (mTORC1).

A Prodrug useful according to the invention is one which is activated following cleavage by an enzyme, for example β-galactosidase, arginase, lysomal enzymes, aldehyde oxidase, aminoacid oxidase, cytochrome P450 reductase, DT-Diaphorase, Tyrosinase, class 2 transferases, thymidine synthase, thymidine phosphorylase, glutathione-S-transferase, deoxycytidine kinase, class 3 hydrolases, carboxylesterase, alkaline phosphatase, β-glucuronidase, class 4 lyases, nitroreductase, carboxy peptidase, penicillin amidase, β-lactamase, cytosine deminase and methionine γ-lysase.

A prodrug is a compound that, upon administration, must undergo chemical conversion before becoming an active pharmacological agent (drug). The chemical conversion of a prodrug to an active drug can entail native metabolic processes, or be carried out by processes present in the patient, or the conversion could result from the action of a pharmacologic agent, or by any other chemical conversion.

A prodrug should have toxicity that is substantially less than that of the active chemical agent, say, for example, about ten-fold less toxicity. For essentially all drug structure families, most structures will not have therapeutically effective activity. Therefore, inactivation of a drug can be accomplished by, for example, conjugation of a six-carbon saccharide to most drugs. Such a conjugated molecule would be efficacious as a prodrug if an enzyme activity can be identified that cleaves the conjugated saccharide and restores drug activity.

Choice of a substrate for synthesizing a carbohydrate-inactivated prodrug would focus on identifying drug structures that have desired biological activity, and especially those drugs which are strongly toxic to SAM cells. A prodrug substrate should also have available functional groups that allow conjugation of inactivating carbohydrate groups. Because the specific target for many drugs is known, along with detailed structures of drugs and their target molecules, such information is highly useful for choosing functional groups to be subjected carbohydrate conjugation with the expectation of reversibly inactivating a drug.

Prodrugs are generated from active drug substrates by conjugation of a carbohydrate moiety to one or more active pharmacophores of an active drug via either direct glycosylation or by means of intermediate labile linkers. The carbohydrates include, but are not limited to, mono-di-, tri-, or oligosaccharides.

Duocarmycin, Adozelesin, Bizelesin, Carzelesin, Doxorubicin, daunorubicin are some examples of cytotoxic drugs that can be administered as prodrugs tethered with galactosides that can be cleaved by β-galactosidases to activate the prodrug. Anthracyclines are a class of drugs currently used to treat several types of cancer. Anthracyclines can cause cancer cell death by binding to proteasomes and cancer cell DNA.

Anthracyclines an also interfere with topoisomerase function, causing accumulation of cancer cell DNA damage. Other useful prodrugs and enzymes that transform prodrugs into therapeutically active molecules include, for example, gal-DNC4 (N-[(4″R,S)-4″-ethoxy-4″-(1′″-O-β-D-galactopyranosyl)butyl]daunorubicin) (transformative enzymes include: β-galactocidase), nucleoside or amino acid analogs such as 5-fluorocytosine (transformative enzymes include: cytosine deaminase), polymerase inhibitors such as Poly-ADP (adenosine diphosphate ribose) ribose polymerase-1 inhibitors (transformative enzymes include: glutathione (GSH), glutathione S-transferase P1 (GSTP1)), CNOB (6-chloro-9-nitro-5-oxo-5H-benzo(a)phenoxazine) (transformative enzymes include: E. coli nitroreductase and its alternative form ChrR6), ganciclovir (transformative enzymes include: herpes simplex 1 virus thymidine kinase), nitrogen mustard 1 glutamates, such as CMDA (4 [(2-chloroethyl)(2-mesyloxyethyl)amino]benzoyl 1-glutamic acid) (transformative enzymes include: carboxypeptidase G2), 6-methylpurine deoxyriboside (transformative enzymes include: purine nucleoside phosphorylase (PNP)), Irinotecan (CPT 11), (5-[aziridin-1-yl]-2,4-dinitrobenzamide) (CB1954) (transformative enzymes include: nitrogen reductase), doxorubicin prodrugs (transformative enzymes include: penicillin-V amidase), 5-fluorocytosine (transformative enzymes include: cytosine-deaminase (CD)), and cyclophosphamide (transformative enzymes include: cytochrome P450 (CYP450))

Additional examples of drugs are paclitaxel, cisplatin, geldanamycin, suberanilohydroxamic acid (suberoyl+anilide+hydroxamic acid abbreviated as SAHA), 9-aminocampthotecin, aniline-mustard, epirubicine, beta-naphthol, nitrogen mustard, indolocarbazole, and rapamycin.

Pharmacological inhibitors of NF-kB, which can selectively repress the secretory phenotype in SAMs, such as inhibitors including but not limited to: recombinant protein super-repressor IkBa, Because certain transcription factors are upregulated in SAMs, such as NFκB, the SAM phenotype can be modulated by exposing the cells to an antagonist that directly reduces the expression or activity of such transcription factors. An agent acts “directly” when the agent (either alone or in combination with one or more other agents) itself specifically modulates the expression or activity of a target molecule, for example, a transcription factor described herein, at the level of the expression of the gene encoding the target molecule or the gene product. As a result, such agents can inhibit the activity or production of NFκB will reduce inflammation associated with the SAM phenotype Agents that inhibit the expression or activity of a transcription factor such as NFκB include, but are not limited to, nucleic acids, polypeptides, and small molecule drugs (e.g., small molecules having a molecular weight of less than 1 kDa). Additionally, the agent may be a metabolite, a carbohydrate, a lipid, or any other molecule that binds or interacts with a gene product of one or more of the foregoing transcription factors.

NF-κB is a heterodimeric protein consisting of a 50 kD subunit (p50) and a 65 kD subunit (p65). The cDNAs for p50 and p65 have been cloned and have been shown to be homologous over a region of 300 amino acids. NF-κB has been speculated as being involved in the immune system processes. This is summarized in, for example, the paper by Baeueurle P. A. and Henkel T. (Annual Reviews in Immunology, 1994, Vol. 12, pages 141-179).

The activation of the transcription factor NF-κB like proteins results from post-translational modification permitting translocation of the preformed transcription factor from the cytoplasm to the nucleus. This translocation is controlled by the phosphorylation and degradation of an inhibitor protein called IκB, which forms a complex with NF-κB, and thereby holds it in the cytoplasm. Stimulation of the cell by appropriate signals leads to modification of IκB which in turn results in its dissociation and/or degradation from NF-κB.

Binding of the IκB protein to NF-κB masks the nuclear localization signal (NLS) of NF-κB. Upon stimulation of the cell with specific agents, which depend on the cell type and stage of cell development, IκB is modified in a way that disables binding to NF-κB, leading to dissociation of NF-κB from IκB.

Preferably in one embodiment, the NF-κB inhibitor is an inhibitor of proteolysis, for example a proteosome inhibitor. In one embodiment preferably the inhibitor is an IκB, especially IκBα is described in, for example, paper by Makarov, Gene Therapy, 1997, Vol. 4, pages 846-852, and in PCT/GB98/02753. Other inhibitors of NF-κB include antisense cDNA or oligonucleotides encoding for any of the known NF-κB subunits, e.g. p50, p65, Rel B. Bondeson et al (1999) Proc. Natl. Acad. Sci. USA 96, 5668 describes an IκB-encoding adenovirus.

In one exemplary embodiment the inhibitor may be PSI, available from Calbiochem. This is known as an inhibitor of proteosomes (Traechner, et al., EMBO J. (1994), Vol. 13, pages 5433-41; Griscavage, et al., PNAS (1996), Vol. 93, pages 3308-12; Bondeson, et al., J Immunol. (1999), Vol. 162, pages 2939-45).

In another exemplary embodiment the inhibitor of NF-kB include ALLN (Jobin, et al., Hepatology (1998), Vol. 27, pages 1285-95); Lactacystin (Delic, et al. (1998), Vol 77, pages 1103-07); C-LFF and Calpain Inhibitors (Neauparfant and Hiscott, Cytokine & Growth Factor Reviews (1996), Vol. 7 pages 175-190); or CVT-134 (Lum, et al., Biochem. Pharmacol (1998), Vol. 55, pages 1391-97) may also be used as inhibitors. Other inhibitors include: Caffeic acid phenethyl ester (Natarajan, et al., PNAS (1992), Vol. 93 pages 9090-95); Pyrrolidine dithiocarbonate (Schreck, et al., J. Exp. Med. (1992), Vol. 175, pages 1181-94); Lovastatin (Guijarro, et al., Nephrol Dial Transplant (1996), Vol. 11, pages 990-996); Aselastine HCL (Yoneda, Japan. J. Pharmacol. (1997), Vol. 73, pages 145-153); Tepaxalin (Kazmi, et al., J Cell. Biochem. (1995), Vol. 57, pages 299-310); (−)-epi gallocatechin-3-gallate (Lin & Lin, Mol. Pharmacol. (1997), Vol. 52, pages 465-472); deoxyspergualin (Tapper, et al., J Immunol. (1995), Vol. 155, pages 2427-36); Phenyl-N-tert-butylnitrone (Kotake, et al., Biochem. Biophys Acta (1998), Vol. 1446, pages 77-84; Quercutin (Sato, et al., J Rheumatol. (1997), Vol 24, pages 1680-84); Cucumin (Chan, Biochem, Pharmacol. (1998), Vol. 55, pages 965-973); or E3330 (Goto, et al., Mol. Pharmacol (1996), Vol. 49, pages 860-873).

The inhibitor may also be a ribozyme which selectively destroy mRNA encoding NF-κB, or an antisense molecule which prevents transcription of NF-κB or an antibody or antibody-like molecule which blocks NF-κB action. These inhibitors are described in more detail below. It will be appreciated that inhibitors of antigen presenting cells (APC), such as dendritic cells (DC) function, may also comprise ribozymes or antisense molecules or antibodies or antibody-like molecules which, for example, inhibit intracellular signaling within the APC.

Preferably the inhibitor is encoded by a nucleic acid sequence, for example within a vector, such as an adenovirus. The nucleic acid sequence encoding the inhibitor is preferably operatively linked to regulatory elements necessary for expression of the sequence. Such vectors may be used for gene therapy to enable the nucleic acid sequence encoding the inhibitor to be inserted into the body of a mammal. Methods of gene therapy, such as by using an adenovirus, are known in the art.

Preferably the inhibitor is a pharmaceutical compound, a few representative examples are tepoxalin, cycloepoxydon, (+)-cycloepoxydon, (−)-cyclo-epoxydon, sodium diethyldithiocarbamate, gliotoxixon, 5-deoxy-Δ.12,14-prostaglandin J2 (see Proc. Natl. Acad. Sci. USA, 97(9), 4844-4849 (2000)), cyclopentenone prostaglandin A1 (PGA1), (see Proc. Natl. Acad. Sci. USA, 94, 746-750 (1997)), cyclopentabenzofurans as described in WO-00/08007, and bortezomib. Other NF-κB inhibitors can be learned from literature and compounds can be tested for NF-κB inhibiting activity using assays known in the art. See for instance Proc. Natl. Acad. Sci. USA, 94, 746-750 (1997).

Furthermore it is contemplated that in the case of a cocktail of inhibitors it is possible that the inhibitors can include a combination of one or more nucleic acids (one or more of which may be directed to a particular transcription factor gene or may be directed to different transcription factor genes), one or more proteins, and/or one or more small molecules.

Exemplary nucleic acid-based modulators include, but are not limited to, RNAs, DNAs, and PNAs. Exemplary RNAs include, for example, antisense RNA, short interfering RNA (siRNA), short hairpin RNA (shRNA), and microRNA (miRNA). In addition, it is contemplated that RNA and DNA aptamers can be used in the practice of the invention.

In certain embodiments, the agent is a siRNA specific to one or more genes encoding a transcription factor. Exemplary synthetic siRNAs include 21 nucleotide RNAs chemically synthesized using methods known in the art (e.g., Expedite RNA phophoramidites and thymidine phosphoramidite (Proligo, Germany)). Synthetic oligonucleotides preferably are deprotected and gel-purified using methods known in the art (see, e.g., Elbashir et al. (2001) GENES DEV. 15: 188-200). Longer RNAs may be transcribed from promoters, such as T7 RNA polymerase promoters, known in the art. A single RNA target, placed in both possible orientations downstream of an in vitro promoter, can transcribe both strands of the target to create a dsRNA oligonucleotide of the desired target sequence.

The antisense nucleic acid may be produced intracellularly by transcription from an exogenous sequence. For example, a vector or a portion thereof is transcribed, producing an antisense nucleic acid (RNA) of the target gene. Such a vector would contain a sequence encoding a portion of the target antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others know in the art, used for replication and expression in vertebrate cells. Expression of the coding sequence, or fragments thereof, can be by any promoter known in the art to act in vertebrate, preferably human cells. Such promoters can be inducible or constitutive. Such promoters include, but are not limited to, the SV40 early promoter region (Bernoist and Chambon, Nature 29:304-310 (1981), the promoter contained in the 3′ long terminal repeat of Rous sarcoma virus (Yamamoto et al., Cell 22:787-797 (1980), the herpes thymidine promoter (Wagner et al., Proc. Natl. Acad. Sci. U.S.A. 78:1441-1445 (1981), the regulatory sequences of the metallothionein gene (Brinster et al., Nature 296:39-42 (1982)).

The resulting siRNAs can be delivered as multiple siRNAs with each siRNA targeting one or more genes. Alternatively, multiple siRNAs can be used to target a target gene (see, for example, U.S. Patent Application Publication No. US2005/0197313, which describes a system for delivering multiple siRNAs to target multiple versions of the same gene). Alternatively, a single siRNA can be used to target multiple genes.

The following sections provide exemplary siRNAs that can be used to reduce the expression of certain transcription factors, including NFκB p65 siRNA (m) sc29411, NFκB p65 (m)-PR, NFκB p65 shRNA Plasmid (m), NFκB p65 shRNA (m) Lentiviral Particles are available from Santa Cruz Biotech, Santa Cruz, Calif.

Inhibitors of Apoptosis

Inhibitors of apoptosis can be initiated through one of two pathways. In the intrinsic pathway the cell kills itself because it senses cell stress, while in the extrinsic pathway the cell kills itself because of signals from other cells. Both pathways induce cell death by activating caspases, which are proteases, or enzymes that degrade proteins. The two pathways both activate initiator caspases, which then activate executioner caspases, which then kill the cell by degrading proteins indiscriminately. Some of common examples of apoptosis inhibitors known in art are discussed below. Rothe et al. (1994) Cell 78, 681-692, report the existence of tumor necrosis factor (TNF) receptor associated proteins which co-immunoprecipitate with a TNF receptor; see also Rothe, et al., pending U.S. patent application Ser. No. 08/446,915 now U.S. Pat. No. 5,741,677. Roy, et al. (1995) Cell 80, 167-178 discloses the gene for a human neuronal apoptosis inhibitory protein. Birnbaum et al. (1994) J Virol 68, 2521-2528 discloses an inhibitor of apoptosis (iap) gene, Op-iap from the Orgyia pseudotsugata nuclear polyhedrosis virus (OpMNPV) with sequence similarity to two other viral genes: Cp-iap derived from Cydia pomonella granulosis virus (CpGV), and iap derived from the Autographa californica nuclear polyhedrosis virus (AcMNPV). Clem and Miller (1994), in Apoptosis II: The Molecular Basis of Apoptosis in Disease, pp 89-110, Cold Spring Harbor Laboratory Press, provides a recent review of apoptosis regulation by insect viruses.

U.S. Pat. No. 6,821,736 describe methods and compositions relating to human cellular inhibitor of apoptosis proteins (c-IAP1/2). The proteins provide a c-IAP specific function, with preferred proteins being capable of modulating the induction of apoptosis; for example, by binding a human tumor necrosis factor receptor associated factor (TRAF).

U.S. Pat. No. 7,816,352 describe pyridinium and quinolinium salts and related compounds have an ability to inhibit glucocorticoid-induced and radiation-induced apoptosis in murine thymocytes. These compounds show a chemoprotective effect and can prevent cell death caused by trauma, ischemia, and side effects of cancer therapy.

U.S. Pat. No. 6,087,395 describe an apoptosis inhibitor containing a 15R-isocarbacyclin derivative or a 15-deoxy-isocarbacyclin derivative which has been known as a ligand specific to prostacyclin receptors of the central nervous system. The inhibitor has an excellent inhibiting action to apoptosis of nerve cells

Inhibitors of CDK8/CDK19

Inhibitors of CDK8/CDK19 may be used according to the invention to block the secretory phenotype in SAMs.

CDK8, along with its closely related isoform CDK 19, is an oncogenic transcription-regulating kinase. The role of CDK8 in cancer is due to its unique function as a regulator of several transcriptional programs involved in carcinogenesis. CDK8 has been identified as an oncogene in melanoma and colon cancer. Higher expression of CDK8 has been associated with worse prognosis in colon cancer. The known cancer-relevant activities of CDK8 include positive regulation of Wnt/β-catenin pathway, growth factor-induced transcription and TGFP signaling. CDK8 was also shown to maintain the pluripotent phenotype of embryonic stem cells and has been associated with the cancer stem cell phenotype. DNA-damaging chemotherapeutic drugs induce TNFa, an activator of the transcription factor NFKB, in endothelial cells and in other cancer-associated stromal elements. Stroma-derived TNFa acts on tumor cells, where it induces NFKB-mediated production of related tumor-promoting cytokines CXCL1 and CXCL2.

CXCL1/2 attracts myeloid cells to the tumor, by binding to CXCR2 receptor on the myeloid cell surface. Myeloid cells then secrete small calcium-binding proteins. PCT/US 12/55064 teaches that CDK8/19 inhibitors inhibit induction of transcription factor NFKB, which mediates the production of multiple tumor-supporting proteins and inflammatory cytokines, and that CDK8/19 inhibitors in particular inhibit NFKB-mediated induction of CXCL1 and CXCL2. US Patent Publication 20120071477 teaches that CDK8/19 inhibitors also prevent the induction of paracrine tumor-promoting activities by DNA damage in normal fibroblasts, and inhibit HIV replication and β-catenin signaling.

CDK8 is also involved in secretory activity of senescent cells in response to chemotherapy. Selective inhibition of CDK8 and CDK19 repressed expression of certain cytokines and growth factors which are released in response to chemotherapy treatment and stimulate tumor growth [Porter et al., PNAS (34) 109: 13799-13804, (2012)]. CDK8 is involved in expression of proinflammatory cytokines such as TNFα and IL6, upon stimulation with exogenous and endogenous factors, such as LPS and other TLR agonists.

The CDK8 and or CDK19 inhibitor may be an antibody, binding polypeptide, small molecule, or polynucleotide. In some embodiments, the CDK8 and or CDK19 inhibitor is an antibody, a small molecule, the small molecule may be a small molecule kinase inhibitor, the small molecule kinase inhibitor may be selected from the group consisting of flavopiridol, sorafenib, staurosporine, cortistatin, cortistatin A, and/or a steroidal alkaloid or derivative thereof.

The CDK8 and or CDK19 inhibitor may induce cell cycle arrest or is capable of promoting differentiation. The CDK8/CDK19 inhibitor may be capable of promoting a change in cell fate and promoting differentiation, indicated by reduced expression of one or more CDK8/CDK19-induced biomarkers of the CDK8/19 gene signature. Other examples of CDK8 inhibitors are described in US20150274726, U.S. Pat. Nos. 9,321,737, 8,685,980 and US 20140309224.

A polynucleotide can serve as a CDK8 antagonist as disclosed in US 2004/0180848. The polynucleotide may be an antisense nucleic acid and/or a ribozyme. The antisense nucleic acids comprise a sequence complementary to at least a portion of an RNA transcript of a CDK8 or CDK19 gene. However, absolute complementarity, although preferred, is not required.

Polynucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. See generally, Wagner, R., 1994, Nature 372:333-335. Thus, oligonucleotides complementary to either the 5′- or 3′-non-translated, non-coding regions of the CDK8 or CDK19 gene, could be used in an antisense approach to inhibit translation of endogenous CDK8 or CDK19 mRNA. Polynucleotides complementary to the 5′ untranslated region of the mRNA should include the complement of the AUG start codon. Whether designed to hybridize to the 5′-, 3′- or coding region of CDK8 or CDK19 mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

The CDK8 or CDK19 antisense nucleic acid of the invention may be produced intracellularly by transcription from an exogenous sequence. For example, a vector or a portion thereof is transcribed, producing an antisense nucleic acid (RNA) of the CDK8 or CDK19 gene. Such a vector would contain a sequence encoding the CDK8 or CDK19 antisense nucleic acid.

Antigen-Specific Binding Polypeptides

Antigen-specific binding polypeptides which bind one or more of CD19, CD11b, and/or F4/80. Macrophages are identifiable by one or more cell surface markers, such as CD19, CD11b and F4/80, as well as Itgam, F4/80 (Emr1), Cd63, Cd68, Csf1r, Lgals3, Itgax, Ly6g, Ly6c1, Il4ra, Cd163, Cd206, Mgl1, Mgl2 (Murray P. J., & Wynn, T. A. (2011) Protective and pathogenic functions of macrophage subsets. Nature Reviews Immunology, 11(11), 723-737). Therefore a polypeptide which binds these cell surface markers will bind a macrophage. Where the macrophage-specific polypeptide is conjugated to a toxin, upon binding of the polypeptide to the macrophage, the macrophage will ingest the toxin and this will kill the macrophage. Production of antigen-specific binding polypeptides are well-known in the art. According to the invention, an antigen-specific binding polypeptide, e.g., an antibody or a fragment of an antibody, a single variable domain, a camelid or VHH, a scaffold having an antigen-binding capability, can be used to target SAMs. It is understood that each antibody directed to a stemness inducing or maintaining transcription factor can be an intact antibody, for example, a monoclonal antibody, an antigen binding fragment of an antibody, or a biosynthetic antibody binding site. Antibody fragments include Fab, Fab′, (Fab′).sub.2 or Fv fragments. The antibodies and antibody fragments can be produced using conventional techniques known in the art. A number of biosynthetic antibody binding sites are known in the art and include, for example, single Fv or sFv molecules, described, for example, in U.S. Pat. Nos. 5,091,513, 5,132,405, and 5,476,786. Other biosynthetic antibody binding sites include bispecific or bifunctional binding proteins, for example, bispecific or bifunctional antibodies, which are antibodies or antibody fragments that bind at least two different antigens. Methods for making bispecific antibodies are known in art and, include, for example, by fusing hybridomas or by linking Fab′ fragments. See, e.g., Songsivilai et al. (1990) CLIN. EXP. IMMUNOL. 79: 315-325; Kostelny et al. (1992) J. IMMUNOL. 148: 1547-1553.

For example, antibodies directed to a molecule present or expressed by SAMs can be conjugated with isotopes or toxins to form conjugates having the ability to kill, or to facilitate the killing of, SAMs or as having the ability to delay, or reduce the likelihood of age-related disorders, and/or maximize healthy lifespan. The cytotoxic agent can be, for example, a nitrogen mustard, gemcitabine, an ethylenimine derivative, an alkyl sulfonate, a nitrosourea, a triazene, a folic acid analog, an anthracycline, a taxane, SN-38, a COX-2 inhibitor, a pyrimidine analog, a purine analog, an antibiotic, an enzyme, an enzyme inhibitor, an epipodophyllotoxin, a platinum coordination complex, a vinca alkaloid, a substituted urea, a methyl hydrazine derivative, an adrenocortical suppressant, a hormone antagonist, taxol, camptothecin, doxorubicin, an alkylating agent, an antimitotic, an antiangiogenic agent, an apoptotic agent, and methotrexate. In an embodiment, the antibody affinity has an EC50 which is at least 10.sup.−6 M, at least 10.sup.−7 M, at least 10.sup.−8 M, or at least 10.sup.−9 M. Antibodies are then tested by ELISA for binding to a selected epitope, with a preferred binding agent preferably having an EC50 by this test that is at least better than 10.sup.−9M.

For use in the methods of the present invention, the antibodies and their binding fragments can be conjugated with other agents that are useful for the intended purpose, e.g., either diagnostic use or medical treatment. Agents appropriate for treating disease include cytotoxic agents or toxins that include chemotherapeutics and radiotherapeutics. For diagnostic purposes, appropriate agents are detectable labels that include radioisotopes or fluorescent markers for whole body imaging, and radioisotopes, enzymes, fluorescent labels and the like for sample testing.

For diagnostics, the detectable labels can be any of the various types used currently in the field of in vitro diagnostics, including particulate labels including biotin/streptavidin, metal sols such as colloidal gold, isotopes such as 1125 or Tc99 presented for instance with a peptidic chelating agent of the N2S2, N3S or N4 type, chromophores including fluorescent markers such as FITC and PE, luminescent markers, phosphorescent markers and the like, as well as enzyme labels that convert a given substrate to a detectable marker, and polynucleotide tags that are revealed following amplification such as by polymerase chain reaction. Suitable enzyme labels include horseradish peroxidase, alkaline phosphatase and the like. For instance, the label can be the enzyme alkaline phosphatase, detected by measuring the presence or formation of chemiluminescence following conversion of 1,2 dioxetane substrates such as adamantyl methoxy phosphoryloxy phenyl dioxetane (AMPPD), disodium 3-(4-(methoxyspiro{1,2-dioxetane-3,2′-(5′-chloro)tricyclo{3.3.1.13,7}decan}-4-yl)phenyl phosphate (CSPD), as well as CDP and CDP-Star® or other luminescent substrates well-known to those in the art, for example the chelates of suitable lanthanides such as Terbium(III) and Europium(III). The detection means is determined by the chosen label. Appearance of the label or its reaction products can be achieved using the naked eye, in the case where the label is particulate and accumulates at appropriate levels, or using instruments such as a spectrophotometer, a luminometer, a fluorimeter, and the like, all in accordance with standard practice.

For therapy, the cytotoxin can be conjugated with the antibody or binding fragment through non-covalent interaction, but more desirably, by covalent linkage either directly or, more preferably, through a suitable linker. In a preferred embodiment, the conjugate comprises a cytotoxin and an antibody. Immunoconjugates of the antibody and cytotoxin are made using a variety of bifunctional protein coupling agents such as N-succinimidyl-3-(2-pyridyldithiol) propionate, iminothiolane, bifunctional derivatives of imidoesters such as dimethyl adipimidate HCl, active esters such as disuccinimidyl suberate, aldehydes such as glutaraldehyde, bis-azido compounds such as bis-(p-diazoniumbenzoyl)-ethylenediamine), diisocyanates such as toluene 2,6-diisocyanate, and bis-active fluorine compounds (such as 1,5-difluoro-2,4-dinitrobenzene). Carbon-14-labeled 1-isothiocyanobenzyl-3-methyldiethylene triaminepentaacetic acid (MX-DTPA) is a chelating agent suitable for conjugation of radionuclide to the antibody.

The cytotoxin component of the immunoconjugate can be a chemotherapeutic agent, a toxin such as an enzymatically active toxin of bacterial, fungal, plant or animal origin such as urease, or fragments thereof, or a small molecule toxin, or a radioactive isotope such as 212Bi, 131I, 131In, 111In, 90Y, and 186Re, or any other agent that acts to inhibit the growth or proliferation of a senescent cell.

Chemotherapeutic Agents

A “chemotherapeutic agent” refers to a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Nicolaou et al., Angew. Chem. Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g., celecoxib or etoricoxib), proteosome inhibitor (e.g., PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors (see definition below); tyrosine kinase inhibitors (see definition below); serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Additional toxins which also serve as chemotherapeutic agents useful in the generation of immunoconjugates include maytansinoids including DM-1 and DM-4, adriamycin, doxorubicin, epirubicin, 5-fluoroouracil, cytosine arabinoside (“Ara-C”), cyclophosphamide, thiotepa, busulfan, cytoxin, taxoids, e.g. paclitaxel, and docetaxel, taxotere, methotrexate, cisplatin, melphalan, vinblastine, bleomycin, etoposide, ifosamide, mitomycin C, mitoxantrone, vincristine, vinorelbine, carboplatin, teniposide, daunomycin, carminomycin, aminopterin, dactinomycin, mitomycins, esperamicins, 5-FU, 6-thioguanine, 6-mercaptopurine, actinomycin D, VP-16, chlorambucil, melphalan, and other related nitrogen mustards. Also included are hormonal agents that act to regulate or inhibit hormone action on tumors such as tamoxifen and onapristone. Toxins and fragments thereof which can be used include diphtheria A chain, nonbonding active fragments of diphtheria toxin, cholera toxin, botulinus toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, sapaonaria, officinalis inhibitor, gelonin, saporin, mitogellin, restrictocin, phenomycin, enomycin, and the tricothcenes. Small molecule toxins include, for example, calicheamicins, maytansinoids including DM-1 and DM-4, palytoxin and CC1065.

In an embodiment, the binding agent, optionally an antibody and/or binding fragment thereof, optionally conjugated to a toxin or a label or the nucleic acid is comprised in a composition. In an embodiment the composition comprises a diluent such as a saline solution for example phosphate buffered saline solution (0.05-1.0%).

Drug Screening.

SAMs can be exposed to a library of test agents individually or in pools to identify those agents or pools of agents having the ability to kill, or to facilitate the killing of, SAMs. Once identified as having the ability to kill, or to facilitate the killing of SAMs the identified agent can be applied to comparable non-SAM cells in comparable concentrations to confirm that the agent has a reduced ability to kill, or to facilitate the killing of non-SAM cells. Those agents having the ability to kill, or to facilitate the killing of, SAMs with a reduced or no ability to kill, or to facilitate the killing of, non-SAMs can be classified as being an agent having the ability to delay, or reduce the likelihood of age-related disorders, and/or maximize healthy lifespan. In some cases, SAMs are obtained from a transgenic mammal provided herein and treated in a manner that results in SAM cell death can be used as positive controls.

In some cases, an agent can be identified as having the ability to kill, or to facilitate the killing of, SAMs or as having the ability to delay, or reduce the likelihood of age-related disorders, and/or maximize healthy lifespan using in vivo techniques. For example, an animal model such as wild-type mice or animals, mice with a p16(Ink4A) promoter driven luciferase gene, as described herein or other mouse or animal models can be used. In such cases, a library of test agents can be administered individually or in pools to the animals (e.g., mice), and the animals (e.g., mice) can be assessed for indications that the test agent is capable of killing, or facilitating the killing of, SAMs or is capable of delaying, or reducing the likelihood of age-related disorders, and/or maximizing healthy lifespan. Indications of SAMcell killing or indications of delayed or reduced likelihood of age-related disorders, and/or indications of maximized healthy lifespan can be detected and assessed as described herein. For example, the ability of an agent to increase the length of lifespan can be assessed comparing treated and untreated mice. Additional screening methods are described in Qi, U.S.20120071349, hereby incorporated by reference.

Dosage, Administration and Pharmaceutical Formulation

The pharmaceutical preparation in some embodiments may be in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents. Preferred pharmaceutical preparations can deliver the compounds of the disclosure in a sustained release formulation.

For a binding agent, composition, or compound according to the invention, the dosage form may optionally be a liquid dosage form. Solutions can be prepared in water suitably mixed with a surfactant such as hydroxypropylcellulose or an emulsifier such as polysorbate. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. Formulations optionally contain excipients including, but not limited to, a buffering agents, an anti-oxidant, a stabilizer, a carrier, a diluent, and an agent for pH adjustment. The pharmaceutical forms suitable for injectable use include sterile aqueous solutions or dispersion and sterile powders for the extemporaneous preparation of sterile injectable solutions or dispersions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl, or benzyl alcohol; alkyl parabens such as methyl or propyl paraben; catechol; resorcinol; cyclohexanol; 3-pentanol; and m-cresol); low molecular weight (less than about 10 residues) polypeptides; proteins such as serum, albumin, gelatin, or immunoglobulins; hydrophilic polymers such as polyvinylpyrrolidone; amino acids such as glycine, glutamine, asparagine, histidine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, PLURONICS or polyethylene glycol (PEG).

In treatment, the dose of agent optionally ranges from about 0.0001 mg/kg to about 100 mg/kg, about 0.01 mg/kg to about 5 mg/kg, about 0.15 mg/kg to about 3 mg/kg, 0.5 mg/kg to about 2 mg/kg and about 1 mg/kg to about 2 mg/kg of the subject's body weight. In other embodiments the dose ranges from about 100 mg/kg to about 5 g/kg, about 500 mg/kg to about 2 mg/kg and about 750 mg/kg to about 1.5 g/kg of the subject's body weight. For example, depending on the type and severity of the disease, about 1 .mu.g/kg to 15 mg/kg (e.g., 0.1-20 mg/kg) of agent is a candidate dosage for administration to the patient, whether, for example, by one or more separate administrations, or by continuous infusion. A typical daily dosage is in the range from about 1 .mu.g/kg to 100 mg/kg or more, depending on the factors mentioned above. For repeated administrations over several days or longer, depending on the condition, the treatment is sustained until a desired suppression of disease symptoms occurs. However, other dosage regimens may be useful. Unit doses can be in the range, for instance of about 5 mg to 500 mg, such as 50 mg, 100 mg, 150 mg, 200 mg, 250 mg and 300 mg. The progress of therapy is monitored by conventional techniques and assays.

Therapeutic use of an agent according to the present invention entails administration, by injection or infusion, to subjects presenting with a disease in which SAMs are targeted.

Indications.

It is anticipated that depletion of endogenous SAMs will have a therapeutic effect and benefit on a variety of aging diseases and conditions, including treatment of cancer in various tissues, including cancers of the lung, prostate, skin, breast, and the like. Such treatment is expected to result in a decrease in the rate of tumor formation directly, and thus indirectly also on the number, size and distribution of responsive cancer cells and tumors.

Depletion of endogenous SAMs also is applicable to treating degenerative disorders that accompany aging. More particularly, SAM depletion is expected to provide improvements in 1) reducing the rate at which adipose tissue is lost, 2) reducing the rate at which muscle fibre diameter is reduced, and 3) reducing the rate at which skin tone deteriorates over time. These effects are likely to be seen more dramatically in aged recipients, i.e. those at an age greater than 50 years, especially those aged greater than 60 years or more, such as 65 years, 70 years and 75 years and greater. Also, candidate recipients include those whose lifestyle imposes age-accelerating effects, including tobacco smokers and users, alcohol and narcotic drug abusers, skin tanning enthusiasts, and the like. These are therefore preferred recipients of the present treatment method.

Particular conditions and diseases that can be treated by the present method include sarcopenia. Sarcopenia is characterized first by a muscle atrophy (a decrease in the size of the muscle), along with a reduction in muscle tissue “quality,” caused by such factors as replacement of muscle fibres with fat, an increase in fibrosis, changes in muscle metabolism, oxidative stress, and degeneration of the neuromuscular junction. Combined, these changes lead to progressive loss of muscle function and frailty.

Other conditions that can be treated by the present method include cataracts, and so-called “signs of aging” such as wrinkling and discoloration of the skin, and overall dermal tone. Treatment by the present method is expected to reduce the rate at which fat and muscle that support skin tone are reduced, so that skin wrinkling also is reduced, delayed or eliminated. As well treatment is expected to have a benefit on the rate at which cataracts form in the eye.

Cell Culture

Primary human neonatal dermal fibroblasts (NDFs; AllCells, LLC) were pooled equally from three separate donors. NDFs were maintained in Dulbecco's modified Eagle Medium (DMEM) with phenol red supplemented with 10% (v/v) FBS (Gibco; Grand Island, N.Y.), 100 units/mL of penicillin, 100 μg/mL of streptomycin and 2 mM L-glutamine, and 1×MEM non-essential amino acids. Cells were cultured in a tissue culture incubator at 37° C. and 5% CO₂. NDFs were maintained at <80% confluency by serial passage after enzymatic dissociation via TrypLE (Thermo Fisher Scientific). GLuc-expressing NDF cells (NDF-GLuc) were puromycin-selected following lentiviral transduction of a PGK1 promoter-driven reporter construct that was synthesized and cloned into pRSIT vector by Genscript (Piscataway, N.Y.). This construct constitutively expresses secreted Gaussia princeps luciferase (“8990” mutant [73]), v5-tagged Histone H3, and puromycin from the same transcript, separated by T2A sequences. NDFs were routinely tested for mycoplasma, and confirmed to be negative using the MycoAlert mycoplasma detection kit (Lonza).

Primary cultures of adipose-derived mesenchymal stromal cells (mAdMSC) were established from the stromal vascular fraction of inguinal adipose tissues of young p16^{LUC} mice, as described [74]. Isolated adherent cultures were maintained in DMEM/F12 medium supplemented with 15% FBS and 1× anti-biotic/anti-mycotic solution (Thermo Fisher Scientific) in a tri-gas tissue culture incubator at 37° C., 5% CO₂ and 3% O₂. Medium was change twice per week, and cell passaging performed when cells reached ˜80-90% confluency. For induction of senescence, passage 1 cells were irradiated in suspension at 20 Gy, and replated cultures were maintained at 21% 02.

Animals

Male and female C57BL/6J mice with hemizygous p16(INK4a) knock-in of firefly luciferase (p16^{Ink4a+/Luc}) were obtained from our breeding colony, originally obtained from Dr. Normal E. Sharpless [40]. Male C57BL/6J wild type mice were obtained from Jackson Laboratories (Bar Harbor, Me.), female NIH Swiss mice [Cr:NIH(S)] were obtained from Charles River (Wilmington, Mass.), and male C.B-Igh-IblcrTac-Prkdcscid/Ros mice (SCID) were obtained from the animal facility at the Roswell Park Cancer Institute (Buffalo, N.Y.).

Animals were provided a commercial rodent diet (5% 7012 Teklad LM-485 Mouse/Rat Sterilized Diet, Harlan) and sterile drinking water ad libitum. All the animals were confined to a limited access facility with environmentally-controlled housing conditions throughout the entire study period and maintained at 18-26° C., 30-70% air humidity, 12-h light/dark cycle. The animals were housed in micro-isolation cages under pathogen-free conditions, and if necessary, acclimatized in the housing conditions for at least 5 days prior to the start of the experiment. Animal usage in this experiment was approved under Institutional Animal Care and Use Committee (IACUC) at the Roswell Park Cancer Institute.

Microcarrier Bead Culture

For microcarrier bead cultures, Dry CytoPore™ 2 beads (GE Healthcare; Marlborough, Mass.) were hydrated and sterilized in 70% ethanol for 15 minutes, washed in PBS, and incubated with complete medium containing 10% FBS overnight at 4° C. Cells were attached to these modified cellulose-based macroporous microcarrier beads by incubating with freshly lifted NDF cell suspensions at a ratio of 5 million cells to 1 mL of packed, hydrated bead volume. Microcarrier bead culture was performed using complete DMEM medium in 250-mL tissue culture-grade vented cap Erlenmeyer flasks at 20-40 mL per 1 mL of hydrated packed bead volume, placed on a rotatory shaker within a tissue culture incubator. Three days after inoculation of CytoPore™ 2 beads, cells were switched into complete medium containing 0.2% FBS (quiescent condition), or exposed to 20 Gy gamma-irradiation via radioactive Cs source (Shepherd MK I-68 gamma-irradiator) (senescent condition), and maintained in culture for an addition 3-4 days, up to 2 weeks with media changed every 3-4 days.

For embedding of irradiated NDF microcarrier cultures into alginate beads, 400 μl of cytopore beads were mixed with 600 μl of 3% alginate solution. After thorough mixing, the suspension was loaded into a 1-mL syringe and mounted to an infusion pump (5-mm). The alginate encapsulation procedure was based on a gas-driven mono-jet device positioned 24-cm above the 100 mM SrCl₂ gelling solution, which was continuously stirred via magnet stir bar (700 rpm). The suspension NDF microcarrier cultures in alginate was sprayed into the gelling solution at an infusion rate of 0.8 mL/min and air flow rate of between 6-9 L/min. Alginate-coated beads were incubated in the gelling solution for 5 minutes, followed by thorough washing in PBS and transfer to warm complete medium. Beads were incubated on shaker in tissue culture incubator for at least 24 hours prior to injection.

Preparation of Solutions for Alginate Embedding

Ultra-pure low-viscosity (20-200 mPas) sodium alginate (PRONOVA UP LVG) powder was purchased from NovaMatrix (Sandvika, Norway). Alginate powder was dissolved in a 1% (w/v) mannitol solution to make a 3% (w/v) solution of sodium alginate. The final solution was filter-sterilized (0.2 μm) and stored at 4° C. A 100 mM solution of strontium chloride (SrCl₂) (Sigma) dissolved in sterile water was used for alginate gelation. NDF cells were synchronized (switch to 0.2% FBS at high confluency overnight) prior to irradiating cells in suspension at 20 Gy. Cells were then re-plated at subconfluent densities in complete medium (10% FBS) for at least 3 days. Irradiated NDF cells were lifted, washed in PBS, and re-suspended in 100 μl of saline. This cell suspension was then mixed with the 3% alginate solution (0.9 mL), and after thorough mixing, immediately loaded into a 1-mL syringe for extrusion. The alginate encapsulation procedure was based on a gas-driven mono-jet device positioned 14-cm above the 100 mM SrCl₂ gelling solution, which was continuously stirred via magnet stir bar (125 rpm). The suspension of NDF cells in alginate was sprayed into the gelling solution at an infusion rate of 0.6 mL/min and air flow rate of 7 L/min. Alginate-coated beads were incubated in the gelling solution for 5 minutes, followed by thorough washing in PBS and transfer to warm complete medium. Beads were incubated on shaker in tissue culture incubator for at least 24 hours prior to injection. Viability of senescent NDF cells embedded in alginate beads was verified by Calcein AM (Thermo Fisher Scientific)/propidium iodide (Sigma) staining of live/dead cells.

Implantation of Senescent NDFs

Microcarrier bead cultures of NDF cells (with or without alginate coating) were washed three times in neat RPMI medium (Gibco; Grand Island, N.Y.) and approximately 300 μl of packed bead volume (2 to 3 million cells) were injected intraperitoneally into isoflurane-anesthetized mice via a 16-gauge needle. Similarly, 300 μl of empty or SC-embedded alginate beads (cells suspended in alginate) were washed three times with saline for injection.

Bioluminescent Imaging

Mice were injected intraperitoneally with a 200 μl solution of 15 mg/mL D-luciferin potassium salt (Syd Labs; Boston, Mass.) in D-PBS without calcium and magnesium. At 10 minutes post-injection, isoflurane-anesthetized mice were placed into the IVIS Spectrum in vivo bioluminescent imaging system (PerkinElmer; Waltham, Mass.) for detection of luciferase activity (60-second exposure). Bioluminescence in p16^{LUC} mice was quantified as total flux (p/s) of luminescent signal from the abdomen using via Living Image® software.

Collection of Peritoneal Lavage and Alginate Beads

Two- to three-weeks post-implantation of alginate beads into the peritoneal cavity, peritoneal lavage and free alginate beads were collected from CO₂-asphyxiated mice. The skin covering the abdomen was dissected away, revealing the intact peritoneal cavity. Mice were then injected i.p. with 7 mL of 2% heat-inactivated FBS in saline using a 27-gauge needle. After gently massaging the abdomen, a 25-gauge needle was used to collect the peritoneal lavage. Lavages were then stored on ice prior to preparation of cell lysates. The cell density of collected peritoneal lavage was measured using Vial-Cassette™, where live and dead counts of nucleated cells from lavage were quantitated on a NucleoCounter® NC-200 (ChemoMetec; Allerod, Denmark) via acridine orange and DAPI staining. The peritoneal lavage was then pelleted (400×g for 5 minutes at 4° C.), re-suspended in BD Pharm Lyse lysing buffer (diluted to 1× in sterile, double-distilled water) purchased from Biosciences (San Jose, Calif.), and incubated in the dark at room temperature for 7 minutes. Three volumes of complete medium were added before pelleting cells and re-suspending in PBS.

For collection of alginate beads, the wall of the abdomen was opened. Beads were then flushed from the peritoneal cavity with saline containing 2% heat-inactivated FBS. Beads were then washed several times in PBS prior to analysis. Mouse immunocytes encapsulating the alginate beads were stained with CyQuant® Direct Cell Proliferation Assay (Thermo Fisher Scientific), and the stained nuclei from viable immunocytes were imaged via fluorescent microscopy.

Preparation of Cellular Lysates

Cell suspensions of lavage were recounted after RBC lysis, and equal numbers of cells were transferred to Eppendorf tubes for centrifugation (400×g for 5 minutes at 4° C.). Cell lysates of peritoneal lavage were obtained by re-suspending equal numbers of lavage cells or ex vivo alginate beads into 1× Reporter Lysis Buffer (Promega) supplemented with 0.5% Triton X-100 (Sigma) and 1:100 Dilution of protease inhibitor cocktail (Sigma). Lysis was performed at room temperature for 5 minutes with agitation, and the samples were then returned to ice. Using gel loading pipette tips, the cell extract was removed from alginate beads and placed into a clean Eppendorf tube. Lysates were clarified via centrifugation (16,000×g for 10 minutes at 4° C.) and stored on ice until use in enzymatic assays. Protein concentration of cell lysates was measured using the Pierce BCA Protein Assay Kit (Thermo Fisher Scientific; Waltham, Mass.) per the manufacturer's instructions.

Flow Cytometry

Before staining, mouse peritoneal lavage cells were treated with BD Pharm Lyse lysing buffer (diluted to 1× in sterile, double-distilled water) for red blood cell lysis, then washed and resuspended in flow cytometry staining buffer (eBioscience; San Diego, Calif.). After blocking with anti-CD16/CD32 antibodies (clone 93, eBioscience) for ten minutes the cells were stained with the following fluorochrome-conjugated antibodies to surface receptors in an 8-color staining combination: FITC-labeled anti-Ly-6G (1A8, Miltenyi Biotec); V500-labeled CD11b (M1/70, BD Horizon), and antibodies from eBioscience: PE-labeled anti-CD335 (29A1.4); PE/Cy5.5-labeled anti-CD19 (eBio1D3); PerCp-eFluor710-labeled anti-CD170 (1RNM44N); APC-labeled anti-CD45.2 (104); APC-eFuor780 labeled F4/80 (BMB); eFluor450-labeled anti-Ly-6C (HK1.4). After 30 minutes incubation on ice in the dark, cells were washed with flow cytometry staining buffer and resuspended in the same buffer. To distinguish dead cells, impermeable DNA stain Bobo3 (Molecular Probes, Eugene, Oreg.) was added to the cell suspension (20 nM final concentration) three minutes before acquisition. All sorting and analysis experiments were performed on Roswell Park Cancer Institute FACS facility custom instruments from BD Immunocytometry systems (FACSAria I or LSRII, respectively) using BD FACS Diva Software (BD Biosciences). Data were collected for 0.2 to 1×10⁶ cells and analyzed with FCS Express 4 (De Novo Software; Glendale, Calif.). To distinguish autofluorescent cells from cells expressing low levels of individual surface markers (in case of CD11b, Ly-6G, F4/80 and CD335 markers), we established upper thresholds for autofluorescence by staining samples with fluorescence-minus-one control stain sets in which a reagent for a channel of interest is omitted. Compensation was performed using single-color controls prepared with OneComp beads (eBioscience), or single-stained cell suspensions and calculated either with FACS Diva Software (in case of sorting) or with FCS Express (for composition analysis).

Immunofluorescence

Tissue samples were placed into plastic molds filled with NEG-50 frozen section medium (Fisher) and snap-frozen in a slurry of 2-methylbutane and dry ice. Fresh-frozen sections 12-μm thick were cut on a CM1900 cryotome (Leica), placed on Histobond slides (StatsLab), dried for 15 min and kept at −20° C. until staining. Before staining, slides were warmed up to room temperature, fixed 5 min with 4% formaldehyde in PBS, and washed 3 times with PBS. Sections were incubated with block solution (5% normal donkey serum, 0.25% triton x-100, PBS) and incubated with rat monoclonal anti-F4/80 antibody conjugated with AlexaFluor488 (BioLegend, 1:50 dilution); rat monoclonal anti-B220 antibody conjugated with Cy5 (eBioscience, 1:50 dilution); and mouse monoclonal anti-smooth muscle actin (SMA) antibody conjugated with Cy3 (Sigma, 1:1000 dilution). All antibodies were diluted in block solution. Stained sections were mounted with ProLong Diamond anti-fade reagent with DAPI (Invitrogen). Sections were analyzed under AxioImager Z1 microscope equipped with epifluorescence and AxioCam MRm digital camera (Carl Zeiss Inc.). Images were captured and processed with AxioVision software (Carl Zeiss Inc., release 4.5.3).

Firefly Luciferase Assay

Luciferase activity of cell lysates was accessed using Bright-Glo™ Luciferase Assay System (Promega; Madison, Wis.) according to manufacturer's instructions with minor modifications. Briefly, cell lysates (as described previously) or cell suspensions in D-PBS were added to a 96-well white plate (OptiPlate-96, Perkin Elmer), followed by the addition of an equal volume of 2× reconstituted Bright-Glo™ Assay Reagent. Acquisition of the luminescent signal was performed on the Infinite® M1000 PRO microplate reader (Tecan; Männedorf, Switzerland). The mean maximum signal from technical replicates was used to estimate the luciferase activity for each sample. Background readings were measured after incubation of equal volumes of 2× luciferase reagent with either D-PBS or lysis buffer (as appropriate). Background signal was subtracted from sample signal, and normalized either by cell number or protein content per reaction. The ratio of background signal to sample signal (signal-to-noise ratio) was calculated for each sample. Sample signals less than 2-fold above background were considered to not be reliably detectable. To estimate detection threshold for a given sample, the background signal was normalized to cell number or protein amount in the reaction.

Gaussia Luciferase Assay

Blood was collected from the saphenous vein (50 μL) into heparinized collection vials (Sarstedt; Nümbrecht, Germany) at regular intervals (twice per week, up to 28 days) from SCID mice bearing NDF cells transduced with a GLuc reporter construct. Plasma was obtained following centrifugation at 10,000×g for 7 minutes at 4° C., and stored at −80° C. with no substantial loss of signal. For measurement of GLuc activity, plasma was diluted 1:10 in PBS containing 0.1% Triton X-100 in a solid white 96-well microplate. Next, two volumes of a 100 μM solution of coelenterazine in 1×PBS supplemented with 0.3M ascorbic acid and 0.1% Triton X100, pH 7.5, was added to the diluted plasma sample and luminescent signal was measured immediately on a microplate luminometer (Tecan) using a 500 ms integration time. Coelenterazine was obtained as a dry powder from NanoLight Technologies (Pinetop, Ariz.); stock solutions at 5 mg/ml in acidified ethanol were stored at −80° C.

Determination of β-gal^{pH6} Activity

β-gal^{pH6} activity of cryosectioned fresh frozen tissue or whole fat tissue was assayed as previously described [75]. Briefly, cryosections were fixed for 5 minutes with 2% (v/v) para-formaldehyde and 0.2% (v/v) glutaraldehyde, washed in PBS, and incubated with 0.1% (v/v) X-Gal in 100 mM citric acid/sodium phosphate buffer (pH 6.0), containing 2 mM MgCl₂, 150 mM NaCl, 5 mM K₃Fe(CN)₆, 5 mM K₄Fe(CN)₆, and 0.02% (v/v) NP-40 at 37° C., and monitored for development of X-Gal product for up to 18 hours. The reaction was stopped by washing slides in PBS, followed by counterstaining with nuclear fast red (Ricca Chemical Company; Arlington, Tex.). Visceral and inguinal fat was processed similarly.

A fluorogenic substrate for β-galactosidase, 4-MUG (4-Methylumbelliferyl β-D-Galactopyranoside; Sigma-Aldrich), was used for the quantitation of enzymatic β-gal activity from cell lysates, assayed under β-gal^{pH6} conditions (i.e. pH 6.0). In a 96-well white microplate, 5 μl of diluted cell lysate was combined with 45 μl of fluorogenic β-gal^{pH6} reaction buffer: 1 mM 4-MUG in 100 mM citric acid/sodium phosphate buffer, pH 6.0, containing 2 mM MgCl₂, 150 mM NaCl and 0.02% NP-40. Kinetics of product formation at room temperature was determined by measuring fluorescence (Ex/Em 360 nm/440 nm) of the reaction mixture at regular intervals over a 2-hour time period. Reaction endpoints were determined by addition of 150 μl of 700 mM sodium carbonate. The rate of reaction (RFU/min) for each sample was determined, and normalized per μg of protein.

Statistical Analysis

All statistical analyses were performed using GraphPad Prism version 5.00 (GraphPad Software, San Diego, Calif.). Statistical comparison of two groups was performed using an unpaired students' two-tailed t-test. For statistical comparisons involving more than two groups, a one-way analysis of variance (ANOVA) with Bonferroni post-hoc test or a Kruskal-Wallis one-way ANOVA with Dunn's post-hoc test (for non-parametric data) was performed to determine differences between groups. Differences were considered statistically significant at p-values less than 0.05: not significant (ns), p>0.05; *, p≤0.05; **, p≤0.01; ***, p≤0.001; ****, p≤0.0001. All data presented as mean±standard error.

Frailty Index

Frailty Index (FI) was determined for individual mice as described previously (Antoch et al. Aging. 2017; 9: 1-12) using a 26 week-old group as a “young mouse” reference. For each mouse, various parameters were measured. These parameters include non-invasive measurements, including age, body weight, grip strength, diastolic blood pressure, and tail blood flow. Additional blood chemistry measurements were also determined, including white blood cell count, neutrophil count, neutrophil percentage, lymphocyte percentage, monocyte percentage, eosinophil percentage, red blood cell count, hemoglobin, hematocrit, mean corpuscular volume, mean corpuscular hemoglobin, mean corpuscular hemoglobin concentration, platelet count, and mean platelet volume. For each parameter mean value and standard deviation were calculated. Animals differing by more than one standard deviation (STDEV) from mean value in any single parameter were excluded from the reference group. Values for each measured parameter were compared with corresponding values for the reference group and assigned a score. The number of deficits an individual mouse expressed was calculated as a ratio of total number of parameters measured and this value is referred to as the frailty index.

Non-Limiting List of Specific Embodiments

In addition to the aspects and embodiments described elsewhere in the present disclosure, the below are a non-limiting list of certain specific embodiments.

1. A compound having the following formula:

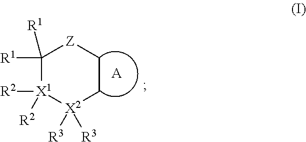

wherein:
A, together with the two carbons from the adjacent ring, is a fused heterocycle;
Z is selected from the group consisting of C═N—OR⁵, wherein R⁵ is H or a prodrug substituent;

### X¹ is C or N;

X² is C, O, or a spacer (e.g., —C(═O)—, —C(N═OR⁶)—, —C(═O)CH₂—, —C(N═OR⁶)CH₂—, —CH₂C(═O)—, —CH₂C(N═OR⁶)—), wherein R⁶ is —H or substituted or unsubstituted C₁ to C₆ alkyl group;
each R¹ is independently selected from the group consisting of H, C₁ to C₆ alkyl group, and C₃ to C₁₈ aryl group, or both R¹ groups may, together with the carbon they are attached to, form a C₃ to C₁₀ spirocycle group;
each R² is independently selected from the group consisting of H, C₁ to C₆ alkyl group, C₃ to C₁₈ aryl group, C₁-C₂₀ heteroaryl group, and C(O)N(R⁷)₂, or both R¹ groups may, together with the carbon they are attached to, form a C₃-C₁₀ spirocycle group, wherein R⁷ is independently selected from the group consisting of —H, substituted or unsubstituted C₁-C₆ alkyl group, substituted or unsubstituted alkynyl group, substituted or unsubstituted aryl group, substituted or unsubstituted heteroaryl group, substituted or unsubstituted saturated heterocyclyl group, substituted or unsubstituted partially-saturated heterocyclyl group, substituted or unsubstituted saturated C₃ to C₂₀ carbocyclyl group and substituted or unsubstituted partially-saturated C₃ to C₂₀ carbocycylyl group, or both R⁹ substituents, together with the N atom they are attached to, form a heterocycle group, which may additionally contains a further heteroatom(s) selected from the group consisting of N, O, and S;
each R³ is independently selected from the group consisting of H, C₁ to C₆ alkyl group, C₃ to C₁₈ aryl group, and C₁ to C₂₀ heteroaryl group; and
any two of R¹, R², R³, and/or substituents of A may be linked together with a linker to form a macrocycle group,
wherein one or more of the heteroatoms, such as, for example, nitrogen and sulfur, may optionally be oxidized to form N-oxides or sulfoxides and sulfones, respectively, and/or one or more nitrogen in one or more heterocycle may be quaternized,
or
a salt (e.g., a pharmaceutically acceptable salt) of the compound.
2. The compound of embodiment 1, wherein A is selected from the group consisting of:

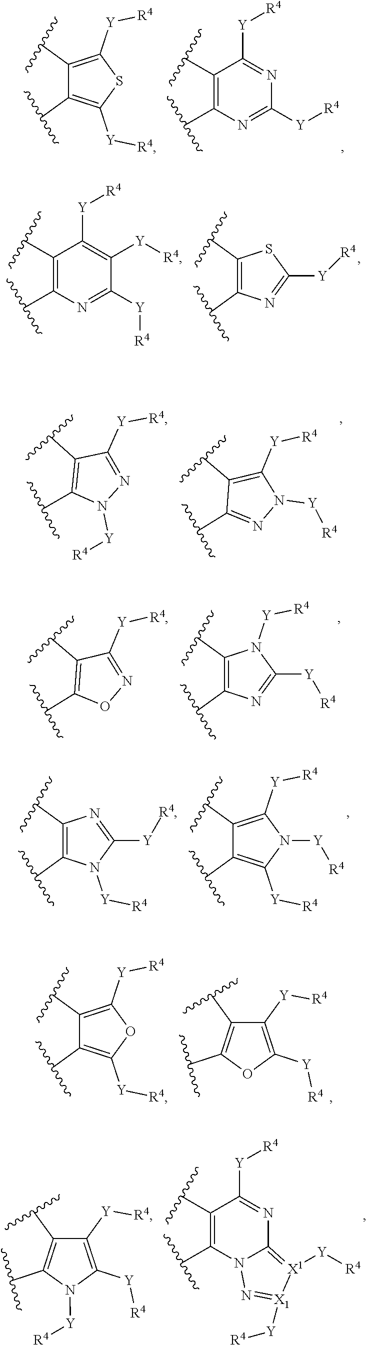

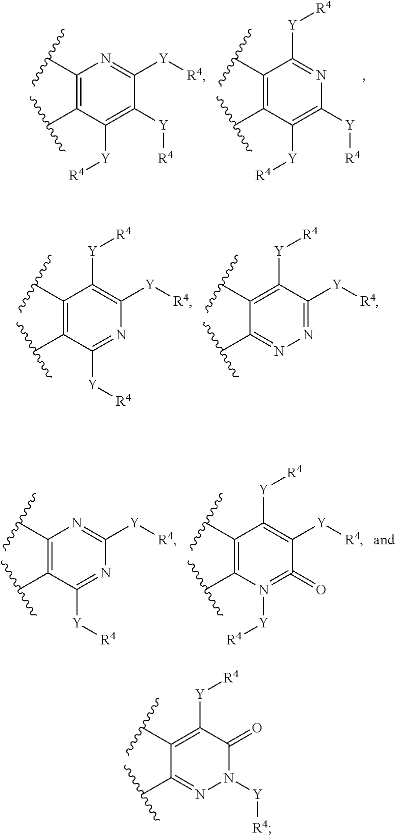

wherein each Y is independently selected from the group consisting of direct bond, O, S, S(═O), S(═O)₂, S(═O)(═NR⁸), C(═O), C(═O)O,

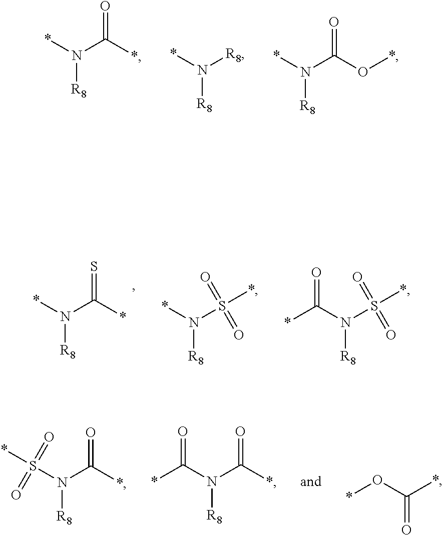

wherein each R⁸ is independently —H or substituted or unsubstituted C₁ to C₆ alkyl group, or C₃ to C₂₀ cycloalkyl group; and each R⁴ is independently selected from the group consisting of H, halide, CN, NO₂, substituted or unsubstituted C₁ to C₆ alkyl group, substituted or unsubstituted C₂ to C₄ alkenyl group, substituted or unsubstituted C₂ to C₄ alkynyl group, substituted or unsubstituted C₁ to C₆ alkoxyl group, substituted or unsubstituted C₁ to C₆ arylalkyl group, substituted or unsubstituted C₁ to C₆ hydroxyalkyl group, substituted or unsubstituted C₁ to C₆ alkoxyalkyl group, substituted or unsubstituted C₃ to C₂₀ cycloalkyl group, substituted or unsubstituted C₃ to C₁₈ aryl group, substituted or unsubstituted C₁ to C₆ cycloalkylalkyl group, substituted or unsubstituted C₁ to C₂₀ heteroaryl group, substituted or unsubstituted C₁ to C₂₀ heterocyclyl group, substituted or unsubstituted C₁ to C₆ heterocyclylalkyl group, substituted or unsubstituted C₁ to C₆ heteroarylalkylgroup, and —N(R⁷)₂.
3. The compound of any one of the preceding embodiments, wherein the compound is selected from the group consisting of:

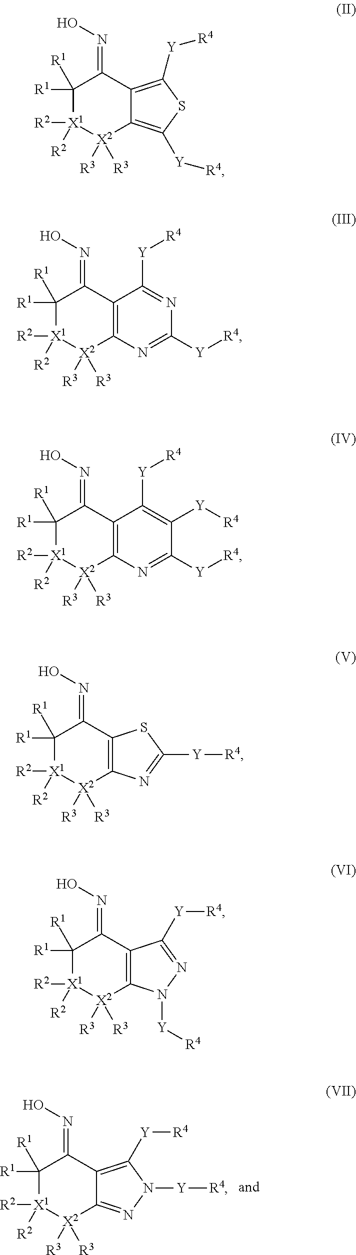

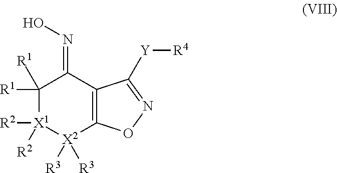

4. A compound of Formula (IIa)

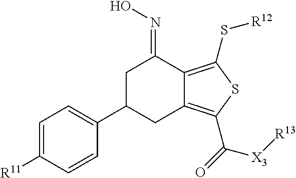

wherein,
R¹¹ is selected from: halogen, substituted or unsubstituted C₁₋₆ alkyl, substituted or unsubstituted C₁₋₆ alkoxyl;
R¹² is selected from: substituted or unsubstituted phenyl, substituted or unsubstituted benzyl, substituted or unsubstituted phenethyl;
X³ is selected from O or NH;
R¹³ is selected from substituted or unsubstituted C₁₋₆ alkyl, substituted or unsubstituted C₃₋₉ cycloalkyl, substituted or unsubstituted phenyl, substituted or unsubstituted benzyl, substituted or unsubstituted methylheterocyclyl, substituted or unsubstituted methyl heteroaryl.
5. A compound of Formula (IIIa)

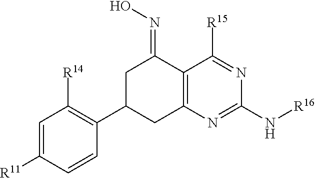

wherein,
R¹⁴ is selected from: halogen, substituted or unsubstituted C₁₋₆ alkyl, substituted or unsubstituted C₁₋₆ alkoxyl, substituted or unsubstituted C6 aryl;
R¹⁵ is selected from: H, substituted or unsubstituted C₁₋₆ alkyl, substituted or unsubstituted C₁₋₆ alkoxyl;
R¹⁶ is selected from: substituted or unsubstituted phenyl, substituted or unsubstituted benzyl, substituted or unsubstituted 3-phenylpropan-1-yl, substituted or unsubstituted 3□ heteroarylpropan-1-yl, substituted or unsubstituted 2-phenoxyethyl, substituted or unsubstituted 2-heteroaryloxyethyl;
R¹⁷ is selected from: halogen, substituted or unsubstituted C₁₋₆ alkyl, substituted or unsubstituted C₃₋₉ cycloalkyl, substituted or unsubstituted C₂₋₄ alkynyl, substituted or unsubstituted C₁₋₆ alkoxyl, substituted or unsubstituted phenyl.
6. A compound of Formula (IVa)

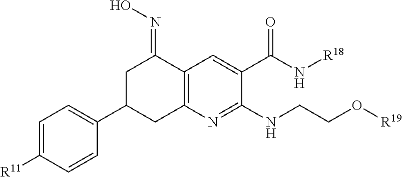

wherein
R¹¹ as defined above;
R¹⁸ selected from substituted or unsubstituted phenyl, substituted or unsubstituted heteroaryl, substituted or unsubstituted C₃₋₉ cycloalkyl;
R¹⁸ selected from substituted or unsubstituted phenyl, substituted or unsubstituted heteroaryl.
7. A compound, wherein the compound is selected from the group consisting of:

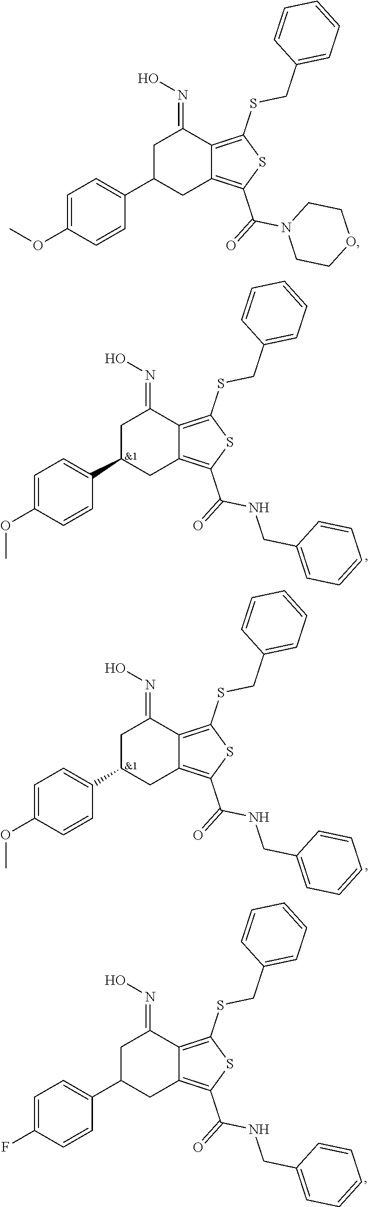

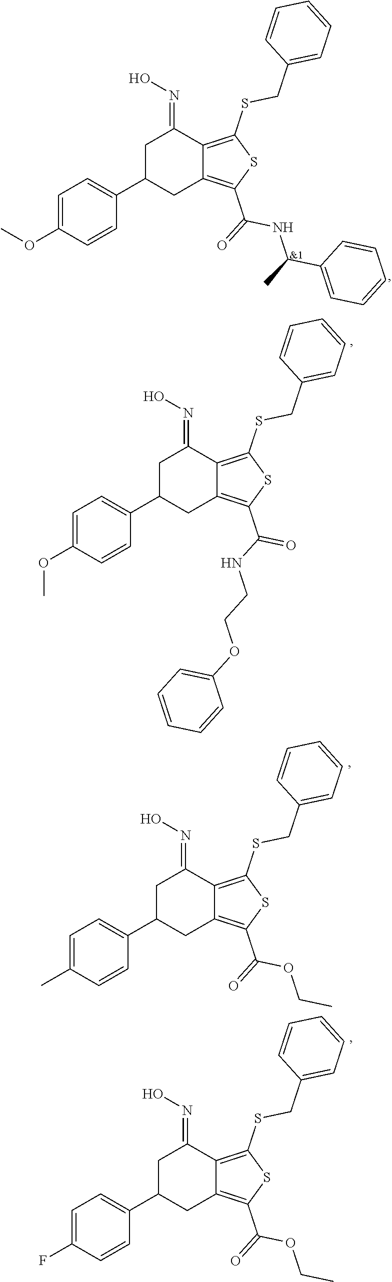

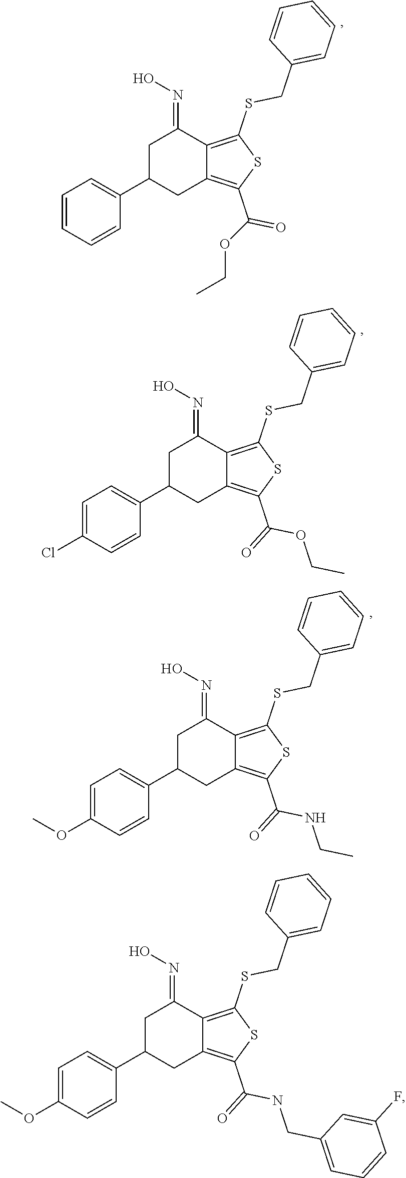

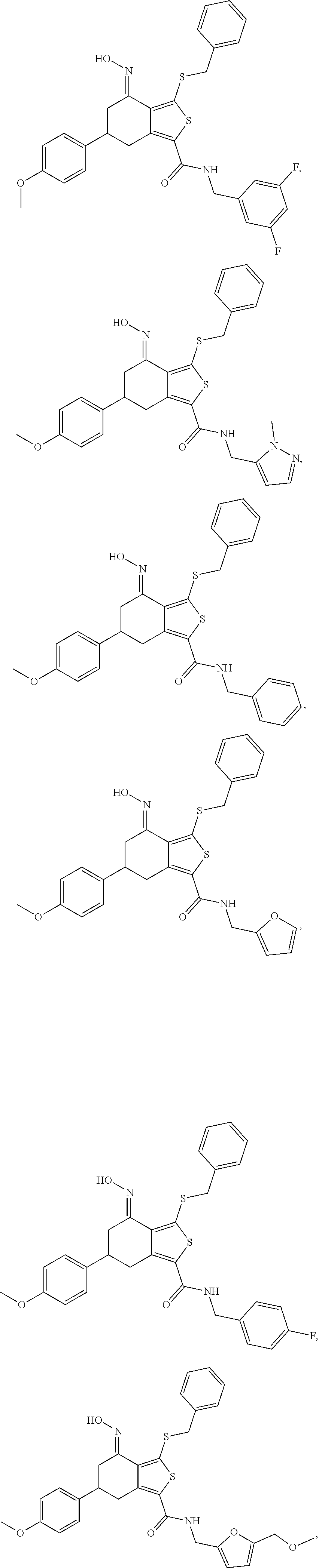

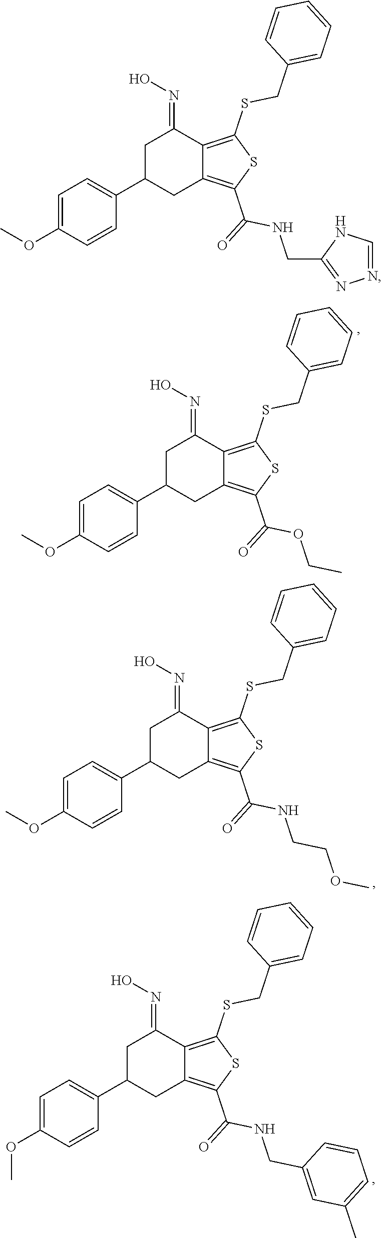

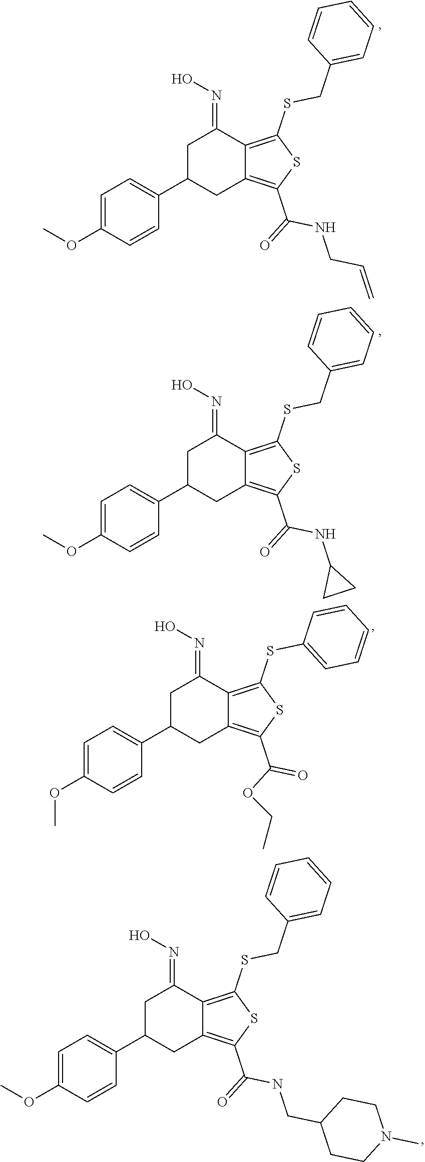

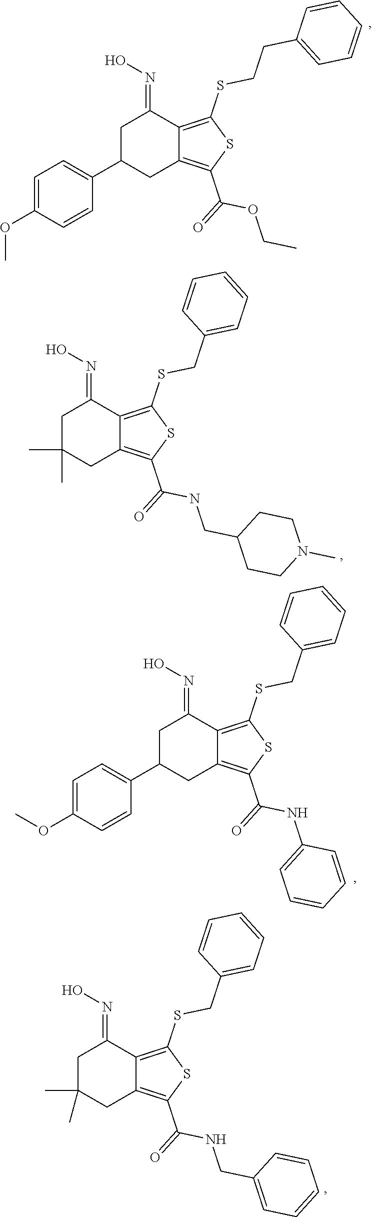

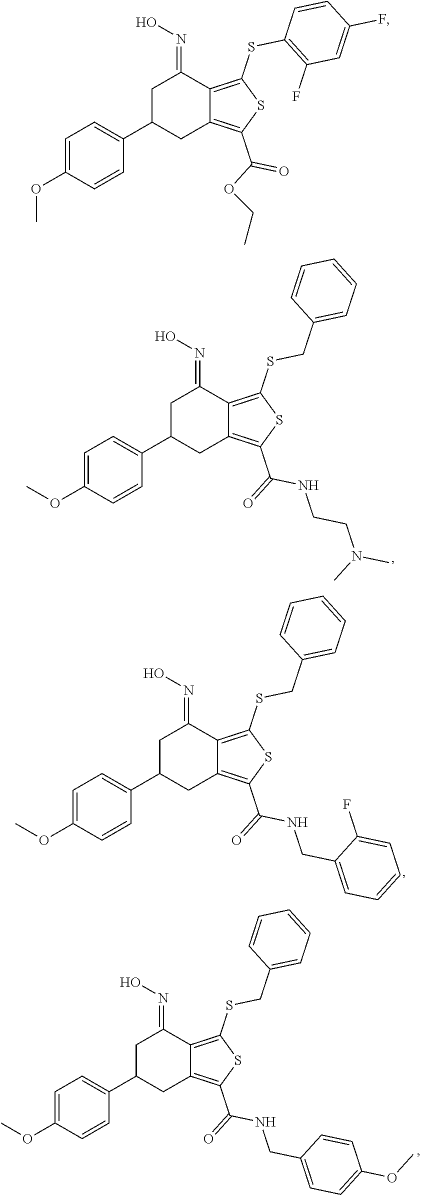

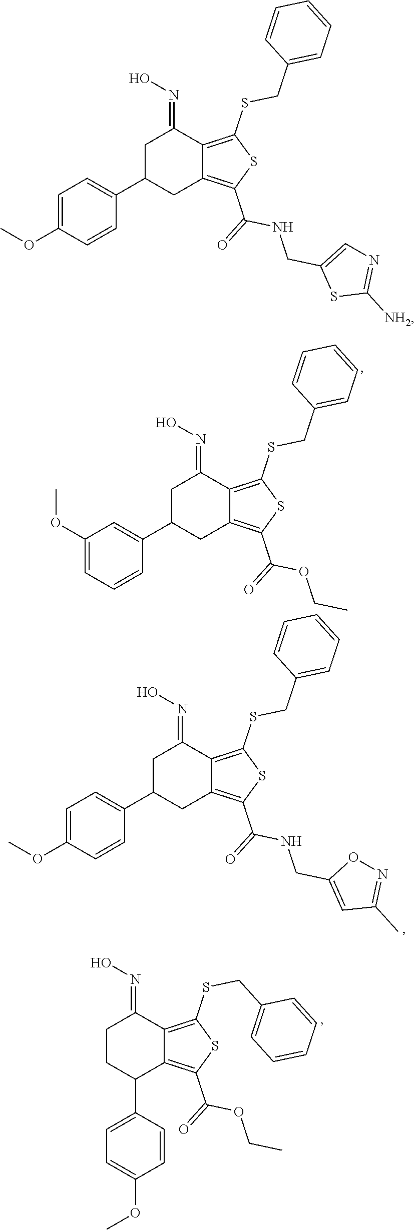

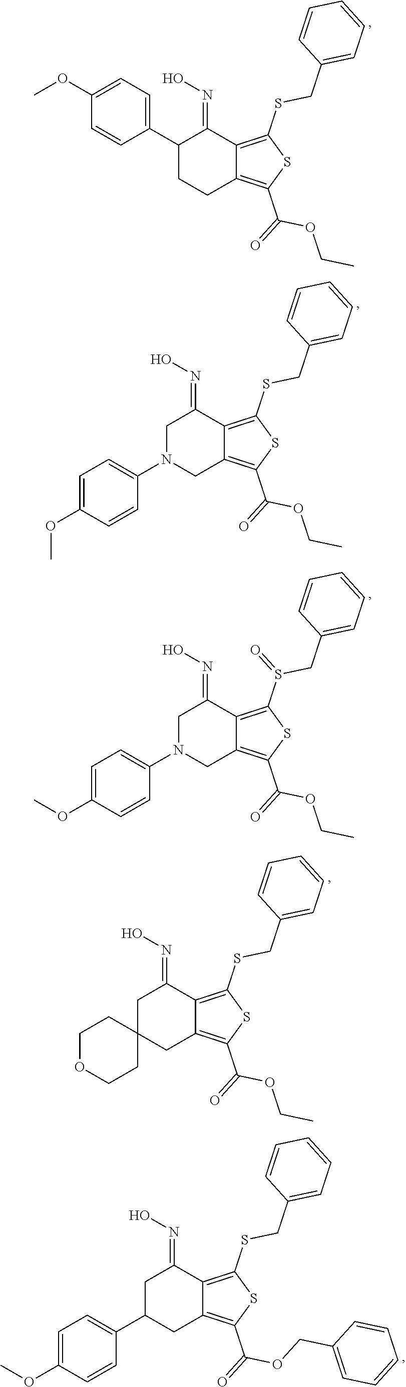

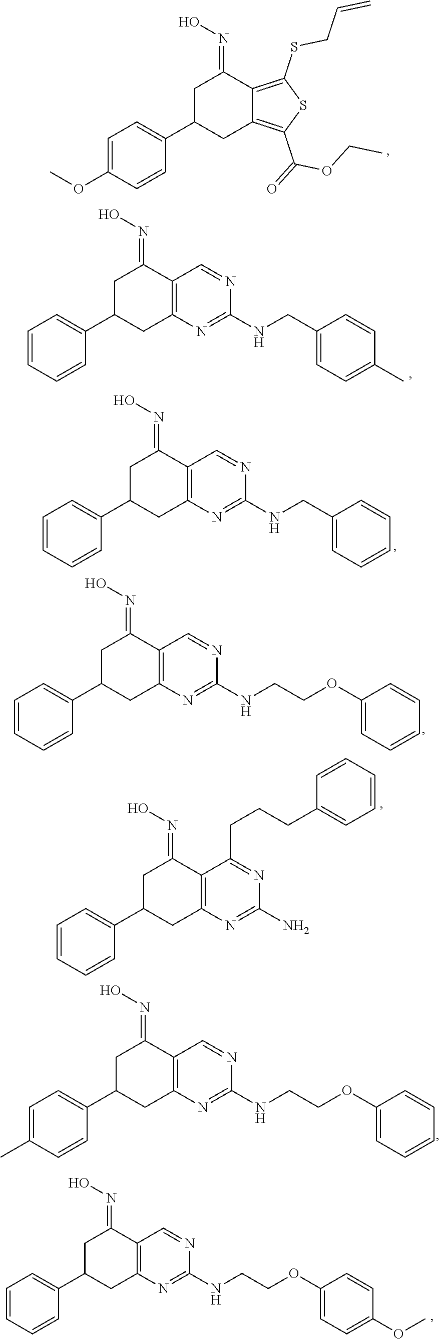

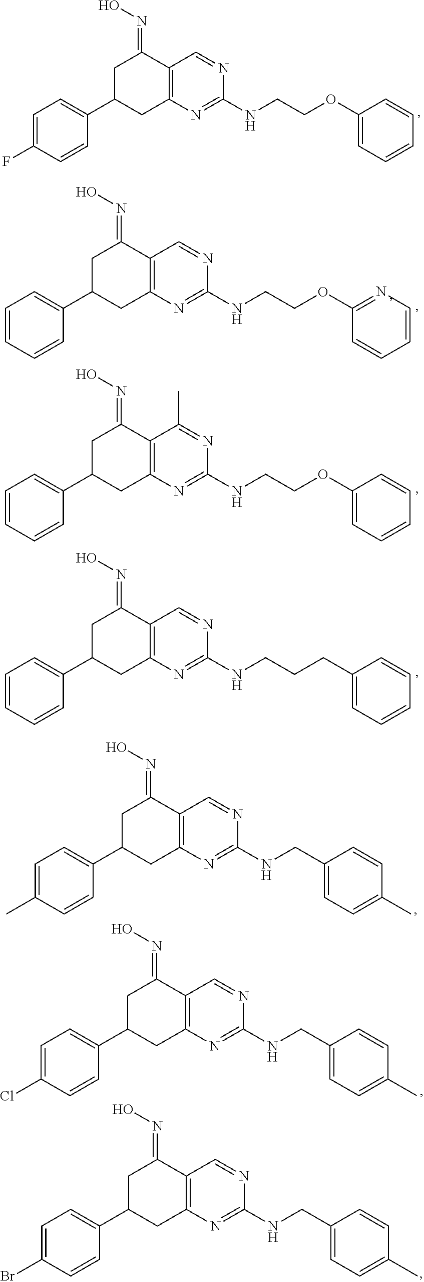

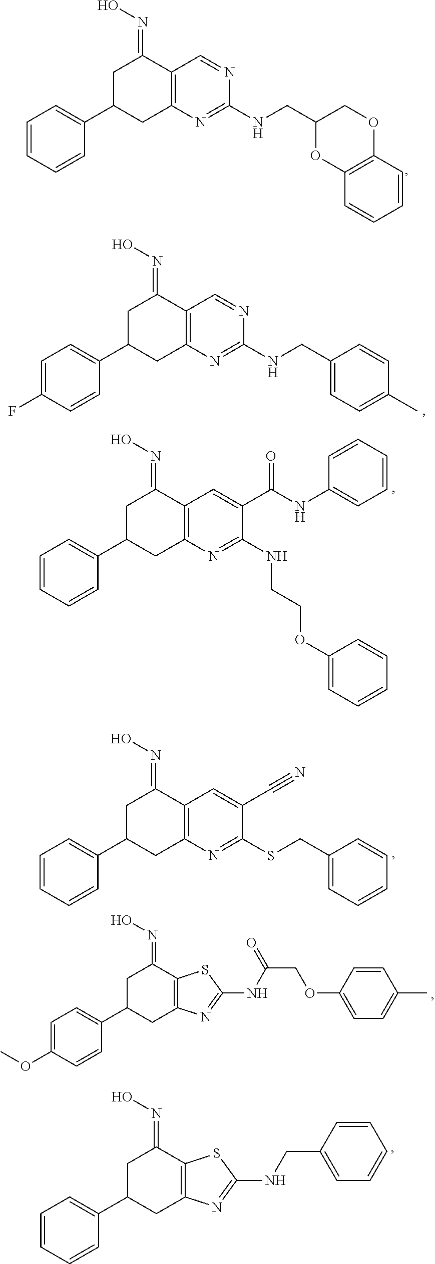

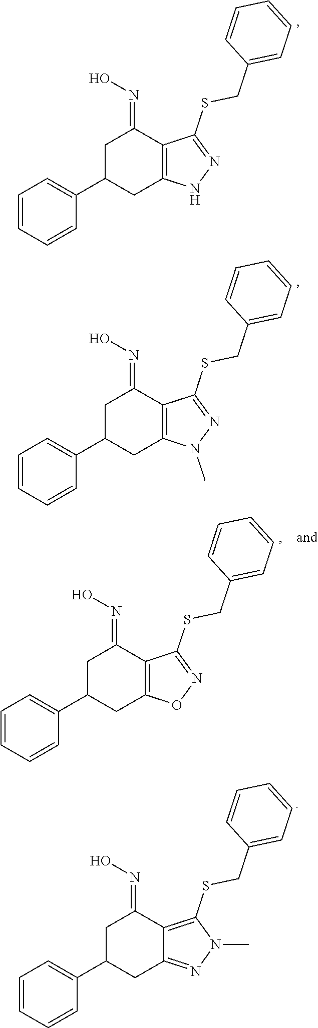

8. A compound, wherein the compound is:

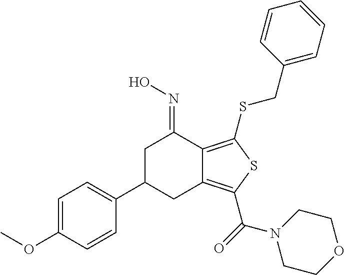 [SMILES: COc1ccc(C2C/C(=N\O)c3c(SCc4ccccc4)sc(C(=O)N4CCOCC4)c3C2)cc1]

9. A compound, wherein the compound is:

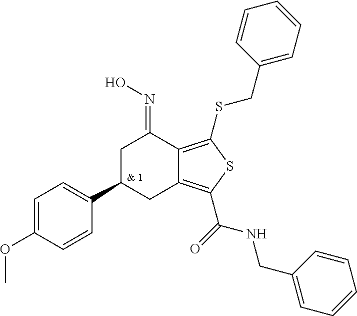 [SMILES: COc1ccc([C@H]2C/C(=N\O)c3c(SCc4ccccc4)sc(C(=O)NCc4ccccc4)c3C2)cc1]

10. A compound, wherein the compound is:

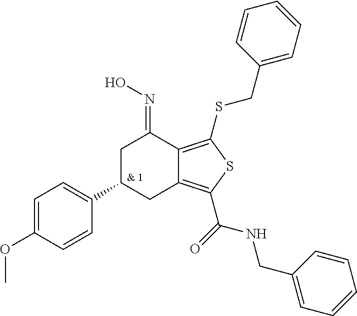 [SMILES: COc1ccc([C@@H]2C/C(=N\O)c3c(SCc4ccccc4)sc(C(=O)NCc4ccccc4)c3C2)cc1]

11. A compound, wherein the compound is:

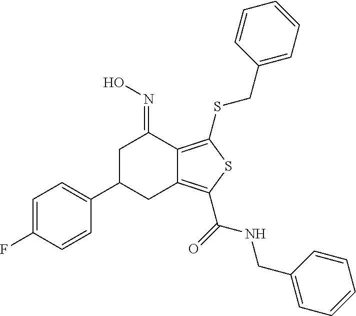 [SMILES: O=C(NCc1ccccc1)c1sc(SCc2ccccc2)c2c1CC(c1ccc(F)cc1)C/C2=N\O]

12. A compound, wherein the compound is:

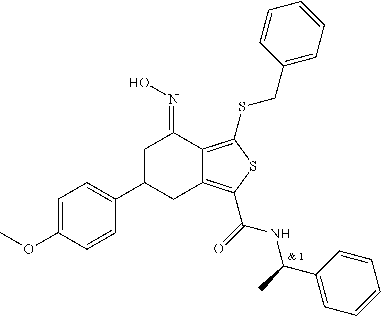 [SMILES: COc1ccc(C2C/C(=N\O)c3c(SCc4ccccc4)sc(C(=O)N[C@H](C)c4ccccc4)c3C2)cc1]

13. A compound, wherein the compound is:

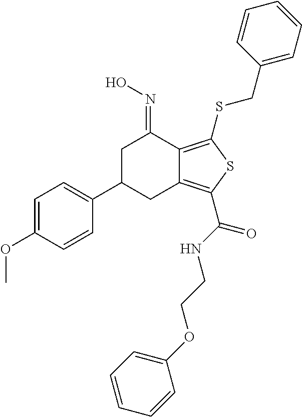 [SMILES: COc1ccc(C2C/C(=N\O)c3c(SCc4ccccc4)sc(C(=O)NCCOc4ccccc4)c3C2)cc1]

14. A compound, wherein the compound is:

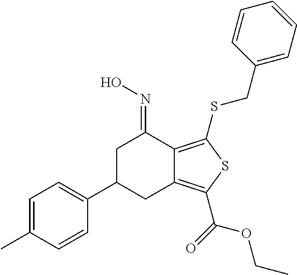 [SMILES: CCOC(=O)c1sc(SCc2ccccc2)c2c1CC(c1ccc(C)cc1)C/C2=N\O]

15. A compound, wherein the compound is:

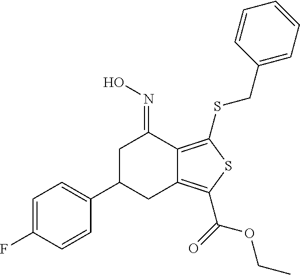 [SMILES: CCOC(=O)c1sc(SCc2ccccc2)c2c1CC(c1ccc(F)cc1)C/C2=N\O]

16. A compound, wherein the compound is:

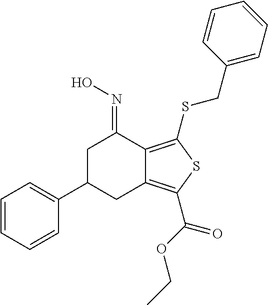 [SMILES: CCOC(=O)c1sc(SCc2ccccc2)c2c1CC(c1ccccc1)C/C2=N\O]

17. A compound, wherein the compound is:

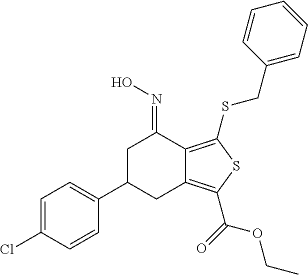 [SMILES: CCOC(=O)c1sc(SCc2ccccc2)c2c1CC(c1ccc(Cl)cc1)C/C2=N\O]

18. A compound, wherein the compound is:

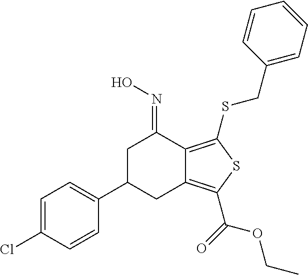 [SMILES: CCOC(=O)c1sc(SCc2ccccc2)c2c1CC(c1ccc(Cl)cc1)C/C2=N\O]

19. A compound, wherein the compound is:

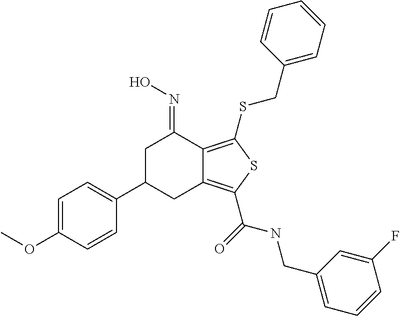 [SMILES: COc1ccc(C2C/C(=N\O)c3c(SCc4ccccc4)sc(C(=O)NCc4cccc(F)c4)c3C2)cc1]

20. A compound, wherein the compound is:

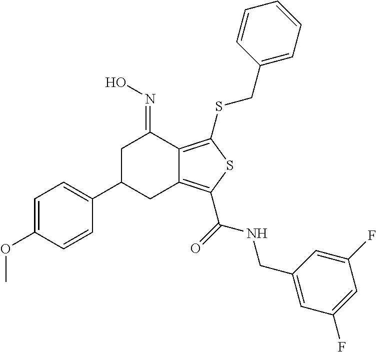 [SMILES: COc1ccc(C2C/C(=N\O)c3c(SCc4ccccc4)sc(C(=O)NCc4cc(F)cc(F)c4)c3C2)cc1]

21. A compound, wherein the compound is:

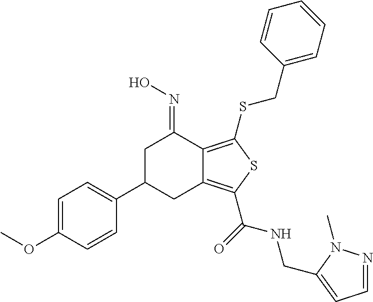 [SMILES: COc1ccc(C2C/C(=N\O)c3c(SCc4ccccc4)sc(C(=O)NCc4ccnn4C)c3C2)cc1]

22. A compound, wherein the compound is:

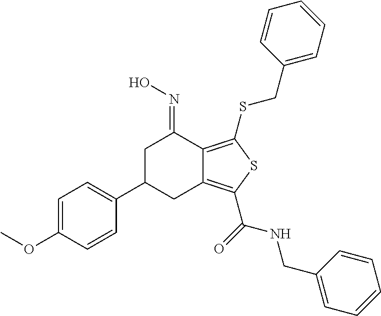 [SMILES: COc1ccc(C2C/C(=N\O)c3c(SCc4ccccc4)sc(C(=O)NCc4ccccc4)c3C2)cc1]

23. A compound, wherein the compound is:

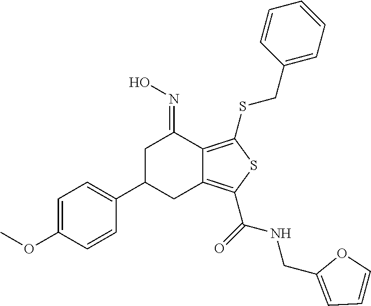 [SMILES: COc1ccc(C2C/C(=N\O)c3c(SCc4ccccc4)sc(C(=O)NCc4ccco4)c3C2)cc1]

24. A compound, wherein the compound is:

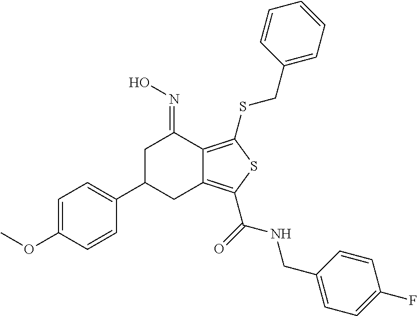 [SMILES: COc1ccc(C2C/C(=N\O)c3c(SCc4ccccc4)sc(C(=O)NCc4ccc(F)cc4)c3C2)cc1]

25. A compound, wherein the compound is:

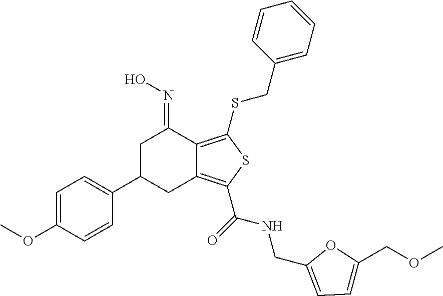

26. A compound, wherein the compound is:

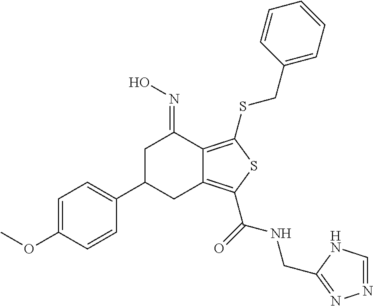 [SMILES: COc1ccc(C2C/C(=N\O)c3c(SCc4ccccc4)sc(C(=O)NCc4nnc[nH]4)c3C2)cc1]

27. A compound, wherein the compound is:

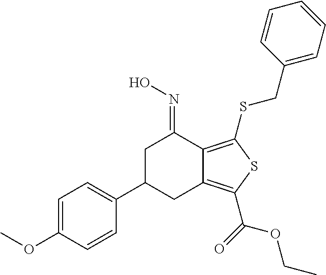 [SMILES: CCOC(=O)c1sc(SCc2ccccc2)c2c1CC(c1ccc(OC)cc1)C/C2=N\O]

28. A compound, wherein the compound is:

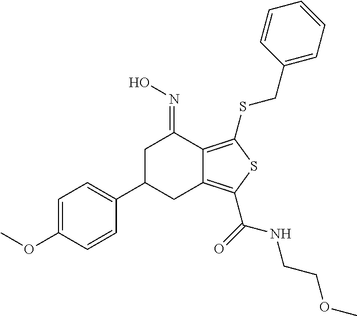

29. A compound, wherein the compound is:

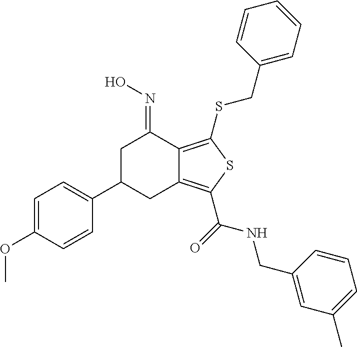 [SMILES: COc1ccc(C2C/C(=N\O)c3c(SCc4ccccc4)sc(C(=O)NCc4cccc(C)c4)c3C2)cc1]

30. A compound, wherein the compound is:

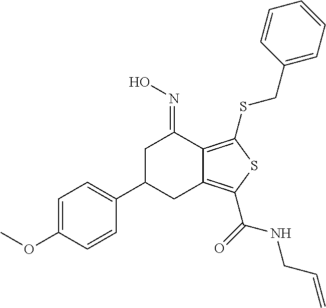 [SMILES: C=CCNC(=O)c1sc(SCc2ccccc2)c2c1CC(c1ccc(OC)cc1)C/C2=N\O]

31. A compound, wherein the compound is:

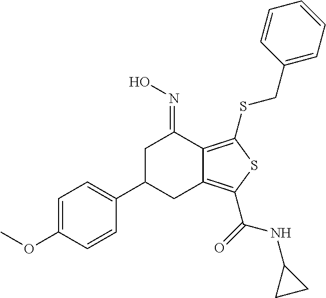 [SMILES: COc1ccc(C2C/C(=N\O)c3c(SCc4ccccc4)sc(C(=O)NC4CC4)c3C2)cc1]

32. A compound, wherein the compound is:

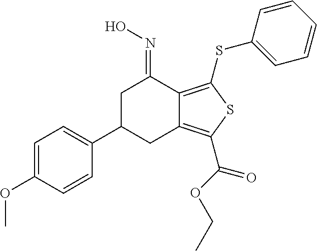 [SMILES: CCOC(=O)c1sc(Sc2ccccc2)c2c1CC(c1ccc(OC)cc1)C/C2=N\O]

33. A compound, wherein the compound is:

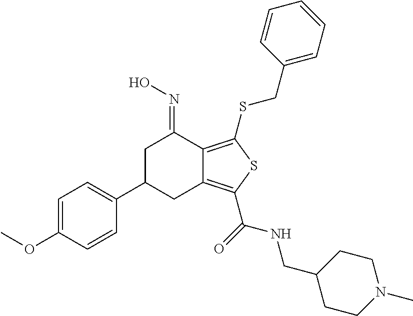

34. A compound, wherein the compound is:

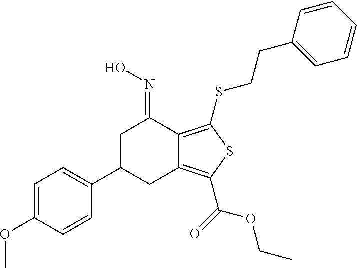 [SMILES: CCOC(=O)c1sc(SCCc2ccccc2)c2c1CC(c1ccc(OC)cc1)C/C2=N\O]

35. A compound, wherein the compound is:

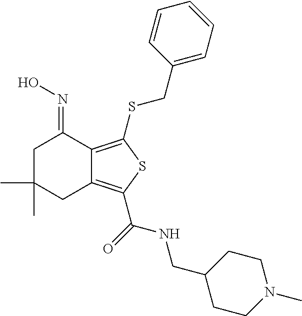

36. A compound, wherein the compound is:

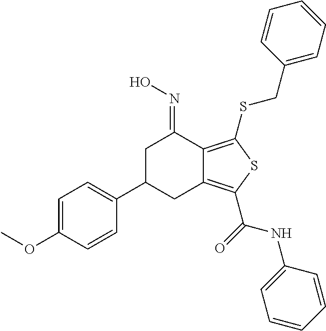 [SMILES: COc1ccc(C2C/C(=N\O)c3c(SCc4ccccc4)sc(C(=O)Nc4ccccc4)c3C2)cc1]

37. A compound, wherein the compound is:

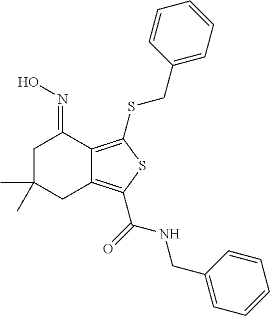 [SMILES: CC1(C)C/C(=N\O)c2c(SCc3ccccc3)sc(C(=O)NCc3ccccc3)c2C1]

38. A compound, wherein the compound is:

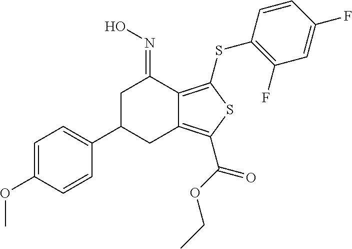 [SMILES: CCOC(=O)c1sc(Sc2ccc(F)cc2F)c2c1CC(c1ccc(OC)cc1)C/C2=N\O]

39. A compound, wherein the compound is:

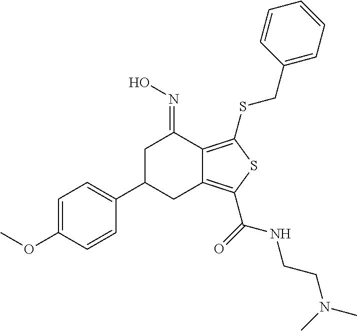

40. A compound, wherein the compound is:

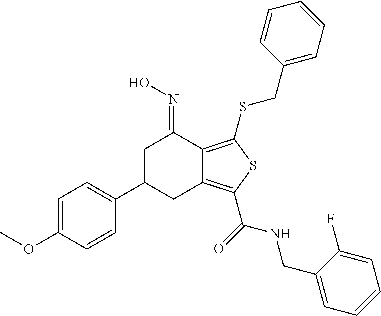 [SMILES: COc1ccc(C2C/C(=N\O)c3c(SCc4ccccc4)sc(C(=O)NCc4ccccc4F)c3C2)cc1]

41. A compound, wherein the compound is:

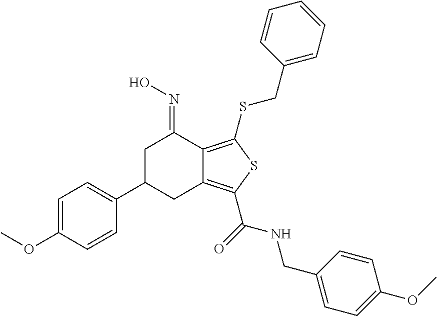

42. A compound, wherein the compound is:

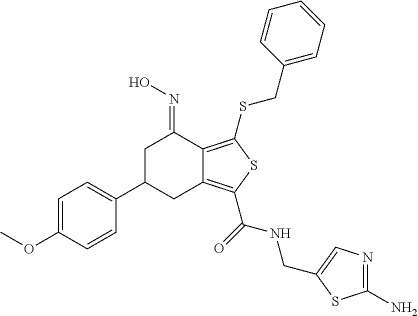 [SMILES: COc1ccc(C2C/C(=N\O)c3c(SCc4ccccc4)sc(C(=O)NCc4cnc(N)s4)c3C2)cc1]

43. A compound, wherein the compound is:

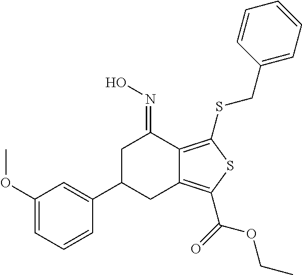 [SMILES: CCOC(=O)c1sc(SCc2ccccc2)c2c1CC(c1cccc(OC)c1)C/C2=N\O]

44. A compound, wherein the compound is:

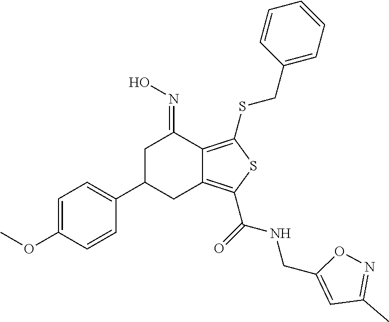

45. A compound, wherein the compound is:

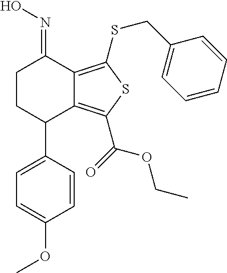 [SMILES: CCOC(=O)c1sc(SCc2ccccc2)c2c1C(c1ccc(OC)cc1)CC/C2=N\O]

46. A compound, wherein the compound is:

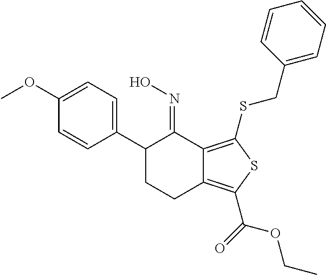 [SMILES: CCOC(=O)c1sc(SCc2ccccc2)c2c1CCC(c1ccc(OC)cc1)/C2=N\O]

47. A compound, wherein the compound is:

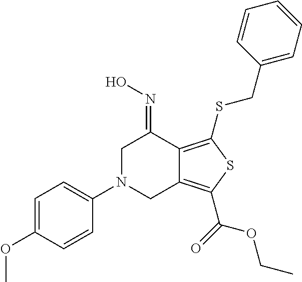 [SMILES: CCOC(=O)c1sc(SCc2ccccc2)c2c1CN(c1ccc(OC)cc1)C/C2=N\O]

48. A compound, wherein the compound is:

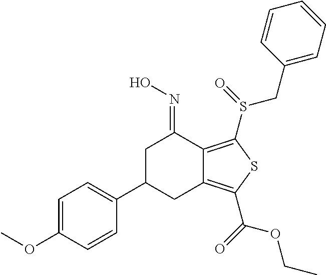 [SMILES: CCOC(=O)c1sc(S(=O)Cc2ccccc2)c2c1CC(c1ccc(OC)cc1)C/C2=N\O]

49. A compound, wherein the compound is:

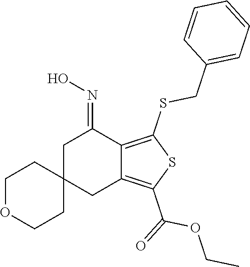 [SMILES: CCOC(=O)c1sc(SCc2ccccc2)c2c1CC1(CCOCC1)C/C2=N\O]

50. A compound, wherein the compound is:

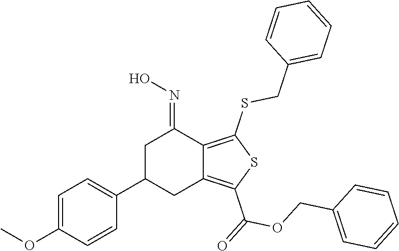 [SMILES: COc1ccc(C2C/C(=N\O)c3c(SCc4ccccc4)sc(C(=O)OCc4ccccc4)c3C2)cc1]

51. A compound, wherein the compound is:

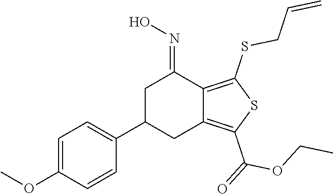 [SMILES: C=CCSc1sc(C(=O)OCC)c2c1/C(=N/O)CC(c1ccc(OC)cc1)C2]

52. A compound, wherein the compound is:

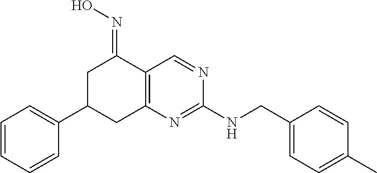 [SMILES: Cc1ccc(CNc2ncc3c(n2)CC(c2ccccc2)C/C3=N\O)cc1]

53. A compound, wherein the compound is:

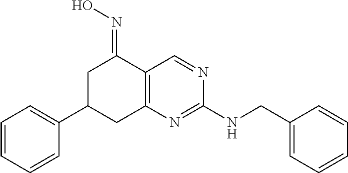 [SMILES: O/N=C1\CC(c2ccccc2)Cc2nc(NCc3ccccc3)ncc21]

54. A compound, wherein the compound is:

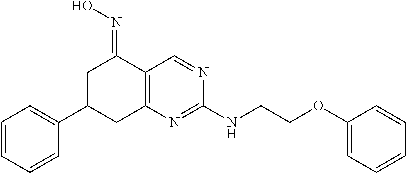 [SMILES: O/N=C1\CC(c2ccccc2)Cc2nc(NCCOc3ccccc3)ncc21]

55. A compound, wherein the compound is:

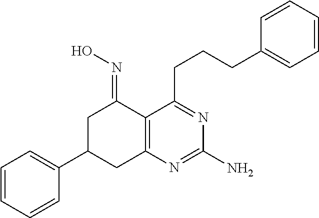 [SMILES: Nc1nc(CCCc2ccccc2)c2c(n1)CC(c1ccccc1)C/C2=N\O]

56. A compound, wherein the compound is:

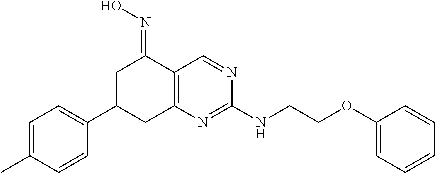 [SMILES: Cc1ccc(C2C/C(=N\O)c3cnc(NCCOc4ccccc4)nc3C2)cc1]

57. A compound, wherein the compound is:

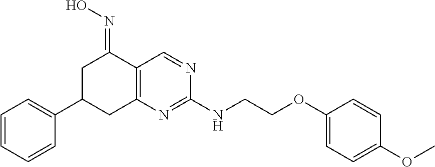 [SMILES: COc1ccc(OCCNc2ncc3c(n2)CC(c2ccccc2)C/C3=N\O)cc1]

58. A compound, wherein the compound is:

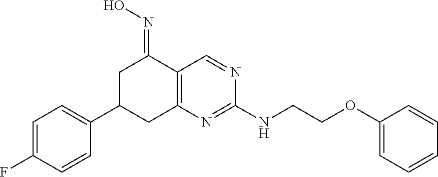 [SMILES: O/N=C1\CC(c2ccc(F)cc2)Cc2nc(NCCOc3ccccc3)ncc21]

59. A compound, wherein the compound is:

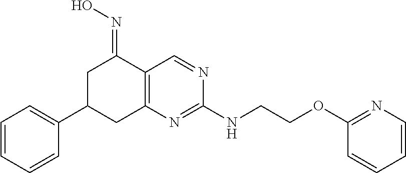 [SMILES: O/N=C1\CC(c2ccccc2)Cc2nc(NCCOc3ccccn3)ncc21]

60. A compound, wherein the compound is:

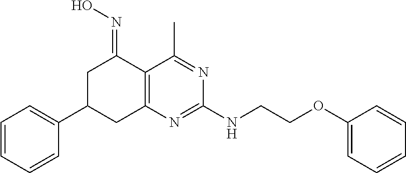 [SMILES: Cc1nc(NCCOc2ccccc2)nc2c1/C(=N/O)CC(c1ccccc1)C2]

61. A compound, wherein the compound is:

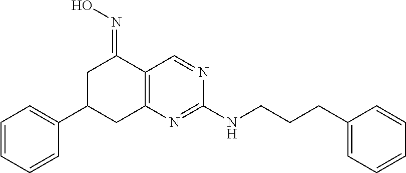 [SMILES: O/N=C1\CC(c2ccccc2)Cc2nc(NCCCc3ccccc3)ncc21]

62. A compound, wherein the compound is:

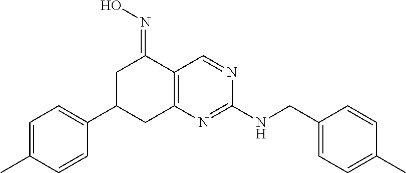

63. A compound, wherein the compound is:

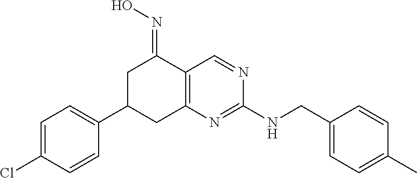 [SMILES: Cc1ccc(CNc2ncc3c(n2)CC(c2ccc(Cl)cc2)C/C3=N\O)cc1]

64. A compound, wherein the compound is:

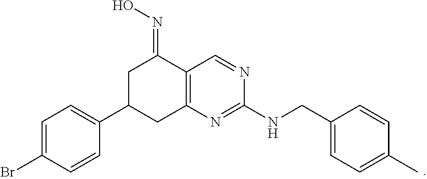 [SMILES: Cc1ccc(CNc2ncc3c(n2)CC(c2ccc(Br)cc2)C/C3=N\O)cc1]

65. A compound, wherein the compound is:

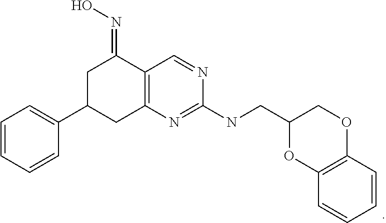 [SMILES: O/N=C1\CC(c2ccccc2)Cc2nc(NCC3COc4ccccc4O3)ncc21]

66. A compound, wherein the compound is:

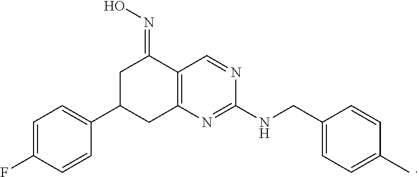 [SMILES: Cc1ccc(CNc2ncc3c(n2)CC(c2ccc(F)cc2)C/C3=N\O)cc1]

67. A compound, wherein the compound is:

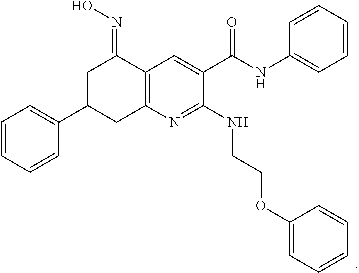 [SMILES: O=C(Nc1ccccc1)c1cc2c(nc1NCCOc1ccccc1)CC(c1ccccc1)C/C2=N\O]

68. A compound, wherein the compound is:

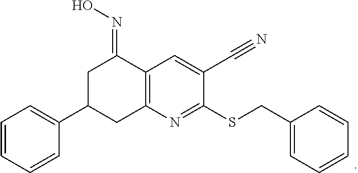 [SMILES: N#Cc1cc2c(nc1SCc1ccccc1)CC(c1ccccc1)C/C2=N\O]

69. A compound, wherein the compound is:

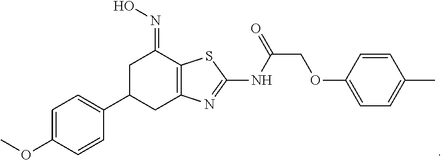

70. A compound, wherein the compound is:

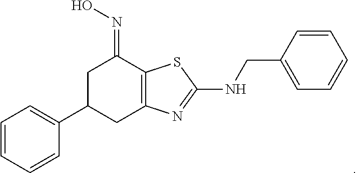 [SMILES: O/N=C1\CC(c2ccccc2)Cc2nc(NCc3ccccc3)sc21]

71. A compound, wherein the compound is:

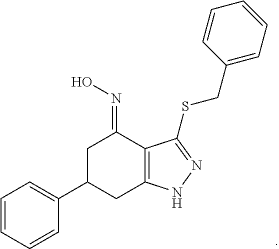 [SMILES: O/N=C1\CC(c2ccccc2)Cc2[nH]nc(SCc3ccccc3)c21]

72. A compound, wherein the compound is:

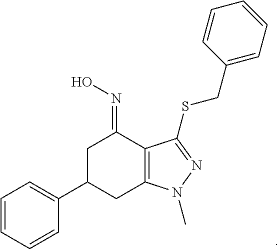 [SMILES: Cn1nc(SCc2ccccc2)c2c1CC(c1ccccc1)C/C2=N\O]

73. A compound, wherein the compound is:

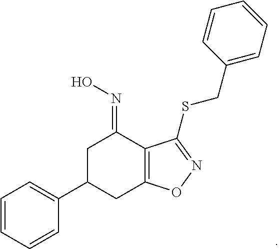 [SMILES: O/N=C1\CC(c2ccccc2)Cc2onc(SCc3ccccc3)c21]

74. A compound, wherein the compound is:

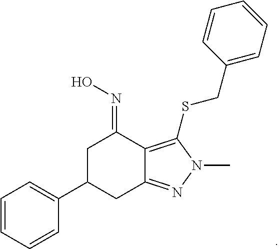 [SMILES: Cn1nc2c(c1SCc1ccccc1)/C(=N/O)CC(c1ccccc1)C2]

75. A compound, wherein the compound is selected from the group consisting of:

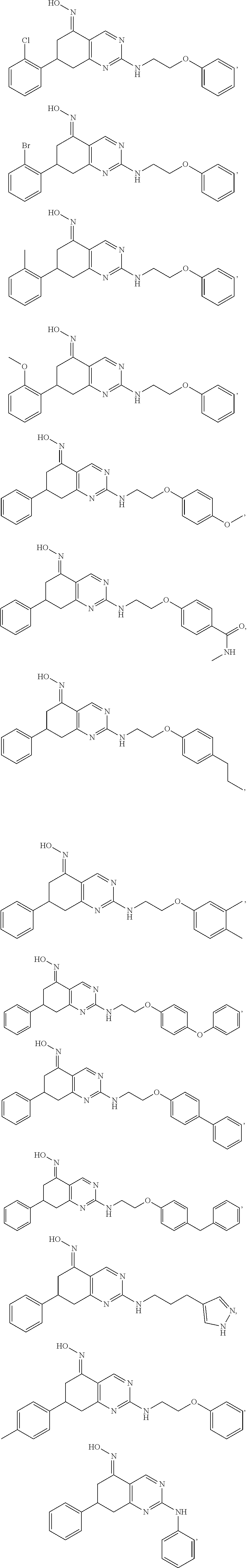

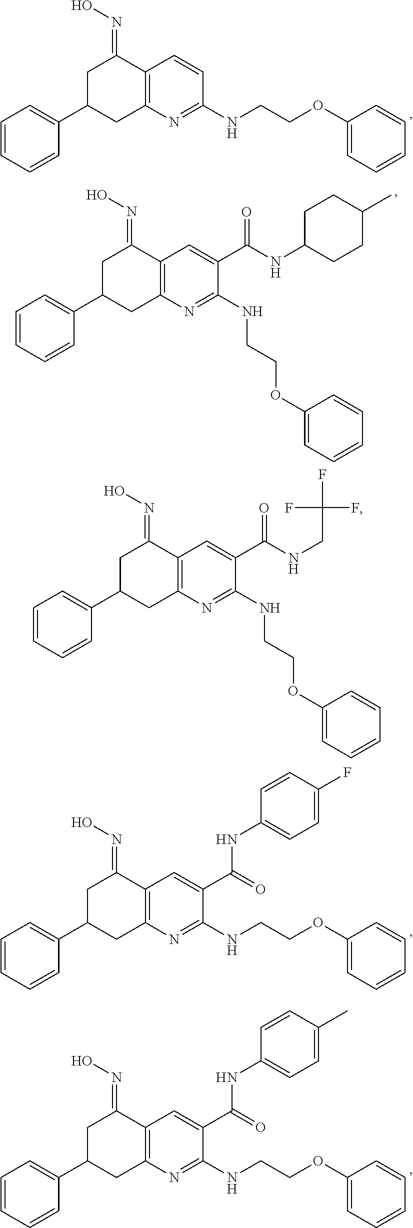

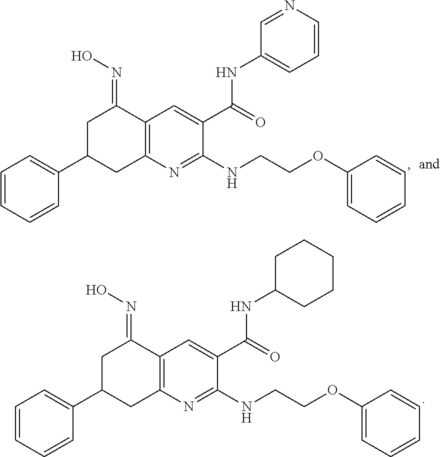 [SMILES: O=C(Nc1cccnc1)c1cc2c(nc1NCCOc1ccccc1)CC(c1ccccc1)C/C2=N\O ; O=C(NC1CCCCC1)c1cc2c(nc1NCCOc1ccccc1)CC(c1ccccc1)C/C2=N\O]

76. A compound, wherein the compound is:

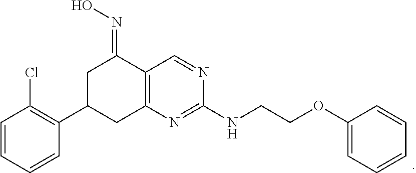 [SMILES: O/N=C1\CC(c2ccccc2Cl)Cc2nc(NCCOc3ccccc3)ncc21]

77. A compound, wherein the compound is:

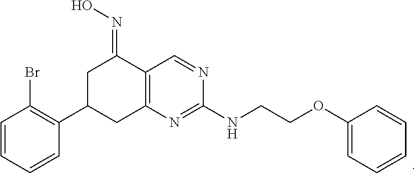 [SMILES: O/N=C1\CC(c2ccccc2Br)Cc2nc(NCCOc3ccccc3)ncc21]

78. A compound, wherein the compound is:

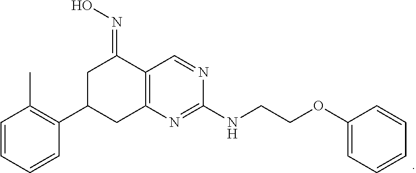 [SMILES: Cc1ccccc1C1C/C(=N\O)c2cnc(NCCOc3ccccc3)nc2C1]

79. A compound, wherein the compound is:

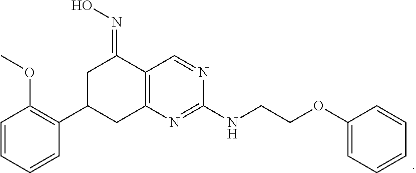 [SMILES: COc1ccccc1C1C/C(=N\O)c2cnc(NCCOc3ccccc3)nc2C1]

80. A compound, wherein the compound is:

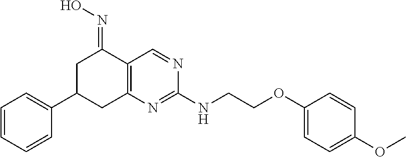 [SMILES: COc1ccc(OCCNc2ncc3c(n2)CC(c2ccccc2)C/C3=N\O)cc1]

81. A compound, wherein the compound is:

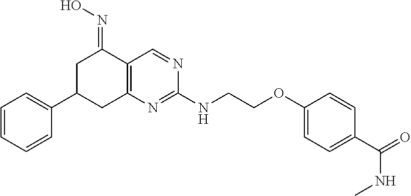 [SMILES: CNC(=O)c1ccc(OCCNc2ncc3c(n2)CC(c2ccccc2)C/C3=N\O)cc1]

82. A compound, wherein the compound is:

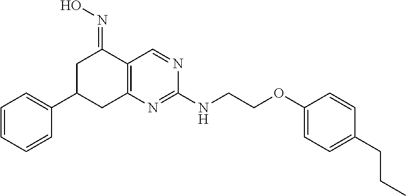 [SMILES: CCCc1ccc(OCCNc2ncc3c(n2)CC(c2ccccc2)C/C3=N\O)cc1]

83. A compound, wherein the compound is:

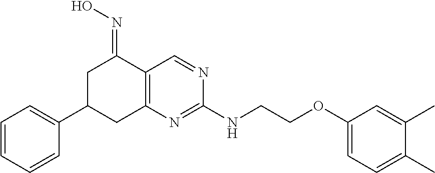 [SMILES: Cc1ccc(OCCNc2ncc3c(n2)CC(c2ccccc2)C/C3=N\O)cc1C]

84. A compound, wherein the compound is:

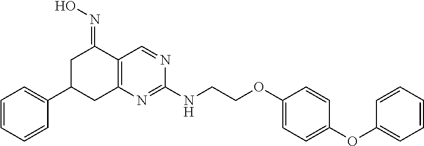 [SMILES: O/N=C1\CC(c2ccccc2)Cc2nc(NCCOc3ccc(Oc4ccccc4)cc3)ncc21]

85. A compound, wherein the compound is:

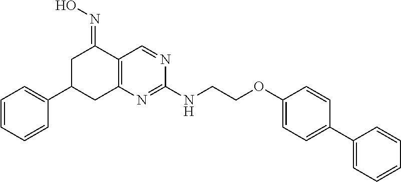 [SMILES: O/N=C1\CC(c2ccccc2)Cc2nc(NCCOc3ccc(-c4ccccc4)cc3)ncc21]

86. A compound, wherein the compound is:

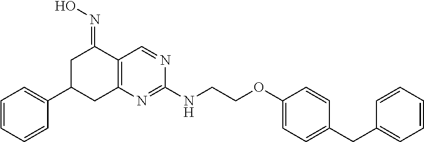 [SMILES: O/N=C1\CC(c2ccccc2)Cc2nc(NCCOc3ccc(Cc4ccccc4)cc3)ncc21]

87. A compound, wherein the compound is:

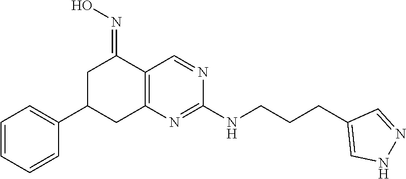 [SMILES: O/N=C1\CC(c2ccccc2)Cc2nc(NCCCc3cn[nH]c3)ncc21]

88. A compound, wherein the compound is:

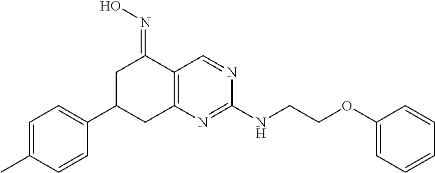 [SMILES: Cc1ccc(C2C/C(=N\O)c3cnc(NCCOc4ccccc4)nc3C2)cc1]

89. A compound, wherein the compound is:

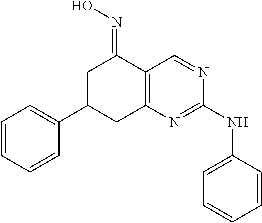 [SMILES: O/N=C1\CC(c2ccccc2)Cc2nc(Nc3ccccc3)ncc21]

90. A compound, wherein the compound is:

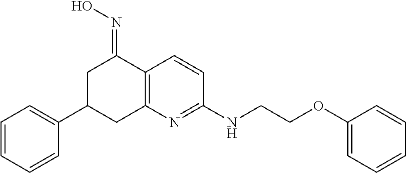 [SMILES: O/N=C1\CC(c2ccccc2)Cc2nc(NCCOc3ccccc3)ccc21]

91. A compound, wherein the compound is:

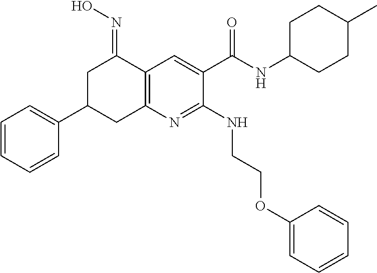 [SMILES: CC1CCC(NC(=O)c2cc3c(nc2NCCOc2ccccc2)CC(c2ccccc2)C/C3=N\O)CC1]

92. A compound, wherein the compound is:

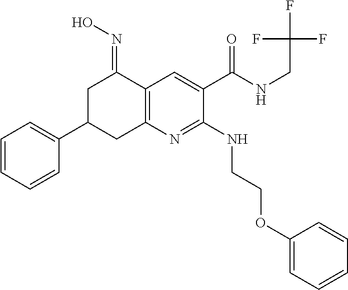 [SMILES: O=C(NCC(F)(F)F)c1cc2c(nc1NCCOc1ccccc1)CC(c1ccccc1)C/C2=N\O]

93. A compound, wherein the compound is:

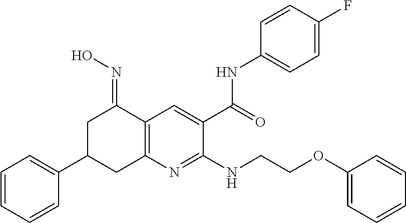 [SMILES: O=C(Nc1ccc(F)cc1)c1cc2c(nc1NCCOc1ccccc1)CC(c1ccccc1)C/C2=N\O]

94. A compound, wherein the compound is:

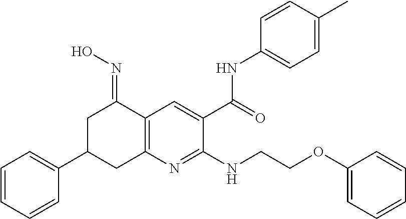 [SMILES: Cc1ccc(NC(=O)c2cc3c(nc2NCCOc2ccccc2)CC(c2ccccc2)C/C3=N\O)cc1]

95. A compound, wherein the compound is:

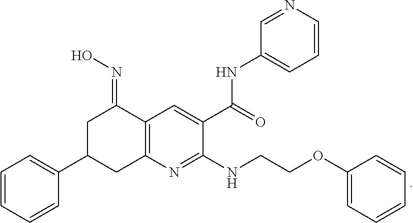 [SMILES: O=C(Nc1cccnc1)c1cc2c(nc1NCCOc1ccccc1)CC(c1ccccc1)C/C2=N\O]

96. A compound, wherein the compound is:

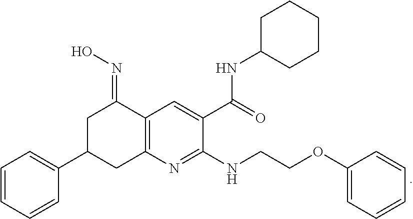 [SMILES: O=C(NC1CCCCC1)c1cc2c(nc1NCCOc1ccccc1)CC(c1ccccc1)C/C2=N\O]

97. A composition comprising an agent, said composition being configured to be ingested or selectively activated by said macrophages.
98. A composition comprising an agent, said composition being configured to be ingested or selectively activated by said macrophages, wherein said composition comprises a delivery vehicle and said agent, wherein the delivery vehicle facilitates the ingestion of the agent by said macrophages.
99. A composition comprising an agent, said composition being configured to be ingested or selectively activated by said macrophages, wherein said composition comprises a delivery vehicle and said agent, wherein the delivery vehicle facilitates the ingestion of the agent by said macrophages, and wherein the delivery vehicle comprises a liposome or a nanoparticle.
100. A composition comprising an agent and a vehicle, wherein the agent comprises one or more compounds of any one of the preceding embodiments and wherein the vehicle comprises a liposome or a nanoparticle.
101. A composition comprising an agent is active or becomes active in macrophages that are β-galactosidase positive and is inactive against non-macrophage cell types.
102. A composition comprising an agent that is active or becomes active in macrophages that are β-galactosidase positive and is inactive against non-macrophage cell types, said composition being configured to be ingested or selectively activated by said macrophages, wherein said composition comprises a delivery vehicle and said agent, wherein the delivery vehicle facilitates the ingestion of the agent by said macrophages.
103. A composition comprising an agent that is active or becomes active in macrophages that are β-galactosidase positive and is inactive against non-macrophage cell types, said composition being configured to be ingested or selectively activated by said macrophages, wherein said composition comprises a delivery vehicle and said agent, wherein the delivery vehicle facilitates the ingestion of the agent by said macrophages, and wherein the delivery vehicle comprises a liposome or a nanoparticle.
104. A composition comprising an agent, wherein said agent comprises a delivery vehicle and a drug which is active when ingested by a cell.
105. A composition comprising an agent, wherein said agent comprises a delivery vehicle and a drug which is active when ingested by a cell, and wherein said drug comprises a poison active inside a cell, a prodrug which is activatable inside a cell, an RNA or polypeptide toxic to a cell, an RNA or polypeptide which inhibits NFκB, or one or more compounds of any of the preceding embodiments.
106. A composition comprising an agent, wherein said agent comprises a delivery vehicle and a drug which is active when ingested by a cell, and wherein said drug comprises a poison active inside a cell, a prodrug which is activatable inside a cell, an RNA or polypeptide toxic to a cell, an RNA or polypeptide which inhibits NFκB, or one or more compounds of any one of the preceding embodiments, and wherein the delivery vehicle comprises a liposome, a nanoparticle, an antibody, an expression vector comprising a gene encoding an RNA or polypeptide toxic to a cell, an expression vector comprising a gene encoding an RNA or polypeptide which inhibits NFκB.
107. The composition of any one of the preceding embodiments, wherein the agent, if mentioned, is selected from the group consisting of TA-4812, OT-82, interferon-alpha, interferon-beta, poly(I:C) RNA, activators of toll-like receptor 3 (TLR3), activators of toll-like receptor 4 (TLR4), ruxolitinib, momelotinib, JAK1/JAK2 inhibitors, AS1517499, STATE inhibitors, IL-4 neutralizing antibodies, IL-4 receptor neutralizing antibodies, IL-13 neutralizing antibodies, IL-13 receptor neutralizing antibodies, TA-08209-1, TA-08210-1 and one or more of the compounds of any one of the preceding embodiments.
108. The composition of any one of the preceding embodiments, wherein the agent if mentioned comprises a prodrug that becomes active in said macrophages following activation within said macrophages that are β-galactosidase positive and is inactive macrophages that are not β-galactosidase positive.
109. The composition of any one of the preceding embodiments, wherein the prodrug, if mentioned, comprises a drug selected from the group consisting of TA-4812, OT-82, interferon-alpha, interferon-beta, poly(I:C) RNA, activators of toll-like receptor 3 (TLR3), activators of toll-like receptor 4 (TLR4), ruxolitinib, momelotinib, JAK1/JAK2 inhibitors, AS1517499, STATE inhibitors, IL-4 neutralizing antibodies, IL-4 receptor neutralizing antibodies, IL-13 neutralizing antibodies, IL-13 receptor neutralizing antibodies, TA-08209-1, TA-08210-1 and one or more of the compounds of embodiments 1-4.
110. The compound or composition of any one of the preceding embodiments, wherein said agent, if mentioned, is capable of killing or reprogramming SAMs.
111. The composition of any one of the preceding embodiments, wherein the agent, if present, comprises a prodrug that becomes active in macrophages that are β-galactosidase positive and is inactive against non-macrophage cell types.
112. A method of selectively killing one or more types of senescent cells in a subject in need thereof, the method comprising administering to the subject a composition comprising a therapeutically effective amount of one or more compounds of any one of embodiments 1-4.
113. A method of selectively killing one or more types of senescent cells in a subject in need thereof, the method comprising administering to the subject a composition comprising a therapeutically effective amount of one or more compounds of any one of embodiments 1-4, wherein the senescent cells are senescent due to telomere shortening (replicative cellular senescence), DNA damaging treatment (ionizing radiation, chemotherapeutic drugs, UV, exposure to oxidative stresses and other types of genotoxic stresses), direct (e.g., mutant RAS) or indirect (inactivation of tumor suppressor PTEN) activation of dominant oncogenes, or conditions resulting in acquisition of pathological properties associated with senescent cells but not involving DNA damage (prolonged growth arrest under conditions prohibiting cell divisions), etc.
114. The method of any one any one of the preceding embodiments, wherein the senescent cells are from an aged organism or age-related pathology.
115. A method for delaying at least one pathological feature of aging in a subject, the method comprising administering a composition comprising a therapeutically effective amount of one or more compounds or compositions of any one of the preceding embodiments.
116. The method of any one of the preceding embodiments, wherein the subject, if mentioned, has received a DNA-damaging therapy or accidental DNA damage.
117. A method of treating an age-related disease or condition, the method comprising administering a composition comprising a therapeutically effective amount of one or more compounds or compositions of any one any one of the preceding embodiments, provided the age-related disease or condition is not cancer.
118. The method any one of the preceding embodiments, wherein the age-related disease or condition, if mentioned, is a degenerative disease or a function-decreasing disorder.
119. A method of killing therapy-induced senescent cells, the method comprising administering a composition comprising a therapeutically effective amount of one or more compounds of any one of the preceding embodiments to a subject that has received DNA-damaging therapy and killing therapy induced-senescent cells in normal and tumor tissues following DNA-damaging therapy.
120. A method for removing senescence-associated macrophages (SAMs) in a mixed population of cells comprising SAMs, the method comprising selectively inducing SAM cell death in said mixed population of cells.
121. A method for removing senescence-associated macrophages (SAMs) in a mixed population of cells comprising SAMs, the method comprising selectively inducing SAM cell death in said mixed population of cells by administering one or more compounds or compositions of any one of the preceding embodiments.
122. A method for removing senescence-associated macrophages (SAMs) in a mixed population of cells comprising SAMs, the comprising selectively inducing SAM cell death in said mixed population of cells, wherein SAM cell death is selectively induced by delivering to said mixed population of cells an agent which is selectively ingested by SAMs such that SAMs are removed from said mixed population.
123. A method for removing senescence-associated macrophages (SAMs) in a mixed population of cells comprising SAMs, the comprising selectively inducing SAM cell death in said mixed population of cells, wherein SAM cell death is selectively induced by delivering to said mixed population of cells an agent which is selectively ingested by SAMs such that SAMs are removed from said mixed population; wherein said agent comprises one or more compounds or compositions of any one of the preceding embodiments.
124. A method of reducing chronic systemic inflammation in an aging adult, comprising administering an agent capable of reducing or eradicating senescence-associated macrophages (SAMs) in a mammal, thereby reducing said inflammation.
125. A method of reducing chronic systemic inflammation in an aging adult, comprising administering an agent capable of reducing or eradicating senescence-associated macrophages (SAMs) in a mammal, thereby reducing said inflammation; wherein said agent comprises one or more compounds or compositions of any one of the preceding embodiments.
126. A method of selectively killing SAMs in a mammal comprising administering to the mammal a pharmaceutical composition comprising an agent which is toxic upon ingestion by a mammalian cell and a delivery vehicle which provides said agent to said cell for ingestion.
127. A method of selectively killing SAMs in a mammal comprising administering to the mammal a pharmaceutical composition comprising an agent which is toxic upon ingestion by a mammalian cell and a delivery vehicle which provides said agent to said cell for ingestion; wherein said pharmaceutical composition comprises one or more compounds or compositions of any one of the preceding embodiments.
128. A method of treating or preventing an age-related disease in a mammal, the method comprising administering an agent capable of reducing, eradicating, or reprogramming senescence-associated macrophages (SAMs) in a mammal, thereby treating said disease.
129. A method of treating or preventing an age-related disease in a mammal, the method comprising administering an agent capable of reducing, eradicating, or reprogramming senescence-associated macrophages (SAMs) in a mammal, thereby treating said disease; wherein said agent comprises one or more compounds or compositions of any one of the preceding embodiments.
130. A method of treating a disease related to or caused by cellular senescence in a mammal comprising administering to the mammal a pharmaceutical composition comprising: (a) an agent that is toxic to a cell upon ingestion and a delivery vehicle.
131. A method of treating a disease related to or caused by cellular senescence in a mammal comprising administering to the mammal a pharmaceutical composition comprising: (a) an agent that is toxic to a cell upon ingestion and a delivery vehicle; wherein said pharmaceutical composition comprises one or more compounds or compositions of any one of the preceding embodiments.
132. A method for identifying an agent that selectively removes SAMs from a mixed population of cells comprising SAMs, said method comprising (a) contacting a plurality of candidate agents with a mixed population of SAMs and senescent cells; and (b) determining selective removal of SAMs from said mixed population, thereby identifying said agent.
133. A method of assessing chronic systemic inflammation in a mammal, the method comprising detecting the presence of senescence-associated macrophages (SAMs) in a mammal, wherein the presence of SAMs is indicative of chronic systemic inflammation.
134. A method of reprogramming senescence-associated macrophages (SAMs) in a mixed population of cells, the method comprising selectively reversing or reducing the SAM phenotype in said mixed population of cells.
135. A method of reprogramming senescence-associated macrophages (SAMs) in a mixed population of cells, the method comprising selectively reversing or reducing the SAM phenotype in said mixed population of cells by administering one or more compounds or compositions of any one of the preceding embodiments.
136. A method of reprogramming senescence-associated macrophages (SAMs) in a mixed population of cells, the method comprising selectively reversing or reducing the SAM phenotype in said mixed population of cells wherein SAM phenotype is selectively reversed or reduced by delivering to the mixed population of cells an agent capable of reducing SAM expression of p16, β-galactosidase, or both.
137. A method of reprogramming senescence-associated macrophages (SAMs) in a mixed population of cells, the method comprising selectively reversing or reducing the SAM phenotype in said mixed population of cells wherein SAM phenotype is selectively reversed or reduced by delivering to the mixed population of cells an agent capable of reducing SAM expression of p16, β-galactosidase, or both; and wherein said agent is one or more compounds or compositions of any one of the preceding embodiments.
138. A method of removing SAMs from a mixed population of cells, the method comprising delivering to the mixed population of cells a first polarization agent capable of modulating the polarization status of the SAMs and a second agent that is toxic the SAMs.
139. A method of removing SAMs from a mixed population of cells, the method comprising delivering to the mixed population of cells a first polarization agent capable of modulating the polarization status of the SAMs and a second agent that is toxic the SAMs, wherein the first polarizing agent causes said SAMs to be more susceptible to the toxic effects of the second agent.
140. A method of removing SAMs from a mixed population of cells, the method comprising delivering to the mixed population of cells a first polarization agent capable of modulating the polarization status of the SAMs and a second agent that is toxic the SAMs, wherein the first agent polarizes SAMs to an M1 phenotype and the second agent is selectively toxic to the M1 phenotype SAMs.
141. A method of removing SAMs from a mixed population of cells, the method comprising delivering to the mixed population of cells a first polarization agent capable of modulating the polarization status of the SAMs and a second agent that is toxic the SAMs, wherein the first agent is selected from the group consisting of interferon-alpha, interferon-beta, interferon-gamma, toll-like receptor 3 (TLR3) agonists, toll-like receptor 4 (TLR4) agonists, and poly(I:C) RNA.
142. A method of removing SAMs from a mixed population of cells, the method comprising delivering to the mixed population of cells a first polarization agent capable of modulating the polarization status of the SAMs and a second agent that is toxic the SAMs, wherein the first agent is selected from the group consisting of interferon-alpha, interferon-beta, interferon-gamma, toll-like receptor 3 (TLR3) agonists, toll-like receptor 4 (TLR4) agonists, and poly(I:C) RNA; and wherein the second agent is a compound or composition of any of the preceding embodiments.
143. The method of any one of the preceding embodiments, wherein said mixed population of cells if mentioned is present in vitro in culture.
144. The method of any one of the preceding embodiments, wherein said mixed population of cells, if mentioned, is present in a mammal.
145. The method of any one of the preceding embodiments, wherein said mammal is a human.
146. The method of any one of the preceding embodiments, wherein SAM cell death, if mentioned, is selectively induced by delivering to said mixed population of cells an agent which is selectively ingested by SAMs by not by SCs such that SAMs are removed from said mixed population.
147. The method of any one of the preceding embodiments, wherein said disease is cancer, age-related disease, tobacco-related disease, or skin wrinkles.
148. The method of any one of the preceding embodiments, wherein said cancer, if mentioned, is prostate cancer, colon cancer, lung cancer, squamous cell cancer of the head and neck, esophageal cancer, hepatocellular carcinoma, gastric cancer, pancreatic cancer, ovarian cancer, or breast cancer.
149. The method of any one of the preceding embodiments, wherein said age-related or tobacco-related disease, if mentioned, is cardiovascular disease, cerebrovascular disease, peripheral vascular disease, Alzheimer's disease, osteoarthritis, cardiac diastolic dysfunction, benign prostatic hypertrophy, aortic aneurysm, emphysema, or diabetes.
150. The method of any one of the preceding embodiments, wherein the method comprises administering or delivering a therapeutically effective amount of one or more compounds or compositions of any one of the preceding embodiments.

### EXAMPLES

The present disclosure will be further described in the following examples, which do not limit the scope of any invention or inventions described in the claims.

### Example 1: Synthesis, Purification and Characterization of Compounds

Synthesis, purification, and characterization of compounds (in some embodiments, SAMolytic compounds) provided herein.

General Synthetic Schemes

Scheme (1). General Synthesis of Oxime Compounds (2).

The mixture of appropriative ketone (1) (1.0 equiv, 0.1 M) and hydroxylamine hydrochloride (10 equiv) in pyridine was refluxed for 12 hours, cooled to r.t. and evaporated to dryness. The residue was treated with cold water, stirred 10-15 minutes, filtered off, washed with water and diethyl ether and dried on air to afford oxime (2) as beige to brown solids in moderate to good yields. Alternatively (2) can be isolated using preparative HPLC method.

General Scheme of Synthesis of Chemotypes A1-4.

Chemotypes A1-4 have been synthesized starting from the 1,3-cyclohexanediones general formula (3) utilizing different multi-steps approaches (Scheme 2).

Scheme 2. Divergent Approach to A1-A4.

General scheme of synthesis of starting 1,3-diketones (3). Starting ketones (3) can be prepared starting from the commercially available carbonyl compounds (4) in the accordance with synthetic Scheme (3) with good yield.

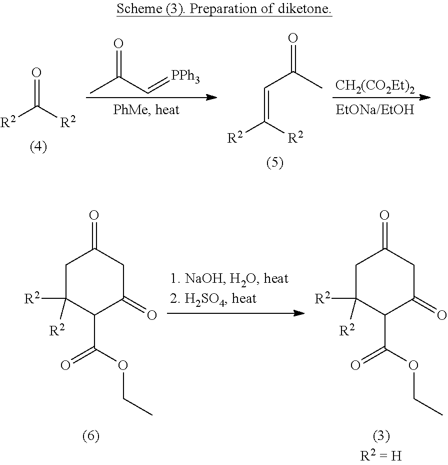

General Scheme of Synthesis of Thiophenes A1.

Thiophenic derivatives (10), (11), (13), (14), (17), (20) and (21) from chemical class A1 were synthesized in the accordance with general Scheme 4 starting from (3).

Scheme (4). Synthesis of Compounds from Class A1.

General Scheme of Synthesis of Pyrimidines A2.

Pyrimidines general formulas (25) and (29) were synthesized using chemical methods A-C as shown at the Scheme (5) starting from the 1,3-diketone (3) and commercially available amines HN(R⁹).

General Scheme of Synthesis of Pyridines A3.

Pyridines A3 were synthesized as shown at the Scheme (6) starting from the commercially available cyanoesters (32) and appropriative amines HN(R⁹).

Scheme (6). Synthesis of Pyridines A3.

General Scheme of Synthesis of Thiazoles A4.

Thiazoles A4 were synthesized as shown at the Scheme (5) starting from the ketone (3).

Scheme (7). Synthesis of Thiazoles A4.

Experimental.

General Experimental Methods. General Chemistry

All solvents and reagents were used as obtained from commercial sources unless otherwise indicated.

LCMS. The LC/MS analysis was done at Agilent 1100 with tandem mass-spectrometer with APCI ionization.

1. Type of HPLC column: Phenomenex Onyx Monolithic C18; 50×4.6 mm; Part No: CHO-7643

2. Solvent for samples dissolution: 50% DMSO, 50% acetonitrile

3. Flow rate: 3.75 mL/minute; column temperature 25° C.

4. Mobile phase: A=0.1% solution of TFA in AcN/Water (2.5:97.5), B=0.1% solution of TFA in acetonitrile

5. Gradient:

| time, minutes | A % | B % |
|---|---|---|
| 0.0 | 100 | 0 |
| 0.2 | 100 | 0 |
| 2.1 | 5 | 95 |
| 2.5 | 5 | 95 |
| 2.2 | 100 | 0 |
| 3.0 | 100 | 0 |

6. Detection: diode array (DAD), 200-800 nm; photodiode array detector. Detection was carried out in the full ultraviolet-visible range from 200 to 800 nm. APCI (+ or/and − ions)—atmospheric pressure chemical ionization ELSD (PL-ELS 2100)

7. Total run time of the method: 3 minutes

8. Injection volume: 1.7 μL.

NMR.

NMR ¹H and ¹³C spectra were recorded on Bruker Avance 400 instrument with operating frequency of 400 and 100 MHz respectively and calibrated using residual undeuterated chloroform (δH=7.28 ppm) and CDCl₃ (δCF=77.16 Ppm), or undeuterated DMSO (δH=2.50 ppm) and DMSO-d6 (δC=39.52 ppm) as internal references. The following abbreviations are used to set multiplicities: s=singlet, d=doublet, t=triplet, q=quartet, m=multiplet, br.=broad.

HPLC Analysis.

The HPLC analysis was done at Agilent 1200 instrument.

1. Synergi Hydro-RP 250×4.6 mm, 4 mkm

2. Flow rate: 1 mL/minute; column temperature—ambient

3. Mobile phase: Acetonitrile/Water (65:35, isocratic flow)

4. Detection at 254 nm wave length

TLC. Reactions were monitored by thin layer chromatography (TLC) carried out on Merck TLC Silica gel plates (60F₂₅₄), using UV light for visualization and basic aqueous potassium permanganate or iodine fumes as a developing agent.

Preparative HPLC isolation. The preparative HPCL isolation of compounds analysis was done at Agilent 1200 HPLC Preparative instrument.

Type of HPLC column: Phenomenex Luna, C18; 100×30 mm;

Solvent for samples dissolution: 50% DMSO, 50% acetonitrile

Flow rate: 30 mL/minute; column temperature 25° C.

Retention time 14 minutes, Total run time of the method: 30 minutes

Mobile phase: 0.1% solution of FA in AcN/Water (35:65), stationary phase: reversed-phase sorbent C18 (L1), 10 nm

Experimental Procedures

General procedure of synthesis of oximes. A mixture of appropriative ketone (0.5 mmol) and hydroxylamine hydrochloride (350 mg, 5 mmol) in pyridine (5 mL) was heated at refluxed for 12 hours), cooled down to r.t. and evaporated to dryness. The dark oily residue was purified using preparative HPLC to afford a title oxime.

### Experimental 1. Ethyl 3-(benzylsulfanyl)-6-(4-methoxyphenyl)-4-oxo-4,5,6,7-tetrahydro-2-benzothiophene-1-carboxylate (47)

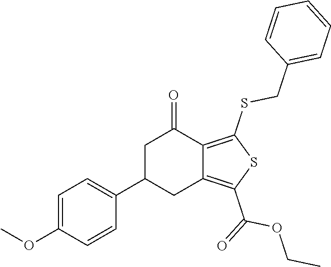 [SMILES: CCOC(=O)c1sc(SCc2ccccc2)c2c1CC(c1ccc(OC)cc1)CC2=O]

5-(4-Methoxyphenyl)cyclohexane-1,3-dione (20.0 g, 91.7 mmol) was dissolved in DMF (92 mL) and NaH (60% dispersion in mineral oil, 4.10 g, 102.5 mmol, 1.1 equiv) was added in several portions with a cooling on a water bath and vigorous stirring. When hydrogen evolution stops CS₂ (28 mL, 0.47 mol, 5 equiv, caution: very toxic!) was added in one portion. The resulting dark red solution was stirred for 5-10 minutes and benzyl chloride (10.7 ml, 93.0 mmol, 1 equiv.) was added dropwise in the course of 5-10 minutes. The reaction mixture was stirred for 30 minutes and Et3N was added in one portion (25.8 ml, 0.185 mol, 2 equiv) followed by dropwise addition of ethyl bromoacetate (10.3 ml, 92.9 mmol, 1 equiv). The reaction mixture was stirred for ˜3-4 hours, diluted with ethanol (400 mL) and filtered off (caution: stench!). The precipitate was washed with ethanol (2×50 mL) and ether (2×100 mL) and dried on air (m=21.80 g). The filtrate was left overnight and the second portion of the title compound precipitates from solution. It was filtered and washed with ethanol and ether to give 5.45 g of the product. Summary yield: 27.25 g (66%).

¹H NMR (DMSO-d₆, 400 MHz): δ=1.25 (t, J=7.0 Hz, 3H), 2.57 (br. d, J=16.4 Hz, 1H), 2.91 (dd, J=16.3, 12.4 Hz, 1H), 3.06 (dd, J=16.9, 11.4 Hz, 1H), 3.28-3.36 (m, 1H), 3.56 (br. d, J=16.9 Hz, 1H), 3.73 (s, 3H), 4.25 (q, J=7.0 Hz, 2H), 4.41 (s, 2H), 6.90 (d, J=8.4 Hz, 2H), 7.26 (d, J=8.4 Hz, 2H), 7.33 (q, J=7.1 Hz, 1H), 7.38 (t, J=7.1 Hz, 2H), 7.49 (d, J=7.5 Hz, 2H).

¹³C NMR (DMSO-d₆, 100 MHz): δ=14.2, 33.5, 38.3, 45.1, 55.0, 60.9, 113.9, 121.2, 127.8, 127.9, 128.7, 129.2, 130.8, 135.2, 135.4, 150.1, 156.1, 158.0, 160.8, 192.7. (One signal is missed in DMSO septet).

¹H NMR (CDCl₃, 400 MHz): δ=1.37 (t, J=7.2 Hz, 2H), 2.75 (dd, J=16.8, 12.2 Hz, 1H), 2.85 (ddd, J=16.9, 4.0, 1.4 Hz, 1H), 3.02 (dd, J=17.4, 11.7 Hz, 1H), 3.34 (tt, J=12.0, 4.0 Hz, 1H), 3.83 (s, 3H), 3.83 (ddd, J=17.3, 4.0, 1.5 Hz, 1H), 4.31 (s, 2H), 4.34 (q, J=7.2 Hz, 2H), 6.90 (d, J=8.7 Hz, 2H), 7.21 (d, J=8.6 Hz, 2H), 7.31-7.41 (m, 2H), 7.47 (d, J=6.6 Hz, 1H).

¹³C NMR (CDCl₃, 100 MHz): δ=14.5, 34.1, 39.9, 40.2, 45.9, 55.4, 61.2, 114.2 (2C), 122.4, 127.9 (2C), 128.2, 128.9 (2C), 129.4 (2C), 131.3, 134.6, 135.2, 150.1, 157.3, 158.6, 161.6, 193.3.

LCMS [M+H+] m/z: 453.0

### Experimental 2. 3-(Enzylsulfanyl)-6-(4-methoxyphenyl)-4-oxo-4,5,6,7-tetrahydro-2-benzothiophene-1-carboxylic acid (48)

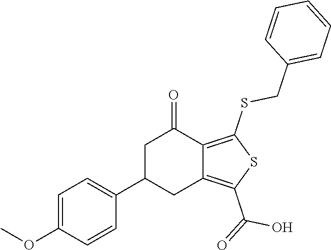 [SMILES: COc1ccc(C2CC(=O)c3c(SCc4ccccc4)sc(C(=O)O)c3C2)cc1]

The ester 47 (26.85 g, 59.3 mmol) was suspended in EtOH (270 mL) and NaOH (11.86 g, 0.297 mole, 5 equiv) solution in water (270 mL) was added in one portion. The reaction mixture was heated reflux for 3 hours (until homogeneous solution was formed). Then it was cooled to room temperature evaporated to the half of the volume and acidified with aqueous HCl (˜30 mL). The resulting precipitate was filtered off, washed several times with water and Et₂O and dried in vacuum. Yield 19.50 g (78%).

¹H NMR (DMSO-d₆, 400 MHz): δ=2.57 (br. d, J=15.9 Hz, 1H), 2.88 (dd, J=15.8, 12.9 Hz, 1H), 3.01 (dd, J=16.9, 11.6 Hz, 1H), 3.32 (t, J=11.4 Hz, 1H), 3.57 (br. d, J=17.0 Hz, 1H), 3.73 (s, 3H), 4.39 (s, 2H), 6.89 (d, J=8.2 Hz, 2H), 7.25 (d, J=8.2 Hz, 2H), 7.32 (dd, J=1.0 Hz, 1H), 7.38 (t, J=1.0 Hz, 2H), 7.49 (d, J=7.1 Hz, 2H), 13.19 (br. s., 1H).

¹³C NMR (DMSO-d₆, 100 MHz): δ=33.6, 38.3, 39.1, 45.1, 55.0, 113.9, 122.8, 127.8, 127.9, 128.7, 129.2, 130.9, 135.3, 135.5, 149.3, 155.3, 158.0, 162.4, 192.8.

### Experimental 3. 3-(benzylsulfanyl)-N-[(3-fluorophenyl)methyl]-6-(4-methoxyphenyl)-4-oxo-4,5,6,7-tetrahydro-2-benzothiophene-1-carboxamide (49)

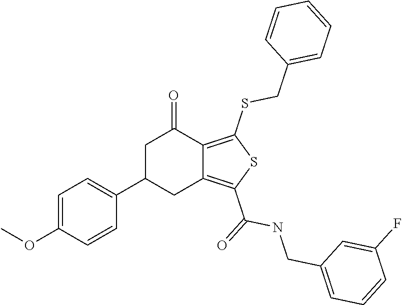 [SMILES: COc1ccc(C2CC(=O)c3c(SCc4ccccc4)sc(C(=O)NCc4cccc(F)c4)c3C2)cc1]

The acid 48 (1.00 g, 2.36 mmol) was suspended in CH₂Cl₂ (24 mL) then one drop of DMF was added followed by large excess of (COCl)₂ (2.03 ml, 23 mmol, 10 equiv, gas evolution!). The mixture was stirred until the homogeneous solution was formed (1-3 hours) and evaporated to dryness. The residue was dissolved in CH₂Cl₂ (24 mL) and (3-fluorophenyl)methanamine (3 equiv) was added in one portion (slightly exothermic!). The reaction mixture was stirred for 1 hour and diluted with water (50 mL). The organic layer was successively washed with 5% NaOH and 5% HCl, dried over Na₂SO₄ and evaporated. The crude yield is 100%. The crude 49 was used on the next step without purification.

### Experimental 4. Ethyl 6-(4-methoxyphenyl)-4-oxo-3-(prop-2-en-1-ylsulfanyl)-4,5,6,7-tetrahydro-2-benzothiophene-1-carboxylate (50)

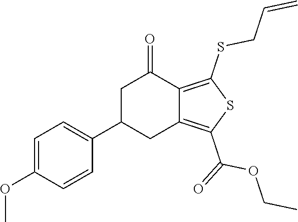 [SMILES: C=CCSc1sc(C(=O)OCC)c2c1C(=O)CC(c1ccc(OC)cc1)C2]

The title compound was synthesized in the accordance with a described above synthetic procedure for the compound 47 starting from 5-(4-methoxyphenyl)cyclohexane-1,3-dione (2.0 g, 9.2 mmol) and using allyl bromide instead of benzyl chloride. Yield: 2.13 g (53%).

### Experimental 5. 1-(Oxan-4-ylidene)propan-2-one (51)

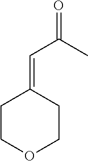

Dimethyl 2-oxopropylphosphonate (52). A 2-liter eggplant type flask was charged with 179.2 g (1.081 mol) of potassium iodide, 300 ml of acetone and 250 ml of acetonitrile, to which 100 g (1.081 mol) of chloroacetone was added, thereby forming a white suspension. Further 134 g (1.081 mol) of trimethylphosphite was added and the resulting mixture was stirred for 6 hours at 20° C. and 4 additional hours at 50° C. After that reaction mixture was filtered through the Celite pad and filtrate was distilled off to obtain a product in the form of brown oil. This oily product was subjected to fractional distillation under reduced pressure (81-85° C. and 0.02 mmHg) to obtain 127.5 g (45%) of title compound.

1-(Oxan-4-ylidene)propan-2-one (51). To a solution at 5° C. of potassium hydroxide (12.85 g, 0.229 mol) in a mixture of water (50 ml) and ethanol (200 ml) was added 52 (38.0 g, 0.229 mol), followed by the dropwise addition of tetrahydropyran-4-one (15 ml, 16.4 g, 0.163 mol). The clear solution was stirred for 5 hours at room temperature. Most of the solvent was removed from the reaction mixture under reduced pressure, and residue was diluted with a mixture of tert-butyl methyl ether and water. The organic layer was separated and aqueous layer was extracted twice with tert-butyl methyl-ether. The combined organic phases were dried over anhydrous sodium sulfate, filtered and resulting filtrate was concentrated in vacuo. The crude 51 was purified by distillation under reduced pressure to give 18.8 g (82%) of product with a boiling point of 102-104° C./12 mbar. ¹H-NMR reveals the product to be a 4:1-mixture of the desired 1-(tetrahydropyran-4-ylidene)propan-2-one and an isomer, 1-(3,6-dihydro-2H-pyran-4-yl)propan-2-one which is used without further purification on the next step.

### Experimental 6. 3-Oxaspiro[5.5]undecane-8,10-dione (53)

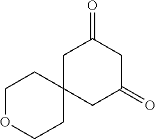 [SMILES: O=C1CC(=O)CC2(CCOCC2)C1]

To a solution of 51 (13.18 g, 94.0 mmol) in ethanol (100 ml) was added diethyl malonate (14.3 ml, 15.09 g; 94.2 mmol) at room temperature, followed by the dropwise addition of a ˜2.72 M solution of sodium ethoxide in ethanol (36.6 ML˜94.1 mmol). The solution was stirred for 3 hours at room temperature, then for 1 hour at reflux. The solvent was removed under reduced pressure and the solid residue was taken up in 12N aqueous sodium hydroxide (150 ml) and stirred at room temperature overnight. The alkaline aqueous mixture was washed once with tert-butyl methyl ether, then acidified to pH 2-3 using concentrated hydrochloric acid and warmed to 70° C. for 2 hours. The aqueous mixture was extracted with ethyl acetate and dichloromethane, and the organic phases were combined, dried over anhydrous sodium sulfate, filtered and the filtrate concentrated in vacuum. The solid residue was taken up in tert-butyl methyl ether and resulting mixture was stirred several hours. Formed off-white solid was collected by filtration to give a title 53. Yield (12.3 g, 22%), m.p. 162-164° C.

### Experimental 6. Ethyl 1-(benzylthio)-7-oxo-2′,3′,5′,6,6′,7-hexahydro-4H-spiro[benzo[c]thiophene-5,4′-pyran]-3-carboxylate (54)

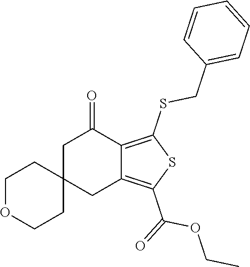 [SMILES: CCOC(=O)c1sc(SCc2ccccc2)c2c1CC1(CCOCC1)CC2=O]

1,3-Dione 53 (10.0 g, 23.9 mmol) was dissolved in DMF (45 mL) and NaH (60% dispersion in mineral oil, 2.05 g, 61.5 mmol, 1.1 equiv) was added in several portions with a cooling on a water bath and vigorous stirring. When hydrogen evolution stops CS₂ (12 mL, 0.12 mol, 5 equiv, caution: very toxic!) was added in one portion. The resulting dark red solution was stirred for 5-10 minutes and benzyl chloride (2.2 ml, 23.9 mmol, 1 equiv) was added dropwise in the course of 5-10 minutes. The reaction mixture was stirred for 30 minutes and Et₃N was added in one portion (12.4 ml, 47.8 mmol, 2 equiv) followed by dropwise addition of ethyl bromoacetate (3.7 ml, 23.9 mmol, 1 equiv). The reaction mixture was stirred for ˜3-4 hours, diluted with ethanol (200 mL) and filtered (caution: stench!). The precipitate was washed with ethanol (2×50 mL) and ether (2×50 mL) and dried in air to give a title compound. Yield: 1.98 g (47%).

### Experimental 7. Ethyl 3-(benzylsulfanyl)-6,6-dimethyl-4-oxo-4,5,6,7-tetrahydro-2-benzothiophene-1-carboxylate (55)

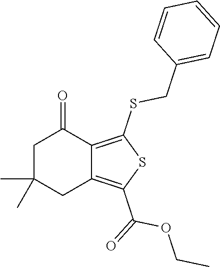 [SMILES: CCOC(=O)c1sc(SCc2ccccc2)c2c1CC(C)(C)CC2=O]

(55) was prepared with the accordance with procedure for 47 starting from dimedone (4 g, 28.6 mmol), 60% NaH in mineral oil (1.14 g, 28.6 mmol), CS₂ (8.7 g, 114 mmol), a solution of BnI in DMF (obtained by stirring for 15 minutes at 60° C. BnCl (2.7 g, 21.3 mmol) and KI (4.8 g, 28.7 mmol) in DMF (30 ml)) and ethyl bromoacetate (6.2 g, 37.1 mmol). The crude product was purified by column chromatography (silica gel, hexane-DCM-EtOAc 10:1:0.27 to 10:1:0.44) to give a solid which was dissolved in boiling EtOH (100 ml) and the solution was maintained at −20° C. overnight. The precipitate was filtered off, washed with EtOH and dried to give a title compound. Yield: 2.35 g, (22%). LCMS [M+H+] m/z 375.0 (100%).

### Experimental 8. 3-(Benzylthio)-6,6-dimethyl-4-oxo-4,5,6,7-tetrahydrobenzo[c]thiophene-1-carboxylic acid (56)

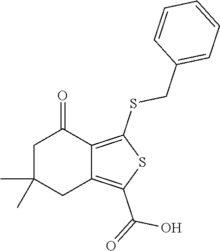 [SMILES: CC1(C)CC(=O)c2c(SCc3ccccc3)sc(C(=O)O)c2C1]

To a solution of 55 (2 g, 5.35 mmol) in a mixture of 1,4-dioxane-EtOH (2.5:1, 70 ml) 10% aqueous NaOH (10 ml) was added and resulting reaction mixture was stirred at r.t. for 5 hours and then maintained overnight. Then organic solvents were removed in vacuo; water (100 ml) followed by 10% aq. H2504 (15 ml) were added and the mixtures was extracted with DCM-EtOH 4:1 mixture (2×100 ml). The combined organic layers were dried over Na₂SO₄, concentrated to a vol. ˜4 ml and ether (20 ml) was added. After crystallization at r.t. was complete, the mixture was further maintained at −20° C. overnight. The formed precipitate was filtered off, washed with ether and dried to give title compound. Yield: 1.57 g (85%). Mother liquor was concentrated to a vol. ˜2 ml and ether (5 ml) was added and the mixture was crystallized as above to give additional portion of 56 (0.09 g, 5%). LCMS [M+H+] m/z 346.9

### Experimental 9. N-benzyl-3-(benzylsulfanyl)-6,6-dimethyl-4-oxo-4,5,6,7-tetrahydro-2-benzothiophene-1-carboxamide (57)

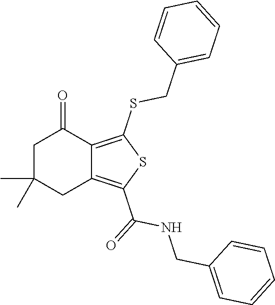 [SMILES: CC1(C)CC(=O)c2c(SCc3ccccc3)sc(C(=O)NCc3ccccc3)c2C1]

To a suspension of 56 (0.15 g, 0.434 mmol) in DCM (3 ml) one drop of DMF and oxalyl chloride (0.100 g, 0.787 mmol) were added and the reaction mixture was stirred at r.t. until the precipitate was completely dissolved (1-2 hours). Then DCM and the excess of oxalyl chloride were carefully removed in vacuo. The residue was dissolved in DCM (3 ml) and resulting solution was added to a stirred at 0° C. solution of benzyl amine (0.65 mmol) and triethylamine (0.065 g, 0.644 mmol) in DCM (3 ml). The reaction mixture was stirred for 15 minutes at 0° C., then cooling bath was removed and reaction mixture was stirred at r.t. for 1 hour. Then reaction was quenched with a 10% aq. K₂CO₃ (20 ml) and extracted with EtOAc (20 ml). An organic layer was washed with 2.5% aq. H₂SO₄ (2×20 ml), dried over Na₂SO₄ and concentrated. The residue was purified by preparative HPLC to give a title amide. Yield: 123 mg (65%).

### Experimental 10. Ethyl 6-(4-methoxyphenyl)-4-oxo-3-phenylmethanesulfonyl-4,5,6,7-tetrahydro-2-benzothiophene-1-carboxylate (58)

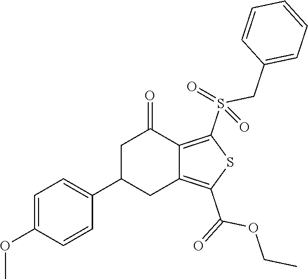 [SMILES: CCOC(=O)c1sc(S(=O)(=O)Cc2ccccc2)c2c1CC(c1ccc(OC)cc1)CC2=O]

Ester 47 (3.00 g, 6.63 mmol) was dissolved in CH₂Cl₂ (66 mL), and m-CPBA (3.75 g, 70%, 15.2 mmol, 2.3 equiv) was added in several portions at r.t. The reaction mixture was stirred for 5 hours at r.t. After that a reaction was quenched with a mixture of 5% Na₂SO₃ and 5% K₂CO₃ aqueous solutions. The organic layer was separated and aqueous was extracted with CH₂Cl₂ (2×50 mL). The combined organic layers were dried over Na₂SO₄ and evaporated to give title sulphone 58 pure enough for the subsequent reactions. Crude yield: 3.22 g, (100%).

### Experimental 11. Ethyl 6-(4-methoxyphenyl)-4-oxo-3-(phenylsulfanyl)-4,5,6,7-tetrahydro-2-benzothiophene-1-carboxylate (59)

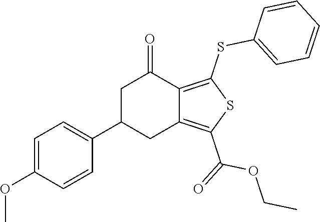 [SMILES: CCOC(=O)c1sc(Sc2ccccc2)c2c1CC(c1ccc(OC)cc1)CC2=O]

Sulphone 58 (1.60 g, 3.3 mmol), was dissolved in acetonitrile (33 ml) and K₂CO₃ (10 mmol, 3 equiv) was added followed by thiophenol (0.51 mL, 4.9 mmol, 1.5 equiv). The reaction was stirred at r.t. until the disappearance of the starting material (TLC control, ˜4 hours). The reaction mixture was diluted with water and extracted with CH₂Cl₂ (3×50 mL). The combined organic layers ware dried over Na₂SO₄ and evaporated. The residue was purified using column chromatography (silica gel, eluent—n-hexane/EtOAc, 3:1). Yield: 1.16 g (80%).

### Experimental 12. Ethyl 3-(benzylsulfanyl)-6-(4-fluorophenyl)-4-oxo-4,5,6,7-tetrahydro-2-benzothiophene-1-carboxylate (60)

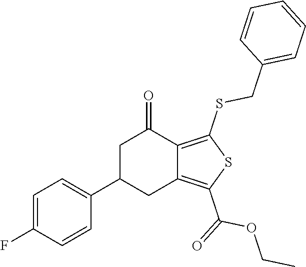 [SMILES: CCOC(=O)c1sc(SCc2ccccc2)c2c1CC(c1ccc(F)cc1)CC2=O]

The title compound was synthesized in the accordance with a described above synthetic procedure for the compound 47 starting from 5-(4-fluorophenyl)cyclohexane-1,3-dione (10 mmol). Yield: 1.89 g (43%).

### Experimental 13. Benzyl 3-(benzylsulfanyl)-6-(4-methoxyphenyl)-4-oxo-4,5,6,7-tetrahydro-2-benzothiophene-1-carboxylate (61)

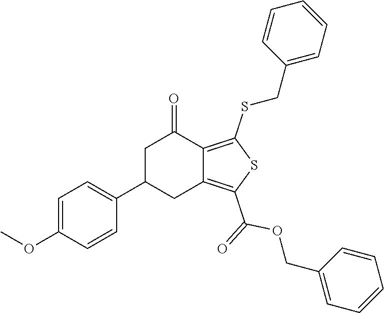 [SMILES: COc1ccc(C2CC(=O)c3c(SCc4ccccc4)sc(C(=O)OCc4ccccc4)c3C2)cc1]

The title compound was synthesized in the accordance with a described above synthetic procedure for the compound 47 starting from 5-(4-methoxyphenyl)cyclohexane-1,3-dione (5 mmol) and benzyl bromoacetate instead of ethyl bromoacetate. Yield: 1.31 g (51%).

### Experimental 14. The mixture (3:1) of ethyl 3-(benzylthio)-7-(4-methoxyphenyl)-4-oxo-4,5,6,7-tetrahydrobenzo[c]thiophene-1-carboxylate (62) and Ethyl 3-(benzylthio)-5-(4-methoxyphenyl)-4-oxo-4,5,6,7-tetrahydrobenzo[c]thiophene-1-carboxylate (63)

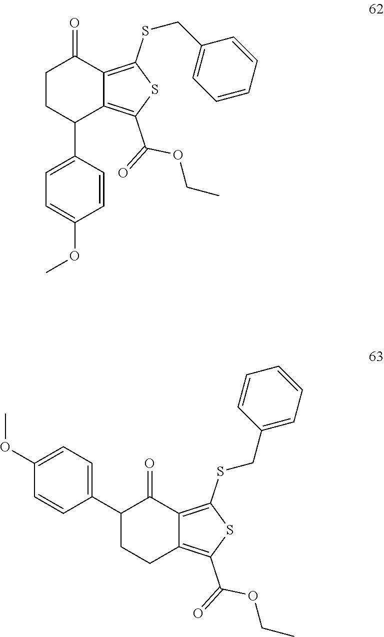 [SMILES: CCOC(=O)c1sc(SCc2ccccc2)c2c1C(c1ccc(OC)cc1)CCC2=O ; CCOC(=O)c1sc(SCc2ccccc2)c2c1CCC(c1ccc(OC)cc1)C2=O]

To a solution of 4-(4-methoxyphenyl)cyclohexane-1,3-dione (0.5 g, 2.29 mmol) in DMF (5 ml) at 0-5° C. NaH (60% suspension in mineral oil, 0.1 g, 2.52 mmol, 1.1 eq) was added, the mixture was stirred for 15 minutes and after that CS₂ (0.87 g, 11.4 mmol, 5 eq) was added and the reaction was stirred for 10 minutes. BnCl (0.29 g, 2.29 mmol, 1 eq) was added, the mixture was stirred for 30 minutes and then Et₃N (0.46 g, 4.55 mmol, 2 eq) was added followed by ethyl bromoacetate (0.385 g, 2.30 mmol, 1 eq). The reaction was stirred for 1 hour at 0-5° C. followed by 2.5 hours at r.t., then it was poured into 0.5 N aq. HCl (50 ml) and extracted with DCM (2×20 ml). Combined organic layers were washed with 0.5 N aq. K₂CO₃ (50 ml) dried with Na₂SO₄ and concentrated. The residue was purified using chromatography (SiO₂, hexane-DCM-EtOAc 6:1:0.35 as eluent) to give ˜5:1 mixture of 2 and 2a (0.38 g, 37%). The mixture was dissolved in DCM (3 ml), hexane 15 ml was added and the solution was maintained at r.t. until initial crystallization was completed. Then the mixture was maintained at 0° C. for 2 hours, the precipitate was filtered off, washed with hexane-Et₂O 3:1 mixture and dried to afford pure 2 (0.165 g, 16%).

Combined liquors from crystallization was concentrated, the residue was dissolved in DCM (1 ml), hexane 8 ml was added and the solution was subjected to crystallization as above at r.t. then at 0° C. and finally at −30° C. Liquors from filtration and washing of second precipitate was concentrated to give ˜3:1 mixture of 62 and 63 (0.06 g, ˜6%) which was used in next step.

### Experimental 15. Ethyl 2-((4-methoxyphenyl)amino)acetate (64)

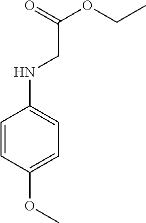

Ethyl chloroacetate (26 mL, 243 mmol) was added in one portion to a vigorously stirred mixture of p-ansidine (30 g, 243 mmol) and K₂CO₃ (33.7 g, 244 mmol) in acetonitrile (240 mL). The reaction mixture was stirred for 2 days at reflux. Reaction was cooled down, filtered off and filtrate was evaporated. The residue was dissolved in the minimal volume of dichloromethane and dry loaded on silica gel (˜100 mL). Title compound was eluted with a mixture of hexanes/EtOAc (from 5:1 to 3:1). Yield: 22.15 g (44%).

### Experimental 16. Ethyl 2-((4-methoxyphenyl)(2-oxopropyl)amino)acetate (65)

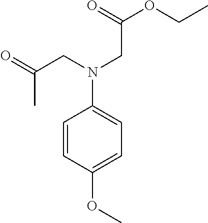 [SMILES: CCOC(=O)CN(CC(C)=O)c1ccc(OC)cc1]

Chloroacetone (20.2 mL, 253 mmol, 2.4 equiv) in was added in one portion to the stirred mixture of ethyl 2-((4-methoxyphenyl)amino)acetate 64 (22.15 g, 106 mmol) and K₂CO₃ (14.6 g, 106 mmol) in anhydrous acetonitrile (106 mL). The resulting mixture was stirred for 30 minutes at r.t. Sodium iodide (˜1 g) was added and reaction was stirred at reflux for 3 days. After cooling down the reaction mixture was filtered off and filtrate was concentrated in vacuo. The crude product was purified by column chromatography on silica gel (eluent—a mixture of hexanes/EtOAc (5:1, 4:1, 3:1). Yield: 23.68 g (84%).

### Experimental 17. 1-(4-Methoxyphenyl)piperidine-3,5-dione (66)

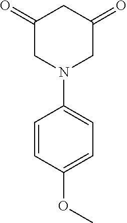 [SMILES: COc1ccc(N2CC(=O)CC(=O)C2)cc1]

To the solution of ethyl 2-((4-methoxyphenyl)(2-oxopropyl)amino)acetate 65 (5.00 g, 18.8 mmol) in dry THF (19 mL) was added dropwise a suspension of potassium tertbutoxide (2.32 g, 20.7 mmol) in dry THF (19 mL) at 0° C. under atmosphere of nitrogen over a period of 10 minutes. The reaction mixture was stirred at r.t. for 15 hours and filtered. The precipitate was suspended in water and HCl (1.73 mL) was added. The title product was extracted with CH₂Cl₂ (3×50 mL), dried over Na₂SO₄, filtered and evaporated. The residue was immediately used on the next step without further purification. Crude yield: 4.00 g (97%).

### Experimental 18. Ethyl 1-(benzylthio)-5-(4-methoxyphenyl)-7-oxo-4,5,6,7-tetrahydrothieno[3,4-c]pyridine-3-carboxylate (67)

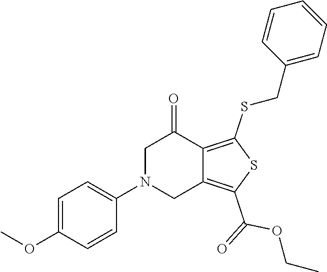 [SMILES: CCOC(=O)c1sc(SCc2ccccc2)c2c1CN(c1ccc(OC)cc1)CC2=O]

1-(4-methoxyphenyl)piperidine-3,5-dione 66 (4.0 g, 18.3 mmol) was dissolved in DMF (18 mL) and NaH (60% dispersion in mineral oil, 0.8 g, 20 mmol, 1.1 equiv) was added in several portions with cooling using a water bath and vigorous stirring. When hydrogen evolution stops CS₂ (5.5 mL, 91.4 mmol, 5 equiv, caution: very toxic!) was added in one portion. The resulting dark red solution was stirred for 5-10 minutes and benzyl chloride (2.1 ml, 18.2 mmol, 1 equiv) was added dropwise in the course of 5-10 minutes. The reaction mixture was stirred for 30 minutes and Et₃N was added in one portion (6.0 ml, 36.6 mol, 2 equiv) followed by dropwise addition of ethyl bromoacetate (2.03 ml, 92.9 mmol, 1 equiv). The reaction mixture was stirred for ˜3-4 hours, and evaporated. The residue was dry loaded on silica (˜50 mL) and eluted with a mixture of hexanes/EtOAc (3:1, 1:1, 0:1). The upper spot was collected and further triturated with a mixture of hexanes/EtOAc 3:1 to give a title product. Yield: 225 mg (3%).

### Experimental 19. 2-((Dimethylamino)methylene)-5-phenylcyclohexane-1,3-dione (68)

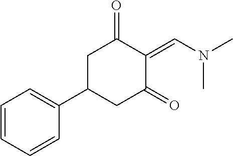 [SMILES: CN(C)C=C1C(=O)CC(c2ccccc2)CC1=O]

The mixture of 5-phenylcyclohexane-1,3-dione (18.82 g, 100 mmol, 1.0 equiv) and DMFDMA (2.0 equiv) was heated at refluxed for 2 hours (the complete dissolution resulted in dark-orange coloration). The reaction mixture was cooled down to r.t., formed precipitate was filtered off and washed with diethyl ether and dried on air to afford a title 68 as yellow solid in almost quantitative yield. The product was used without additional purification on the next step.

### Experimental 20. 2-((4-Methylbenzyl)amino)-7-phenyl-7,8-dihydroquinazolin-5(6H)-one (69)

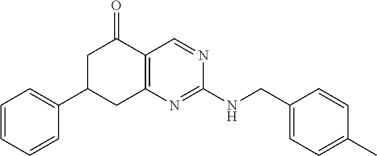 [SMILES: Cc1ccc(CNc2ncc3c(n2)CC(c2ccccc2)CC3=O)cc1]

A mixture of 68 (2 mmol, 1.0 equiv, 0.5 M), 1-(4-methylbenzyl)guanidine hydrochloride (3 mmol, 1.5 equiv) and K₂CO₃ (3 mmol, 1.5 equiv) in isopropanol was heated at reflux for 16 hours, cooled down to r.t. and diluted with 5 volumes of cold water. The resulting suspension was stirred at r.t. for 10-15 minutes. Formed precipitate was filtered off, subsequently washed with cold water, diethyl ether and dried on air to afford a title compound as beige solid. Yield: 0.385 g (56%).

### Experimental 21. 1-(2-Phenoxyethyl)guanidine (70)

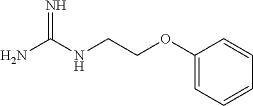 [SMILES: N=C(N)NCCOc1ccccc1]

A mixture of 2-phenoxyethan-1-amine (10 mmol, 1.0 equiv), 1H-pyrazole-1-carboxamidine hydrochloride (15 mmol, 1.5 equiv, 1 M) and DIPEA (1.5 equiv) in 1,4-dioxane/H₂O (2:1) was refluxed for 8 hours, cooled down to r.t. and evaporated to dryness. An orange oily residue was treated with water and resulting solution was saturated with solid K₂CO₃ to induce the precipitation of free guanidine. Formed precipitate was filtered off, washed with cold water and dried on a rotary evaporator to afford a title compound. Yield: 0.878 g (49%).

### Experimental 22. 2-((2-Phenoxyethyl)amino)-7-phenyl-7,8-dihydroquinazolin-5(6H)-one (71)

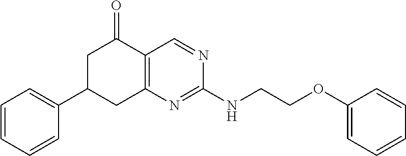 [SMILES: O=C1CC(c2ccccc2)Cc2nc(NCCOc3ccccc3)ncc21]

The title compound was synthesized in the accordance with a described above synthetic procedure for the compound 69 starting from compound 68 (2 mmol) and guanidine 70 (3 mmol). Yield: 0.489 g (69%).

### Experimental 23. 5-Phenyl-2-(4-phenylbutanoyl)cyclohexane-1,3-dione (72)

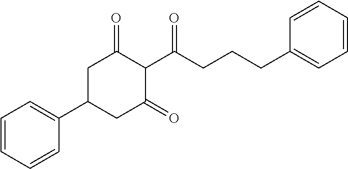 [SMILES: O=C(CCCc1ccccc1)C1C(=O)CC(c2ccccc2)CC1=O]

To a solution of 5-phenylcyclohexane-1,3-dione (20 mmol, 1.0 equiv, 0.5 M) and Et₃N (22 mmol, 1.1 equiv) in CH₂Cl₂ 4-phenylbutanoyl chloride (21 mmol, 1.05 equiv) was added dropwise under cold water bath cooling. The resulting solution was stirred at r.t. for 4 hours. Et₃N (40 mmol, 2.0 equiv) followed by acetone cyanohydrin (0.1 equiv) were added and resulting reaction mixture was stirred at r.t. overnight, diluted with CH₂Cl₂ and washed with an equivalent volume of 2-3 N HCl aq. The aqueous phase was extracted once with CH₂Cl₂. The combined organic fractions were dried over an anhydrous Na₂SO₄ and evaporated to dryness. Orange oily residue was refluxed in boiling ether and hot solution was carefully decanted from a small amount of undissolved material. The solvent was evaporated to dryness to afford a title compound as a yellow solid. Yield: 6.35 g (95%).

### Experimental 24. 5-Phenyl-2-(4-phenyl-1-(pyrrolidin-1-yl)butylidene)cyclohexane-1,3-dione (73)

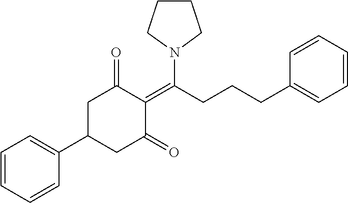 [SMILES: O=C1CC(c2ccccc2)CC(=O)C1=C(CCCc1ccccc1)N1CCCC1]

To a yellow solution of compound 72 (6.35 g, 19 mmol, 1.0 equiv, 0.5 M) in CHCl₃ pyrrolidine (23 mmol, 1.2 equiv) was added in one portion and stirred at r.t. overnight. The orange reaction mixture was diluted with CH₂Cl₂ and washed with water (2 volumes). The organic fraction was dried over anhydrous Na₂SO₄ and evaporated to dryness at low vacuum to prevent foaming. A residue was treated with boiling ether. Formed precipitate was filtered off and dried on air to afford a yellow-colored solid of title compound. Yield: 6.70 g (91%).

### Experimental 25. 2-Amino-7-phenyl-4-(3-phenylpropyl)-7,8-dihydroquinazolin-5(6H)-one (74)

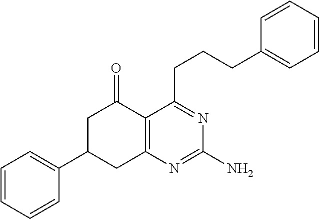 [SMILES: Nc1nc(CCCc2ccccc2)c2c(n1)CC(c1ccccc1)CC2=O]

A mixture of 73 (387 mg, 1 mmol, 1.0 equiv, 0.2 M), guanidine hydrochloride (144 mg, 1.5 mmol, 1.5 equiv) and K₂CO₃ (1.5 mmol) in 1,4-dioxane was refluxed for 30 hours, cooled down to the r.t. and diluted with 5 volumes of cold water. Resulting slurry was stirred at r.t. for 15 minutes, formed precipitate was filtered off, washed with cold water and diethyl ether and then dried on air to afford a title compound as a beige solid. Yield: 0.102 g (27%).

### Experimental 26. Ethyl 3-ethoxy-3-iminopropanoatehydrochloride (75)

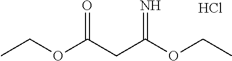

To a mixture of ethyl cyanoacetate (92.3 ml, 850 mmol), diethyl ether (50 ml), ethanol (96%, 50 ml) and water (12.3 ml) in a three neck flask (connected to a bubble counter for the evolving gas) under argon, cooled in an ice bath was added slowly thionyl chloride (59.2 ml, 805 mmol), keeping the inner temperature between 0° C. and 5° C. After the completeness of addition the mixture was stirred in an ice bath for 2 hours. The stirring was continued at room temperature for 4 hours. Thereafter the flask was cooled in a freezer (−20° C.) over night. Next day the precipitate was filtered off by suction, washed quickly in portions with diethyl ether (450 ml) and dried in vacuo. Yield: 144 g (84%). The crude product was stored under argon in a freezer (−20° C.) and used without further purification.

### Experimental 27. Ethyl 3-imino-3-((3-phenoxypropyl)amino)propanoate (76)

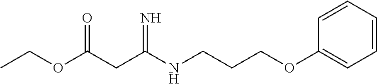 [SMILES: CCOC(=O)CC(=N)NCCCOc1ccccc1]

To the solution of potassium carbonate (42.4 g, 306.7 mmol) in water (120 ml) was added ethyl 3-ethoxy-3-iminopropanoate hydrochloride 75 (150 mmol) in several portion. The resulting mixture was extracted with diethyl ether (3×100 ml), dried over anhydrous sodium sulfate and evaporated in vacuo. The resulting oily residue was dissolved in 150 mL of ethyl alcohol, then 3-phenoxypropan-1-amine (22.68 g, 150 mmol) was added and reaction mixture was heated under reflux for 8 hours. After cooling down to the room temperature solvent was evaporated in vacuo, and a title compound was used in next steps without purification. Crude yield is 100%.

### Experimental 28. Ethyl 5-oxo-2-((2-phenoxyethyl)amino)-7-phenyl-5,6,7,8-tetrahydroquinoline-3-carboxylate (77)

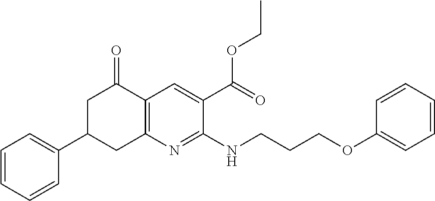 [SMILES: CCOC(=O)c1cc2c(nc1NCCCOc1ccccc1)CC(c1ccccc1)CC2=O]

Sodium (6.3 g, 127.3 mmol) was dissolved in 200 ml of absolute ethanol and compound 76 (33.65 g, 127.3 mmol) was added. The resulting mixture was stirred at r.t. for 15 minutes and compound 68 (23.8 g, 97.9 mmol) was added. The reaction mixture was stirred at reflux for 4 hr. After cooling down to the room temperature, reaction mixture was evaporated in vacuo and water was added. Precipitated solid was filtered off, washed with diethyl ether and dried in vacuum to give a title compound as a yellow solid. Yield: 18.28 g (42%).

### Experimental 29. 5-Oxo-2-((2-phenoxyethyl)amino)-7-phenyl-5,6,7,8-tetrahydroquinoline-3-carboxylic acid (78)

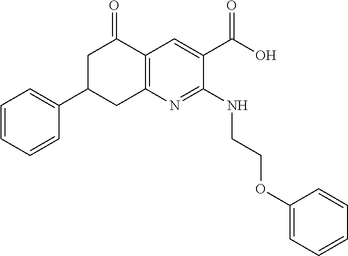 [SMILES: O=C1CC(c2ccccc2)Cc2nc(NCCOc3ccccc3)c(C(=O)O)cc21]

To the solution 77 (15.07 g, 33.9 mmol) in 150 ml of ethanol was added a solution of sodium hydroxide (2.7 g, 67.8 mmol) in 150 ml of water. Reaction mixture was heated at reflux for 6 hr, cooled down and evaporated in vacuum, then solution of acetic acid (67.8 mmol) in 200 ml of water was added, formed white precipitate was filtered off, washed with diethyl ether and dried under vacuum. Yield: 12.71 g (90%).

### Experimental 30. 5-oxo-2-((2-phenoxyethyl)amino)-N,7-diphenyl-5,6,7,8-tetrahydroquinoline-3-carboxamide (79)

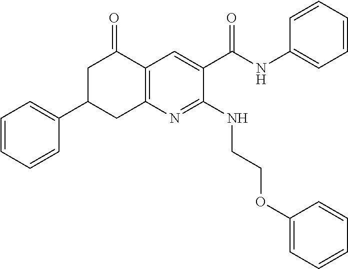 [SMILES: O=C1CC(c2ccccc2)Cc2nc(NCCOc3ccccc3)c(C(=O)Nc3ccccc3)cc21]

To the solution of 78 (500 mg, 1.2 mmol) in anhydrous dimethylformamide (10 ml), 568 mg (1.5 mmol) N,N,N′,N′-tetramethyl-O-(1H-benzotriazol-1-yl)uronium hexafluorophosphate and 265 μl (1.9 mmol) of trimethylamine were added and resulting mixture was stirred at room temperature for 15 minutes. Then aniline (1.6 mmol) was added and reaction was stirred overnight at r.t. The reaction mixture was poured into the water; precipitated solid was filtered off, washed with diethyl ether and dried in vacuum to give a title compound as a white powder. Yield: 0.23 g (39%).

### Experimental 31. 5-Oxo-7-phenyl-2-sulfanyl-5,6,7,8-tetrahydroquinoline-3-carbonitrile (80)

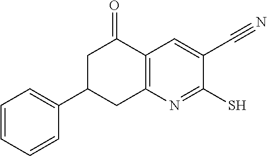 [SMILES: N#Cc1cc2c(nc1S)CC(c1ccccc1)CC2=O]

To the solution of 2-cyanoethanethioamide (2.0 g, 20.5 mmol) in dry DMF (40 ml) sodium hydride (41.1 mmol, 60% suspension in mineral oil) was slowly added at room temperature in several portions. Resulting red solution was stirred at room temperature for 15 minutes and compound 68 (4.72 g, 20.5 mmol) was added. The resulting mixture was stirred at room temperature for 24 hours and then quenched with a mixture of ethanol (20 ml) and water (20 ml) and acidified with hydrochloric acid to pH 4-5. Formed suspension was stirred at room temperature for 24 hours and poured into saturated solution of sodium chloride, filtered off, consequently washed with water, diethyl ether and dried in vacuum to give a title compound as a yellowish powder. Yield: 2.3 g (40%).

### Experimental 32. 2-(Benzylthio)-5-oxo-7-phenyl-5,6,7,8-tetrahydroquinoline-3-carbonitrile (81)

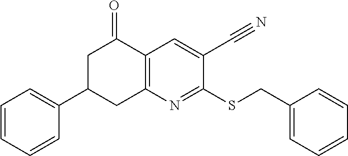 [SMILES: N#Cc1cc2c(nc1SCc1ccccc1)CC(c1ccccc1)CC2=O]

To the vigorously stirred suspension of compound 80 (500 mg, 1.8 mmol) in ethanol (10 ml) sodium acetate (295 mg, 3.6 mmol) and benzyl bromide (2 mmol) were added at r.t. and resulting reaction mixture was heated at reflux for 2 hours. A reaction was cooled down, volatilities were evaporated and dark oily residue was triturated with water. Formed precipitate was filtered off, consequently washed with water, diethyl ether and dried in vacuum to give a title compound as a dark-brown solid. Yield: 0.29 g (44%).

### Experimental 33. 2-Bromo-5-(4-methoxyphenyl)cyclohexane-1,3-dione (82)

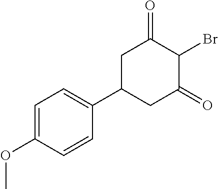 [SMILES: COc1ccc(C2CC(=O)C(Br)C(=O)C2)cc1]

To the vigorously stirred suspension of 5-(4-methoxyphenyl)cyclohexane-1,3-dione (2.18 g, 10 mmol) in acetic acid (20 ml) bromine (0.52 ml, 10 mmol) was added dropwise. The suspension became clear for a short time, and then the light yellow precipitate started to form. The newly formed suspension was stirred at r.t. for additional 2 hours. The reaction mixture was filtered off and precipitate was carefully washed with diethyl ether twice and dried thoroughly on air to give a title compound as a yellow powder. Yield: 2.74 g (92%).

### Experimental 34. 2-Bromo-5-phenylcyclohexane-1,3-dione (83)

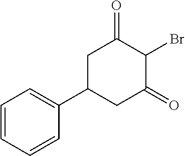 [SMILES: O=C1CC(c2ccccc2)CC(=O)C1Br]

The title compound was synthesized in the accordance with a described above synthetic procedure for the compound 82 starting from 5-phenylcyclohexane-1,3-dione (10 mmol). Yield: 2.56 g (96%).

### Experimental 35. 2-Amino-5-(4-methoxyphenyl)-5,6-dihydrobenzo[d]thiazol-7(4H)-one (84)

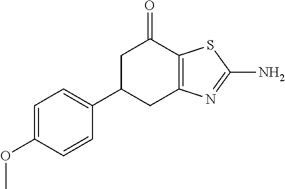 [SMILES: COc1ccc(C2CC(=O)c3sc(N)nc3C2)cc1]

To the stirred suspension of compound 82 (2.74 g, 9.2 mmol) in isopropyl alcohol (20 ml) a thiourea (1.4 g, 18.4 mmol) and potassium carbonate (18.4 mmol) were added. The resulting mixture was heated at reflux for 12 hours (the intensity of yellow color of the mixture slightly increased during this time). The reaction mixture was filtered off, and precipitate was washed with a small portion of diethyl ether. A filtered and washed precipitate was suspended in water (20 mL), heated to reflux, cooled down to rt and agitated at 0° C. for 1 hour. Newly formed precipitate was filtered off, washed with a small portion of diethyl ether and dried thoroughly in vacuo, to give a title compound as a pale yellow powder. Yield: 1.16 g (46%).

### Experimental 36. 2-(Benzylamino)-5-phenyl-5,6-dihydrobenzo[d]thiazol-7(4H)-one (85)

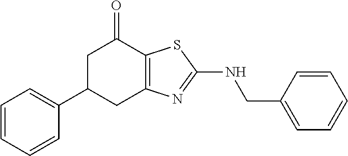 [SMILES: O=C1CC(c2ccccc2)Cc2nc(NCc3ccccc3)sc21]

The title compound was synthesized in the accordance with a described above synthetic procedure for the compound 84 starting from compound 83 (2.56 g, 9.6 mmol). Yield: 1.73 g (54%).

### Experimental 38. 3-(Benzylthio)-N-ethyl-6-(4-methoxyphenyl)-4-oxo-4,5,6,7-tetrahydrobenzo[c]thiophene-1-carboxamide (87)

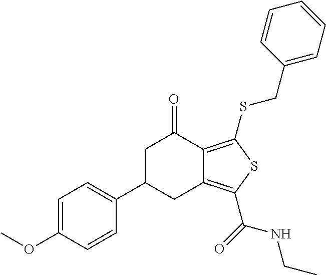 [SMILES: CCNC(=O)c1sc(SCc2ccccc2)c2c1CC(c1ccc(OC)cc1)CC2=O]

The title compound was synthesized in the accordance with a described above synthetic procedure for the compound 49 starting from acid 48 (1.0 g, 2.36 mmol) and using ethyl amine hydrochloride (20 eq) as an amine component. Yield: 0.543 g (51%).

### Experimental 39. 3-(Benzylthio)-6-(4-methoxyphenyl)-1-(morpholine-4-carbonyl)-6,7-dihydrobenzo[c]thiophen-4(5H)-one (88)

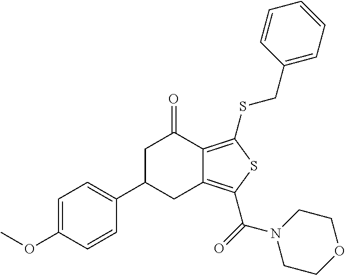 [SMILES: COc1ccc(C2CC(=O)c3c(SCc4ccccc4)sc(C(=O)N4CCOCC4)c3C2)cc1]

The title compound was synthesized in the accordance with a described above synthetic procedure for the compound 49 starting from acid 48 (1.0 g, 2.36 mmol) and using morpholine (10 eq) as an amine component. Yield: 0.50 g (43%).

### Experimental 40. N-benzyl-3-(benzylthio)-6-(4-methoxyphenyl)-4-oxo-4,5,6,7-tetrahydrobenzo[c]thiophene-1-carboxamide (89)

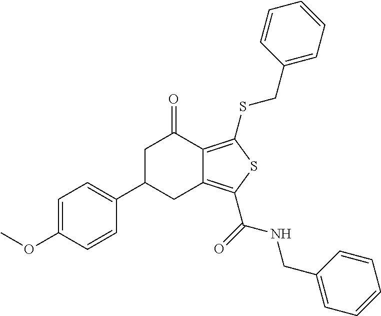 [SMILES: COc1ccc(C2CC(=O)c3c(SCc4ccccc4)sc(C(=O)NCc4ccccc4)c3C2)cc1]

The title compound was synthesized in the accordance with a described above synthetic procedure for the compound 49 starting from acid 48 (1.0 g, 2.36 mmol) and using benzyl amine (3 eq) as an amine component. Yield: 0.763 g (63%).

### Experimental 41. 3-(Benzylthio)-N-(furan-2-ylmethyl)-6-(4-methoxyphenyl)-4-oxo-4,5,6,7-tetrahydrobenzo[c]thiophene-1-carboxamide (90)

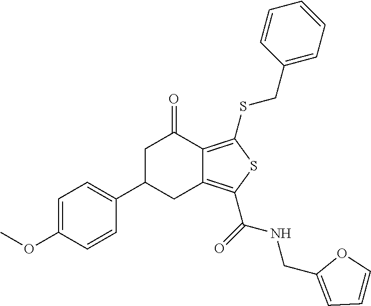 [SMILES: COc1ccc(C2CC(=O)c3c(SCc4ccccc4)sc(C(=O)NCc4ccco4)c3C2)cc1]

The title compound was synthesized in the accordance with a described above synthetic procedure for the compound 49 starting from acid 48 (1.0 g, 2.36 mmol) and using furan-2-ylmethanamine (3 eq) as an amine component. Yield: 0.392 g (33%).

### Experimental 37. N-(5-(4-methoxyphenyl)-7-oxo-4,5,6,7-tetrahydrobenzo[d]thiazol-2-yl)-2-(p-tolyloxy)acetamide (86)

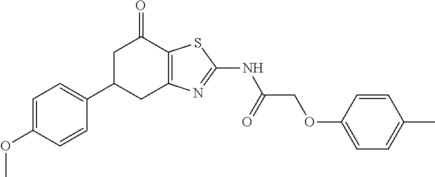 [SMILES: COc1ccc(C2CC(=O)c3sc(NC(=O)COc4ccc(C)cc4)nc3C2)cc1]

To a solution of 2-(p-tolyloxy)acetic acid (183 mg, 1.1 mmol) in dimethylformamide (5 mL) N,N,N′,N′-tetramethyl-O-(1H-benzotriazol-1-yl)uronium hexafluorophosphate (HBTU, 1.3 equiv) and triethylamine (1.5 equiv) were added. The mixture was stirred for 10 minutes and amine 84 (274 mg, 1 mmol) was added. The resulting suspension was stirred overnight, then cold water was added (the reaction was slightly exothermic). The crude product precipitated from the mixture was filtered off, washed with cold water and dried thoroughly. The crude product was re-crystallized from the hot methanol to give a title compound as a light yellow powder. Yield: 0.14 g (39%).

### Example A1.1. Ethyl 3-(benzylthio)-4-(hydroxyimino)-6-p-tolyl-4,5,6,7-tetrahydrobenzo[c]thiophene-1-carboxylate

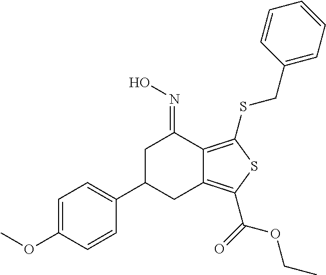 [SMILES: CCOC(=O)c1sc(SCc2ccccc2)c2c1CC(c1ccc(OC)cc1)C/C2=N\O]

A title compound was obtained from the ketone 47 in the accordance with general method of synthesis of oximes. Yield: 138 mg (59%).

¹H NMR (400 MHz, DMSO-d₆) δ=1.26 (t, J=7.1 Hz, 3H), 2.55 (d, J=11.6 Hz, 1H), 2.87-3.02 (m, 2H), 3.14 (dd, J=17.1, 2.2 Hz, 1H), 3.53 (br. d, J=14.4 Hz, 1H), 3.74 (s, 3H), 4.24 (q, J=7.1 Hz, 1H), 4.34 (dd, J=13.5, 12.0 Hz, 1H), 6.90 (d, J=8.7 Hz, 1H), 7.28-7.40 (m, 3H), 7.47 (d, J=7.2 Hz, 1H), 11.23 (s, 1H).

LCMS (ESI): ˜100%, [M+H]⁺=468.00. t_{R}=2.17 minutes.

### Example A1.2. Ethyl-3-(allylthio)-4-(hydroxyimino)-6-(4-methoxyphenyl)-4,5,6,7 tetrahydrobenzo[c]thiophene-1-carboxylate

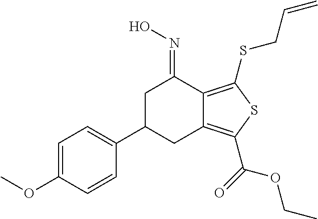 [SMILES: C=CCSc1sc(C(=O)OCC)c2c1/C(=N/O)CC(c1ccc(OC)cc1)C2]

A title compound was obtained from the ketone 50 in the accordance with general method of synthesis of oximes. Yield: 132 mg (63%).

¹H NMR (400 MHz, DMSO-d₆) δ=1.25 (t, J=7.1 Hz, 3H), 2.45-2.57 (m, 1H), 2.84-3.03 (m, 2H), 3.16 (d, J=15.3 Hz, 1H), 3.52 (d, J=15.0 Hz, 1H), 3.74 (s, 3H), 3.76 (d, J=6.7 Hz, 2H), 4.23 (q, J=7.0 Hz, 2H), 5.26 (d, J=10.0 Hz, 1H), 5.42 (d, J=17.0 Hz, 1H), 5.89-6.04 (ddd, J=16.8, 10.0, 6.7, 6.7 Hz, 1H), 6.89 (d, J=8.6 Hz, 2H), 7.25 (d, J=8.4 Hz, 2H), 11.28 (s, 1H).

LCMS (ESI): ˜100%, [M+H]⁺=418.00. t_{R}=2.08 minutes.

### Example A1.3. Ethyl-1-(benzylthio)-7-(hydroxyimino)-2′,3′,5′,6,6′,7-hexahydro-4H-spiro[benzo[c]thiophene-5,4′-pyran]-3-carboxylate

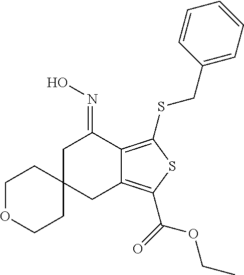 [SMILES: CCOC(=O)c1sc(SCc2ccccc2)c2c1CC1(CCOCC1)C/C2=N\O]

A title compound was obtained from the ketone 54 in the accordance with general method of synthesis of oximes. Yield: 50 mg (23%).

¹H NMR (400 MHz, DMSO-d₆) δ=1.21-1.24 (m, 2H), 1.31 (t, 3H, J=7.3 Hz), 1.33-1.37 (m, 2H), 1.40-1.42 (m, 2H), 2.58 (s, 1H), 2.65 (s, 1H), 3.07 (s, 1H), 3.21 (s, 1H), 3.53-3.57 (m, 4H), 4.16-4.19 (m, 1H), 4.30 (q, 2H, J=7.0 Hz), 4.41 (s, 1H), 7.29-7.40 (m, 2H), 7.45-7.49 (m, 1H), 11.28 (s, 1H).

APCI-MS: ˜100%, [M+H]⁺=432.00, t_{R}=2.58 minutes.

### Example A1.4. N-benzyl-3-(benzylthio)-4-(hydroxyimino)-6,6-dimethyl-4,5,6,7-tetrahydrobenzo[c]thiophene-1-carboxamide

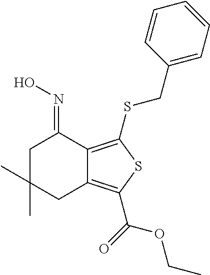 [SMILES: CCOC(=O)c1sc(SCc2ccccc2)c2c1CC(C)(C)C/C2=N\O]

A title compound was obtained from the ketone 57 in the accordance with general method of synthesis of oximes. Yield: 160 mg (71%).

¹H NMR: (DMSO, 400 MHz) δ=0.92 (s, 6H), 2.46 (s, 2H), 2.84 (s, 2H), 4.27 (s, 2H), 4.41 (d, J=5.6 Hz, 2H), 7.20-7.40 (m, 8H), 7.41-7.50 (m, 2H), 8.40 (t, J=6.3 Hz, 1H), 11.10 (s, 1H).

LCMS (ESI): 100.0%, [M+H]⁺=451.0; t_{R}=1.99 minutes.

### Example A1.5. Ethyl 4-(hydroxyimino)-6-(4-methoxyphenyl)-3-(phenylthio)-4,5,6,7-tetrahydro-benzo[c]thiophene-1-carboxylate

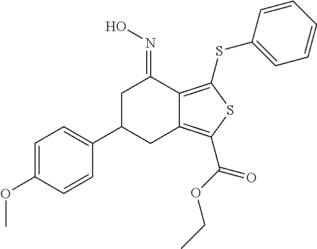 [SMILES: CCOC(=O)c1sc(Sc2ccccc2)c2c1CC(c1ccc(OC)cc1)C/C2=N\O]

A title compound was obtained from the ketone 59 in the accordance with general method of synthesis of oximes. Yield: 122 mg (54%).

¹H NMR (400 MHz, CDCl₃) δ=1.26 (t, J=7.1 Hz, 3H), 1.70 (br. s., 1H), 2.56 (dd, J=17.2, 12.5 Hz, 1H), 2.86 (dd, J=16.9, 12.0 Hz, 1H), 3.05 (dddd, J=16.0, 8.2, 3.9, 3.8 Hz, 1H), 3.52 (dt, J=17.2, 2.0 Hz, 1H), 3.76 (dt, J=16.8, 1.7 Hz, 1H), 3.83 (s, 3H), 4.23 (q, J=7.1 Hz, 2H), 6.90 (d, J=8.6 Hz, 2H), 7.24 (d, J=8.7 Hz, 2H), 7.47-7.57 (m, 3H), 7.71 (dd, J=7.5, 1.7 Hz, 2H).

LCMS (ESI): 100.0%, [M+H]⁺=454.0; t_{R}=2.16 minutes.

### Example A1.6. 3-(Benzylthio)-N-(3-fluorobenzyl)-4-(hydroxyimino)-6-(4-methoxyphenyl)-4,5,6,7-tetrahydrobenzo[c]thiophene-1-carboxamide

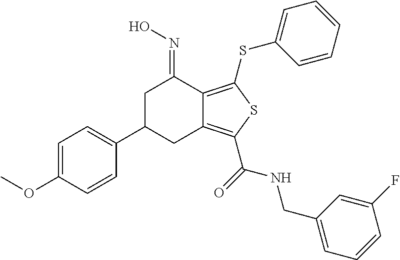 [SMILES: COc1ccc(C2C/C(=N\O)c3c(Sc4ccccc4)sc(C(=O)NCc4cccc(F)c4)c3C2)cc1]

A title compound was obtained from the ketone 49 in the accordance with general method of synthesis of oximes. Yield: 219 mg (80%).

¹H NMR (400 MHz, DMSO-d6) δ=2.42-2.53 (m, 1H, overlapped with DMSO-d5), 2.86-2.98 (m, 2H), 3.13 (d, J=18.6 Hz, 1H), 3.37 (m., 1H, overlapped with water signal), 3.72 (s, 3H), 4.29 (d, J=2.8 Hz, 2H), 4.38 (d, J=5.7 Hz, 2H), 6.87 (d, J=8.7 Hz, 2H), 7.01-7.13 (m, 3H), 7.25 (d, J=8.6 Hz, 2H), 7.30 (d, J=7.2 Hz, 1H), 7.35 (t, J=7.3 Hz, 3H), 7.45 (d, J=7.2 Hz, 2H), 8.48 (t, J=5.9 Hz, 1H), 11.19 (s, 1H).

LCMS (ESI): 100.0%, [M+H]⁺=547.0; t_{R}=1.95 minutes.

### Example A1.7. Ethyl 3-(benzylthio)-6-(4-fluorophenyl)-4-(hydroxyimino)-4,5,6,7-tetrahydrobenzo[c]-thiophene-1-carboxylate

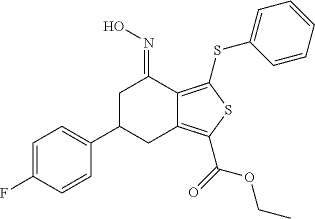 [SMILES: CCOC(=O)c1sc(Sc2ccccc2)c2c1CC(c1ccc(F)cc1)C/C2=N\O]

A title compound was obtained from the ketone 60 in the accordance with general method of synthesis of oximes. Yield: 148 mg (65%).

¹H NMR (400 MHz, CDCl₃) δ=1.25 (t, J=7.0 Hz, 3H), 2.56 (dd, J=16.8, 11.8 Hz, 1H), 2.88-2.99 (m, 1H), 3.00-3.10 (m, 1H), 3.17 (dd, J=16.7, 2.5 Hz, 1H), 3.56 (d, J=15.0 Hz, 1H), 4.24 (q, J=7.1 Hz, 2H), 4.30-4.40 (m, 2H), 7.17 (t, J=8.9 Hz, 2H), 7.29-7.43 (m, 5H), 7.47 (d, J=7.2 Hz, 2H), 11.27 (s, 1H).

LCMS (ESI): 100.0%, [M+H]⁺=456.0; t_{R}=2.27 minutes.

### Example A1.8. Benzyl 3-(benzylthio)-4-(hydroxyimino)-6-(4-methoxyphenyl)-4,5,6,7-tetrahydrobenzo[c]-thiophene-1-carboxylate

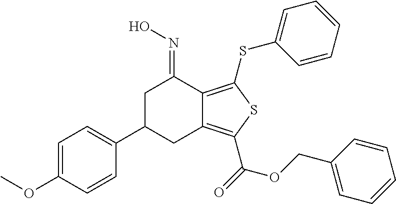 [SMILES: COc1ccc(C2C/C(=N\O)c3c(Sc4ccccc4)sc(C(=O)OCc4ccccc4)c3C2)cc1]

A title compound was obtained from the ketone 61 in the accordance with general method of synthesis of oximes. Yield: 148 mg (56%).

¹H NMR (400 MHz, CDCl₃) δ=2.52-2.58 (m, 1H), 2.88-3.04 (m, 2H), 3.15 (d, J=18.6 Hz, 1H), 3.56 (d, J=13.2 Hz, 1H), 3.74 (s, 3H), 4.34 (s, 2H), 5.29 (s, 2H), 6.90 (d, 2H), 7.25 (d, J=7.7 Hz, 2H), 7.29-7.43 (m, 8H), 7.46 (d, J=7.3 Hz, 2H), 11.23 (s, 1H).

LCMS (ESI): 100.0%, [M+H]⁺=530.0; t_{R}=2.11 minutes.

### Example A1.9. Ethyl 3-(benzylthio)-4-(hydroxyimino)-7-(4-methoxyphenyl)-4,5,6,7-tetrahydrobenzo[c]-thiophene-1-carboxylate

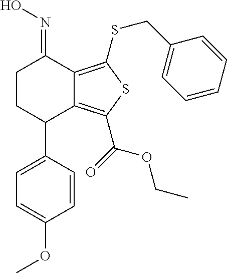 [SMILES: CCOC(=O)c1sc(SCc2ccccc2)c2c1C(c1ccc(OC)cc1)CC/C2=N\O]

To a solution of a mixture of ketones 62 and 63 (0.06 g, 0.133 mmol) in pyridine (3 ml) NH₂OHxHCl (0.15 g, 2.16 mmol, 16 eq) was added and the mixture was stirred 5 hours at 70° C. The reaction was cooled to r.t., pyridine was evaporated in vacuo, and residue was treated with 5% aq. H₂SO₄ (50 ml) and extracted with DCM (2×20 ml). The organic layers were combined, washed with H₂O (50 ml), dried over Na₂SO₄ and concentrated. The residue was subjected to preparative chromatography to give a title compound with ˜65% purity (without contamination of isomeric product). Yield: 5 mg (8%).

¹H NMR: (DMSO, 400 MHz) δ=1.16 (t, J=7.3 Hz, 3H), 1.80-2.09 (m, 3H), 2.86 (d, J=16.4 Hz, 1H), 3.69 (s, 3H), 4.08-4.19 (m, 2H), 4.37 (q, J=11.7 Hz, 2H), 4.99 (s, 1H), 6.83 (s, 4H), 7.31-7.42 (m, 3H), 7.45-7.52 (m, 2H), 11.24 (s, 1H).

LCMS (ESI): 80.98%, [M+H]⁺=468.0; t_{R}=2.08 minutes.

### Example A1.10. Ethyl-3-(benzylthio)-4-(hydroxyimino)-5-(4-methoxyphenyl)-4,5,6,7-tetrahydrobenzo[c]thiophene-1-carboxylate

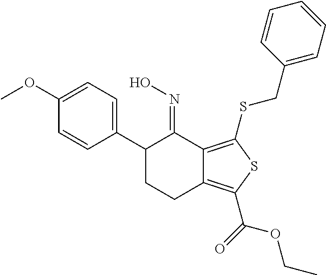 [SMILES: CCOC(=O)c1sc(SCc2ccccc2)c2c1CCC(c1ccc(OC)cc1)/C2=N\O]

To a solution of a mixture of ketones 62 and 63 (0.15 g, 0.332 mmol) in pyridine (3 ml) NH₂OHxHCl (0.23 g, 3.31 mmol, 10 eq) was added and the mixture was stirred 20 hours at 90° C. The reaction was cooled to r.t., pyridine was evaporated in vacuo, the residue was treated with 5% aq. H₂SO₄ (50 ml) and extracted with DCM (2×20 ml). The organic layers were combined, washed with H₂O (50 ml), dried over Na₂SO₄ and concentrated. The residue was purified using chromatography (SiO₂, hexane-DCM-Et₂O 5:1:0.6 mixture as eluent) to give ˜9:1 mixture of title A1.10 and starting ketone 63. This mixture was dissolved in DCM (2 ml) and n-hexane (4 ml) was added and resulting solution was maintained at r.t. until crystallization was initiated. A mixture was cooled down at −30° C. overnight. The precipitate was filtered off, washed with hexane-Et₂O 3:1 mixture and dried to afford a title compound. Yield: 90 mg.

LCMS (ESI): [M+H]⁺=468.0; t_{R}=2.20 minutes.

### Example A1.11. Ethyl 1-(benzylthio)-7-(hydroxyimino)-5-(4-methoxyphenyl)-4,5,6,7-tetrahydro-thieno[3,4-c]pyridine-3-carboxylate

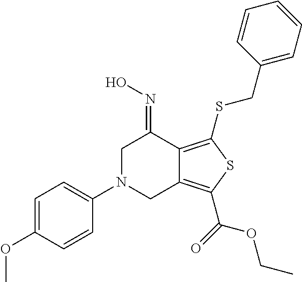 [SMILES: CCOC(=O)c1sc(SCc2ccccc2)c2c1CN(c1ccc(OC)cc1)C/C2=N\O]

Ketone 18 (172 mg, 0.38 mmol) was dissolved in pyridine (3.8 mL) and NH₂OH*HCl (78 mg, 1.13 mmol, 3 equiv) was added. The mixture was heated at reflux for 6 hours and evaporated. The residue was dry loaded on silica gel (˜10 mL) and eluted with a mixture of hexanes/EtOA to give title compound. Yield: 10 mg (6%).

¹H NMR (400 MHz, CDCl₃) δ=1.25 (t, J=7.2 Hz, 3H), 3.77 (s, 3H), 4.27 (s, 2H), 4.36 (d, 2H), 4.39 (s, 2H), 4.75 (s, 2H), 6.83 (d, J=9.0 Hz, 2H), 6.97 (d, J=9.0 Hz, 2H), 7.29-7.38 (m, 3H), 7.39-7.45 (m, 2H), 7.55-7.69 (m, 1H).

LCMS (ESI): ˜100%, [M+H]⁺=469.00. t_{R}=2.13 minutes.

### Example A1.12. (3-(Benzylthio)-N-ethyl-4-(hydroxyimino)-6-(4-methoxyphenyl)-4,5,6,7-tetrahydrobenzo[c]thiophene-1-carboxamide

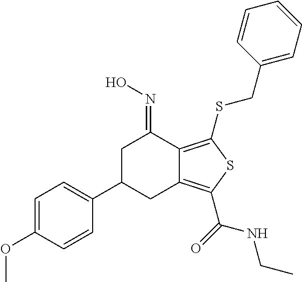 [SMILES: CCNC(=O)c1sc(SCc2ccccc2)c2c1CC(c1ccc(OC)cc1)C/C2=N\O]

A title compound was obtained from the ketone 87 in the accordance with general method of synthesis of oximes. Yield: 148 mg (56%).

1H NMR (DMSO-d₆, 400 MHz): δ=1.06 (t, J=7.1 Hz, 3H), 2.42-2.50 (m, 1H), 2.84-2.99 (m, 2H), 3.10-3.26 (m, 3H), 3.28-3.40 (m, 2H), 3.74 (s, 3H), 4.29 (dd, J=14.6, 12.0 Hz, 2H), 6.90 (d, J=8.6 Hz, 2H), 7.26 (d, J=8.7 Hz, 2H), 7.31 (d, J=7.2 Hz, 1H), 7.33-7.40 (m, 2H), 7.46 (d, J=7.3 Hz, 2H), 7.88 (t, J=5.5 Hz, 1H), 11.17 (s, 1H).

### Example A1.13. (3-(Benzylthio)-4-(hydroxyimino)-6-(4-methoxyphenyl)-4,5,6,7tetrahydrobenzo[c]thiophen-1-yl)(morpholino)methanone

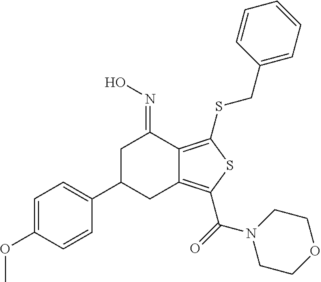 [SMILES: COc1ccc(C2C/C(=N\O)c3c(SCc4ccccc4)sc(C(=O)N4CCOCC4)c3C2)cc1]

A title compound was obtained from the ketone 88 in the accordance with general method of synthesis of oximes. Yield: 120 mg (47%).

### Example A1.14. N-benzyl-3-(benzylthio)-4-(hydroxyimino)-6-(4-methoxyphenyl)-4,5,6,7-tetrahydrobenzo[c]thiophene-1-carboxamide

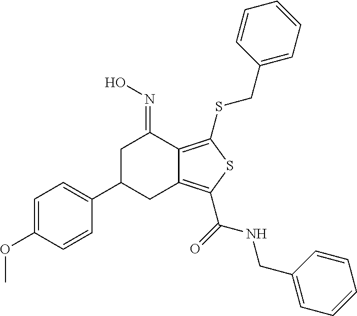 [SMILES: COc1ccc(C2C/C(=N\O)c3c(SCc4ccccc4)sc(C(=O)NCc4ccccc4)c3C2)cc1]

A title compound was obtained from the ketone 89 in the accordance with general method of synthesis of oximes. Yield: 132 mg (50%).

### Example A1.15. 3-(Benzylthio)-N-(furan-2-ylmethyl)-4-(hydroxyimino)-6-(4-methoxyphenyl)-4,5,6,7-tetrahydrobenzo[c]thiophene-1-carboxamide

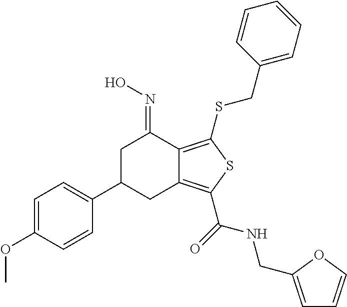 [SMILES: COc1ccc(C2C/C(=N\O)c3c(SCc4ccccc4)sc(C(=O)NCc4ccco4)c3C2)cc1]

A title compound was obtained from the ketone 90 in the accordance with general method of synthesis of oximes. Yield: 60 mg (23%).

### Example A2.1. 2-((4-Methylbenzyl)amino)-7-phenyl-7,8-dihydroquinazolin-5(6H)-one oxime

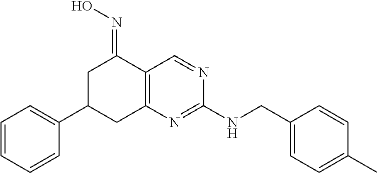 [SMILES: Cc1ccc(CNc2ncc3c(n2)CC(c2ccccc2)C/C3=N\O)cc1]

A title compound was obtained from the ketone 69 in the accordance with general method of synthesis of oximes. Yield: 140 mg (78%).

¹H NMR (400 MHz, DMSO-d₆) δ=2.25 (s, 3H), 2.65-2.83 (m, 1H), 2.88-3.25 (m, 3H), 3.34 (CH, 1H, overlapping with water), 4.47 (s, 2H), 6.92-7.49 (m, 9H), 7.92 (s, 1H), 8.69 (s, 1H), 10.94 (s, 1H).

LCMS (ESI): ˜100%, [M+H]⁺=359.00, t_{R}=1.66 minutes.

### Example A2.2. 2-((2-Phenoxyethyl)amino)-7-phenyl-7,8-dihydroquinazolin-5(6H)-one oxime

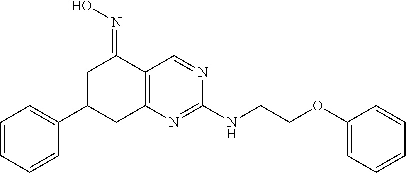 [SMILES: O/N=C1\CC(c2ccccc2)Cc2nc(NCCOc3ccccc3)ncc21]

A title compound was obtained from the ketone 71 in the accordance with general method of synthesis of oximes. Yield: 168 mg (90%).

¹H NMR (400 MHz, DMSO-d₆) δ=2.53-2.55 (m, 1H); 2.75-2.78 (m, 1H); 2.99-3.03 (m, 1H); 3.10-3.18 (m, 2H); 3.66 (s, 2H); 4.08 (s, 2H); 6.92-6.96 (m, 3H); 7.25-7.27 (m, 3H); 7.34 (s, 4H); 7.58 (s, 1H); 8.72 (s, 1H); 10.29 (s, 1H); 10.97 (s, 1H).

APCI-MS: ˜100%, [M+H]⁺=375.00, t_{R}=1.61 minutes.

### Example A2.3. 2-Amino-7-phenyl-4-(3-phenylpropyl)-7,8-dihydroquinazolin-5(6H)-one oxime

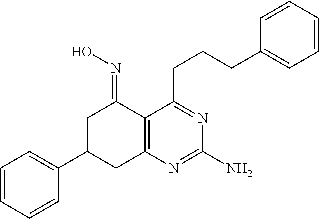 [SMILES: Nc1nc(CCCc2ccccc2)c2c(n1)CC(c1ccccc1)C/C2=N\O]

A title compound was obtained from the ketone 74 in the accordance with general method of synthesis of oximes. Yield: 100 mg (54%).

¹H NMR (400 MHz, DMSO-d₆) δ=1.91 (m, 2H), 2.53-2.60 (m, 1H), 2.60-2.67 (m, 2H), 2.70 (d, J=15.3 Hz, 1H), 2.86-3.08 (m, 4H), 3.16 (d, J=17.2 Hz, 1H), 6.71 (br. s, 2H), 7.11-7.44 (m, 10H), 10.95 (s, 1H).

LCMS (ESI): 93.42N, [M+H]⁺=373.00; t_{R}=1.51 minutes.

### Example A3.1. 5-(Hydroxyimino)-2-((2-phenoxyethyl)amino)-N,7-diphenyl-5,6,7,8-tetrahydroquinoline-3-carboxamide

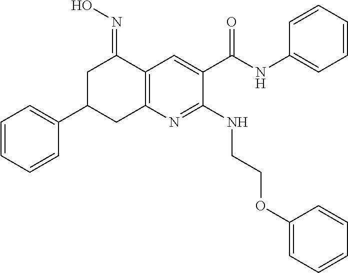 [SMILES: O=C(Nc1ccccc1)c1cc2c(nc1NCCOc1ccccc1)CC(c1ccccc1)C/C2=N\O]

A title compound was obtained from the ketone 79 in the accordance with general method of synthesis of oximes. Yield: 106 mg (43%).

¹H NMR (400 MHz, DMSO-d₆) δ=2.55-2.62 (m, 1H), 2.91-3.24 (m, 4H), 3.79-3.84 (m, 2H), 4.12-4.17 (m, 2H), 6.79 (t, J=6.7 Hz, 1H), 6.98 (d, J=7.5 Hz, 2H), 7.07 (t, J 6.9 Hz, 1H), 7.21-7.40 (m, 11H), 7.68 (d, J=7.3 Hz, 1H), 8.25 (br. s, 1H), 8.45 (br. s, 1H), 10.48 (s, 1H), 10.94 (s, 1H)

LCMS (ESI): [M+H]⁺=493.00. t_{R}=1.87 minutes.

### Example A3.2. 2-(Benzylthio)-5-(hydroxyimino)-7-phenyl-5,6,7,8-tetrahydroquinoline-3-carbonitrile

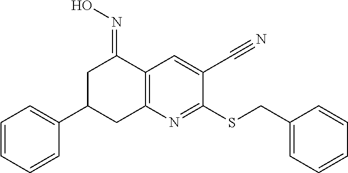 [SMILES: N#Cc1cc2c(nc1SCc1ccccc1)CC(c1ccccc1)C/C2=N\O]

A title compound was obtained from the ketone 81 in the accordance with general method of synthesis of oximes. Yield: 121 mg (63%).

¹H NMR: (DMSO, 400 MHz) δ=2.61-2.72 (m, 1H), 3.05-3.27 (m, 4H), 4.32 (q, J=14.7 Hz, 2H), 7.16-7.29 (m, 4H), 7.30-7.42 (m, 6H), 8.20 (s, 1H), 11.37 (s, 1H).

LCMS (ESI): ˜70%, [M+H₂O+H]⁺=403.7, t_{R}=1.88 minutes.

### Example A4.1. N-(7-(Hydroxyimino)-5-(4-methoxyphenyl)-4,5,6,7-tetrahydrobenzo[d]thiazol-2-yl)-2-(p-tolyl)acetamide

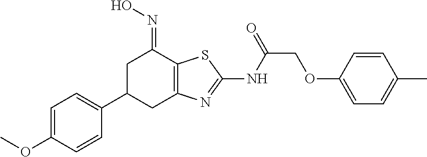

A title compound was obtained from the ketone 86 in the accordance with general method of synthesis of oximes. Yield: 24 mg (11%).

¹H NMR (400 MHz, DMSO-d₆) δ=2.31 (s, 3H), 2.82-3.02 (m, 2H), 3.10-3.18 (m, 1H), 3.22-3.33 (m, 2H), 3.36-3.58 (m, 2H), 3.71 (s, 2H), 3.82 (s, 3H), 4.71 (d, 2H, J=12 Hz), 6.85-6.91 (m, 4H), 7.13 (d, 2H, J=6.4 Hz), 7.22 (d, 2H, J=8.0 Hz).

APCI-MS: ˜100%, [M+H]⁺=438.00, t_{R}=2.38 minutes.

### 2-(Benzylamino)-5-phenyl-5,6-dihydrobenzo[d]thiazol-7(4H)-one oxime

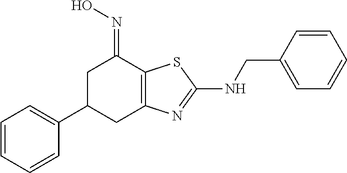 [SMILES: O/N=C1\CC(c2ccccc2)Cc2nc(NCc3ccccc3)sc21]

A title compound was obtained from the ketone 85 in the accordance with general method of synthesis of oximes. Yield: 129 mg (74%).

¹H NMR (400 MHz, DMSO-d₆) δ=2.59 (dd, J=14.5, 3.4 Hz, 1H), 2.70 (dd, J=14.4, 12.1 Hz, 1H), 2.82-2.94 (m, 2H), 3.15-3.24 (m, 1H), 4.23+4.47 (PhCH2 rotamers in 1:3 ratio, d, J=5.9 Hz, 2H), 7.19-7.38 (m, 10H), 8.40 (t, J=5.7 Hz, NH), 10.68 (s, 1H).

LCMS (ESI): ˜100%, [M+H]⁺=350.00. t_{R}=2.19 minutes.

### Example 2: Assessment of Human Senescent Versus Non-Senescent Cells

The fate and biological effects of human senescent versus non-senescent cells were assessed following implantation into young Severe Combined Immune Deficiency (SCID) mice. Reporter cells were generated of human neonatal dermal fibroblast (NDF) expressing secreted Gaussia luciferase (GLuc), allowing for cell survival to be monitored in vivo via measurement of GLuc activity from collected plasma [43,44]. Next, microcarrier bead cultures carrying these GLuc-expressing NDFs (NDF-GLuc) were produced, and cells were made quiescent via serum starvation (0.2% FBS) or senescent via 20 Gy gamma-irradiation. We inoculated 2-3×10⁶ cells into the peritoneal cavity of SCID mice via injection of ˜300 μl of packed bead volume, and cell survival via GLuc secretion was monitored from regular blood collections over 28 days. Quiescent and senescent NDFs exhibited a similar decrease in GLuc signal from plasma up to day 7 post-injection, where 30% of GLuc activity remained compared to the activity observed 24 hours after initial cell implantation. After day 7 however, GLuc activity from SC xenografts decreased at a faster rate than for quiescent cells, suggesting that these SCs are cleared more efficiently. By day 28, less than 1% of the GLuc signal remained from SCs observed on day 1 (a 5.2-fold lower compared to quiescent cells). Faster clearance of SC than non-SC suggested the activity of a specific mechanism(s) targeting SC that presumably involves the innate immune response (since adaptive immunity is compromised in SCID mice).

### Example 3: Assessment of Senescent Cell Killing

To identify components of innate immunity responsible for SC killing, we utilized a model where SCs could persist in recipient mice being unreachable for immunocytes. It involves embedding SC into beads made of alginate, a nanoporous gel that provides a physical barrier from the immune system, i.e. preventing rapid clearance of cells, while allowing for the exchange of nutrients and waste, as well as the release of cell secretions, e.g. cytokines [45-47]. Senescent NDF-GLuc-containing microcarrier beads were coated in alginate gel and injected into SCID mice, and GLuc activity was monitored to evaluate whether alginate indeed conferred protection to the SCs. By day 7, a similar loss of GLuc activity was observed compared to unprotected senescent and quiescent NDF-GLuc cells, reflecting a non-selective decrease in transplanted cell numbers likely due to in vivo adaptation. However, unlike bare cells, the presence of alginate stabilized GLuc activity observed in plasma, providing a stable signal for up to 3 additional weeks.

Alginate is a vital part of this model, required for SC cell survival in vivo. Since cells can be directly embedded into alginate, there was no need to pre-establish cells on microcarrier prior to coating. For future experiments we simplified our model, where senescent NDFs generated in vitro via 20 Gy irradiation were embedded directly into alginate beads as a single-cell suspension. Viability of the embedded cells was assessed via Calcein-AM and propidium iodide for the staining of live and dead cells, respectively. The procedure for generating alginate beads was optimized for >90% SC viability immediately following embedding. In addition, viability was stable during in vitro culture prior to inoculation of beads into the peritoneal cavity. Further, the alginate-embedded SCs were further characterized, and determined to be positive for senescence-associated β-galactosidase (β-gal^{pH6}) activity.

The activity of secreted GLuc in mouse plasma indicates that small proteins (GLuc; 25 kDa) were reliably released from the alginate beads and systemically detected in peripheral blood. In addition, these initial experiments demonstrated that alginate-coated senescent NDFs survive in the peritoneal cavity for a prolonged time, where they remained functionally active, as inferred by stable GLuc secretion. Protection of SC from rapid death by embedding in alginate beads strongly argued in favor of the involvement of cellular mechanism in SC eradication in vivo. To identify SC killers, we analyzed the content of cells attracted to the peritoneal cavity of mice carrying implanted alginate beads with SC.

To study immune system components attracted by SC secretions, 300 μl of alginate beads containing 1.5 million senescent NDF cells were inoculated into mice via intraperitoneal injection and harvested 14 days later, along with the intraperitoneal lavage, for the analysis of cellular content. Since similar results were obtained in SCID, NIH Swiss and C57BL/6 mice, here and below we only show data generated in C57BL/6.

While empty alginate beads stayed unchanged during 14 days following i.p. injection, SC-embedded beads were found to be surrounded by dense capsules formed by multiple layers of cells. Enzymatic staining of whole beads using protocol for detection of SC indicated that the capsules possessed high β-gal^{pH6} activity that at cellular level was associated with a large subset of capsule-forming cells. Further characterization of cells dissociated from the beads revealed that the beads were comprised mostly of macrophages, eosinophils and neutrophils. Indeed, immunofluorescent staining of cryosectioned beads revealed that a large portion of cells were F4/80-positive, a well-known and widely-used marker for mature murine macrophages. With the large proportion of F4/80- and β-gal^{pH6}-positive cells, our data suggests that macrophages are likely to possess β-gal^{pH6} activity in the presence of SCs.

The use of p16(Ink4a) reporter mice in several models of senescence and aging demonstrate that the appearance of cells in their tissues positive for β-gal^{pH6} is associated with elevated proportion of p16-expressing cells. However, the identity of these cells in vivo are not well-defined. We sought to test whether β-gal^{pH6}-positive immunocytes responding to SCs would be associated with elevated p16 expression. To do so, alginate-embedded SC xenografts were implanted into young p16^{LUC} reporter mice of the C57BL/6 background (hemizygous knock-in; p16^{INK4a/Luc}), and monitored for p16 induction via increased bioluminescent signal. Surprisingly, the presence of SCs induced a 13.1-fold increase in luminescent signal from the abdomen after 12 days in vivo. At the same time, no significant increase was observed in mice bearing empty alginate beads, indicating that the response elicited by SCs is not simply due to a foreign body response against alginate beads. Overall, a 10.1-fold increase in luminescence induction was observed between groups bearing empty beads compared to those bearing SC xenografts (p≤0.01). Together, these findings demonstrate that the release of SC secretions from alginate beads strongly induces p16(Ink4a) expression.

In order to identify which cell population(s) contribute to p16(Ink4a) and β-gal^{pH6} signal in response in this model, SCs-elicited immunocytes from the peritoneal cavity of p16^{LUC} mice were collected 2 weeks after SC inoculation. FACS analysis of live-stained peritoneal infiltrates revealed cells to be of hematopoietic origin (≥90% CD45⁺) and comprised of three main cell types (constituting ˜75% of lavage). These cells were sorted into four populations for subsequent determination of luciferase and β-gal^{pH6} activity: 1) B lymphocytes (CD19⁺; ˜26%), 2) eosinophils (CD19⁻ CD11b⁺ CD170⁺; ˜17%), 3) macrophages (CD19⁻ CD11b⁺ F4/80⁺; ˜46%), and 4) a pool of remaining cell populations (11%), including neutrophils (CD11b⁺ Ly-6G⁺), CD11b⁻ cells (e.g. T lymphocytes) and CD11b⁺ F4/80⁻ cells (e.g. monocytes, NK cells). Analysis of cell lysates from these sorted populations revealed a substantial enrichment of both markers in macrophages. In fact, macrophages were the only cell population to provide a detectable luciferase activity, which was more than 6-fold higher per cell than other populations. Similarly, macrophages were more 10.5-fold enriched for β-gal^{pH6} activity per cell. Given their abundance, these data suggest that macrophages almost exclusively contribute to the luminescent signal and β-gal^{pH6} activity from SC-elicited infiltrates found within the peritoneal lavage. Thus, we demonstrate that macrophages elicited by SCs possess two common attributes of SCs: increased expression of p16 and β-gal^{pH6}.

### Example 4: Selective Removal of Senescence-Associated Macrophages (SAMs) from a Mixed Population of Cells

p16^{LUC} mice bearing SC xenografts were inoculated with SCs, and after 15 days, were treated intraperitoneally with a liposomal formulation of clodronate, a reagent commonly used for selective depletion of phagocytes (e.g. macrophages) [48,49]. For mice bearing alginate-embedded SCs treated with vehicle control (empty liposomes in PBS), signal continued to increase 2.3-fold compared to the pre-treatment measurements. In contrast, clodronate treatment resulted in a 2.2-fold decrease in bioluminescence. Thus, the combined effect of clodronate treatment was a 5.1-fold decrease in bioluminescent signal from p16^{LUC} mice compared to the vehicle-treated group (p≤0.0001). In mice bearing empty beads, which do not induce p16, a non-significant decrease was observed upon clodronate treatment. Cells with phagocytic activity, i.e. macrophages, are responsible for the majority of p16(Ink4a) induction in response to senescent NDFs. This finding is consistent with our previous observation that macrophages from lavage are the single contributing source of luciferase activity.

To confirm that the loss of in vivo bioluminescence in mice upon treatment with clodronate is a result of macrophage clearance, peritoneal lavage from mice bearing alginate-embedded SCs was collected for analysis following administration of vehicle or clodronate liposomes. First, the effects of clodronate treatment on cell yields from peritoneal lavage were determined. While no difference in lavage cell yields was observed between naïve mice and vehicle-treated mice bearing empty alginate beads (˜5×10⁶ cells), for vehicle-treated mice inoculated with alginate-embedded SCs, a large number of cells infiltrated into the peritoneal cavity (26.4×10⁶ cells; a >5-fold increase in cell yield over control groups; p≤0.0001). Clodronate treatment of mice bearing alginate-embedded SCs resulted in decreased yields from peritoneal lavage, with cell counts similar to control groups (3.2×10⁶ cells).

Clodronate Treatment

Liposomal clodronate (Clodrosome®; 5 mg/mL) and liposomal vehicle in PBS (Encapsome®) were obtained from Encapsula NanoSciences (Brentwood, Tenn.). These liposomal formulations or PBS control (as indicated) were administered to mice as 200 μl injections per mouse (20-40 mg/kg), either intraperiteonally or intravenously. Injections were administered twice per mouse, 2-4 days apart. For experiments utilizing alginate beads, mice were administered two intraperitoneal injections, with collection of lavage and alginate beads 1-2 days after the last injection. For treatment of old p16^{LUC} mice, mice received the first injection intraperiteonally, and the second injection intravenously via tail vein was administered 2 days later. Adipose tissues (inguinal or perigonadal visceral fat) were collected from mice 1-2 days after the last treatment.

For in vitro treatment of clodronate and related controls, cell cultures were washed once with medium, then incubated with medium supplemented with a 1:100 dilution of liposomal clodronate suspension (50 μg/mL clodronate), liposomal PBS control or D-PBS control (non-treated). Cells were maintained for 20 hours at standard conditions during incubation with treatments. Plates were then washed twice with D-PBS and photographed. Cells were then lifted for determination of luciferase activity.

Clearance of macrophages from clodronate-treated mice was confirmed in mice bearing alginate-embedded SCs, where flow cytometric analysis showed that the proportion of F4/80⁺ cells in lavage was reduced from 26% in vehicle-treated mice to less than 1% following clodronate treatment. This is consistent with our observation that clodronate treatment effectively depleted F4/80-positive cells from the spleens of these mice. To assess the impact of clodronate treatment on the presence of luciferase and β-gal^{pH6} activity in mice bearing SCs, cell lysates from peritoneal lavage infiltrates were assayed. Luciferase activity was found to be undetectable from the lavage of all mice that received clodronate, a >47-fold reduction between vehicle- and clodronate-treated groups (p≤0.01). Loss of luciferase activity from the lavage of clodronate-treated mice was accompanied by a concomitant decrease in β-gal^{pH6} activity, a 37-fold reduction compared to vehicle-treated mice (p≤0.01). Thus, consistent with analysis from sorted cell populations from lavage, in vivo clodronate treatment of mice bearing alginate-embedded SCs resulted in the depletion of p16(Ink4a)/β-gal^{pH6}-positive macrophages from the peritoneal cavity.

The invention contemplates selective removal of SAMs from a mixed population of cells whether removal is a reduction in the number of SAMS (e.g., 80%) or complete elimination of SAMs from a given mixed population. For example, Clodronate treatment efficiently depleted a major portion of luminescent signal measured in vivo (˜80% reduction compared to vehicle-treated). However, residual luminescent signal remained, approximately 6-fold over initial baseline measurements (i.e. prior to SC injection). Thus, we sought to identify additional sources of luciferase signal in vivo. Histological analysis of alginate beads from vehicle- and clodronate-treated mice bearing SC revealed that clodronate was inefficient at eliminating F4/80-positive cells from the cells encapsulating the alginate beads, suggesting that liposomes may be incapable of accessing inner cell layers of the capsule. Cells dissociated from alginate beads were analyzed on flow cytometry to determine cell composition, confirming that clodronate was unable to exert a significant effect on the composition of encapsulating cells from vehicle- and clodronate-treated mice. Consistent with our previous observations that macrophages were the single greatest contributor to luciferase and β-gal^{pH6} activity from peritoneal lavage, the inability of clodronate to successfully deplete macrophages from alginate beads was associated with unaltered levels of luciferase and β-gal^{pH6} activity measured from lysates. Taken together, this data strongly suggests that the bioluminescent signal remaining in vivo following clodronate treatment is due to the continued presence of macrophages surrounding alginate beads.

### Example 5: Clodronate Liposomes Reduce SAMs in Chronologically Aged Mice

Our data demonstrate that macrophages, through increased number and/or activation state, are capable of generating a bioluminescent signal easily detectable via bioluminescent imaging. We next evaluated whether macrophages contribute to the increased luminescent signal in chronologically aged p16^{LUC} mice in vivo. Bioluminescent signal from of 90-week old p16^{LUC} mice was 9.0-fold higher than 13-week old mice. To determine if macrophages contribute to this increased signal, bioluminescence was measured before and after phagocyte depletion via clodronate liposomes. First, mice were distributed equally across treatment groups based on baseline measurements of signal intensity. Since it is not known how ingestion of vehicle liposomes may affect bioluminescence in phagocytes in old p16^{LUC} mice, both PBS alone and vehicle liposomes (in PBS) were administered as controls. While slight increases were observed in old mice after treatment with either vehicle liposomes or PBS (˜20% increase), the group receiving clodronate displayed a decrease in luciferase signal (˜22%). Overall, clodronate-treatment resulted in a 1.5-fold difference in 16 expression between both control groups of old mice (p≤0.05). The percent of old mice with >20% decrease in luciferase activity following treatment was 0% and 17% for PBS and vehicle liposome groups, respectively, compared to 67% of mice in the clodronate-treated group. These findings demonstrate that phagocytes contribute to p16(Ink4a) promoter-driven reporter signal in chronologically aged mice.

Adipose tissue is thought to be the major source of SCs and chronic inflammation in chronologically aged mice [15,23]. Old mice possess depots of white adipose tissue that stain positive for β-gal^{pH6} activity compared to young mice [23]. To determine whether macrophages contribute to the pool of β-gal^{pH6}+cells in adipose tissue of chronologically aged mice, 90-week old p16^{LUC} mice were treated with liposomes containing PBS (vehicle) or clodronate, and inguinal and visceral (perigonadal) depots of white adipose tissues were collected for analysis. Whole pieces of fat stained for β-gal^{pH6} activity revealed that that clearance of phagocytes resulted in a strong reduction in staining in clodronate-treated mice compared to vehicle-treated. Similar results were obtained after treatment of aged wild type mice of the same background and opposite gender (male C57BL/6; 64 weeks old).

Clodronate liposomes have been shown to deplete macrophages from various depots of white adipose tissues in mice [48,49]. To confirm that in vivo clodronate treatment was successful in clearing β-gal^{pH6}-positive cells, β-gal^{pH6}-stained fat was analyzed via light microscopy following nuclear counterstain. In 90-week old mice treated with PBS as a control, we observed the presence of β-gal^{pH6}-positive cells in situ, interspersed between mature adipocytes in visceral adipose tissue. In contrast, in adipose tissue of young mice (13 weeks old) treated with PBS, cells were present in the same location within the adipose tissue (assessed via nuclear stain), but these cells were negative for β-gal^{pH6} staining. These results confirmed that β-gal^{pH6}-positive cells in adipose tissue accumulated with age. The treatment of old mice with vehicle liposomes had little effect on the presence of β-gal^{pH6}-positive cells interspersed between adipocytes; however, these cells were stained more intensely compared to PBS-treated aged mice. Unlike the other three groups, treatment with clodronate resulted in the absence of both β-gal^{pH6}-positive cells and nuclear staining between mature adipocytes, indicating that these cells were cleared by clodronate. Similar findings were observed in inguinal adipose tissue from the same mice (data not shown).

Recent literature attributes the age-dependent increase in β-gal^{pH6}-positive cells in adipose tissue to the appearance of senescent pre-adipocytes. However, we demonstrate that phagocytic cells contribute to the vast majority of β-gal^{pH6} staining in chronologically aged mice. To assess whether senescent adipose-derived mesenchymal stromal cells possess sufficient phagocytic activity to be cleared by clodronate, we first established primary cultures from the adipose-derived stromal vascular fraction of p16^{LUC} mice. After the first passage of these cultures, minimal macrophage contamination was observed (<5%). Senescence induction of primary cells in vitro (20 Gy gamma-irradiation followed by 10 days in culture) gave rise to common features of SCs, including loss of proliferation, enlarged morphology, and positive β-gal^{pH6} staining. In addition these cultures displayed a 9.7-fold increase (p≤0.0001) in p16(Ink4a) promoter-driven luminescence between early passage cultures (352±17.1 p/s/cell×10³) compared to senescent cultures (3,404±89.8 p/s/cell×10³). However, in vitro treatment of SC cultures with liposomal clodronate had no effect on the appearance or viability of these SC cultures, nor an effect on luciferase activity, as compared to non-treated and vehicle liposome-treated SC cultures. As expected, this same dose of clodronate efficiently cleared adherence-selected peritoneal macrophages from naïve mice. The mechanism of clodronate-dependent clearance of β-gal^{pH6}-positive cells requires a sufficient level of phagocytic activity, a phenotype not previously associated with senescent stroma. These data strongly suggest that the β-gal^{pH6}-positive cells observed in adipose tissue in situ are macrophages and/or other professional phagocytes.

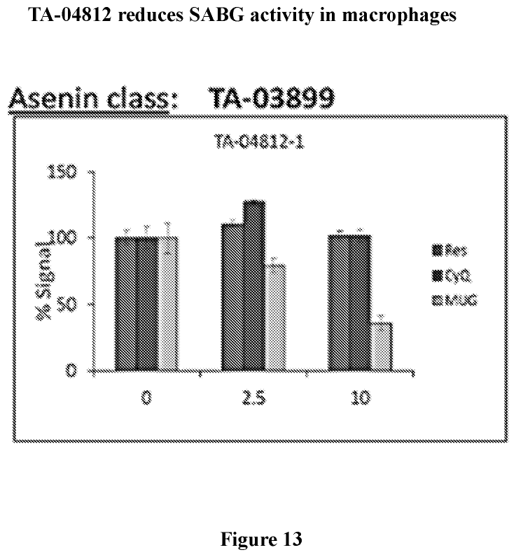

In addition, aged 102 week old female C57BL/6J p16^{Ink4a/Luc} mice were administered 200 μl of liposomal clodronate (Clodrosome®, 5 mg/ml) intraperitoneally (i.p.) on day one of the experiment and 200 μl of liposomal clodronate intravenously (i.v.) on day three of the experiment (FIG. 13A). Control mice were administered 200 μl of liposomal vehicle in PBS (Encapsome®) intraperitoneally (i.p.) on day one of the experiment and 200 μl of liposomal vehicle in PBS intravenously (i.v.) on day three of the experiment. On day ten of the experiment, organs from mice treated with clodronate liposomes and PBS control liposomes (vehicle only) were visualized using an IVIS bioluminescent imager in the presence of D-luciferin (300 μg/mL), and luciferase signal was quantitated from liver, spleen, fat, pancreas, kidney, lungs, GI tract with lymph nodes, and heart (FIG. 13B). Quantitation of luciferase signal (total flux; p/s) from visceral fat from several mice revealed a 13-fold decrease in luciferase signal upon treatment with clodronate liposomes compared to mice treated with vehicle alone (FIG. 13C). These results demonstrate that a significant portion of luciferase signal in visceral fat comes from phagocytic cells. We previously demonstrated that the vast majority of cleared cells from adipose tissue using liposomal clodronate appear to be macrophages, and thus, likely the bearer of luciferase signal.

### Example 6: Removal of SAMs from a Mixed Population of Cells

Generally, the SAMs may be selectively removed from a Mixed Population of Cells, as follows. An Agent (5 mg/mL) and vehicle are mixed in PBS. The formulation or a PBS control (as indicated) is administered to mice as 200 μl injections per mouse (20-40 mg/kg), either intraperiteonally or intravenously. Injections are administered twice per mouse, 2-4 days apart. For experiments utilizing alginate beads, mice are administered two intraperitoneal injections, with collection of lavage and alginate beads 1-2 days after the last injection. For treatment of old p16^{LUC} mice, mice receive the first injection intraperiteonally, and the second injection intravenously via tail vein is administered 2 days later. Adipose tissues (inguinal or perigonadal visceral fat) are collected from mice 1-2 days after the last treatment.

For in vitro treatment of agent and related controls, cell cultures are washed once with medium, then incubated with medium supplemented with a 1:100 dilution of vehicle/agent suspension (50 μg/mL), liposomal PBS control or D-PBS control (non-treated). Cells are maintained for 20 hours at standard conditions during incubation with treatments. Plates are then washed twice with D-PBS and photographed. Cells are then lifted for determination of luciferase activity.

Clearance of macrophages from agent-treated mice is confirmed in mice bearing alginate-embedded SCs, where flow cytometric analysis will show that the proportion of F4/80+ cells in lavage is reduced from about 20-40% in vehicle-treated mice to less than 1-10% following agent treatment. Spleen cells also can be assayed for F4/80+ and are expected to show a reduction. To assess the impact of agent treatment on the presence of luciferase and β-galβ-gal^{pH6} activity in mice bearing SCs, cell lysates from peritoneal lavage infiltrates are assayed. Luciferase activity may be found to be undetectable from the lavage of mice that received the agent, and preferably will show at least a 10-fold if not greater than a 47-fold reduction between vehicle- and agent-treated groups. Loss of luciferase activity from the lavage of treated mice is expected to be accompanied by a concomitant decrease in β-gal^{pH6} activity. Thus, in vivo agent treatment of mice bearing alginate-embedded SCs should result in the partial or complete depletion of p16(Ink4a)/β-gal^{pH6}-positive macrophages from the peritoneal cavity.

It is also contemplated that this assay system can be used to screen for and identify many types of agents useful to remove, reduce or eradicate SAMs, including but not limited to: siRNAs, shRNAs, antisense oligonucleotides, antibodies, adzymes, aptamers, proteins, and small molecules.

### Example 7: Selective Removal of SAMs from a Mixed Population of Cells Using NFκB p65 shRNA (m) Lentiviral Particles

NFκB p65 shRNA (m) Lentiviral Particles are available from Santa Cruz Biotech, Santa Cruz, Calif. Lentiviral Particles generally contain three to five expression constructs each encoding target-specific 19-25 nt (plus hairpin) shRNA designed to knockdown gene expression. Therefore, the lentiviral particle serves as the delivery vehicle (which are recognized by macrophages as foreign) and the NFκB shRNA sequences (which correspond to NFκB p65 siRNA/siRNA Gene Silencer sequences) serves as to toxic agent. 15 μl of a 200 μl viral stock containing 1×10⁶ infectious units of virus (IFU) is administered to mice as 200 μl injections per mouse (20-40 mg/kg), either intraperiteonally or intravenously. Injections are administered twice per mouse, 2-4 days apart. For experiments utilizing alginate beads, mice are administered two intraperitoneal injections, with collection of lavage and alginate beads 1-2 days after the last injection. For treatment of old p16^{LUC} mice, mice receive the first injection intraperiteonally, and the second injection intravenously via tail vein is administered 2 days later. Adipose tissues (inguinal or perigonadal visceral fat) are collected from mice 1-2 days after the last treatment.

For in vitro treatment of NFκB shRNA lentiviral construct and related controls, cell cultures are washed once with medium, then incubated with medium supplemented with a 1:100 dilution of NFκB shRNA lentiviral construct suspension (50 μg/mL), liposomal PBS control or D-PBS control (non-treated). Cells are maintained for 20 hours at standard conditions during incubation with treatments. Plates are then washed twice with D-PBS and photographed. Cells are then lifted for determination of luciferase activity.

Clearance of macrophages from NFκB shRNA lentiviral construct-treated mice is confirmed in mice bearing alginate-embedded SCs, where flow cytometric analysis will show that the proportion of F4/80⁺ cells in lavage is reduced from about 20-40% in vehicle-treated mice to less than 1-10% following NFκB shRNA lentiviral construct treatment. Spleen cells also can be assayed for F4/80+ and are expected to show a reduction. To assess the impact of NFκB shRNA lentiviral construct treatment on the presence of luciferase and β-gal β-gal^{pH6} activity in mice bearing SCs, cell lysates from peritoneal lavage infiltrates are assayed. Luciferase activity may be found to be undetectable from the lavage of mice that received the NFκB shRNA lentiviral construct, and preferably will show at least a 10-fold if not greater than a 47-fold reduction between NFκB shRNA lentiviral construct-treated groups. Loss of luciferase activity from the lavage of treated mice is expected to be accompanied by a concomitant decrease in β-gal^{pH6} activity. Thus, in vivo NFκB shRNA lentiviral construct treatment of mice bearing alginate-embedded SCs should result in the partial or complete depletion of p16(Ink4a)/β-gal^{pH6}-positive macrophages from the peritoneal cavity.

It is also contemplated that this assay system can be used to screen for and identify many types of agents useful to remove, reduce or eradicate SAMs, including but not limited to: siRNAs, shRNAs, antisense oligonucleotides, antibodies, adzymes, aptamers, proteins, and small molecules.

Therefore, this example describes how to remove SAMs from a mixed population of cells using an shRNA specific for NFκB p65 in a delivery vehicle which is a viral particle.

### Example 8: P2PDox Conjugated to a F4/80 Antibody Cleavable Toxin Useful According to the Invention to Selectively Remove SAMs

The Prodrug P2PDox is converted to the ultratoxic drug 2-pyrrolinodoxorubicin (2PDox), which has a potent cytotoxic effect on the targeted cell. Antibody-drug conjugate (ADC) formulations containing an antibody conjugated to P2Pdox may be used according to the invention, wherein the antibody serves as a delivery vehicle to SAMs, once ingested, the SAMs cleave P2Pdox to its toxic form 2Pdox. Govindan et al. US 20160166708 describe conjugates of pro-2-pyrrolinodoxorubicin (P2PDox). P2PDox may be conjugated to an antibody, antigen-binding antibody fragment, or a targetable construct or other targeting molecule that can deliver the P2PDox to selected target cells. In this case, an antibody can be specific for CD19, CD11b or F4/80 may be conjugated to P2Pdox. The Anti-Mouse F4/80 Antibody, Clone BM8, APC is a rat monoclonal IgG2a antibody against human, mouse F4/80, APC-conjugated. The BM8 antibody reacts with the F4/80 antigen (also termed Ly-71 in mouse and EMR-1 in human), an 160 kDa transmembrane glycoprotein belonging to the EGF-TM7 family of G-protein-coupled receptors. F4/80 is considered a marker of choice for the identification of mature tissue macrophages, being broadly but variably expressed by this cell type in the liver (Kupffer cells), skin (Langerhans cells), bone marrow stroma, pancreas, thymus, spleen (red pulp), lung, and other tissues. F4/80 expression levels increase following activation of macrophages.

Alternatively, instead of an APC-conjugated P2Pdox, P2PDox may be carried in liposomes, where upon ingestion, SAMs will convert P2Pdox to 2Pdox.

SAMs may be removed from a Mixed Population of Cells using an APC conjugated P2Pdox (StemCell Technologies, Cat. No. 60027AZ.1) (25 μg, 0.2 mg/ml) in PBS. The formulation or a PBS control (as indicated) is administered to mice as 1 μg in 200 μl injections per mouse (20-40 mg/kg), either intraperiteonally or intravenously. Injections are administered twice per mouse, 2-4 days apart. For experiments utilizing alginate beads, mice are administered two intraperitoneal injections, with collection of lavage and alginate beads 1-2 days after the last injection. For treatment of old p16^{LUC} mice, mice receive the first injection intraperiteonally, and the second injection intravenously via tail vein is administered 2 days later. Adipose tissues (inguinal or perigonadal visceral fat) are collected from mice 1-2 days after the last treatment.

For in vitro treatment of APC-conjugated P2Pdox and related controls, cell cultures are washed once with medium, then incubated with medium supplemented with a 1:100 dilution of APC-conjugated P2Pdox suspension (50 μg/mL), liposomal PBS control or D-PBS control (non-treated). Cells are maintained for 20 hours at standard conditions during incubation with treatments. Plates are then washed twice with D-PBS and photographed. Cells are then lifted for determination of luciferase activity.

Clearance of macrophages from APC-conjugated P2Pdox-treated mice is confirmed in mice bearing alginate-embedded SCs, where flow cytometric analysis will show that the proportion of F4/80⁺ cells in lavage is reduced from about 20-40% in vehicle (antibody alone)-treated mice to less than 1-10% following APC-conjugated P2Pdox treatment. Spleen cells also can be assayed for F4/80+ and are expected to show a reduction. To assess the impact of APC-conjugated P2Pdox treatment on the presence of luciferase and β-gal β-gal^{pH6} activity in mice bearing SCs, cell lysates from peritoneal lavage infiltrates are assayed. Luciferase activity may be found to be undetectable from the lavage of mice that received the APC-conjugated P2Pdox, and preferably will show at least a 10-fold if not greater than a 47-fold reduction between vehicle- and APC-conjugated P2Pdox-treated groups. Loss of luciferase activity from the lavage of treated mice is expected to be accompanied by a concomitant decrease in β-gal^{pH6} activity. Thus, in vivo APC-conjugated P2Pdox treatment of mice bearing alginate-embedded SCs should result in the partial or complete depletion of p16(Ink4a)/β-gal^{pH6}-positive macrophages from the peritoneal cavity.

Therefore, this example describes how to remove SAMs from a mixed population of cells using a prodrug toxin conjugated to an APC delivery vehicle.

### Example 9: Selective Removal of SAMs from a Mix Population of Cells Using a Lipofectamine/siRNA which Suppresses GST-.pi. and Akt RNAs

Gst-pi and Akt are genes involved in apoptosis. Suppressing expression of Gst-pi and Akt has the effect of inducing apoptosis. Knockdown of gene expression of GST-.pi. and Akt can be performed using siRNAs specific for these genes.

Niitsu, Yoshiro et al., US 20150328248 describe RNAi agents for achieving GST-pi and Akt knockdown, and demonstrate the ability of these agents to do so. GST-.pi. siRNA and/or Akt siRNA-Lipofectamine RNAiMAX is obtained from Life Technologies.

A Lipofectamine/siRNA mixed solution for transfection was prepared as follows. First, 35 .mu.L of Lipofectamine RNAiMAX and 965 .mu.L of OPTI-MEM (Sigma) were mixed to thus prepare a Lipofectamine solution. Subsequently, a predetermined amount of 10 .mu.M siRNA was diluted to 1 mL with OPTI-MEM to thus prepare an siRNA solution (for example, when preparing a siRNA solution having a final concentration of 30 nM for use, 36 .mu.L of 10 .mu.M siRNA and 964 .mu.L of OPTI-MEM were mixed), and this was mixed with the lipofectamine solution and allowed to stand at room temperature for 15 minutes. The following GST-pi siRNA was used: Sense chain: GGGAGGCAAGACCUUCAUUtt (SEQ ID No: 1); Antisense chain: AAUGAAGGUCUUGCCUCCCtg (SEQ ID No: 2) (Uppercase letters indicate RNA and lowercase letters indicate DNA) Akt siRNA: Akt siRNA I (Cell Signaling Technology, #6211) Control siRNA: AllStars Neg. Control siRNA 1027281 (QIAGEN). The Lipofectamine/siRNA mixed solution was prepared by the same method as above using a mixture of 36 .mu.L of 10 .mu.M GST-.pi. siRNA, 36 .mu.L of 10 .mu.M Akt siRNA, and 928 .mu.L of OPTI-MEM as an siRNA solution.

To remove SAMs from a mixed cell population, these same lipofectamine-siRNAs or a PBS control (as indicated) is administered to mice as 10 μg in 200 μl injections per mouse (20-40 mg/kg), either intraperiteonally or intravenously. Injections are administered twice per mouse, 2-4 days apart. For experiments utilizing alginate beads, mice are administered two intraperitoneal injections, with collection of lavage and alginate beads 1-2 days after the last injection. For treatment of old p16^{LUC} mice, mice receive the first injection intraperiteonally, and the second injection intravenously via tail vein is administered 2 days later. Adipose tissues (inguinal or perigonadal visceral fat) are collected from mice 1-2 days after the last treatment.

For in vitro treatment of Lipofectamine-siRNAs directed to GST-pi and/or AKt and related controls, cell cultures are washed once with medium, then incubated with medium supplemented with a 1:100 dilution of Lipofectamine-siRNAs directed to GST-pi and/or Akt suspension (50 μg/mL), liposomal PBS control or D-PBS control (non-treated). Cells are maintained for 20 hours at standard conditions during incubation with treatments. Plates are then washed twice with D-PBS and photographed. Cells are then lifted for determination of luciferase activity.

Clearance of macrophages from Lipofectamine-siRNAs directed to GST-pi and/or AKt-treated mice is confirmed in mice bearing alginate-embedded SCs, where flow cytometric analysis will show that the proportion of F4/80⁺ cells in lavage is reduced from about 20-40% in vehicle (antibody alone)-treated mice to less than 1-10% following Lipofectamine-siRNAs directed to GST-pi and/or AKt treatment. Spleen cells also can be assayed for F4/80+ and are expected to show a reduction. To assess the impact of Lipofectamine-siRNAs directed to GST-pi and/or AKt treatment on the presence of luciferase and β-gal β-gal^{pH6} activity in mice bearing SCs, cell lysates from peritoneal lavage infiltrates are assayed. Luciferase activity may be found to be undetectable from the lavage of mice that received the Lipofectamine-siRNAs directed to GST-pi and/or Akt, and preferably will be at least a 10-fold if not greater than a 47-fold reduction between vehicle- and -treated groups. Loss of luciferase activity from the lavage of Lipofectamine-siRNAs directed to GST-pi and/or AKt-treated mice is expected to be accompanied by a concomitant decrease in β-gal^{pH6} activity. Thus, in vivo Lipofectamine-siRNAs directed to GST-pi and/or Akt treatment of mice bearing alginate-embedded SCs should result in the partial or complete depletion of p16(Ink4a)/β-gal^{pH6}-positive macrophages from the peritoneal cavity.

Therefore, this example describes how to remove SAMs from a mixed population of cells using an siRNA contained in a liposome delivery vehicle.

### Example 10: Selective Removal of SAMs from a Mix Population of Cells Using a Small Molecule 1,3-Dihydro-1-(1-((4-(6-phenyl-1H-imidazo[4,5-g]quinoxalin-7-yl)phenyl)methyl)-4-piperidinyl)-2H-benzimidazol-2-one) Combined with Lipofectamine/siRNA which Suppresses GST-.pi

Niitsu, Yoshiro et al., US 20150328248 also reported that the Akt Inhibitor VIII (Merck #124018, 1,3-Dihydro-1-(1-((4-(6-phenyl-1H-imidazo[4,5-g]quinoxalin-7-yl)phenyl)methyl)-4-piperidinyl)-2H-benzimidazol-2-one), which is a small molecule, is effective in knocking down Akt and suppressing apoptosis. It is known that Akt Inhibitor VIII binds to a Pleckstrin homology (PH) region of Akt to thus cause allosteric inhibition, and although it does not inhibit Akt3 it selectively blocks activation and phosphorylation of Akt1 and Akt2.

To remove SAMs from a mixed cell population, these same Akt Inhibitor VIII (Merck #124018) or a PBS control (as indicated) is administered to mice as 0.1 μg in 200 μl injections per mouse (20-40 mg/kg), either intraperiteonally or intravenously. Injections are administered twice per mouse, 2-4 days apart. For experiments utilizing alginate beads, mice are administered two intraperitoneal injections, with collection of lavage and alginate beads 1-2 days after the last injection. For treatment of old p16^{LUC} mice, mice receive the first injection intraperiteonally, and the second injection intravenously via tail vein is administered 2 days later. Adipose tissues (inguinal or perigonadal visceral fat) are collected from mice 1-2 days after the last treatment.

For in vitro treatment of Akt Inhibitor VIII (Merck #124018) and related controls, cell cultures are washed once with medium, then incubated with medium supplemented with a 1:100 dilution of Akt Inhibitor VIII (Merck #124018), PBS control or D-PBS control (non-treated). Cells are maintained for 20 hours at standard conditions during incubation with treatments. Plates are then washed twice with D-PBS and photographed. Cells are then lifted for determination of luciferase activity.

Clearance of macrophages from Akt Inhibitor VIII (Merck #124018)-treated mice is confirmed in mice bearing alginate-embedded SCs, where flow cytometric analysis will show that the proportion of F4/80⁺ cells in lavage is reduced from about 20-40% in vehicle (antibody alone)-treated mice to less than 1-10% following Akt Inhibitor VIII treatment. Spleen cells also can be assayed for F4/80+ and are expected to show a reduction. To assess the impact of Akt Inhibitor VIII treatment on the presence of luciferase and β-gal β-gal^{pH6} activity in mice bearing SCs, cell lysates from peritoneal lavage infiltrates are assayed. Luciferase activity may be found to be undetectable from the lavage of mice that received the Akt Inhibitor VIII, and preferably will show at least a 10-fold if not greater than a 47-fold reduction between vehicle- and Akt inhibitor VIII-treated groups. Loss of luciferase activity from the lavage of treated mice is expected to be accompanied by a concomitant decrease in β-gal^{pH6} activity. Thus, in vivo Akt Inhibitor VIII treatment of mice bearing alginate-embedded SCs should result in the partial or complete depletion of p16(Ink4a)/β-gal^{pH6}-positive macrophages from the peritoneal cavity.

Therefore, this example shows how to remove SAMS from a mixed cell population using a small molecule which inhibits gene involved in apoptosis. Examples 11-14 pertain to selective removal of senescent cells including macrophages. These compounds are tested further for removal of SAMS according to example 5.

### Example 11

This example provides examples of the use of compounds of the present disclosure.

Compound A1.1 selectively kills senescent human neonatal dermal fibroblasts (NDF) cells in a dose- and time-dependent manner.

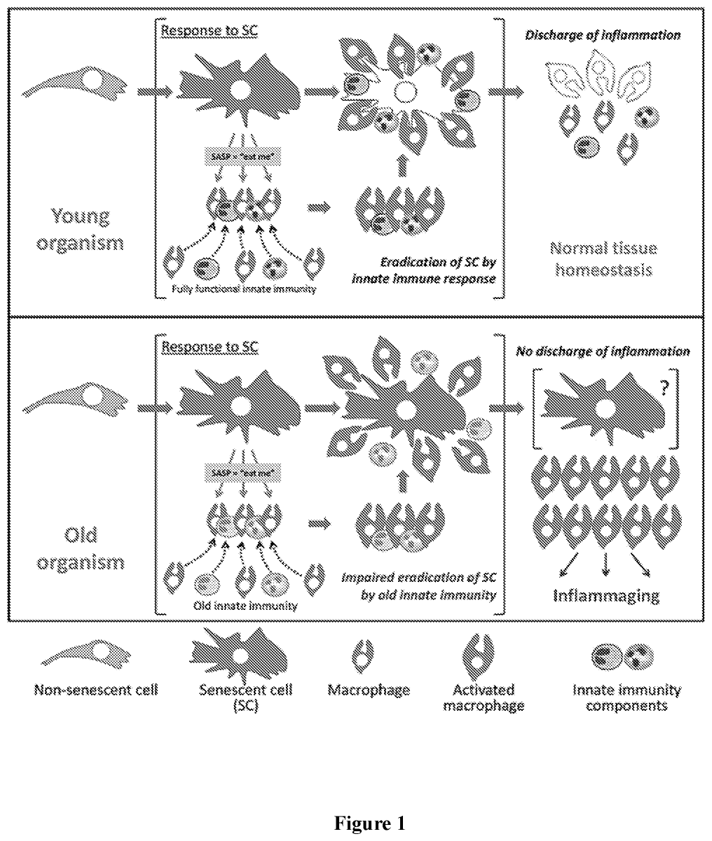

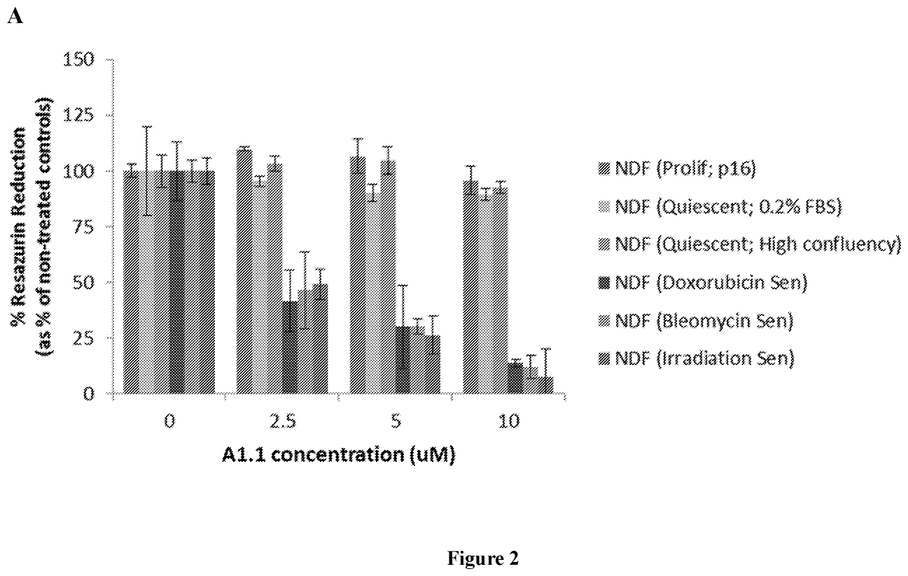

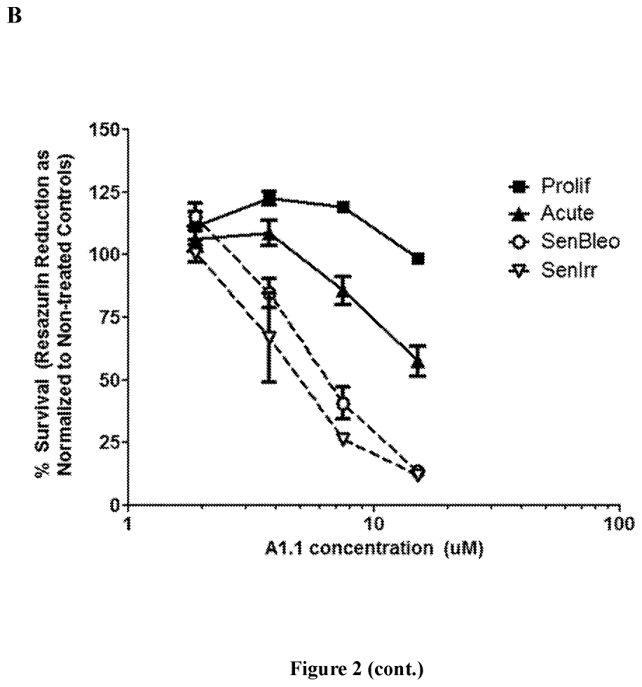

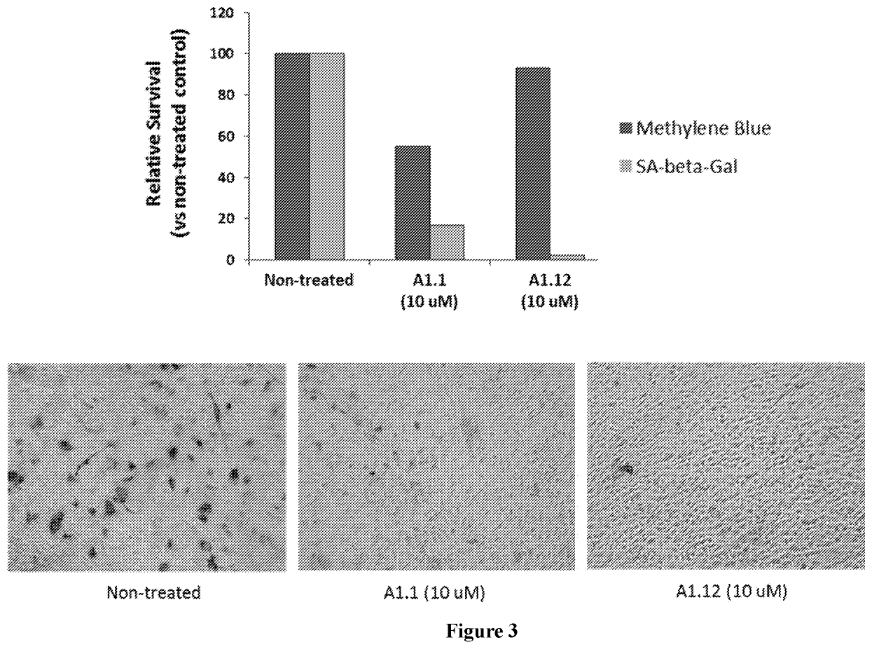

Normal proliferating and quiescent NDF cells, and the cells made senescent via exposure to genotoxic agents (doxorubicin, bleomycin and gamma-irradiation) were incubated with increasing concentrations of A1.1. At 72 hours post-treatment cell viability was measured via resazurin viability assay. Concentrations of A1.1 up to 10 μM had no significant effect on proliferating or quiescent NDF cells (FIG. 1A, 1B). However, concentrations as low as 2.5 μM A1.1 significantly reduced the number of viable cells in all three types of senescent cells.

A1.1 selectively kills senescent human neonatal dermal fibroblasts (NDF) cells in time-dependent manner discriminating between acute genotoxic stress and senescence (FIG. 1B). Acutely damaged NDF cells were irradiated with 4 Gy gamma-irradiation 24 hours before A1.1 treatment. Senescent NDF cells were treated with bleomycin or irradiated with 15 Gy and incubated for 14 days to induce senescence before treatment with A1.1. Both types of senescent cells demonstrated greater sensitivity to A1.1 comparing to acutely damaged or proliferating cells.

### Example 12

This example provides examples of the use of compounds of the present disclosure.

Cytotoxicity of A1.1 analogs against bleomycin-induced senescent human NDFs and rat dermal fibroblasts (RDFs). Activity of analogs was assessed from dose-response curves against irradiation- or bleomycin-induced senescent and proliferating cells to calculate IC50s (uM). The fold difference of proliferating versus senescent cell IC50s was used to assess the level of specific anti-senescent activity of the compounds.

For generation senescent human neonatal dermal fibroblasts (NDFs) and adult rat dermal fibroblasts (RDFs), cells are treated at ˜10 ug/ml bleomycin for 3 days (for bleomycin-induced senescence) or irradiated once at 20 Gy using a gamma irradiator (Cs source), followed by at least 7 day recovery in fresh medium for induction of senescence. Senescent and non-senescent (proliferating) cells for each cell line are plated into 384-w plate, followed by 72 hour exposure to a dose titration of compounds. Viability measurements are estimated using the resazurin viability assay. Using GraphPad Prism 5, the IC50 values are calculated from the non-linear regression of log-transformed compound concentrations versus dose-response normalized to background and non-treated controls.

Table of biological activity of selected examples against senescent and proliferating cell lines.

TABLE 1
IC50 values of anti-senescent compounds against normal
and senescent human (NDF) and rat (RDF) cells.

| | IC50, μM/L | | | | | |
|---|---|---|---|---|---|---|
| Example | NDFsen.bleo | NDFsen.irr | NDFprolif | RDFsen.bleo | RDFsen.irr | RDFprolif |
| A1.1 | <5 | <10 | >20 | <10 | <5 | >20 |
| A1.4 | <10 | nt | >20 | nt | nt | nt |
| A1.5 | <10 | <10 | >10 | <5 | <5 | >10 |
| A1.6 | <5 | <5 | >20 | <5 | <5 | >20 |
| A1.7 | <2.5 | <2.5 | >10 | <2.5 | <2.5 | >5 |
| A1.8 | <10 | nt | >20 | nt | nt | nt |
| A1.9 | <20 | <20 | >20 | >20 | <20 | >20 |
| A1.12 | <5 | nt | <10 | <5 | nt | <10 |
| A1.14 | <5 | <5 | >20 | <2.5 | <2.5 | >20 |
| A1.15 | <5 | <5 | >20 | <2.6 | <5 | >20 |
| A2.1 | <5 | <5 | >20 | <5 | <5 | >20 |
| A2.2 | <2.5 | <5 | >20 | <2.5 | <2.5 | >20 |
| A2.3 | <20 | <20 | >20 | <15 | nt | >20 |
| A3.1 | <2.5 | <2.5 | <5 | <2.5 | <2.5 | >20 |
| A3.2 | <15 | <10 | >20 | <5 | <2.5 | >20 |
| A4.1 | <5 | <2.5 | >5 | <5 | <5 | <10 |
| A4.2 | <15 | <15 | >20 | <10 | <10 | >20 |

### Example 13

This example provides examples of the use of compounds of the present disclosure.

TABLE 2
Small-molecule A1.1 exerts selective anti-senescent
cytotoxicity across a diverse set of senescent cells.

| | | | | Activity | Activity | |
|---|---|---|---|---|---|---|
| | | | | against | against | |
| | | | | Senescent | Proliferating | |
| | Description of | Cell line/ | Senescence | Cells | Cells | Selectivity |
| Species | cells | strain | Induction | (IC50; uM) | (IC50; uM) | (Prolif/Sen) |
| Human | Primary dermal | NDF | Bleomycin | 3.5 | 18.7 | 5.3 |
| | fibroblasts | | Irradiation | 6.5 | 18.7 | 2.9 |
| | | | H2O2 | 3.2 | 18.7 | 5.9 |
| | | | H-Ras | 8.9 | 18.7 | 2.1 |
| | | | Etoposide | 3.8 | 18.7 | 4.9 |
| | | | Doxorubicin | 5.1 | 18.7 | 3.7 |
| | | | Busulfan | 3.6 | 18.7 | 5.3 |
| | | | Replicative | 10.5 | 18.7 | 1.8 |
| | | | (late passage) | | | |
| | Primary | AdMSC | Bleomycin | 5.6 | 12.6 | 2.3 |
| | adipose-derived | | | | | |
| | mesenchymal | | | | | |
| | stromal cells | | | | | |
| | Melanoma | SK-Mel-103 | Bleomycin | 5.6 | 18.5 | 3.3 |
| | Lung | A549 | Bleomycin | 4.5 | 16.5 | 3.7 |
| | adenocarcinoma | | Irradiation | 3.7 | 16.5 | 4.5 |
| Rat | Primary dermal | RDF | Bleomycin | 4.4 | >20 | >4.6 |
| | fibroblasts | | Irradiation | 4.5 | >20 | >4.4 |
| Mouse | Primary lung | mLF | Bleomycin | 7.1 | 10.6 | 1.5 |
| | fibroblasts | | Irradiation | 4.0 | 10.6 | 2.7 |
| | Primary | mSQ | Bleomycin | 6.7 | 14.7 | 2.2 |
| | adipose-derived | | Irradiation | 9.9 | 14.7 | 1.5 |
| | mesenchymal | | | | | |
| | stromal cells | | | | | |

Table 2 demonstrates that A1.1 exerts cytotoxic activity against various types of senescent cells, selectively eliminating SAMs from several mammalian species, from different tissue types, and against senescence induced by different means. Anti-senescent selectivity is observed in primary human dermal fibroblasts (NDFs) induced to senescence via genotoxic stress, oxidative stress and replicative stress. In addition, human, rat and mouse cells senesced via bleomycin and irradiation all show sensitivity towards A1.1 treatment compared to A1.1-treated naïve cell controls.

### Example 14

This example provides examples of the use of compounds of the present disclosure.

A1.1 and A1.12 selectively kill senescent prostate cells in a model of dominant negative PTEN-induced senescence.

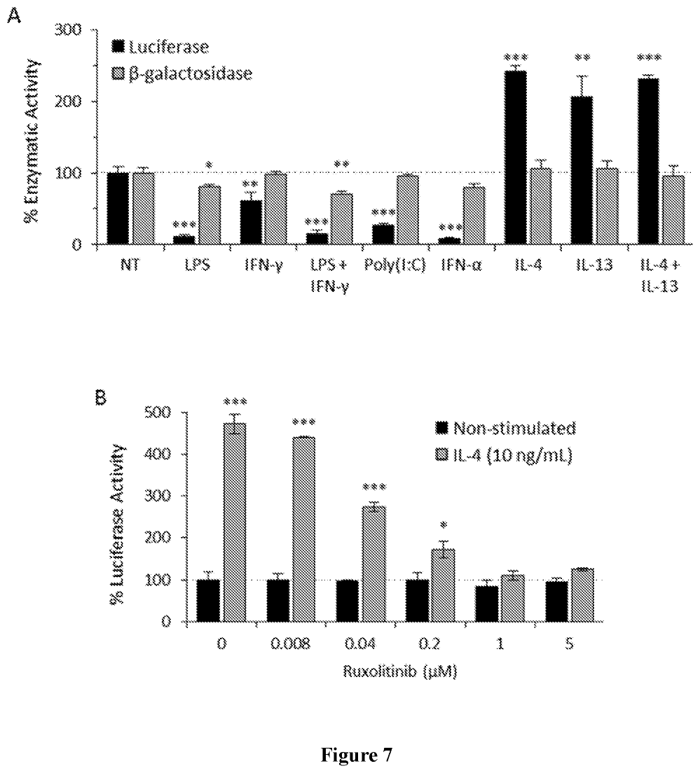

Senescent cells are strongly associated with benign prostate hyperplasia (BPH) and the development of prostate cancer [Vital, P. et al. (2014) The Senescence-Associated Secretory Phenotype Promotes Benign Prostatic Hyperplasia. Am J Pathol, 184(3): 721-731; Burton, D. et al. (2013). Androgen Deprivation-Induced Senescence Promoted Outgrowth of Anderogen-Refractory Protstate Cancer Cells. PLOS One, 8(6):e68003]. Loss of PTEN, frequently observed in BPH and prostate cancer, has been shown to induce senescence in prostate cells. We sought to evaluate the efficacy of our compounds in a model of RWPE-1 normal prostate epithelial cells transduced with a construct for inducible loss of PTEN function [doxycycline-inducible expression of a dominant-negative PTEN (dnPTEN) mutant]. In the presence of doxycycline, a fraction of the population of transduced cells became senescent, while cells bypassing senescence continued to grow until a confluent monolayer was formed. Visualization of senescent cells via staining with SA-β-Gal revealed positively stained senescent cells within a monolayer of non-senescent SA-β-Gal-negative cells. Exposure of this model to A1.1 and analog A1.12 revealed the selective elimination of senescent cells (assessed by decreased number of SA-β-Gal-positive cells), while showing a minimal decrease in the overall cell mass comprised mostly of proliferating cells (as assessed by quantification of methylene blue staining) (FIG. 7). A1.1 appeared to inhibit growth of proliferating cells during treatment resulting in a decrease in methylene blue staining, while the analog A1.12 showed little effect against proliferating cells in this model.

### Example 15

This example provides examples of the use of compounds of the present disclosure.

A1.1 and analogs reduce tumor burden in a model of senescence-enhanced B16 lung metastasis.

The link between senescent stromal cells and cancer progression has been demonstrated in several models both in vitro and in vivo, supporting the hypothesis that the accumulation of senescent cells contributes to the increased rates of cancer observed with age. Several senescence-associated factors have been implicated in this phenomenon, including the senescent secretory phenotype (i.e. secretion of soluble factors (i.e. cytokines, growth factors, and matrix remodeling components), as well as signaling associated with direct cellular contact with the senescent cells. Senescence-induced changes in tissue microenvironment promote the establishment and growth of tumorigenic cell lines, and most notably, have been shown to induce a tumorigenic phenotype in otherwise non-tumorigenic cells [Liu, D. & Horsby, P. (2007). Senescent human fibroblasts increase the early growth of xenograft tumors via matrix metalloproteinase secretion. Cancer Res 67: 3117-3126; Papadopoulou, A. & Kletsas, D. (2011). Human lung fibroblasts prematurely senescent after exposure to ionizing radiation enhance the growth of malignant lung epithelial cells in vitro and in vivo. International Journal of Oncology, 39: 989-999; Krtolica, A. et al. (2001). Senescent fibroblasts promote epithelial cell growth and tumorigenesis: a link between cancer and aging. PNAS, 98(21): 12072-12077].

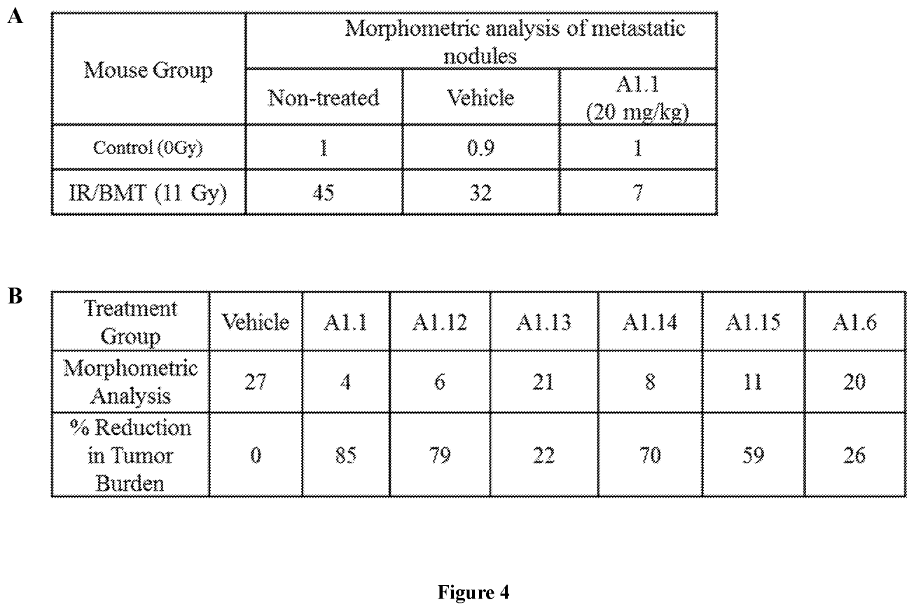

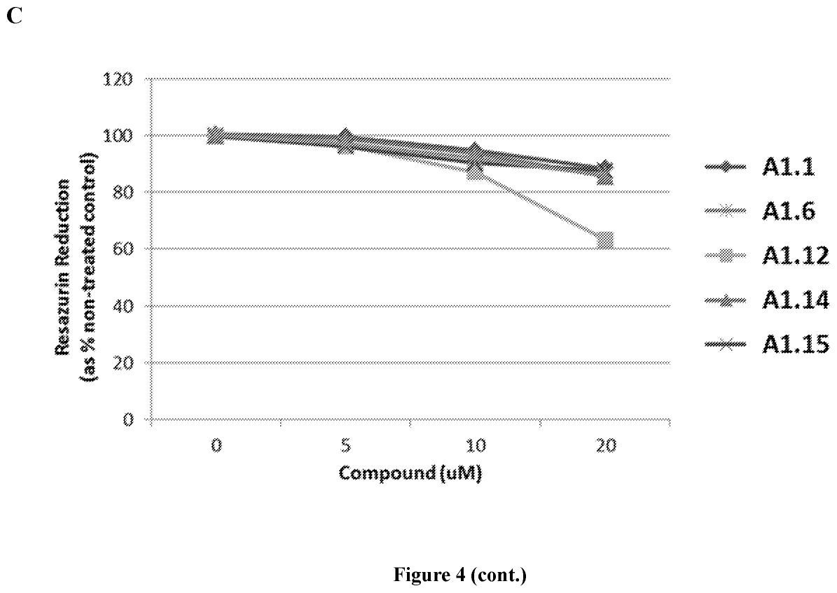

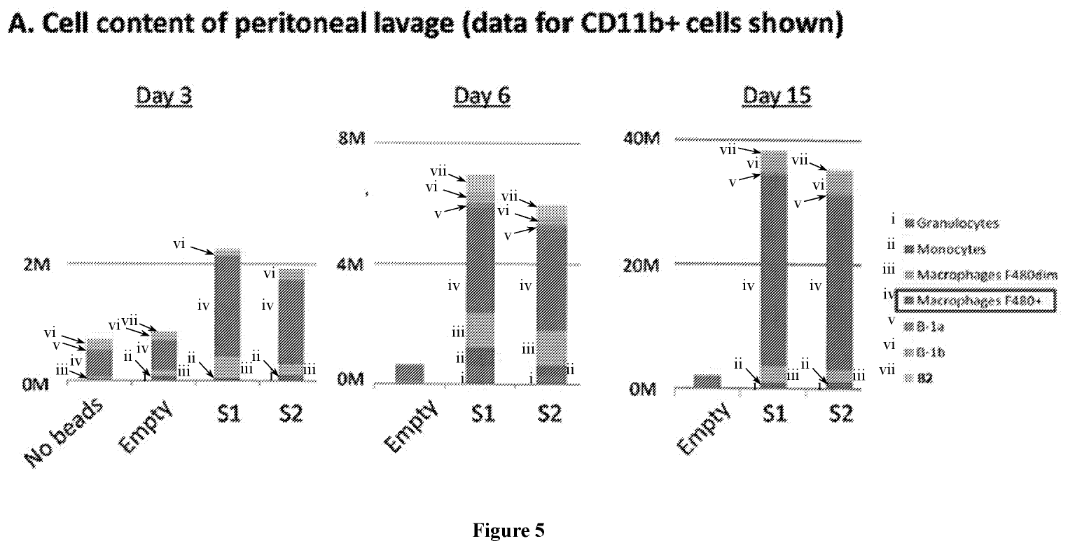

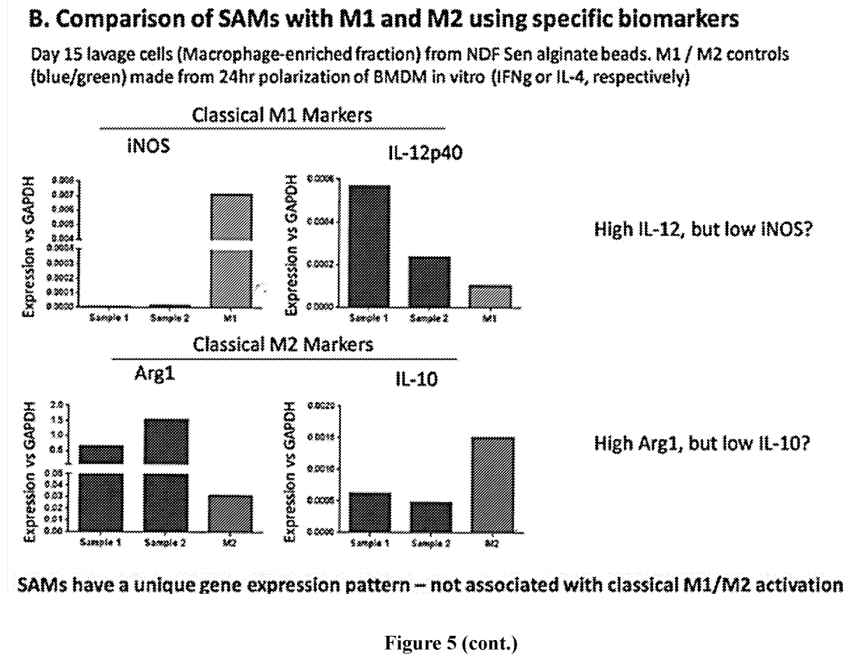

The role of senescent cells in the promotion of metastatic potential of cancer cells has also been described [Patent WO 2013090645 A1; Campisi, J. (2015). Cancer and Aging: Rival Demons]. As demonstrated by Judith Campisi, the clearance of senescent cells via a genetic construct reversed the effect of senescence-mediated B16 lung metastasis in mice, where senescence was induced in vivo via whole body irradiation. To test the efficacy of A1.1, irradiated (11 Gy followed by bone-marrow transplantation) and non-irradiated C57BL/6 mice received B16 melanoma cells via tail vein injection were subsequently (72 hours later) administered A1.1 (20 mg/kg) intraperitoneally once a day for 5 consecutive days. Irradiated mice displayed a 45-fold increase in B16 tumor burden in the lungs (as assessed by morphometric analysis) compared to non-irradiated mice (FIG. 4A). Treatment with A1.1 resulted in a 4.3-fold reduction of B16 metastatic load in the lungs of irradiated mice, as compared to vehicle-treated control mice. Treatment with A1.1 did not affect the tumor burden in non-irradiated animals, indicating that the decrease in tumor burden in irradiated animals is not due to the direct effects of A1.1 on B16 cell growth/establishment. Consistent with these results, A1.1 did not inhibit B16 growth in vitro (FIG. 4C). Together, these data suggest that A1.1 shows selective anti-senescent activity in vivo.

Synthesized analogs were also tested in vivo, to assess whether optimized structures are capable of showing activity against senescence-mediated B16 lung metastasis in irradiated mice (FIG. 4B). Analogs A1.1, A1.12, A1.13, A1.14, A1.15, and A1.6 were administered at 20 mg/kg intraperitoneally for 5 consecutive days. In addition to A1.1, treatment with analogs A1.12, A1.14 and A1.15 at 20 mg/kg reduced the metastatic burden in the lungs of irradiated mice by more than 2-fold. Analogs A1.13 and A1.6 did not show a substantial decrease in B16 metastasis in irradiated mice. In vitro testing of these analogs revealed that compounds exerting in vivo effect in the senescence-enhanced B16 metastasis model showed minimal effect on the growth of B16 cells up to 20 uM (FIG. 4C). Taken together, these data suggest that A1.1 analogs show selective anti-senescent activity in vivo.

### Example 16: Characterization of SAM Polarization Class

Macrophages are generally divided into two classes depending on their predominant activity: M1 and M2. M1 macrophages (also named classically activated macrophages), are “warriors” responsible for elimination of foreign agents (including tumor cells) both directly and through recruitment and activation of other immune cells (for example, T-killer). M2 macrophages are “healers” in that they enhance tissue regeneration and enforce wound healing. M1 and M2 macrophages can be distinguished based on their differential expression of molecular markers. M1 macrophages express high levels of nitric oxide synthase (iNOS) and interleukin-12 subunit beta (IL-12p40), whereas M2 macrophages express high levels of arginase (Arg1) and IL-10.

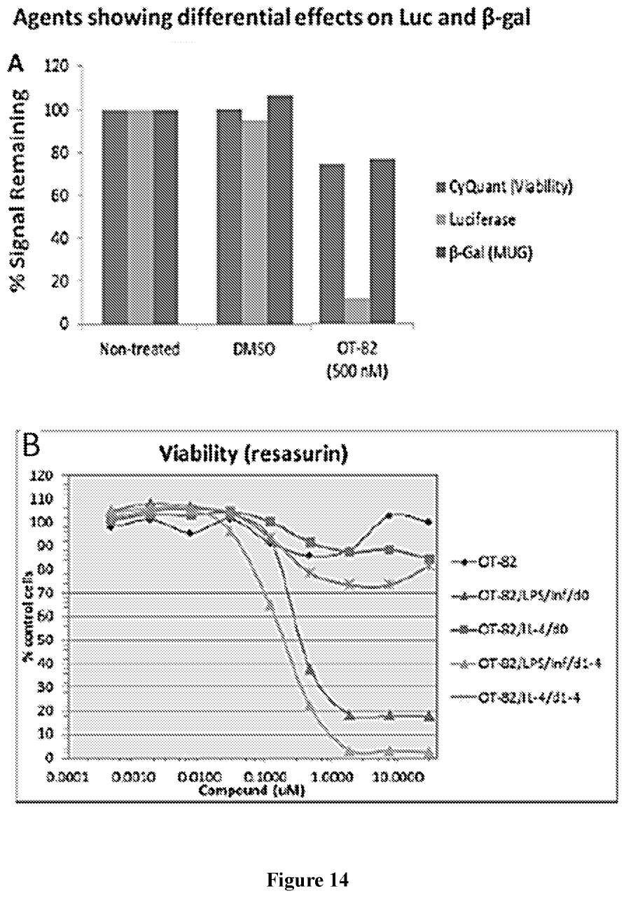

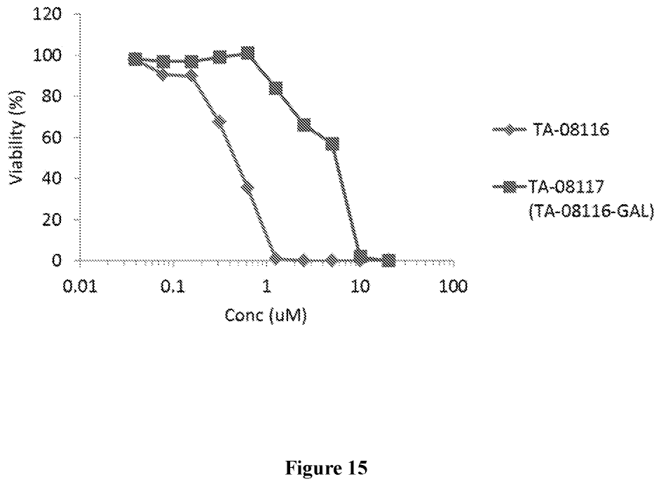

In order to determine whether SAMs exhibit the properties of either M1 or M2 macrophages, the following studies were performed. C57BL/6J mice were injected with alginate beads embedded with senescent NDF cells. The cell content of peritoneal lavage was then characterized in duplicate at day 3, 6, and 15 following injection with SC-containing beads. As negative controls, peritoneal lavage was characterized from mice that were not injected, or who had been injected with empty beads containing no cells. Analysis of peritoneal lavage cell content revealed that at day 3 following injection, CD11b⁺ cells were significantly enriched for F4/80⁺ macrophages in mice injected with SC-containing beads compared to mice injected with empty beads (FIG. 14A). An increase in F4/80⁺ macrophages continued to be observed at days 6 and 15 following SC-containing bead injection (FIG. 14A).

Macrophage enriched fractions of day 15 lavage cells were assayed for production of the M1-specific biomarkers iNOS and IL-12p40 and the M2-specific biomarkers Arg1 and IL-10 and compared to M1 and M2 controls made from 24 hour polarization of BMDM in vitro. Unlike M1 macrophages, which produce high levels of iNOS and IL-12p40, and unlike M2 macrophages, which produce high levels of Arg1 and IL-10, SAMs produce high levels of IL-12p40 and Arg1 and low levels of iNOS and IL-10 (FIG. 14B).

In conclusion, we have found that SAMs can acquire a set of properties that does not allow them to be assigned to M1 or to M2 macrophage classes. Namely, they produce high levels of IL-12, similar to M1 macrophages, but do not produce iNOS. At the same time they are positive for arginase (a striking feature of M2 macrophages), but synthesize only low amounts of IL-10 (similar to that of M1 macrophages).

### Example 17: SAMs are not Senescent

Proliferation Arrest of a Population of Macrophages Largely Represented SAMs is Reversible

M1 and M2 macrophages are believed to be incapable of division. Irreversible growth arrest is a striking feature of senescent cells. In order to determine whether SAMs are arrested irreversibly, we plated macrophages (representing the vast majority of cells recruited) attached to SC-carrying alginate beads in tissue culture plates and cultivated them in the presence of M-CSF (macrophage colony-stimulating factor) in order to enforce their differentiation. Cells adhesive to the beads were then stained with antibodies against nuclear protein Ki-67, a widely accepted marker of cell proliferation, as well as with antibodies against mouse macrophage marker F4/80 and arginase, which have been shown to be largely expressed by SAMs.

The cell population which outgrows from alginate beads in culture is represented solely by macrophages expressing F4/80 (purple) and Arginase 1 (green) and part of them express Ki-67 (red) which indicates that they retain the ability to divide. Thus, at least part of the population of cells, mostly represented by SAMs, is capable of proliferation.

Expression of p16(Ink4a) and Beta-Galactosidase in Macrophages is Independent of p53

SAMs express two markers also present in senescent mesenchymal cells, increased p16(Ink4a) and senescence associated beta-galactosidase (SAβG) expression. A recent publication (Childs et al. Science. 2016 Oct. 28; 354(6311):472-477) has identified an additional source of p16⁺/SAβG⁺ macrophages in atherosclerotic plaques. These results were interpreted as evidence of macrophage senescence in vivo. However, our data suggests that the expression of p16(Ink4a) is a functional component of macrophage polarization and response.

The tumor suppressor protein p53 (encoded by Trp53) is a crucial cell cycle regulator. It has been widely reported that p53 is required for DNA damage-induced growth arrest and cellular senescence, which results in constitutive elevation of p16(Ink4a) and SAβG expression. To investigate whether the acquisition of these traits in macrophages is a consequence of macrophage senescence, we utilized an in vivo model previously shown to generate p16(Ink4a)/SAβG-positive macrophages in wild type mice (i.e. intraperitoneal injection of alginate-encapsulated SCs), and determined whether macrophages with these traits appear in p53-knockout mice. Since p53 is required for senescence induction, underlying the acquisition of p16(Ink4a) and SAβG markers, we sought to determine whether p53 is required for expression of these markers by SAMs. To do so, we evaluated the response to intraperitoneal injection of SC-containing alginate beads in p53 knockout mice (p53^{-/-}), assessing p16(Ink4a) and β-galactosidase expression in elicited cells compared to those elicited in wild type mice (p^{53+/+}).

Two weeks after injection of SC-containing alginate beads, the thickness, cell density, and abundance of macrophage marker F4/80 of immunocytes surrounding SC-containing alginate beads in p53-knockout mice was unaffected compared to wild type mice. Similarly, we found that p53 deficiency did not diminish the elevated number of elicited cells recovered from the peritoneal lavage. Together, these data suggest that immunocyte infiltration and response does not require p53-dependent pathways. Next, we examined whether p53 was required for the induction of p16(Ink4a) and SAβG in this model. Immunocyte capsules from p53^{+/+} and p53^{-/-} strains revealed similar expression of p16(Ink4a) mRNA and β-galactosidase activity as quantitatively measured via 4-MUG hydrolysis. In agreement with these results, SAβG staining revealed no differences in the presence or absence of p53. Based on these data, expression of p16(Ink4a) and SAβG in macrophages is independent of p53 status and thus likely acquired through a senescence-independent mechanism.

Our data indicates that p53 deficiency does not abrogate induction of p16(Ink4a) nor β-galactosidase expression in SAMs. Thus, p53 is not required for the expression of either marker in macrophages, unlike senescence induction of mesenchymal cells.

SAMs do not Exhibit Cytoplasmic Localization of HMGB1, a Marker of Mesenchymal SCs

The relocalization of HMGB1 from the nucleus to the cytoplasm (and extracellular space via secretion) was identified as a marker of cellular senescence in stromal cells (Davalos, A. R. et al. J Cell Biol. 2013 May 13; 201(4):613-29). We investigated the localization of HMGB1 in SAMs elicited by SC-containing alginate beads to determine whether these p16/SAβG-positive macrophages express additional mesenchymal SC markers. Cells isolated from alginate bead capsules were separated into 2 main populations via FACS: stromal cells (CD45⁻) and hematopoietic cells (CD45⁺). Macrophages (SAMs), obtained by enrichment of CD45⁺ fraction through adherence selection, displayed localization of HMGB1 in the nucleus similar to proliferating CD45− stromal cells. In contrast, irradiation-induced senescent CD45− cells, a positive control for senescence, displayed cytoplasmic localization of HMGB1. Thus, despite expression of p16(Ink4a) and SAβG in SAMs, they lack additional markers of senescence such as cytoplasmic HMBG1. These data demonstrate that SAMs are not senescent, in accordance with p53-independence of acquisition of p16 and SAβG. Notably, secretion of HMGB1 (and cytoplasmic localization) is not unique to mesenchymal SCs, as it is well-known to be a marker of activated macrophages (Chen, G. et al. (2004). Journal of Leukocyte Biology. 76(5), 994-1001). Thus, HMGB1 is an additional senescence-associated marker in mesenchymal cells that have different functions and are differentially regulated in macrophages.

In summary, multiple markers associated with mesenchymal SCs have been described in macrophages (and other hematopoietic cells) to performed different functions, playing integral roles in innate immunity. The markers include p16(Ink4a), p21(Cip1), p19(Arf), p53, CDKs, HMGB1, and SAβG, which have been shown to play a role in macrophage differentiation, polarization and/or response to physiological immunomodulatory stimuli.

### Example 18: Reversible Modulation of p16(Ink4a) in Response to Macrophage Polarization

p16(Ink4a)- and SAβG-Positive Macrophages Remain Responsive to Polarizing Stimuli

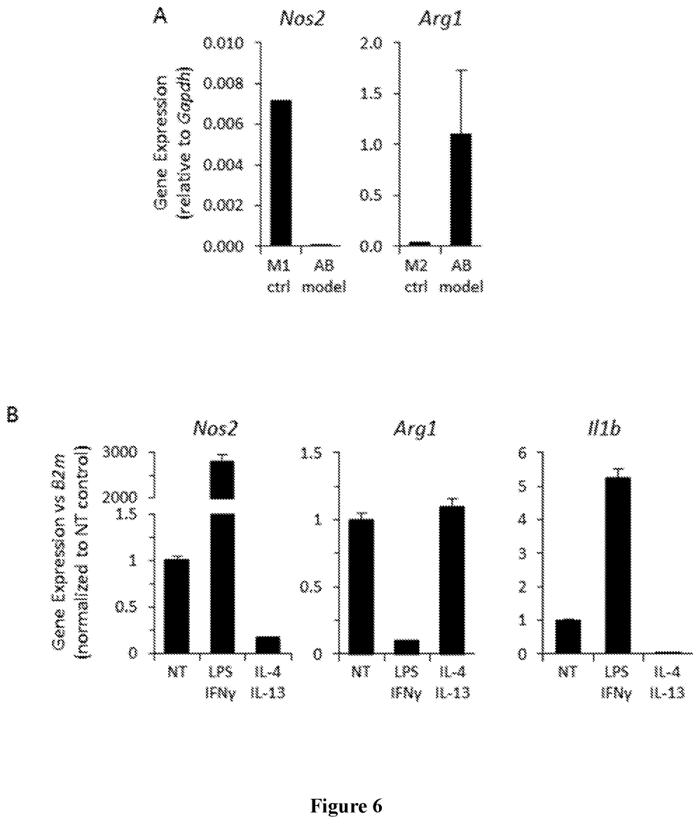

Macrophage phenotype/polarization is categorized by gene expression profiles associated with M1 and M2 activation states. Given that p16(Ink4a) expression was recently demonstrated to modulate macrophage polarization, we sought to characterize the polarization state (M1 or M2) of alginate bead model-elicited macrophages via expression of conventional markers of M1 and M2 phenotypes (Nos2 and Arg1, respectively). As controls, bone marrow-derived macrophages (BMDM) polarized via IFN-γ (M1) or IL-4 (M2) were also analyzed. Alginate bead model-elicited macrophages showed low expression of Nos2 (>500-fold lower than M1-polarized BMDM), while Arg1 was markedly upregulated (>20-fold compared to M2-polarized BMDM) (FIG. 6A). Based on these expression profiles, these p16(Ink4a)/SAβG-positive macrophages possess an M2-like phenotype.

Macrophages normally exhibit highly plastic phenotypes, demonstrating reversible polarization upon challenge with immunomodulatory stimuli. Therefore, we sought to determine whether p16(Ink4a)/SAβG-positive macrophages exhibit a reversible, physiologically responsive state, or if the co-expression of p16(Ink4a) and SAβG exists as part of a permanently acquired phenotype (such as a senescent or other refractory state). To discriminate between these two possibilities, alginate bead model-elicited macrophages were stimulated with M1- and M2-inducing stimuli (LPS/IFN-γ and IL-4/IL-13, respectively) to determine the responsiveness of M1/M2-associated gene expression. Stimulation with LPS/IFN-γ induced high expression of Nos2 (2,800-fold induction) and decreased expression of Arg1 (10-fold) (FIG. 6B), consistent with repolarization towards an M1 phenotype. In contrast, stimulation of these M2-like macrophages with IL-4/IL-13 resulted in a >5-fold decrease in Nos2 expression, with elevated Arg1 expression remaining unchanged, consistent with the maintenance of an M2-like state. Macrophage polarization also affects the expression of cytokines, which facilitate macrophage interactions with the microenvironment. Modulation of M1/M2 markers was consistent with expression of M1-associated pro-inflammatory cytokine IL-1β, which increased with LPS/IFN-γ stimulation (5-fold) and decreased with IL-4/IL-13 (>20-fold) (FIG. 7B).

Thus, p16(Ink4a)/SAβG-positive macrophages remain responsive to immunomodulatory stimuli and are capable of modulating gene expression consistent with repolarization of macrophage phenotypes.

Reversible Modulation of p16(Ink4a) and SAβG in Response to Macrophage Polarization

While studies utilizing p16(Ink4a)-deficient macrophages clearly demonstrate a role for p16(Ink4a) in macrophage polarization, the regulation of p16(Ink4a) expression in response to polarizing agents has not yet been described. To determine whether macrophage polarization regulates p16(Ink4a) and SAβG expression, peritoneal lavage from p16^{Ink4a/Luc} mice elicited by our alginate bead model were stimulated with M1- and M2-inducing agents and subsequently, luciferase activity (driven by endogenous p16(Ink4a) promoter) and β-galactosidase activity (assayed via 4-MUG hydrolysis) were measured (FIG. 7A). Stimulation towards M1 polarization with TLR4 agonist LPS (1 ng/mL) decreased luciferase activity relative to non-treated control (80-90% decrease), with a slight but statistically significant decrease in β-galactosidase activity (20-30% decrease). Analysis of p16(Ink4a) and β-galactosidase (G1b1) expression via qRT-PCR confirmed substantial downregulation following LPS treatment. Consistently, LPS decreased the intensity of SAβG staining in treated macrophages. The response of macrophages to LPS was rapid, with a significant decrease in luciferase activity observed within 8 hours (˜40% decrease). The co-treatment of IFN-γ with LPS, classically used for M1 induction, showed similar effects to that of LPS alone (FIG. 7A). Stimulation with additional M1-inducing agents, including type I and II interferons (IFN-α and IFN-γ, respectively) and Toll-like receptor 3 (TLR3) agonist Poly(I:C), decreased luciferase activity without affecting β-gal activity (FIG. 7A). These data demonstrate that M1-polarizing stimuli decreases p16(Ink4a) and SAβG expression in macrophages.

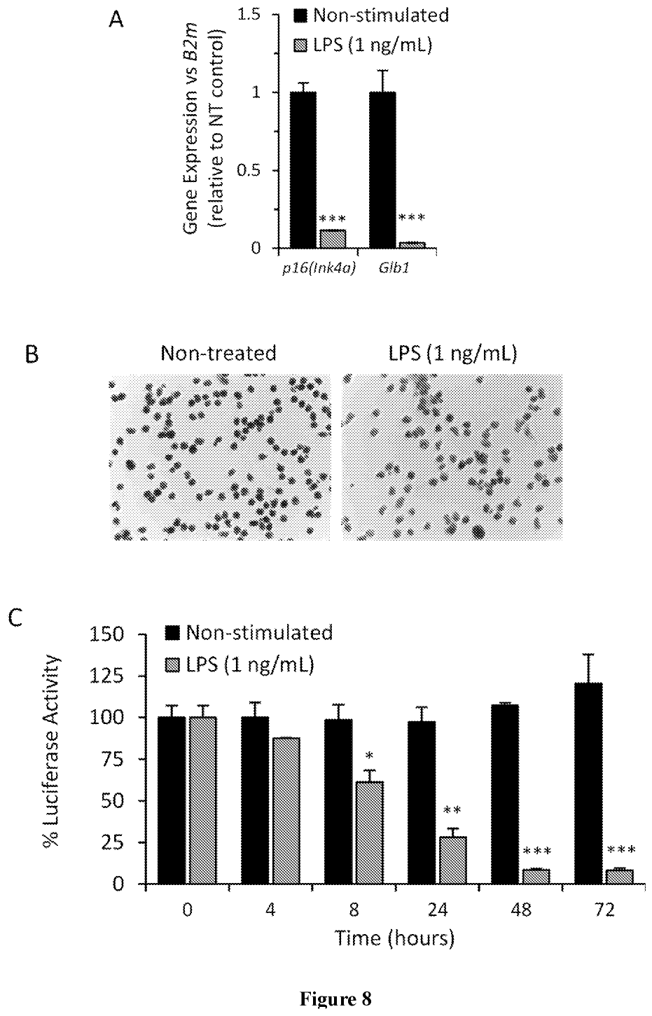

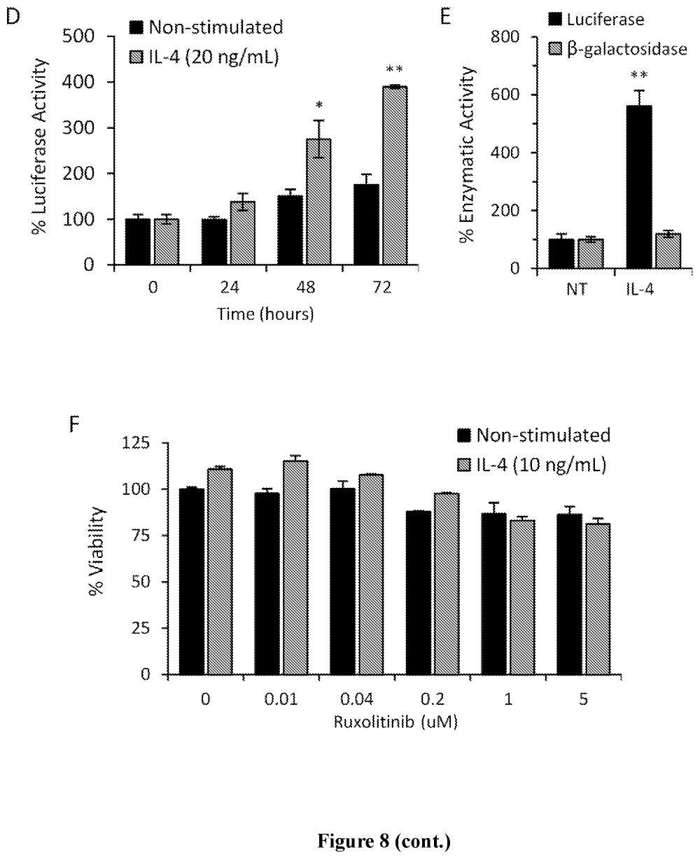

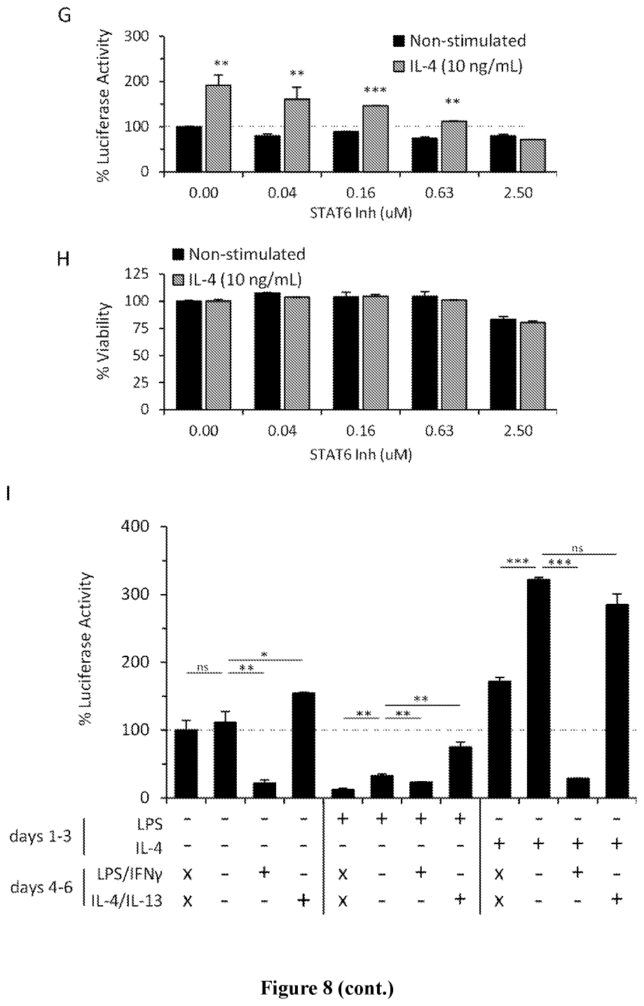

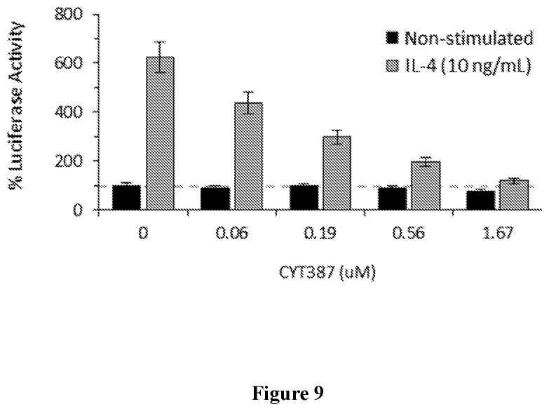

In contrast to these observations, stimulation with M2-polarizing cytokines IL-4 and/or IL-13 increased luciferase activity (2.1- to 2.4-fold increase) without modulating β-gal activity (FIG. 7A). However, IL-4-induced luciferase activity exhibited delayed kinetics (>48 hours). Similar findings were observed for peritoneal macrophages elicited by proteose peptone, indicating that the increased luciferase activity in vitro is not specific to our alginate bead model-elicited macrophages. Further, the IL-4-dependent increase in luciferase activity was abrogated by inhibitors of canonical IL-4-induced JAK-STAT signaling, including JAK1/2 inhibitor ruxolitinib and STAT6 inhibitor AS1517499, without affecting viability (FIG. 7B and FIGS. 8F-H). Similar to ruxolitinib and STAT6 inhibitor AS1517499, CYT387 (momelotinib) abrogates IL-4-induced p16(Ink4a) expression (FIG. 9). No effect on p16(Ink4a) expression was observed on macrophages treated with these inhibitors in the absence of IL-4. These data indicate that p16(Ink4a) expression is increased in response to M2-polarizing cytokines IL-4 and IL-13.

Macrophages are capable of dramatic shifts in gene expression, exhibiting high plasticity in phenotypes in response to environmental stimuli. To further characterize the association of p16(Ink4a) expression with macrophage polarization states, alginate bead model-elicited macrophages were polarized and subsequently challenged with stimuli (e.g. M1 to M2, or M2 to M1) to test the effects of repolarization on p16(Ink4a) expression. We found that p16(Ink4a) expression was reversibly modulated, showing elevated expression levels in response to M2 challenge and decreased expression levels in response to M1 challenge.

Poly(I:C) Abrogates Alginate Bead Model- and Age-Dependent Increases in p16(Ink4a) Expression In Vivo

Our data demonstrate that M1-inducing agents downregulate p16(Ink4a) expression in macrophages ex vivo. We next investigated whether p16(Ink4a) expression was susceptible to M1-mediated regulation in vivo using immunotherapeutic agent Poly(I:C). Upon intraperitoneal injection of alginate-encapsulated cells, p16^{Iak4a/Luc} mice accumulate luciferase-positive macrophages that can be monitored in vivo via the IVIS imaging system. Following a 10 day induction of bioluminescence, mice treated intraperitoneally with Poly(I:C) (2 and 10 mg/kg) showed a complete abrogation of signal, while the signal remained unchanged in vehicle-treated mice. Analysis of peritoneal lavage cells from Poly(I:C)-treated mice revealed a >6-fold decrease in luciferase activity per cell compared to non-treated controls, with no difference in the proportion of CD11b⁺ F4/80⁺ macrophages. Further, treatment with Poly(I:C) had no effect on β-galactosidase activity nor viability (>95%) of lavage cells, consistent with in vitro data. Together, these data suggest that Poly(I:C) modulates p16(Ink4a) expression in macrophages in vivo. We similarly found that IFN-α abrogates elevated p16(Ink4a) expression in vivo using the same experimental alginate bead model.

Given that macrophages contribute to the elevated p16(Ink4a) expression observed in aged mice, we investigated whether this expression was susceptible to immunomodulatory regulation. Chronologically aged mice (83-weeks old) were treated with Poly(I:C) and the luminescence of individual organs was quantitated via IVIS. Poly(I:C)-treated mice displayed a ˜4-fold reduction in luminescence from visceral adipose tissue, with no significant effects on other tissues, including spleen, lungs, kidney and liver. Poly(I:C) significantly decreased luciferase activity from the isolated stromal vascular fraction from these mice compared to saline-treated controls, whether normalized per cell or per gram of fat. Poly(I:C) treatment induced an infiltration of F4/80⁺ and SAβG⁺ macrophages in Poly(I:C)-treated mice, consistent with a greater yield of F4/80+ cells quantitated from the stromal vascular fraction of cells via flow cytometry. Thus, the observed decrease in the luciferase signal in adipose tissue upon treatment with Poly(I:C) was not accompanied by the depletion of macrophages. These data suggest that p16(Ink4a) expression in adipose tissue macrophages from aged mice is able to be modulated in response to immunomodulatory stimuli.

Thus, these data demonstrate that p16(Ink4a) is capable of being reversibly modulated by pathways associated with classical M1/M2 polarization.

### Example 19: Immunomodulatory Regulation of p16(Ink4a) and SAβG is not an Inherent Property of Senescent Cells

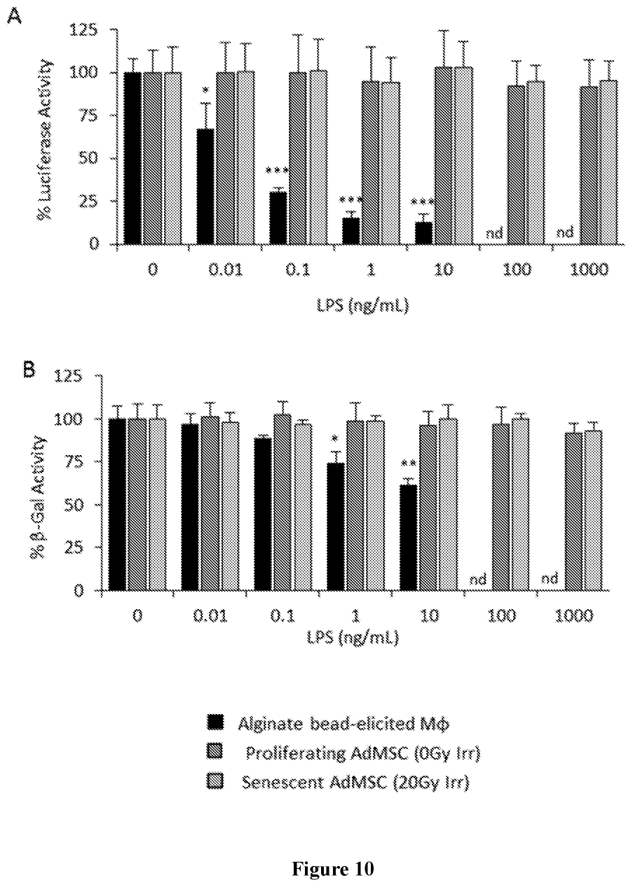

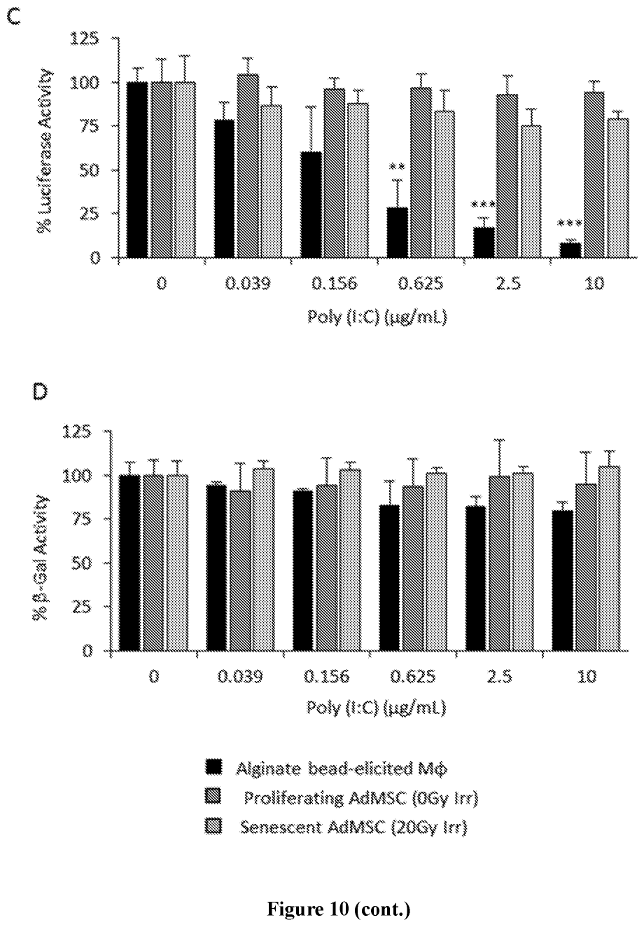

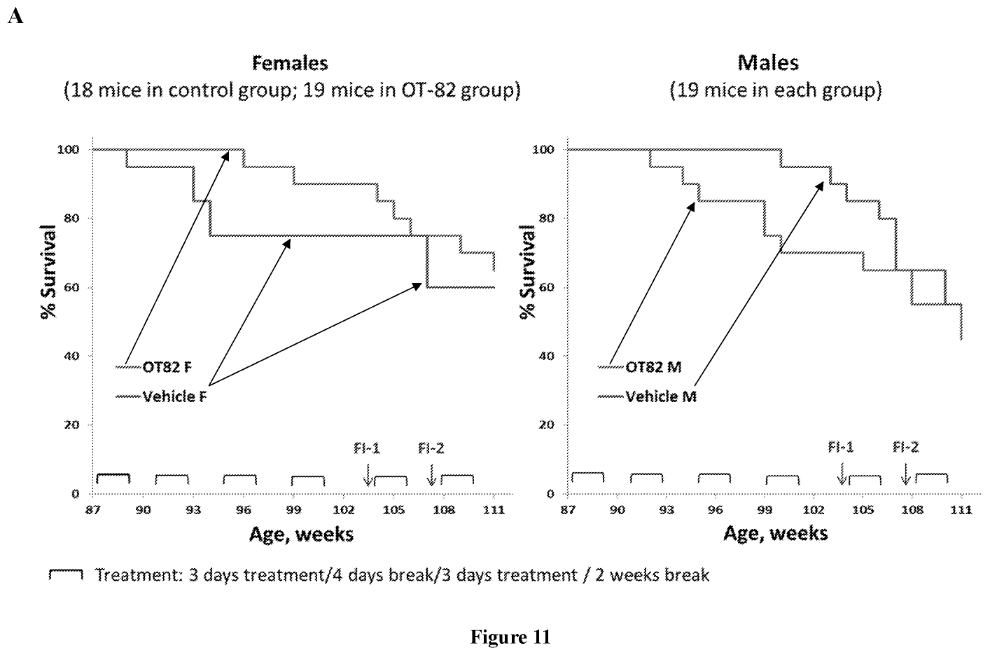

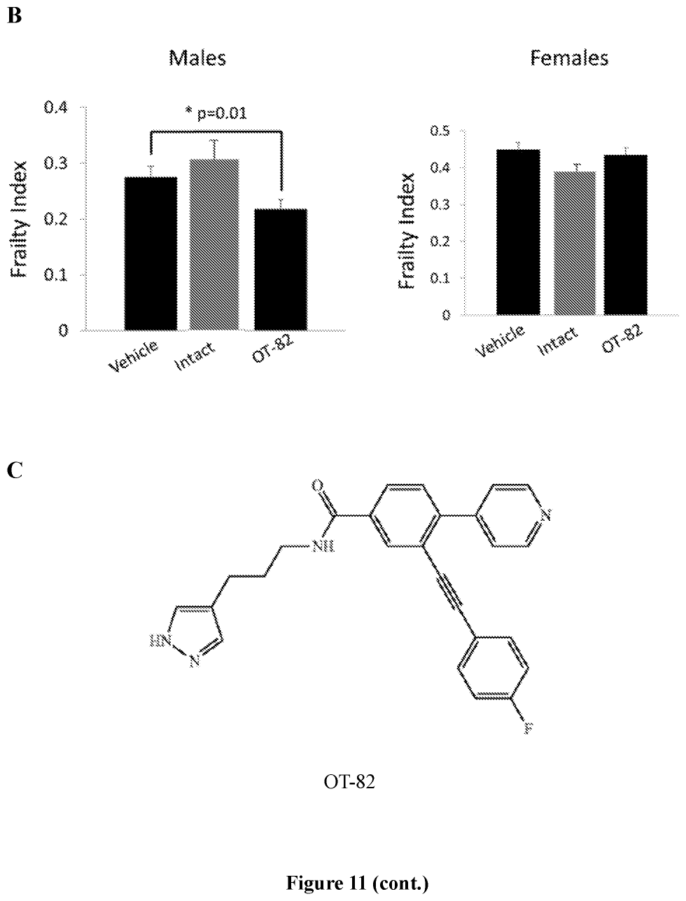

The immunomodulatory regulation of p16(Ink4a) and SAβG in macrophages suggests distinct roles in macrophage physiology. We investigated whether the mechanisms regulating the expression of these traits in macrophages were similar to those found in SCs. Using cells isolated from p16^{Ink4a/Luc} mice, the dose-dependent response of luciferase and β-galactosidase activities to immunomodulatory stimuli in macrophages was compared to that of proliferating and irradiation-induced senescent adipose-derived mesenchymal stromal cells. Characterization of irradiated cells revealed phenotypes consistent with cellular senescence, including an enlarged morphology, positive SAβG staining, and increased p16(Ink4a) expression (assessed via luciferase) and β-galactosidase activity (assessed via 4-MUG hydrolysis). LPS stimulation of alginate bead model-elicited macrophages reduced luciferase activity, with maximal inhibition (˜85%) observed at concentrations ≥1 ng/mL (FIG. 10A). In addition, β-galactosidase activity was reduced by 40% at 10 ng/mL (FIG. 10B).

In contrast, luciferase and β-galactosidase activities were unaltered in senescent and proliferating mesenchyme in response to LPS (up to 1 μg/mL) (FIGS. 10A&B). The effect of Poly(I:C) was also tested, revealing a dose-dependent decrease in luciferase activity that was selective to macrophages, while no effect was observed against β-galactosidase activity (FIGS. 10C&D). Additional agents (IFN-γ, IFN-α, IL-4, IL-13) also demonstrated selective regulation of p16(Ink4a) in macrophages, lacking a differential response between senescent and proliferating mesenchymal cells. Together, these data indicate that the elevated expression of p16(Ink4a) and SAβG in macrophages is regulated via mechanisms distinct from those acquired through senescence.

In conclusion, macrophages possess specific pathways for regulation of p16(Ink4a) and β-galactosidase that are responsive to several immunomodulatory agents, not found in senescent cells.

### Example 20: Assessment of DNA Damage in SAMs

DNA damage is a phenotype associated with senescent cells. To determine if DNA damage was associated with p16 expression in SAMs, DNA damage marker gamma-H2AX was assessed in alginate bead-elicited lavage and capsule cells. Approximately 20-fold greater p16^{Luc} signal per capsule cell was observed compared to lavage cells. Therefore, if DNA damage was associated with p16 expression, capsule cells would be expected to have a large accumulation of this marker. However, analysis of samples via western blot and immunofluorescence revealed that gamma-H2AX is not abundant in SAMs, and that a low percentage (˜6%) of gamma-H2AX-positive cells is consistent among samples. As a control for the appearance of gamma-H2AX in macrophages, peptone-elicited lavage cells (M0 macrophages), when acutely irradiated at 20 Gy, show an increase in gamma-H2AX.

In conclusion, in contrast to the phenotype of senescent cells, SAM phenotype is not associated with the accumulation of DNA damage.

### Example 21: Assessment of the Effect of Compounds on Survival and Frailty Index

Selected compounds were analyzed for their effects on survival and the frailty index of chronologically aging mice.

Compound OT-82 (product of Oncotaris, Buffalo, N.Y.) (disclosed as compound TT-03582 in U.S. Publication 2016/0347748 (published Dec. 1, 2016)) has revealed positive effects on the frailty index (FI) of males (FIG. 27B) with no effects on survival rates: 65% of OT-82 treated females (60% in vehicle group) and 45% of OT-82 treated males (55% in vehicle control group) have survived after 6 courses of treatment, reaching 111 weeks of age (24^{th} week of experiment) (FIG. 27A).

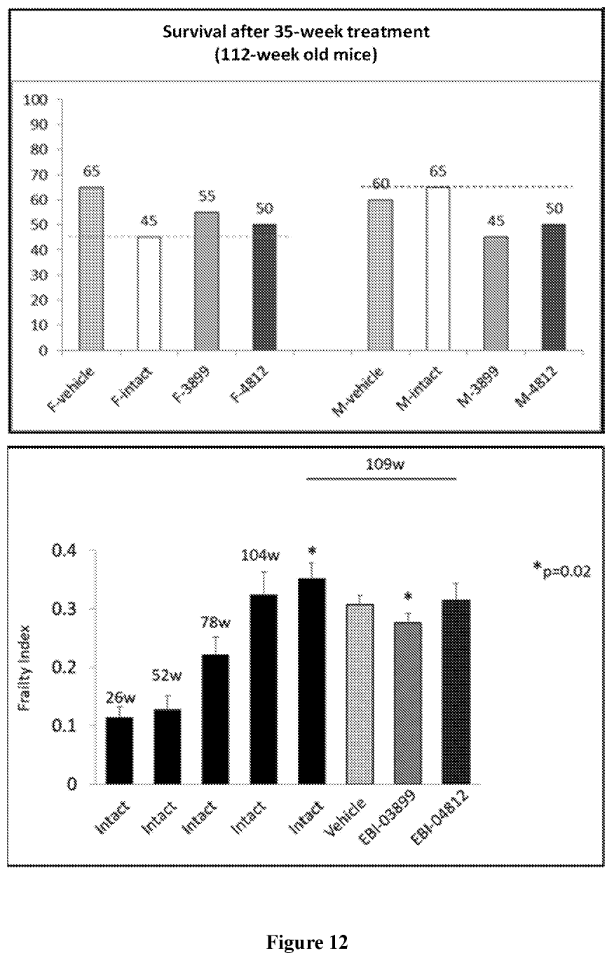

Asenins TA-3899 and TA-4812 were evaluated for their effects on survival and frailty index in a model of chronologic aging mice. Treatment lasted for 20 weeks (weeks 77-97 of age). At 112 weeks of age, there was no statistical significance in survival rates between the groups (FIG. 12). Frailty index of males was measured at week 109. We observed a statistically significant decrease in FI of males treated with TA-3899 compared to intact animals (FIG. 12), suggesting that TA-3899 may be effective treatment for age-related diseases.

### Example 22: Compounds Capable of Reprogramming SAM Phenotype

SAMs possess a phenotype that is characterized by expression of the biomarkers p16(Ink4a) and β-galactosidase. To test whether this phenotype could be reprogrammed, C57BL/6Jp16^{Ink4a/Luc} mice possessing the luciferase gene under the control of the p16(Ink4a) promoter, were injected with SC-containing alginate beads. After injection (2-3 weeks), SAMs were isolated from peritoneal lavage and treated with various compounds in order to assess their ability to alter expression of p16(Ink4a) and β-galactosidase. Compounds tested include Asenins; ABT-263 (navitoclax) (an inhibitor of anti-apoptotic proteins); nicotinamide phosphoribosyltransferase (NAMPT) inhibitor OT-82 (product of Oncotaris, Buffalo, N.Y.) (disclosed as compound TT-03582 in U.S. Publication 2016/0347748 (published Dec. 1, 2016)), an inhibitor of NAD⁺ synthesis, which decreases energy metabolism, inhibits poly(ADP-ribose) polymerase (PARP) and sirtuins' activity and therefore might imitate caloric restriction); rapatar (shown to inhibit cellular transition to senescence); CDK8 inhibitors (kinases that presumably regulate activation of NF-κB in SCs); as well as compounds which are capable of re-programming or stimulating macrophages, e.g., IL-4, IL-10, IL-13, M-CSF, Toll-like receptor 3 agonists [such as poly(I:C)] and Toll-like receptor 4 agonists (such as LPS).

Compounds were identified that were capable of reprogramming SAM phenotype, while having low cytotoxicity, e.g., those shown in Table 3. Asenins, some falling within the class of TA-3899, were found to be capable of decreasing SAM expression of β-galactosidase while having low toxicity. Agent OT-82 was found to have low SAM toxicity and to be able to selectively decrease p16(Ink4a) expression, while having no effect on β-galactosidase activity.

We next sought to determine whether immunomodulatory agents, and other small molecules, were capable of selectively modulating p16(Ink4a) expression in SAMs by comparing their effects on p16 expression in SAMs to that of mouse lung fibroblasts (mLF) and mouse visceral adipose tissue-derived fibroblasts (mVAT). Dose-response testing (Table 3) indicates that p16 is differentially regulated in macrophage compared to both proliferating and senescent (20 Gy irradiation) fibroblasts from the same p16^{Inka/Luc} mice. Immunomodulatory agents cause little to no change in p16 expression in fibroblasts. For example, mouse fibroblasts are reported to respond to LPS and IL-4 (expressing TLR4 and IL-4R, respectively), yet these agents are unable to modulate p16 in fibroblasts. This suggests differential tissue-specific response. This data, taken with the lack of DNA damage markers, suggests that p16 expression in macrophages is not linked to senescence as it is for fibroblasts and that induction of p16 expression in macrophages and senescent cells is explained by different mechanisms.

TABLE 3
Modulation of p16(Ink4a) in macrophages (SAMs from NDF alginate
beads) and mouse lung and adipose tissue-derived fibroblasts (with and
without 20Gy irradiation for senescence induction)

| Reagent | | | mLF and mVAT | |
|---|---|---|---|---|
| Class | Agent | SAMs | Proliferating | Senescent |
| TLR | LPS | decrease | no change | no change |
| agonists | Poly (I:C) | decrease | no change | no change |
| Cytokines | IFN-gamma | weak | weak | weak |
| | | decrease | decrease | decrease |
| | IFN- | decrease | weak | weak |
| | alpha/beta | | decrease | decrease |
| | IL-4 | increase | no change | no change |
| | IL-13 | increase | no change | no change |
| TA-03899 | TA-03899 | decrease | no change | no change |
| | TA-04812 | decrease | no change | no change |
| OT-82 | OT-82 | decrease | no change | no change |
| JAK/STAT | Ruxolitinib, | abrogates IL-4 | no change | no change |
| inhibitors | CYT387, | induced | | |
| | STAT6 | increase in | | |
| | inhibitor | p16 | | |
| | | expression | | |
| AMPK | Metformin | decrease | not | not |
| activator | | | determined | determined |

### Asenins

FIG. 13 shows β-galactosidase SAM phenotype reprogramming results for asenin TA-4812. SAMs were isolated from peritoneal lavage of mice injected with SC-containing beads and treated with varying concentrations of TA-4812. Cell proliferation was assessed using the CyQuant assay (CyQUANT® Direct Cell proliferation Assay, ThermoFisher Scientific, Waltham, Mass., Catalog # C35011, performed according to manufacturer's instructions). β-galactosidase production was assessed using the MUG assay, a quantitative assay to determine the level of β-galactosidase expression using 4-methylumbelliferylgalactopyranoside (MUG) as substrate. Cell viability was also determined using the resazurin assay. TA-4812 was found to be able to reduce β-galactosidase production by SAM cells at concentrations of 2.5 to 10 μM, while having no effect on cell viability.

### OT-82

OT-82 is a product of Oncotartis (Buffalo, N.Y.) (U.S. Publication 2016/0347748 (published Dec. 1, 2016)) and is a NAMPT inhibitor which blocks de novo synthesis of NAD⁺, thereby decreasing energy metabolism and inhibiting PARP (involved in DNA repair) and sirtuins. Through these mechanisms, OT-82 may be a multifunctional modulator of the aging processes and a useful agent for treating some age-related diseases, e.g., inflammation, cancer, and diabetes.

To test whether OT-82 was capable of reprogramming SAM phenotype, p16^{L}″ mice possessing the luciferase gene under the control of the p16 promoter, were injected with SC-containing alginate beads. After injection (2-3 weeks), SAMs were isolated from peritoneal lavage and treated with OT-82 at 500 nM. Following treatment with OT-82, cell viability was determined using the CyQuant assay, β-galactosidase production was determined using the MUG assay, and p16 expression was determined through p16 promoter driven luciferase expression. As shown in FIG. 30A, OT-82, while being almost non-toxic and having little effect on β-galactosidase expression, significantly downregulates expression of p16 in SAMs.

In addition, OT-82 inhibits both basal and inducible activity of NF-κB, as determined by luciferase reporter in HT1080 cells carrying an IPTG-inducible gene p21 (FIG. 30B).

Further studies were conducted to assess the toxicity of OT-82 on SAM cells using the resazurin cell viability assay (FIG. 30C). OT-82 is not toxic to SAMs by itself, nor is OT-82 toxic to SAMs in combination with the M2-phenotype inducer IL-4. However the combination of OT-82 with inducers of the M1-phenotype (LPS and interferon-gamma) is toxic to these M1-polarized SAMs, likely through inhibition of NF-κB, which can no longer support resistance to pro-apoptotic signals from tumor necrosis factor (TNF) activated by LPS treatment. Thus, OT-82 is toxic to M1-polarized SAMs but not M2-polarized SAMs.

It is contemplated that SAMs can removed from a mixed population of cells by combination therapy in which SAMs' polarization status is modulated by a first agent in order to make them more susceptible to the toxic effects of a second agent. In some embodiments, the second agent is selectively toxic to M1-polarized SAMs. In some examples, the second agent is selectively toxic to M2-polarized SAMs.

### Example 23: Targeted Therapeutics and Methods of SAM Elimination

Encapsulated β-Galactosidase Inducible Prodrugs

A hallmark of SAM phenotype is a high expression β-galactosidase. We sought to test whether a galactose-linked toxin could serve as a prodrug to selectively target and kill SAMs expressing β-galactosidase, where, upon exposure to β-galactosidase, galactose will be cleaved, freeing the toxin. The cytotoxic prodrug would therefore only be active in cells that were β-galactosidase positive and not in cells that were not β-galactosidase positive.

In order to test whether SAMs are able to activate a galactose-linked cytotoxic prodrug, two cytotoxic compounds were tested in parallel, TA-08209-1 (a kinase inhibitor) and TA-08210-1, a galactose-linked analog of TA-08209-1. After 72 hours treatment the SAMs viability was measured using the resazurin viability assay. It was found that the galactose-linked analog, TA-08210-1, while effective at killing SAMs, was less effective than TA-08209-1, displaying approximately a 10-fold higher IC₅₀ (FIG. 31). The addition of galactose may limit the effectiveness of the compound by limiting the cell permeability of the analog. To improve macrophages targeting delivery of the galactose-linked compound we encapsulated TA-08210-1 into liposomes which are similar in composition and size to the clodronate liposomes.

To confirm the targeted delivery of encapsulated galactose-linked prodrug to tissue macrophages and its subsequent activation by β-galactosidase, mice were injected intraperitoneally with 200 μL of fluorescent control DiD-containing liposomes (for labeling of phagocytic cells via fluorescent dye) (Fluoroliposome®-DiD, Clodrosome SKU #8913) and C12FDG-containing liposomes (for assessing beta-galactosidase activity within phagocytic cells; final concentration of 1 mg/mL C12FDG in final formulation). C12FDG (5-dodecanoylaminofluorescein di-β-D-galactopyranoside (ThermoFisher, Catalog # D2893) is a fluorogenic beta-galactosidase substrate (FDG) that is retained within cells. All liposome preparations were produced by Encapsula NanoSciences (www.encapsula.com). At 3 hours post-injection, thin samples (2-3 mm) of whole visceral adipose tissue were fixed 4 hours in 4% formaldehyde in PBS at 4° C., washed in PBS overnight, incubated with block solution (PBS with 5% normal rat serum and 0.25% triton x-100) 1 hour at room temperature and stained with rat monoclonal antibodies against F4/80 (AlexaFluor647-conjugated) for 4 hours at room temperature. Antibodies were diluted 1:50 in blocking solution. Nuclei were counterstained with DAPI (Invitrogen). Samples were washed 1 hour with PBS, cleared and mounted in glycerol. All images were acquired with AxioImager Z1 (Carl Zeiss Inc.; Oberkochen, Germany) microscope (bright field for morphology and SAβG; epifluorescence for F4/80 and DAPI staining) using AxioVision software (Zeiss).

Visceral adipose tissue from mice visualized for fluorescence of DiD (purple), hydrolysis product of C12FDG (green), and macrophage marker F4/80 (red) is evaluated. Merged image with DAPI (4′,6-diamidino-2-phenylindole, dihydrochloride) nuclear counterstain (blue) is also evaluated. Our results confirm that practically all phagocytic DiD⁺ cells in visceral fat are β-Gal⁺ and F4/80⁺ adipose tissue macrophages.

It is contemplated that one or more encapsulated prodrugs may be used to target SAMs. In some embodiments, the encapsulated prodrug is selectively phagocytosed by macrophages and not non-macrophage cell types. In some examples, the prodrug is activated by β-galactosidase and, upon phagocytosis, becomes active in β-galactosidase positive SAMs. In some embodiments, the activated prodrug is toxic to SAMs. In some examples, the activated prodrug is able to reprogram SAM phenotype.

Synthesis of TA-08209-1 and TA-08210-1

General experimental methods. Unless otherwise indicated, all reagents and solvents were purchased from commercial sources and used without further purification. Moisture or oxygen sensitive reactions were conducted under an atmosphere of argon or nitrogen gas.

LCMS analysis. The LCMS analysis was done at Agilent 1100 with tandem mass-spectrometer with APCI ionization.
1. Type of HPLC column: Phenomenex Onyx Monolithic C18; 50×4.6 mm; Part No: CHO-7643
2. Solvent for samples dissolution: 50% DMSO, 50% acetonitrile
3. Flow rate: 3.75 mL/min; column temperature 25° C.
4. Mobile phase: A=0.1% solution of TFA in AcN/Water (2.5:97.5), B=0.1% solution of TFA in acetonitrile
5. Gradient:

| time, min. | A % | B % |
|---|---|---|
| 0.0 | 100 | 0 |
| 0.2 | 100 | 0 |
| 2.1 | 5 | 95 |
| 2.5 | 5 | 95 |
| 2.2 | 100 | 0 |
| 3.0 | 100 | 0 |

6. Detection: diode array (DAD), 200-800 nm; photodiode array detector. Detection was carried out in the full ultraviolet-visible range from 200 to 800 nm. APCI (+ or/and − ions)—atmospheric pressure chemical ionization ELSD (PL-ELS 2100)
7. Total run time of the method: 3 min
8. Injection volume: 1.7 μL.

NMR. NMR ¹H and ¹³C spectra were recorded on Bruker Avance 400 instrument with operating frequency of 400 and 100 MHz respectively and calibrated using residual undeuterated chloroform (δH=7.28 ppm) and CDCl₃ (δC=77.16 ppm), or undeuterated DMSO (δH=2.50 ppm) and DMSO-d6 (δC=39.52 ppm) as internal references. The following abbreviations are used to set multiplicities: s=singlet, d=doublet, t=triplet, q=quartet, m=multiplet, br.=broad.

HPLC analysis. The HPLC analysis was done at Agilent 1200 instrument.
1. Synergi Hydro-RP 250×4.6 mm, 4 mkm
2. Flow rate: 1 mL/min; column temperature—ambient
3. Mobile phase: Acetonitrile/Water (65:35, isocratic flow)
4. Detection at 254 nm wave length.

TLC. Reactions were monitored by thin layer chromatography (TLC) carried out on Merck TLC Silica gel plates (60F₂₅₄), using UV light for visualization and basic aqueous potassium permanganate or iodine fumes as a developing agent.

Preparative HPLC. The preparative HPLC isolation of compounds analysis was done at Agilent 1200 HPLC Preparative instrument.
1. Type of HPLC column: Phenomenex Luna, C18; 100×30 mm;
2. Solvent for samples dissolution: 50% DMSO, 50% acetonitrile
3. Flow rate: 30 mL/min; column temperature 25° C.
4. Retention time 14 min, Total run time of the method: 30 min
5. Mobile phase: 0.1% solution of FA in AcN/Water (35:65), stationary phase: reversed-phase sorbent C18 (L1), 10 nm.

2,6-Dichloro-9-isopropyl-9H-purine (1)

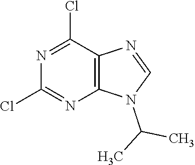

To a solution of 2,6-dichloro-9H-purine (20.0 g, 106 mmol) in DMSO (111 mL) was added K₂CO₃ (44.0 g, 318.8 mmol, 3 equiv) and 2-iodopropane (54 mL, 541 mmol, 5 equiv). After 8 h stirring, water was added and the solution was extracted with AcOEt (3×100 mL). The organic layers were assembled, washed with brine, dried (Na₂SO₄), concentrated, and chromatographed on a silica gel column (hexane/AcOEt 1:1, long column). The upper spot was collected, then the rest of the material was washed with EtOAc, and purified again to give a white solid. M=7.69+6.19 g. Yield=57%.

1H NMR (CDCl₃): δ 1.65 (d, 6H, J=6.8 Hz), 4.91 (hept, 1H, J=6.8 Hz), 8.17 (s, 1H).

### 2-Chloro-6-[4-(2-pyridyl)phenylmethylamino]purine (2)

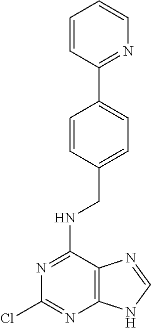 [SMILES: Clc1nc(NCc2ccc(-c3ccccn3)cc2)c2nc[nH]c2n1]

To a solution of 2,6-dichloro-9H-purine (7.62 g, 33 mmol) in 33 mL of n-BuOH were added the (4-(pyridin-2-yl)phenyl)methanamine (6.07 g, 33 mmol) and NEt3 (7.00 mL, 50.2 mmol, 1.5 equiv). After 3 h heating at 110° C., the mixture was cooled to 20° C., and the solid was filtrated, washed with 5 mL of cold EtOH, and dried in vacuum to give impure solid which was used as such. M=7.91 g. Yield=71%.

1H NMR (400 MHz, DMSO-d6): δ ppm 4.69 (s, 2H), 7.31 (dd, 5.3 Hz, 1H), 7.45 (d, J=8.1 Hz, 2H), 7.78-7.87 (m, 1H), 7.90 (d, J=8.1 Hz, 1H), 8.02 (d, J=7.9 Hz, 2H), 8.15 (br. s., 1H), 8.63 (d, J=3.9 Hz, 1H), 8.75 (br. s., 1H), 12.93 (br. s., 1H).

### N2-((1RS,4RS)-4-Aminocyclohexyl)-9-isopropyl-N6-(4-(pyridin-2-yl)benzyl)-9H-purine-2,6-diamine (3, TA-08124-1)

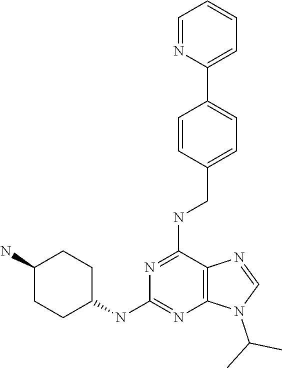

A mixture of chloropurine 2 and appropriate amine (10 equiv) was heated under N2 at 160° C. for 8 h. After cooling, H₂O (100 mL) was added, and the mixture was extracted with DCM (3×50 mL). The organic layer was dried, and evaporated until dryness. The residue was purified by column chromatography, eluent EtOAc (for unreacted Cl-purine), then CHCl₃/MeOH(NH₃) 20:1 (long column).

M=984 mg. Yield=69%.

1H NMR (400 MHz, CHLOROFORM-d) δ ppm 1.10-1.33 (m, 4H), 1.52 (d, J=6.8 Hz, 6H), 1.88 (d, J=11.4 Hz, 2H), 1.96 (br. s., 2H), 2.13 (d, J=5.1 Hz, 2H), 2.63-2.73 (m, 1H), 3.69-3.81 (m, 1H), 4.62 (q, J=6.6 Hz, 2H), 4.83 (br. s., 2H), 6.11 (br. s., 1H), 7.21 (ddd, 5.0, 1.6 Hz, 1H), 7.42-7.51 (m, 3H), 7.66-7.76 (m, 2H), 7.94 (d, J=8.2 Hz, 2H), 8.67 (d, J=4.4 Hz, 1H).

Synthesis of Conjugates of D-Galactose with TA-08210-1

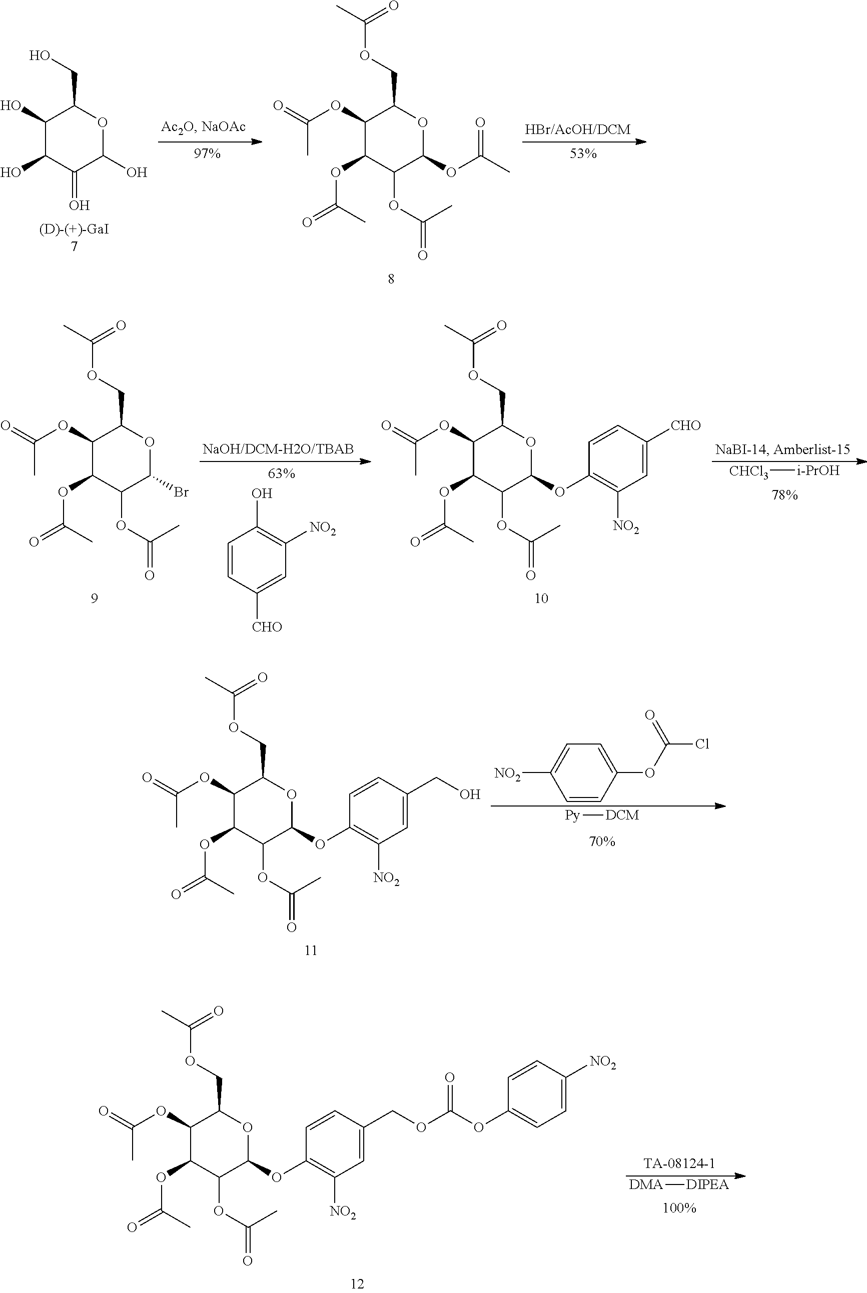

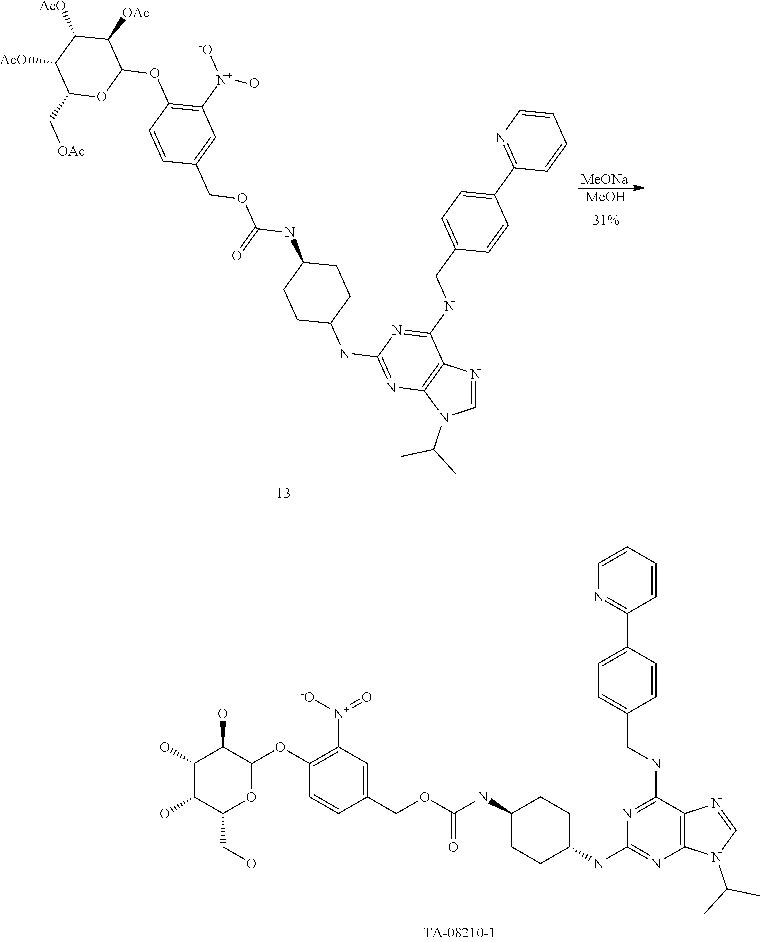

### 1,2,3,4,6-Penta-O-acetyl-β-D-galactopyranoside (8)

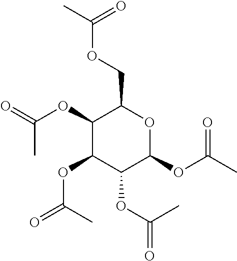 [SMILES: CC(=O)OC[C@H]1O[C@@H](OC(C)=O)[C@H](OC(C)=O)[C@@H](OC(C)=O)[C@H]1OC(C)=O]

Sodium acetate (10.0 g, 120 mmol) was added in acetic anhydride 200 mL was heated to 120° C. for 30 min, then D-galactopyranose (7) (19.30 g, 107 mmol) was added portionwise in the solution under stirring. After 1 h of the reaction at 120° C., the mixture was cooled and poured into cold water, extracted with dichloromethane. The organic layer was washed with saturated NaHCO₃ solution to pH=7, saturated brine and water, successively. Then, the organic phase was dried with sodium sulfate and concentrated. The resulting syrup was recrystallized with diethyl ether. Compound (2) as a white solid was obtained by vacuum drying: yield 18.3 g, 44%, mp. 143-144° C., Rf=0.5 (hexane/ethyl acetate=1:1). The filtrate was concentrated under reduced pressure to give 21.38 g. of an α- and β-mixture of D-galactopyranose pentaacetate and D-galactofuranose pentaacetate. Thus, the overall synthesis yield was 39.68 g. (95%).

### 2,3,4,6-Tetra-O-acetyl-α-D-galactopyranosyl bromide (9)

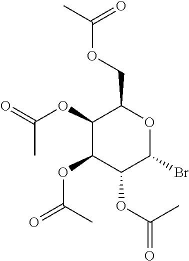 [SMILES: CC(=O)OC[C@H]1O[C@H](Br)[C@H](OC(C)=O)[C@@H](OC(C)=O)[C@H]1OC(C)=O]

1,2,3,4,6-Penta-O-acetyl-β-D-galactopyranoside (8.0 g, 0.021 mol) was dissolved in dichloromethane (200 mL). A solution of hydrobromic acid/acetic acid (33%, 46 mL) was added dropwise with stirring. The reaction monitored by TLC (hexane/ethyl acetate=1/1) was continued for 20 h in the dark. The mixture was diluted by adding dichloromethane and then poured into ice-water with stirring when the reaction was completed. The organic layer was separated and washed with saturated sodium bicarbonate and saturated brine, respectively. The solvent was removed by rotary evaporation after drying with anhydrous sodium sulfate. The crude product was purified by flash chromatography (solvents: hexane/ethylacetate=3:1). The resulting syrup was recrystallized with hexane. Compound (3) as a white solid was obtained by vacuum drying: yield 4.53 g, 53%, mp. 86-87° C., Rf=0.80 (hexane/ethyl acetate=1:1).

A similar synthesis was carried out with a mixture of an α- and β-mixture of D-galactopyranose pentaacetate and D-galactofuranose pentaacetate. As a result, the compound (9) was prepared from the mixture after purification in a yield of 25%.

### 1-(4-Formyl-2-nitrophenyl)-2,3,4,6-tetra-O-acetyl-β-D-galactopyranoside (10)

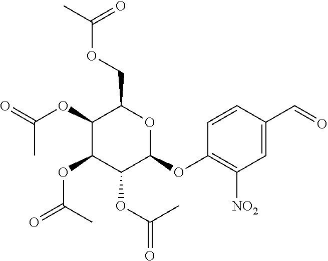 [SMILES: CC(=O)OC[C@H]1O[C@@H](Oc2ccc(C=O)cc2[N+](=O)[O-])[C@H](OC(C)=O)[C@@H](OC(C)=O)[C@H]1OC(C)=O]

A solution of 2,3,4,6-tetra-O-acetyl-α-D-galactopyranosyl bromide (9) (1.00 g, 2.4 mmol) in CH₂Cl₂ (20 mL) was added dropwise to a vigorously stirred CH₂Cl₂—H₂O biphasic mixture (pH 8-9) of 4-hydroxy-3-nitrobenzaldehyde (0.406 g, 2.4 mmol) and tetrabutylammonium bromide (TBAB) (232 mg, 0.7 mmol) in CH₂Cl₂ (20 mL) and water (20 mL) containing 0.11 g (2.7 mmol) of NaOH over a period of 1 h at 40° C., and the stirring continued for additional 5-6 h until TLC showed that the reaction was completed. The product was extracted with CH₂Cl₂, washed with NaOH (20 mL, 5%), brine, respectively, dried with anhydrous Na₂SO₄, and then concentrated under reduced pressure. The residue was purified by column chromatography on silica gel to give a syrup, which was recrystallized with diethyl ether. Compound (10) as a white solid was obtained by vacuum drying: yield 0.757 g, 63%, mp. 200-201° C., Rf=0.25 (hexane/ethyl acetate=1:1).

### 1-(4-Hydroxymethyl-2-nitrophenyl)-2,3,4,6-tetra-O-acetyl-β-D-galactopyranoside (11)

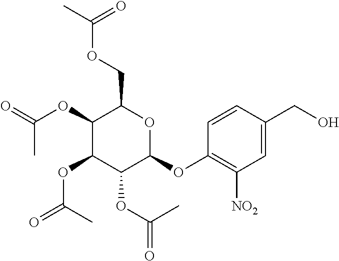 [SMILES: CC(=O)OC[C@H]1O[C@@H](Oc2ccc(CO)cc2[N+](=O)[O-])[C@H](OC(C)=O)[C@@H](OC(C)=O)[C@H]1OC(C)=O]

Benzaldehyde (10) (2.49 g, 5.0 mmol, 1.0 equiv.) was dissolved in CHCl₃ (37.0 mL) and iPrOH (8.0 mL), silica gel (42-60 mesh, 5.97 g) and ion exchanger Amberlyst-15 (75 mg) added and the mixture cooled to 0° C. Within 2 h freshly powdered NaBH₄ (0.393 g, 10.4 mmol, 2.1 equiv.) was added portionwise and stirring continued at 0° C. for further 1.5 h. The reaction was quenched by addition of ice-cold sat. NH₄Cl solution, filtered over Cilite and transferred to a separatory funnel. After phase separation, the aqueous layer was extracted with CH₂Cl₂, the combined organic layers were washed with ice-water and brine, dried with anhydrous Na₂SO₄, and then concentrated under reduced pressure. The residue was recrystallized with diethyl ether. Compound (11) as a white solid was obtained by vacuum drying: yield 1.959 g, 78%, mp. 202-205° C., Rf=0.15 (hexane/ethyl acetate=1:1).

### 2,3,4,6-Tetra-O-acetyl-[2-nitro-4-(4-nitrophenoxycarbonyloxymethyl)phenyl]-β-D-galactopyranoside (12)

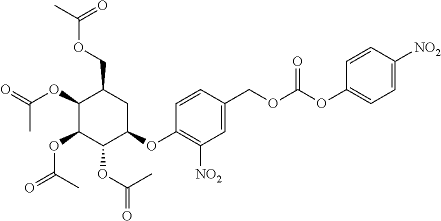 [SMILES: CC(=O)OC[C@H]1C[C@@H](Oc2ccc(COC(=O)Oc3ccc([N+](=O)[O-])cc3)cc2[N+](=O)[O-])[C@H](OC(C)=O)[C@@H](OC(C)=O)[C@H]1OC(C)=O]

To a solution of the benzyl alcohol (10) (800 mg, 1.60 mmol, 1.0 equiv.) and pyridine (253 mg, 0.258 ml, 3.20 mmol, 2.0 equiv.) in CH₂Cl₂ (40.0 ml) at 0° C. p-nitrophenylchloroformate (645 mg, 3.20 mmol, 2.0 equiv.) was added and the mixture was stirred for 2 h at 0° C. Silica gel (1.0 g) was added directly and the solvents were removed. The residue was purified by column chromatography on silica gel (gradient: hexane/ethyl acetate=2:1→1:2) as colourless foam, with was recrystallized with diethyl ether-hexane. Compound (12) as a white solid was obtained by vacuum drying: yield 0.742 g, 70%, mp. 145° C., Rf=0.80 (hexane/ethyl acetate=1:1).

¹H NMR, CDCl₃, 400 MHz: 8.28 (d, 2H, J=9.2 Hz, H12), 7.90 (d, 1H, J=2.0 Hz, H9), 7.61 (dd, 1H, J=8.8 Hz, J=2.4 Hz, H8), 7.41 to 7.36 (m, 3H, H7+11), 5.56 (dd, 1H, J=10.4 Hz, J=8.0 Hz, H2), 5.47 (d, 1H, J=2.8 Hz, H4), 5.28 (bs, 2H, H10), 5.13 to 5.09 (m, 2H, H1+3), 4.26 (dd, 1H, J=11.2 Hz, J=6.8 Hz, H6), 4.18 to 4.07 (m, 2H, H5+6), 2.19 (s, 3H, HAc), 2.13 (s, 3H, H—Ac), 2.07 (s, 3H, H—Ac), 2.02 (s, 3H, H—Ac).

¹³C NMR, CDCl₃, 400 MHz: 170.4, 170.3, 169.5, 155.4, 152.4, 149.8, 145.7, 141.4, 134.0, 130.3, 125.6, 125.5, 121.8, 120.0, 100.7, 71.6, 70.6, 69.0, 67.8, 66.7, 61.4, 20.8 (×2), 20.7.

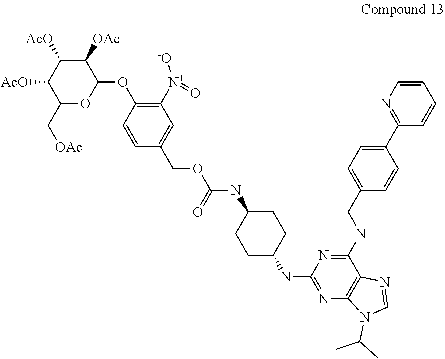

TA-08124-1 (984 mg, 2.16 mmol) was dissolved in DMA (21 mL, 0.1 M) and activated carbonate (12) (2.87 g, 4.32 mmol, 2 equiv) was added followed by DIPEA (1.88 mL, 10.8 mmol, 5 equiv). The reaction mixture was heated at reflux for 1 hour and evaporated in vacuum. The residue was dissolved in DCM and dry loaded on a small amount of silica. Purification by column chromatography yielded a desired product. Eluent: hexane/EtOAc (for elution of p-nitrophenol), then pure EtOAc (first elution of decomposed activated carbamate, then product). M=2.13 g. Yield=100%.

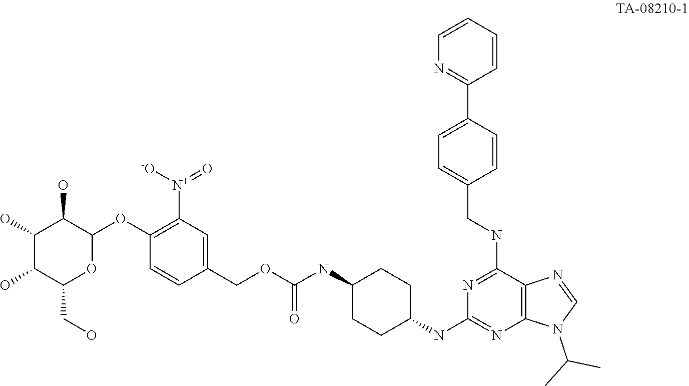

Fresh MeONa solution was prepared by dissolving sodium (2.3 g) in absolute MeOH (100 mL). The MeONa solution was cooled to room temperature and added to the flask containing protected compound (2.13 g) was dissolved in this solution. Stirring was continued for 1 hour at RT and the solution was neutralized with acetic acid (5.72 mL). The reaction mixture was diluted with brine (100 mL) and extracted with EtOAc (3×100 mL). The combined organic layer was shaken was brine (100 mL) and dried over Na₂SO₄. Organic layer was evaporated and the residue was purified by pHPLC to give a target compound. M=547 mg. Yield=31%.

1H NMR (400 MHz, DMSO-d6) δ ppm 1.11-1.33 (m, 4H), 1.44 (d, J=6.6 Hz, 6H), 1.68-1.99 (m, 4H), 3.17-3.54 (m, 8H), 3.58-3.71 (m, 4H), 4.44-4.54 (m, 1H), 4.63 (br. s., 3H), 4.99 (s, 2H), 5.03 (d, J=7.7 Hz, 1H), 6.09 (d, J=7.0 Hz, 1H), 7.25 (d, J=7.8 Hz, 1H), 7.30 (ddd, J=6.6, 5.5, 1.0 Hz, 1H), 7.42 (t, J=9.0 Hz, 3H), 7.60 (dd, J=8.8, 2.0 Hz, 1H), 7.78 (s, 1H), 7.82-7.86 (m, 2H), 7.87-7.91 (m, 1H), 7.99 (d, J=8.2 Hz, 2H), 8.62 (dt, 0.8 Hz, 1H).

APCI MS [M+H]⁺ 814.62, 457.29.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### REFERENCES

1. Cannizzo E S, Clement C C, Sahu R, Follo C, Santambrogio L. Oxidative stress, inflamm-aging and immunosenescence. J Proteomics. 2011; 74: 2313-2323.
2. Larbi A, Franceschi C, Mazzatti D, Solana R, Wikby A, Pawelec G. Aging of the immune system as a prognostic factor for human longevity. Physiology (Bethesda). 2008; 23: 64-74.
3. Sagiv A, Krizhanovsky V. Immunosurveillance of senescent cells: The bright side of the senescence program. Biogerontology. 2013; 14: 617-628.
4. Campisi J, Andersen J K, Kapahi P, Melov S. Cellular senescence: a link between cancer and age-related degenerative disease? Semin Cancer Biol. 2011; 21: 354-359.
5. Chung H Y, Cesari M, Anton S, Marzetti E, Giovannini S, Seo A Y, Carter C Y, BP LC. Molecular Inflammation: Underpinnings of Aging and Age-related Diseases. Ageing Res Rev. 2009; 8: 18-30.
6. Hohensinner P J, Goronzy J J, Weyand C M. Telomere dysfunction, autoimmunity and aging. Aging Dis. 2011; 2: 524-537.
7. Franceschi C, Bonafè M, Valensin S, Olivieri F, De Luca M, Ottaviani E, De Benedictis G. Inflamm-aging. An evolutionary perspective on immunosenescence. Ann N Y Acad Sci. 2000; 908: 244-254.
8. Franceschi C, Capri M, Monti D, Giunta S, Olivieri F, Sevini F, Panourgia M P, Invidia L, Celani L, Scurti M, Cevenini E, Castellani G C, Salvioli S. Inflammaging and anti-inflammaging: A systemic perspective on aging and longevity emerged from studies in humans. Mech Ageing Dev. 2007; 128: 92-105.
9. Cevenini E, Monti D, Franceschi C. Inflamm-ageing. Curr Opin Clin Nutr Metab Care. 2013; 16: 14-20.
10. Franceschi C, Campisi J. Chronic inflammation (inflammaging) and its potential contribution to age-associated diseases. J Gerontol A Biol Sci Med Sci. 2014; 69: S4-9.
11. Collerton J, Martin-Ruiz C, Davies K, Hilkens C M, Isaacs J, Kolenda C, Parker C, Dunn M, Catt M, Jagger C, von Zglinicki T, Kirkwood T B L. Frailty and the role of inflammation, immunosenescence and cellular ageing in the very old: Cross-sectional findings from the Newcastle 85+ Study. Mech Ageing Dev. 2012; 133: 456-466.
12. Vasto S, Candore G, Balistreri C R, Caruso M, Colonna-Romano G, Grimaldi M P, Listi F, Nuzzo D, Lio D, Caruso C. Inflammatory networks in ageing, age-related diseases and longevity. Mech Ageing Dev. 2007; 128: 83-91.
13. Howcroft T K, Campisi J, Louis G B, Smith M T, Wise B, Wyss-Coray T, Augustine A D, McElhaney J E, Kohanski R, Sierra F. The role of inflammation in age-related disease. Aging (Albany N.Y.). 2013; 5: 84-93.
14. Kennedy B K, Berger S L, Brunet A, Campisi J, Cuervo A M, Epel E S, Franceschi C, Lithgow G J, Morimoto R I, Pessin J E, Rando T A, Richardson A, Schadt E E, et al. Geroscience: linking aging to chronic disease. Cell. 2014; 159: 709-713.
15. Tchkonia T, Morbeck D E, Von Zglinicki T, Van Deursen J, Lustgarten J, Scrable H, Khosla S, Jensen M D, Kirkland J L. Fat tissue, aging, and cellular senescence. Aging Cell. 2010; 9: 667-684.
16. Erusalimsky J D, Kurz D J. Cellular senescence in vivo: its relevance in ageing and cardiovascular disease. Exp Gerontol. 2005; 40: 634-42.
17. Kuilman T, Peeper D S. Senescence-messaging secretome: SMS-ing cellular stress. Nat Rev Cancer. 2009; 9: 81-94.
18. Ovadya Y, Krizhanovsky V. Senescent cells: SASPected drivers of age-related pathologies. Biogerontology. 2014; 15: 627-642.
19. Coppé J-P, Patil C K, Rodier F, Sun Y, Munoz D P, Goldstein J, Nelson P S, Desprez P-Y, Campisi J. Senescence-associated secretory phenotypes reveal cell-nonautonomous functions of oncogenic RAS and the p53 tumor suppressor. PLoS Biol. 2008; 6: 2853-2868.
20. Zhu Y, Armstrong J L, Tchkonia T, Kirkland J L. Cellular senescence and the senescent secretory phenotype in age-related chronic diseases. Curr Opin Clin Nutr Metab Care. 2014; 17: 324-328.
21. Baker D J, Wijshake T, Tchkonia T, LeBrasseur N K, Childs B G, van de Sluis B, Kirkland J L, van Deursen J M. Clearance of p16Ink4a-positive senescent cells delays ageing-associated disorders. Nature. 2011; 479: 232-236.
22. Demaria M, Ohtani N, Youssef S A, Rodier F, Toussaint W, Mitchell J R, Laberge R M, Vijg J, VanSteeg H, Doll?? MET, Hoeijmakers J H J, deBruin A, Hara E, et al. An essential role for senescent cells in optimal wound healing through secretion of PDGF-AA. Dev Cell. 2014; 31: 722-733.
23. Baker D J, Childs B G, Durik M, Wijers M E, Sieben C J, Zhong J, Saltness R A, Jeganathan K B, Casaclang Verzosa G, Pezeshki A, Khazaie K, Miller J D, Van Deursen J M. Naturally occurring p16 Ink4a-positive cells shorten healthy lifespan. Nature. 2016; 530: 1-5.
24. Dimri G P, Lee X H, Basile G, Acosta M, Scott C, Roskelley C, Medrano E E, Linskens M, Rubelj I, Pereirasmith O, Peacocke M, Campisi J. A Biomarker That Identifies Senescent Human-Cells in Culture and in Aging Skin in-Vivo. Proc Natl Acad Sci USA. 1995; 92: 9363-9367.
25. Lin A W, Barradas M, Stone J C, van Aelst L, Serrano M, Lowe S W. Premature senescence involving p53 and p16 is activated in response to constitutive MEK/MAPK mitogenic signaling. Genes Dev. 1998; 12: 3008-3019.
26. Childs B G, Baker D J, Kirkland J L, Campisi J, van Deursen J M. Senescence and apoptosis: dueling or complementary cell fates? EMBO Rep. 2014; 15: 1139-1153.
27. Serrano M, Lin A W, McCurrach M E, Beach D, Lowe S W. Oncogenic ras provokes premature cell senescence associated with accumulation of p53 and p16(INK4a). Cell. 1997; 88: 593-602.
28. Campisi J. Aging, cellular senescence, and cancer. Annu Rev Physiol. 2013; 75: 685-705.
29. Lowe S W, Cepero E, Evan G. Intrinsic tumour suppression. Nature. 2004; 432: 307-315.
30. Krizhanovsky V, Xue W, Zender L, Yon M, Hernando E, Lowe S W. Implications of cellular senescence in tissue damage response, tumor suppression, and stem cell biology. Cold Spring Harbor Symposia on Quantitative Biology. 2008. p. 513-522.
31. Krishnamurthy J, Torrice C, Ramsey M R, Kovalev G I, A1-Regaiey K, Su L, Sharpless N E. Ink4a/Arf expression is a biomarker of aging. J Clin Invest. 2004; 114: 1299-1307.
32. Ohtani N, Yamakoshi K, Takahashi A, Hara E. The p16INK4a-RB pathway: molecular link between cellular senescence and tumor suppression. J Med Invest. 2004; 51: 146-153.
33. Debacq-Chainiaux F, Erusalimsky J D, Campisi J, Toussaint O. Protocols to detect senescence-associated beta-galactosidase (SA-betagal) activity, a biomarker of senescent cells in culture and in vivo. Nat Protoc. 2009; 4: 1798-1806.
34. Yang N C, Hu M L. The limitations and validities of senescence associated-beta-galactosidase activity as an aging marker for human foreskin fibroblast Hs68 cells. Exp Gerontol. 2005; 40: 813-819.
35. Gu L, Kitamura M. Sensitive Detection and Monitoring of Senescence-Associated Secretory Phenotype by SASP-RAP Assay. PLoS One. 2012; 7(12): e52305.
36. Pospelova T V, Demidenko Z N, Bukreeva E I, Pospelov V A, Gudkov A V, Blagosklonny M V. Pseudo-DNA damage response in senescent cells. Cell Cycle. 2009; 8: 4112-4118.
37. Shokolenko I N, Wilson G L, Alexeyev M F. Aging: A mitochondrial DNA perspective, critical analysis and an update. World J Exp Med. 2014; 4: 46-57.
38. Bauer M E, Jeckel C M M, Luz C. The role of stress factors during aging of the immune system. Ann N Y Acad Sci. 2009; 1153: 139-152.
39. Childs B G, Durik M, Baker D J, van Deursen J M. Cellular senescence in aging and age-related disease: from mechanisms to therapy. Nat Med. 2015; 21: 1424-1435.
40. Burd C E, Sorrentino J A, Clark K S, Darr D B, Krishnamurthy J, Deal A M, Bardeesy N, Castrillon D H, Beach D H, Sharpless N E. Monitoring tumorigenesis and senescence in vivo with a p16 INK4a-luciferase model. Cell. 2013; 152: 340-351.
41. Sagiv A, Biran A, Yon M, Simon J, Lowe S W, Krizhanovsky V. Granule exocytosis mediates immune surveillance of senescent cells. Oncogene. 2013; 32: 1971-1977.
42. Hoenicke L, Zender L. Immune surveillance of senescent cells-biological significance in cancer- and non-cancer pathologies. Carcinogenesis. 2012; 33: 1123-1126.
43. Wurdinger T, Badr C, Pike L, de Kleine R, Weissleder R, Breakefield X O, Tannous B A. A secreted luciferase for ex vivo monitoring of in vivo processes. Nat Methods. 2008; 5: 171-173.
44. Chung E, Yamashita H, Au P, Tannous B A, Fukumura D, Jain R K. Secreted Gaussia luciferase as a biomarker for monitoring tumor progression and treatment response of systemic metastases. PLoS One. 2009; 4(12): e8316.
45. Schneider S, Feilen P J, Brunnenmeier F, Minnemann T, Zimmermann H, Zimmermann U, Weber M M. Long-term graft function of adult rat and human islets encapsulated in novel alginate-based microcapsules after transplantation in immunocompetent diabetic mice. Diabetes. 2005; 54: 687-693.
46. Mazzitelli S, Borgatti M, Breveglieri G, Gambari R, Nastruzzi C. Encapsulation of eukaryotic cells in alginate microparticles: Cell signaling by TNF-alpha through capsular structure of cystic fibrosis cells. J Cell Commun Signal. 2011; 5: 157-165.
47. de Vos P, Faas M M, Strand B, Calafiore R. Alginate-based microcapsules for immunoisolation of pancreatic islets. Biomaterials. 2006. p. 5603-5617.
48. Bu L, Gao M, Qu S, Liu D. Intraperitoneal injection of clodronate liposomes eliminates visceral adipose macrophages and blocks high-fat diet-induced weight gain and development of insulin resistance. AAPS J. 2013; 15: 1001-1011.
49. Feng B, Jiao P, Nie Y, Kim T, Jun D, van Rooijen N, Yang Z, Xu H. Clodronate liposomes improve metabolic profile and reduce visceral adipose macrophage content in diet-induced obese mice. PLoS One. 2011; 6: 1-12.
50. Burton D G A, Krizhanovsky V. Physiological and pathological consequences of cellular senescence. Cell Mol Life Sci. 2014; 71: 4373-4386.
51. Tchkonia T, Zhu Y, van Deursen J, Campisi J, Kirkland J L. Cellular senescence and the senescent secretory phenotype: therapeutic opportunities. J Clin Invest. 2013; 123: 966-972.
52. Yosef R, Pilpel N, Tokarsky-Amiel R, Biran A, Ovadya Y, Cohen S, Vadai E, Dassa L, Shahar E, Condiotti R, Ben-Porath I, Krizhanovsky V. Directed elimination of senescent cells by inhibition of BCL-W and BCL-XL. Nat Commun. 2016; 7: 11190.
53. Kirkland J L, Tchkonia T. Clinical strategies and animal models for developing senolytic agents. Exp Gerontol. 2015; 68: 19-25.
54. Roos C M, Zhang B, Palmer A K, Ogrodnik M B, Pirtskhalava T, Thalji N M, Hagler M, Jurk D, Smith L A, Casaclang-Verzosa G, Zhu Y, Schafer M J, Tchkonia T, et al. Chronic senolytic treatment alleviates established vasomotor dysfunction in aged or atherosclerotic mice. Aging Cell. 2016; [Epub ahead of print].
55. Chang J, Wang Y, Shao L, Laberge R M, Demaria M, Campisi J, Janakiraman K, Sharpless N E, Ding S, Feng W, Luo Y, Wang X, Aykin-Burns N, et al. Clearance of senescent cells by ABT263 rejuvenates aged hematopoietic stem cells in mice. Nat Med. 2016; 22: 78-83.
56. Zhu Y, Tchkonia T, Fuhrmann-Stroissnigg H, Dai H M, Ling Y Y, Stout M B, Pirtskhalava T, Giorgadze N, Johnson K O, Giles C B, Wren J D, Niedernhofer L J, Robbins P D, et al. Identification of a novel senolytic agent, navitoclax, targeting the Bcl-2 family of anti-apoptotic factors. Aging Cell. 2016; 15: 428-35.
57. Van Rooijen N, Sanders A. Liposome mediated depletion of macrophages: mechanism of action, preparation of liposomes and applications. J Immunol Methods. 1994; 174: 83-93.
58. Rooijen N Van, Hendrikx E. Liposomes for Specific Depletion of Macrophages from Organs and Tissues. Methods in Molecular Biology. 2010; 605: 189-203.
59. Burton D G A, Faragher R G A. Cellular senescence: from growth arrest to immunogenic conversion. Age (Dordr). 2015; 37: 27.
60. Green D R, Oguin T H, Martinez J. The clearance of dying cells: table for two. Cell Death Differ. 2016; 23: 1-12.
61. Ortega-Gomez A, Perretti M, Soehnlein O. Resolution of inflammation: An integrated view. EMBO Mol Med. 2013; 5: 661-674.
62. Headland S E, Norling L V. The resolution of inflammation: Principles and challenges. Semin Immunol. 2015; 27: 149-160.
63. Biran A, Perelmutter M, Gal H, G. A. Burton D, Ovadya Y, Vadai E, Geiger T, Krizhanovsky V. Senescent cells communicate via intercellular protein transfer. Genes Dev. Cold Spring Harbor Laboratory Press; 2014; 29: 791-802.
64. Xue W, Zender L, Miething C, Dickins R A, Hernando E, Krizhanovsky V, Cordon-Cardo C, Lowe S W. Senescence and tumour clearance is triggered by p53 restoration in murine liver carcinomas. Nature. 2007; 445: 656-660.
65. Bursuker I, Rhodes J M, Goldman R. Beta-galactosidase—an indicator of the maturational stage of mouse and human mononuclear phagocytes. J Cell Physiol. 1982; 112: 385-390.
66. Rhodes J M, Blom J. Cytochemical localization of beta-galactosidase in resident and inflammatory peritoneal macrophages from C57BL mice. Histochemistry. 1986; 86: 159-64.
67. Cudejko C, Wouters K, Fuentes L, Hannou S A, Paquet C, Bantubungi K, Bouchaert E, Vanhoutte J, Fleury S, Remy P, Tailleux A, Chinetti-Gbaguidi G, Dombrowicz D, et al. p16INK4a deficiency promotes IL-4-induced polarization and inhibits proinflammatory signaling in macrophages. Blood. 2011; 118: 2556-2566.
68. Fuentes L, Wouters K, Hannou S A, Cudejko C, Rigamonti E, Mayi T H, Derudas B, Pattou F, Chinetti-Gbaguidi G, Staels B, Paumelle R. Downregulation of the tumour suppressor p16 INK4A contributes to the polarisation of human macrophages toward an adipose tissue macrophage (ATM)-like phenotype. Diabetologia. 2011; 54: 3150-3156.
69. Murakami Y, Mizoguchi F, Saito T, Miyasaka N, Kohsaka H. p16(INK4a) exerts an anti-inflammatory effect through accelerated IRAK1 degradation in macrophages. J Immunol. 2012; 189: 5066-5072.
70. Kearney A Y, Anchang B, Plevritis S, Felsher D W. ARF: connecting senescence and innate immunity for clearance. Aging (Albany, N.Y.). 2015; 7: 613-615.
71. Traves P G, Lopez-Fontal R, Luque A, Hortelano S. The tumor suppressor ARF regulates innate immune responses in mice. J Immunol. 2011; 187: 6527-6538.
72. Zhu Y, Tchkonia T, Pirtskhalava T, Gower A, Ding H, Giorgadze N, Palmer A K, Ikeno Y, Borden G, Lenburg M, O'Hara S P, LaRusso N F, Miller J D, et al. The Achilles' Heel of Senescent Cells: From Transcriptome to Senolytic Drugs. Aging Cell. 2015 14: 644-658.
73. Kim S B, Suzuki H, Sato M, Tao H. Superluminescent variants of marine luciferases for bioassays. Anal Chem. 2011; 83: 8732-8740.
74. Sung J H, Yang H M, Park J B, Choi G S, Joh J W, Kwon C H, Chun J M, Lee S K, Kim S J. Isolation and characterization of mouse mesenchymal stem cells. Transpl Proc. 2008; 40: 2649-2654.
75. Schafer M J, White T A, Evans G, Tonne J M, Verzosa G C, Stout M B, Mazula D L, Palmer A K, Baker D J, Jensen M D, Torbenson M S, Miller J D, Ikeda Y, et al. Exercise Prevents Diet-induced Cellular Senescence in Adipose Tissue. Diabetes. 2016; 65: 1606-1615.
76. Davalos A R, Coppe J P, Campisi J, Desprez P Y. Senescent cells as a source of inflammatory factors for tumor progression. Cancer Metastasis Rev. 2010 June; 29(2):273-83.
77. Collado M, Serrano M. Senescence in tumours: evidence from mice and humans. Nat Rev Cancer. 2010 January; 10(1):51-7.
78. Muñoz-Espin D, Serrano M. Cellular senescence: from physiology to pathology. Nat Rev Mol Cell Biol. 2014 July; 15(7):482-96.
79. Childs B G, Durik M, Baker D J, van Deursen J M. Cellular senescence in aging and age-related disease: from mechanisms to therapy. Nat Med. 2015 December; 21(12):1424-35.
80. Baker D J, Childs B G, Durik M, Wijers M E, Sieben C J, Zhong J, Saltness R A, Jeganathan K B, Verzosa G C, Pezeshki A, Khazaie K, Miller J D, van Deursen J M. Naturally occurring p16(Ink4a)-positive cells shorten healthy lifespan. Nature. 2016 Feb. 11; 530(7589): 184-9.
81. Baker D J, Childs B G, Durik M, Wijers M E, Sieben C J, Zhong J, Saltness R A, Jeganathan K B, Verzosa G C, Pezeshki A, Khazaie K, Miller J D, van Deursen J M. Naturally occurring p16(Ink4a)-positive cells shorten healthy lifespan. Nature. 2016 Feb. 11; 530(7589):184-9.
82. Sharpless N E (August 2009). “Expression of p16(INK4a) in peripheral blood T-cells is a biomarker of human aging”. Aging Cell 8 (4): 439-48.
83. Robles S J, Adami G R. Agents that cause DNA double strand breaks lead to p16INK4a enrichment and the premature senescence of normal fibroblasts. Oncogene. 1998; 16: 1113-23.
84. Serrano M, Lin A W, McCurrach M E, Beach D, Lowe S W. Oncogenic ras provokes premature cell senescence associated with accumulation of p53 and p16INK4a. Cell. 1997; 88: 593-602.

All references referred to herein are incorporated in their entirety. Various embodiments of the present invention may be characterized by the potential claims listed in the paragraphs following this paragraph (and before the actual claims provided at the end of this application). These potential claims form a part of the written description of this application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public.

The embodiments of the invention described above are intended to be merely exemplary; numerous variations and modifications will be apparent to those skilled in the art. All such variations and modifications are intended to be within the scope of the present invention as defined in any appended claims.

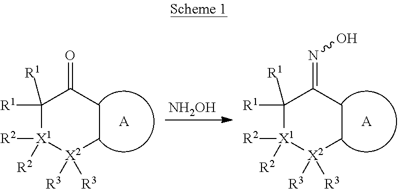

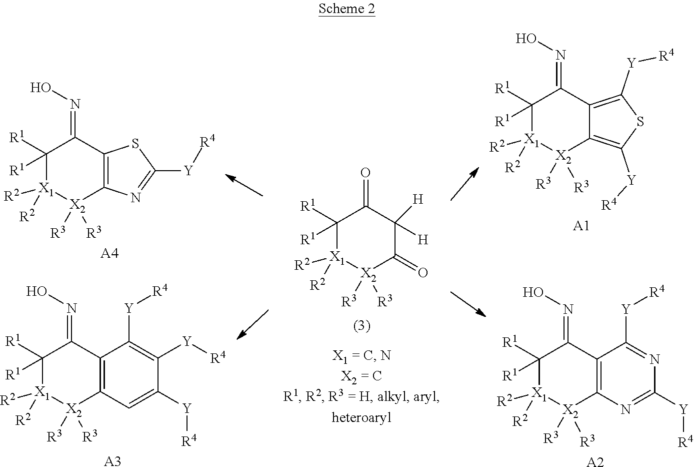

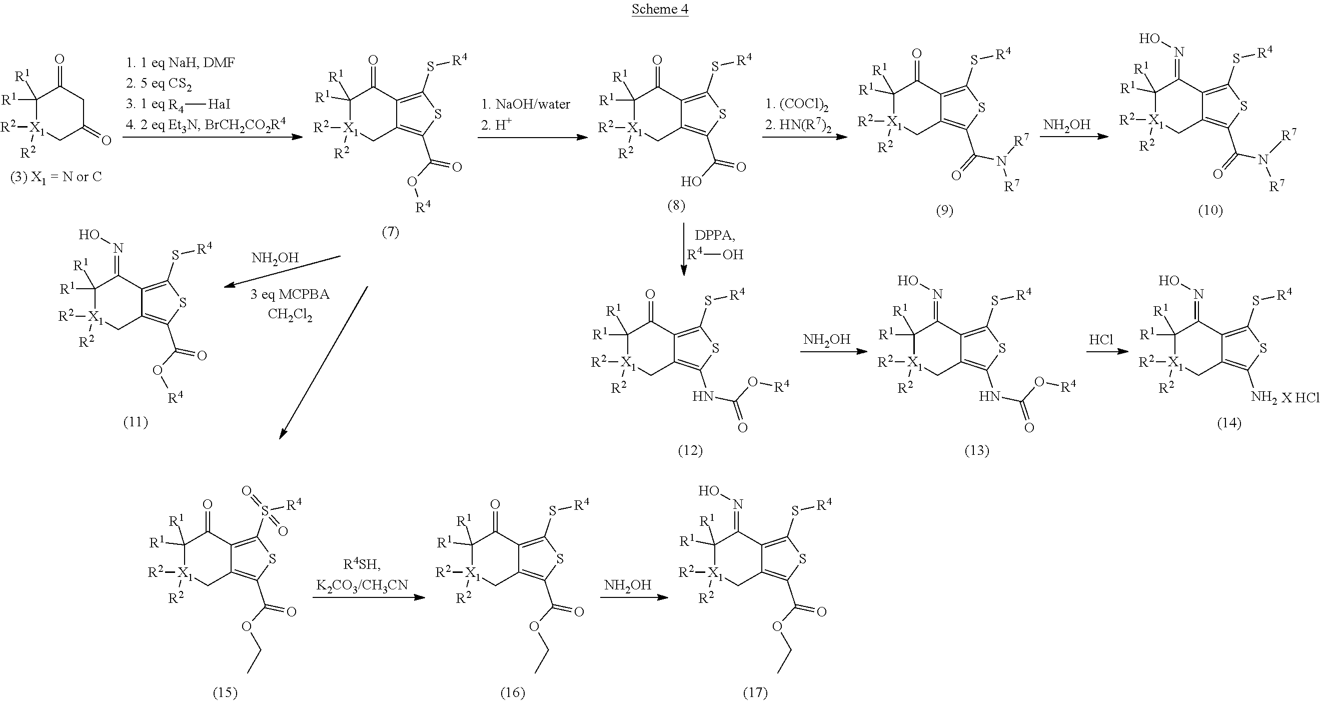

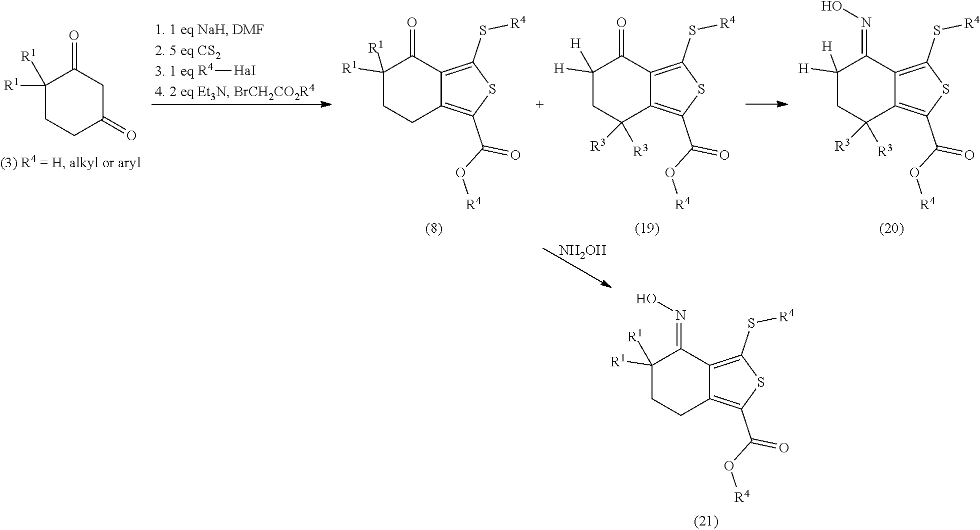

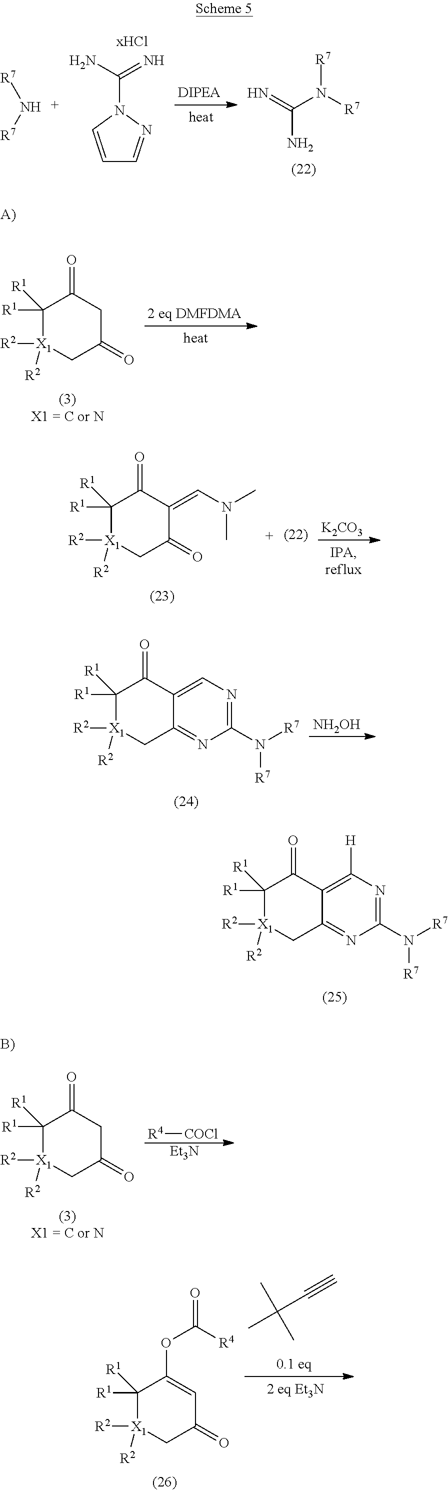

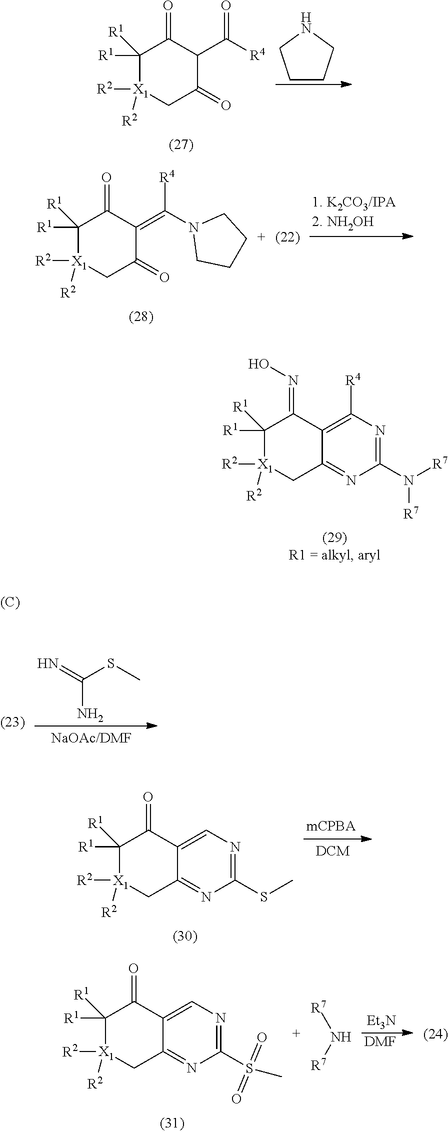

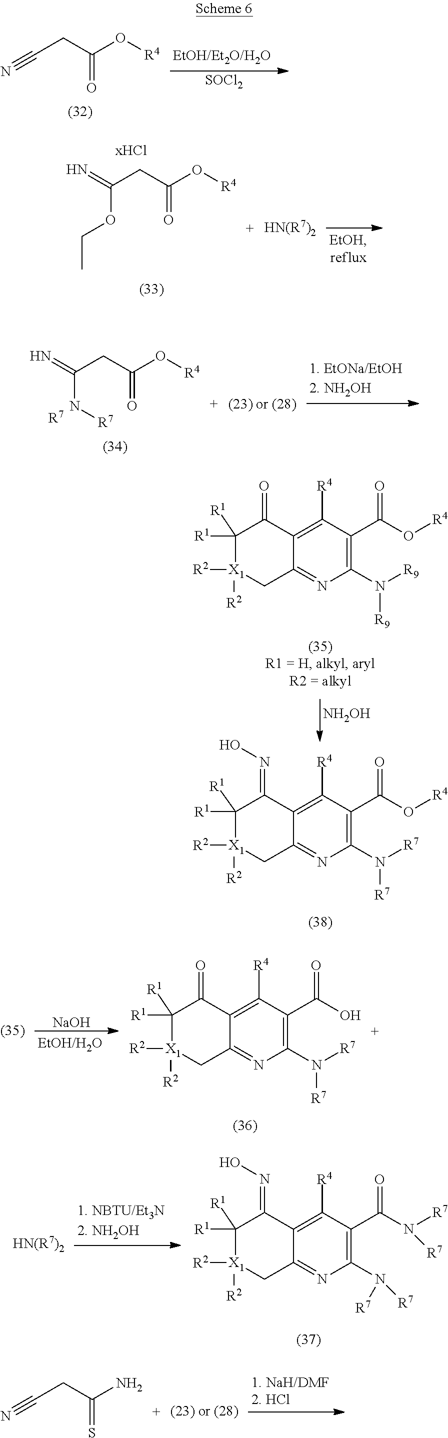

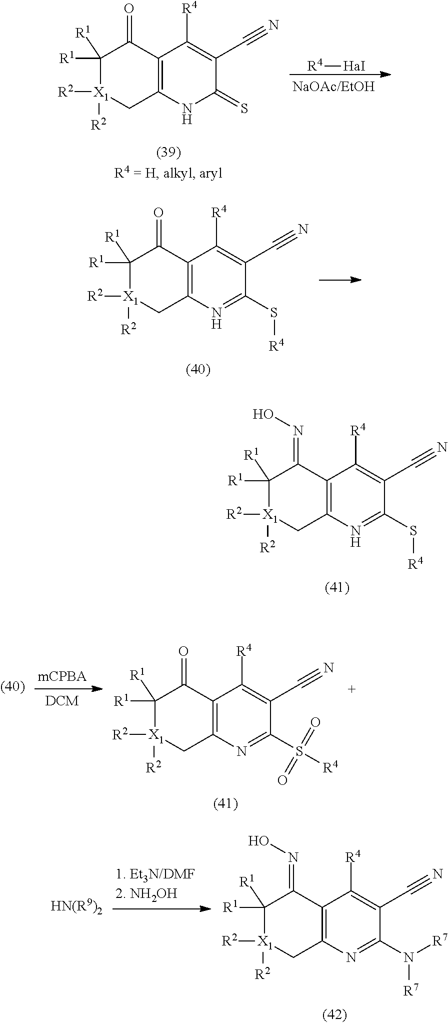

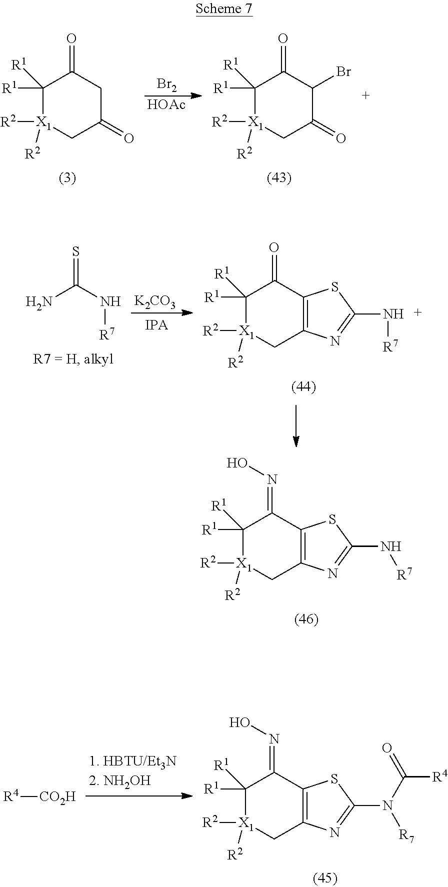

1. A compound having the following formula: 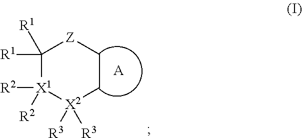
wherein:
A, together with the two carbons from the adjacent ring, is a fused heterocycle;
Z is selected from the group consisting of C═N—OR⁵, wherein R⁵ is H or a prodrug substituent;
X¹ is C or N;
X² is C, O, or a spacer, wherein R⁶ is —H or substituted or unsubstituted C₁ to C₆ alkyl group;
each R¹ is independently selected from the group consisting of H, C₁ to C₆ alkyl group, and C₃ to C₁₈ aryl group, or both R¹ groups may, together with the carbon they are attached to, form a C₃ to C₁₀ spirocycle group;
each R² is independently selected from the group consisting of H, C₁ to C₆ alkyl group, C₃ to C₁₈ aryl group, C₁-C₂₀ heteroaryl group, and C(O)N(R⁷)₂, or both R¹ groups may, together with the carbon they are attached to, form a C₃-C₁₀ spirocycle group, wherein R⁷ is independently selected from the group consisting of —H, substituted or unsubstituted C₁-C₆ alkyl group, substituted or unsubstituted alkynyl group, substituted or unsubstituted aryl group, substituted or unsubstituted heteroaryl group, substituted or unsubstituted saturated heterocyclyl group, substituted or unsubstituted partially-saturated heterocyclyl group, substituted or unsubstituted saturated C₃ to C₂₀ carbocyclyl group and substituted or unsubstituted partially-saturated C₃ to C₂₀ carbocycylyl group, or both R⁹ substituents, together with the N atom they are attached to, form a heterocycle group, which may additionally contains a further heteroatom(s) selected from the group consisting of N, O, and S;
each R³ is independently selected from the group consisting of H, C₁ to C₆ alkyl group, C₃ to C₁₈ aryl group, and C₁ to C₂₀ heteroaryl group; and
any two of R¹, R², R³, and/or substituents of A may be linked together with a linker to form a macrocycle group,
wherein one or more of the heteroatoms, such as, for example, nitrogen and sulfur, may optionally be oxidized to form N-oxides or sulfoxides and sulfones, respectively, and/or one or more nitrogen in one or more heterocycle may be quaternized,
or
a salt of the compound.

2. The compound of claim 1, wherein A is selected from the group consisting of: 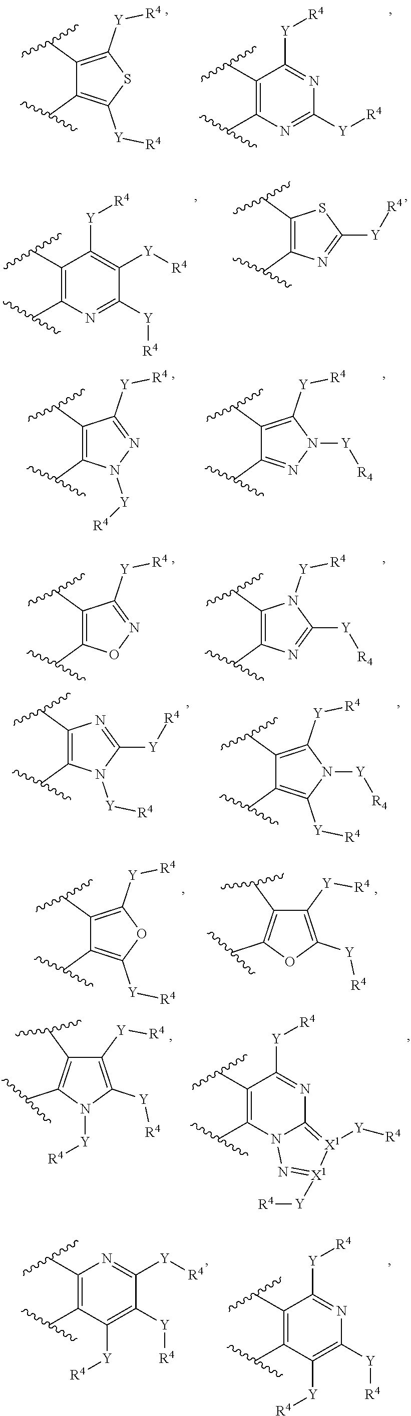 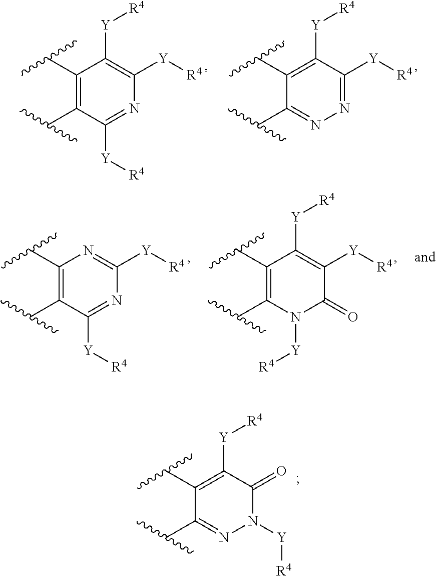
wherein each Y is independently selected from the group consisting of direct bond, O, S, S(═O), S(═O)₂, S(═O)(═NR⁸), C(═O), C(═O)O, 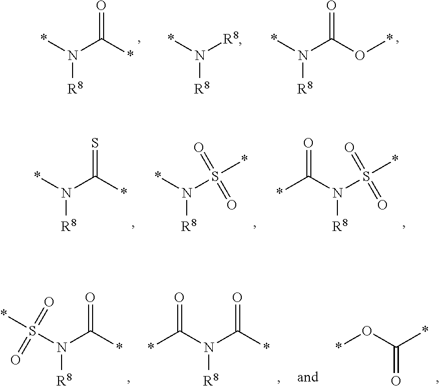
wherein each R⁸ is independently —H or substituted or unsubstituted C₁ to C₆ alkyl group, or C₃ to C₂₀ cycloalkyl group; and each R⁴ is independently selected from the group consisting of H, halide, CN, NO₂, substituted or unsubstituted C₁ to C₆ alkyl group, substituted or unsubstituted C₂ to C₄ alkenyl group, substituted or unsubstituted C₂ to C₄ alkynyl group, substituted or unsubstituted C₁ to C₆ alkoxyl group, substituted or unsubstituted C₁ to C₆ arylalkyl group, substituted or unsubstituted C₁ to C₆ hydroxyalkyl group, substituted or unsubstituted C₁ to C₆ alkoxyalkyl group, substituted or unsubstituted C₃ to C₂₀ cycloalkyl group, substituted or unsubstituted C₃ to C₁₈ aryl group, substituted or unsubstituted C₁ to C₆ cycloalkylalkyl group, substituted or unsubstituted C₁ to C₂₀ heteroaryl group, substituted or unsubstituted C₁ to C₂₀ heterocyclyl group, substituted or unsubstituted C₁ to C₆ heterocyclylalkyl group, substituted or unsubstituted C₁ to C₆ heteroarylalkylgroup, and —N(R⁷)₂.

3. The compound of claim 1, wherein the compound is selected from the group consisting of:

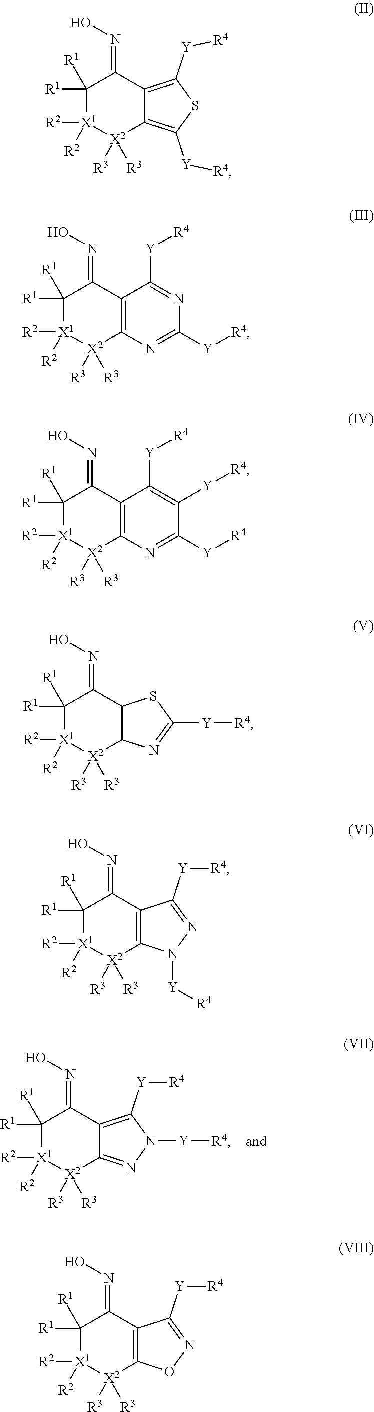

4-8. (canceled)

9. A method for removing senescence-associated macrophages (SAMs) in a mixed population of cells comprising SAMs, the method comprising selectively inducing SAM cell death in said mixed population of cells.

10. The method of claim 9, wherein SAM cell death is selectively induced by delivering to said mixed population of cells an agent which is selectively ingested by SAMs such that SAMs are removed from said mixed population.

11-18. (canceled)

19. A method of treating or preventing an age-related disease in a mammal, the method comprising administering an agent capable of reducing, eradicating, or reprogramming senescence-associated macrophages (SAMs) in a mammal, thereby treating said disease.

20. The method of claim 19, wherein said disease is cancer, age-related disease, tobacco-related disease, or skin wrinkles.

21. (canceled)

22. A method for identifying an agent that selectively removes SAMs from a mixed population of cells comprising SAMs, said method comprising (a) contacting a plurality of candidate agents with a mixed population of SAMs and senescent cells; and (b) determining selective removal of SAMs from said mixed population, thereby identifying said agent.

23. (canceled)

24. A method of reprogramming senescence-associated macrophages (SAMs) in a mixed population of cells, the method comprising selectively reversing or reducing the SAM phenotype in said mixed population of cells.

25. The method of claim 24, wherein SAM phenotype is selectively reversed or reduced by delivering to the mixed population of cells an agent capable of reducing SAM expression of p16, β-galactosidase, or both.

26. The method of claim 25, wherein the agent is selected from the group consisting of TA-4812, OT-82, a compound of any one of claims 1-7, interferon-alpha, interferon-beta, poly(I:C) dsRNA mimetic, activators of toll-like receptor 3 (TLR3), activators of toll-like receptor 4 (TLR4), ruxolitinib, momelotinib, JAK1/JAK2 inhibitors, AS1517499, STATE inhibitors, IL-4 neutralizing antibodies, IL-4 receptor neutralizing antibodies, IL-13 neutralizing antibodies, and IL-13 receptor neutralizing antibodies.

27-33. (canceled)

34. The compound of claim 1, wherein the spacer is —C(═O)—, —C(N═OR⁶)—, —C(═O)CH₂—, —C(N═OR⁶)CH₂—, —CH₂C(═O)—, or —CH₂C(N═OR⁶)—.

35. The compound of claim 1, wherein the salt is a pharmaceutically acceptable salt.

36. The method of claim 9, wherein said agent comprises a delivery vehicle and a drug which is active when ingested by a cell.


SEQUENCE LISTING (2 sequences)

SEQ ID NO: 1 - DNA, 21 bp, Artificial Sequence
  Akt siRNA
gggaggcaagaccuucauutt

SEQ ID NO: 2 - DNA, 21 bp, Artificial Sequence
  Akt siRNA
aaugaaggucuugccuccctg